(19) United States
(12) Patent Application Publication
(10) Pub. No.: US 2009/0098074 A1
(43) Pub. Date: Apr. 16, 2009

(54) Keratin-Binding Effector Molecules Containing Reactive Dyes

(75) Inventors: Heiko Barg, Speyer (DE); Burghard Liebmann, Bensheim (DE); Martin Volkert, Ludwigshafen (DE); Arne Ptock, Ludwigshafen (DE); Laszlo Somogyi, Limburgerhof (DE); Heike Reents, Speyer (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/095,153

(22) PCT Filed: Nov. 23, 2006
(86) PCT No.: PCT/EP06/68823

§ 371 Date: May 28, 2008

(30) Foreign Application Priority Data
Dec. 1, 2005 (EP) 05111581.4
Jun. 30, 2006 (EP) 06116402.6

(51) Int. Cl. A61K 8/64; C07K 14/435; A61Q 1/02; A61Q 5/00

(52) U.S. Cl. 424/63; 530/350; 8/428

(57) ABSTRACT

The invention relates to keratin-binding effector molecules comprising reactive dyes and keratin-binding polypeptides and to their use in hair-dyeing preparations. The present invention further relates to a method of dyeing hair.

The invention relates to keratin-binding effector molecules comprising reactive dyes and keratin-binding polypeptides, and to their use in hair-coloring preparations and to the hair-coloring preparations themselves. The present invention further relates to a method of coloring hair.

### PRIOR ART

Compositions for coloring hair (hair colorants) are divided into three classes depending on their color resistance: temporary hair colorants which last only 1-2 hair washes, semipermanent hair colorants which have to be renewed after 8-10 hair washes, and permanent hair colorants which cannot be washed out.

Temporary and semipermanent hair colorants are referred to as nonoxidative. Here, the dyes position themselves on the keratin in the hair or penetrate into the hair fiber. With permanent hair colorants, the most widespread hair colorants by far, the colors are formed directly on or in the hair from colorless precursors as a result of a chemical reaction in the presence of hydrogen peroxide, which serves as oxidizing agent. Here, the hair is completely colored through, the color cannot be washed out. These hair colorants are referred to as oxidative hair colorants.

Permanent hair coloring is very resistant to hair washing, the effect of light and other hair-treatment methods. It is the most widespread and has a market share of about 80% among the hair colorants. It only needs to be topped up about every month due to hair growth. With this coloring system, the dyes are formed directly on and in the hair, as a result of chemical reactions to which the uncolored intermediates or precursors are subjected. Oxidation reactions and coupling processes and condensations occur here which are brought about by hydrogen peroxide in the presence of ammonia or monoethanolamine. The use of hydrogen peroxide as oxidizing agent is therefore required because it not only initiates the dye formation, but at the same time also destroys the melanin pigments in the hair and in so doing brings about bleaching for which reason this coloring procedure is also referred to as a lightening coloration. The permanent hair colorants in principle also include the so-called self-oxidizing dyes, which are oxidized even by atmospheric oxygen.

Hair colorants are usually in the form of aqueous—preferably thickened—solutions or emulsions and, besides dyes, comprise, for example, fatty alcohols and/or other oil components, emulsifiers and surfactants, and if appropriate alcohols.

Oxidation hair colorants generally consist of two components, namely

(A) the dye carrier mass comprising the dyes and
(B) the oxidizing agent preparation.

These components are mixed shortly prior to application and then applied to the fibers to be dyed. Customary application forms of such permanent or oxidation hair colorants are cream hair colors and hair-coloring gels.

According to studies, 35 percent of women and ten percent of men in industrialized countries regularly color their hair. However, chemical hair colors have been in dispute for a prolonged period since health risks are not ruled out.

Thus, with each hair coloring, hair damage also takes place at the same time. The coloring pigments have to be incorporated through the predective squamous layer into the cortical layer of the hair.

As already explained above, hair colors comprise two components: an oxidizing agent and a coloring cream. The aromatic amines present in the hair colors have come in for criticism, particularly recently. During the coloring, these are thought to pass into the body via the head and are broken down in the liver. In the bladder, the degradation products are then sometimes converted to carcinogenic substances.

A recent study by Dartmouth Medical School in the “International Journal of Cancer” (A. Andres: Int J Cancer 2004; 109: 581-586) sees a possible connection between hair colorations and bladder cancer: women who regularly use permanent hair colorants had, on average, a 1.5 to 2.3-fold increased risk of suffering from urinary bladder cancer than women who did not color their hair.

Permanent hair colors, which have not been tested with regard to their acceptability, are to be banned in the future in the EU. At the moment, that would affect half of all hair colorants.

An object of this invention was to provide new types of dermocosmetic active ingredient compounds for application to skin or hair, specifically for coloring skin, hair or nails, and methods of producing same. Advantageously, active ingredient compounds were to be identified which have a keratin-binding property and are additionally suitable for producing cosmetic compositions and/or hair colorants. In addition, it was an object of the present invention to identify suitable compounds which can be coupled to a polypeptide with keratin-binding properties via a covalent bond. In particular, it was an object of the present invention to provide an innovative method for coloring hair, preferably a reversible coloring, in which the hair is damaged as little as possible.

Surprisingly, these objects could be achieved as described below by coupling chemical dyes to keratin-binding polypeptides.

### SUMMARY OF THE INVENTION

In a first embodiment, the invention relates to keratin-binding effector molecules comprising (a) at least one reactive dye (i) and (b) at least one keratin-binding polypeptide (ii).

In a preferred embodiment, the specified keratin-binding effector molecules comprise at least one keratin-binding polypeptide (ii) which has a binding affinity to human skin keratin, hair keratin or nail keratin.

Preferably, the keratin-binding polypeptide (ii) specified under (b) comprises
(a) at least one polypeptide with one of the sequences shown in SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170, or
(b) a polypeptide which is at least 40% identical to at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170 and is able to bind keratin.

Preferably, the keratin-binding polypeptide (ii) used according to the invention is encoded by a nucleic acid molecule comprising at least one nucleic acid molecule chosen from the group consisting of:
a) nucleic acid molecule which encodes a polypeptide comprising the sequence shown in SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170;
b) b) nucleic acid molecule which comprises at least one polynucleotide of the sequence shown in SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 167 or 169;
c) nucleic acid molecule which encodes a polypeptide according to the sequences SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170;
d) nucleic acid molecule with a nucleic acid sequence corresponding to at least one of the sequences according to SEQ ID No.: 1, 3, 5, 7, 9, 11, 13, 15, 17, 19, 21, 23, 25, 27, 20, 31, 33, 35, 37, 30, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 167 or 169 or a nucleic acid molecule derived therefrom by substitution, deletion or insertion which encodes a polypeptide which is at least 40% identical to at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170 and is able to bind to keratin;
e) nucleic acid molecule which encodes a polypeptide which is recognized by a monoclonal antibody directed toward a polypeptide which is encoded by the nucleic acid molecules as in (a) to (c);
f) nucleic acid molecule coding for a keratin-binding Protein which hybridizes under stringent conditions with a nucleic acid molecule as in (a) to (c);
g) nucleic acid molecule coding for a keratin-binding Protein which can be isolated from a DNA bank using a nucleic acid molecule as in (a) to (c) or part fragments thereof comprising at least 15 nt (nucleotides), preferably 20 nt, 30 nt, 50 nt, 100 nt, 200 nt or 500 nt, as probe under stringent hybridization conditions.
h) nucleic acid molecule which can be produced by backtranslation of one of the amino acid sequences shown in the sequences SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170.

In a further preferred embodiment of the present invention, the specified keratin-binding effector molecules comprise at least one reactive dye (ii), which has at least one reactive anchor, chosen from the group consisting of

(a) a group of the formula 1 activatable under alkaline conditions

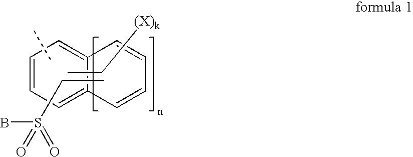

in which
[symbol] means the bond to the dye molecule (D),
X is an electron-withdrawing radical or is an alkyl, —O-alkyl, cyano, hydroxy, halogen radical or H,
k is 1, 2 or 3,
n is 0 or 1 and
B is a group —CH═CH₂ or a group —CH₂—CH₂-Q or —NH—CH₂—CH₂-Q or —NH—CH═CH₂, in which Q is a group which can be cleaved off under alkaline conditions, and
   (b) an acrylamido anchor of formula 2,

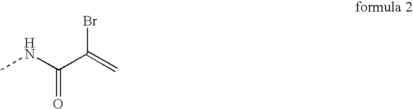

in which
[symbol] means the bond to the dye molecule (D), and
   (c) one of the compounds according to formulae 3, 4 or 5,

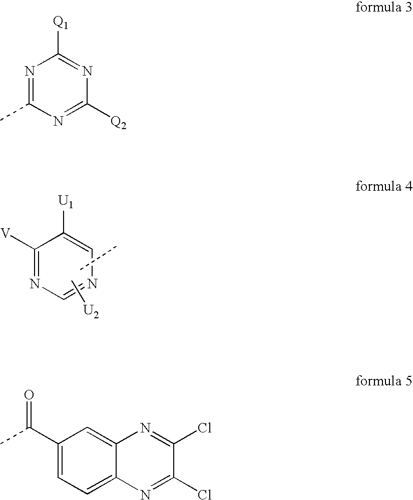

in which [symbol]
means the bond to the dye molecule (D),
V is fluorine or chlorine:
U₁, U₂, independently of one another, are fluoro, chloro or hydrogen; and
Q₁, Q₂, independently of one another, are chloro, fluoro, cyanamide, hydroxy, (C₁-C₆)-alkoxy, phenoxy, sulfophenoxy, mercapto, (C₁-C₆)-alkylmercapto, pyridino, carboxypyridino, carbamoylpyridino, or a group of the general formula (6) or (7),

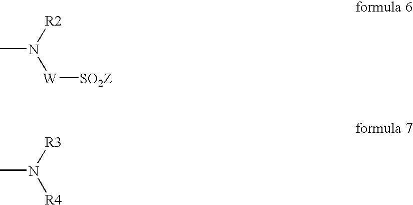

in which R² is hydrogen or (C₁-C₆)-alkyl, sulfo-(C₁-C₆)-alkyl, or phenyl, which is unsubstituted or substituted by (C₁-C₄)-alkyl, (C₁-C₄)-alkoxy, sulfo, halogen, carboxy, acetamido, ureido; R³ and R⁴, independently of one another, have one of the meanings of R², or are a group of the general formula (8),

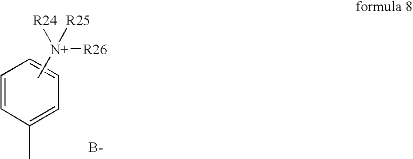

or form a cyclic ring system of the formula —(CH₂)ⱼ—, where j is 4 or 5, or alternatively —(CH₂)₂-E-(CH₂)₂—, where E is oxygen, sulfur, sulfo, —NR⁵— where R^{5′}=(C₁-C₆)-alkyl;
W is phenylene which is unsubstituted or substituted by one or two substituents, such as (C₁-C₄)-alkyl, (C₁-C₄)-alkoxy, carboxy, sulfo, chloro, bromo, or is (C₁-C₄)-alkylene-arylene or (C₂-C₆)-alkylene, which may be interrupted by oxygen, sulfur, sulfo, amino, carbonyl, carbonamido, or is phenylene-CONH-phenylene which is unsubstituted or substituted by (C₁-C₄)-alkyl, (C₁-C₄)-alkoxy, hydroxy, sulfo, carboxy, amido, ureido or halogen, or is naphthylene which is unsubstituted or substituted by one or two sulfo groups; and
Z is a group —CH═CH₂ or a group —CH₂—CH₂-Q or —NH—CH₂—CH₂-Q or —NH—CH═CH₂, in which Q is a group which can be cleaved off under alkaline conditions;
R²⁴, R²⁵ and R²⁶ are (C₁-C₄)-alkyl or (C₁-C₄)-hydroxyalkyl;
B— is the equivalent of an anion, such as hydrogensulfate, sulfate, fluoride, chloride, bromide, dihydrogenphosphate, hydrogenphosphate, phosphate, hydroxide or acetate.

In a moreover preferred embodiment of the present invention, the keratin-binding effector molecule comprises a reactive dye comprising a reactive anchor according to formula 1, where at least one of the radicals present therein is a group SO₃H.

Furthermore, the invention preferably relates to keratin-binding effector molecules which comprise a reactive dye which comprises a reactive anchor according to formula 1 and in which B in formula 1 is CH═CH₂, a group CH₂—CH₂—O—SO₃H or is CH₂—CH₂CL.

According to the invention, preference is given to the keratin-binding effector molecules described above in which the group of formula I which is activatable under alkaline conditions is bonded to the dye molecule (D) via a group —NH—, —N═N—, —NH—C(O)—, —NH—SO₂— or —N(R)—, where R is alkyl. In a particularly preferred embodiment, they are keratin-binding effector molecules which comprise dyes chosen from the group of dyes of the phthalocyanine series, anthraquinone dyes, azo dyes, formazane dyes, dioxazine dyes, actidine dyes, xanthene dyes, polymethine dyes, stilbene dyes, sulfur dyes, triarylmethane dyes, benzopyran dyes, dibenzanthrone dyes and the metal complexes of these dyes.

According to the invention, preference is also given to keratin-binding effector molecules comprising at least one reactive dye which comprises a reactive anchor chosen from the following radicals (1-1) to (1-43).

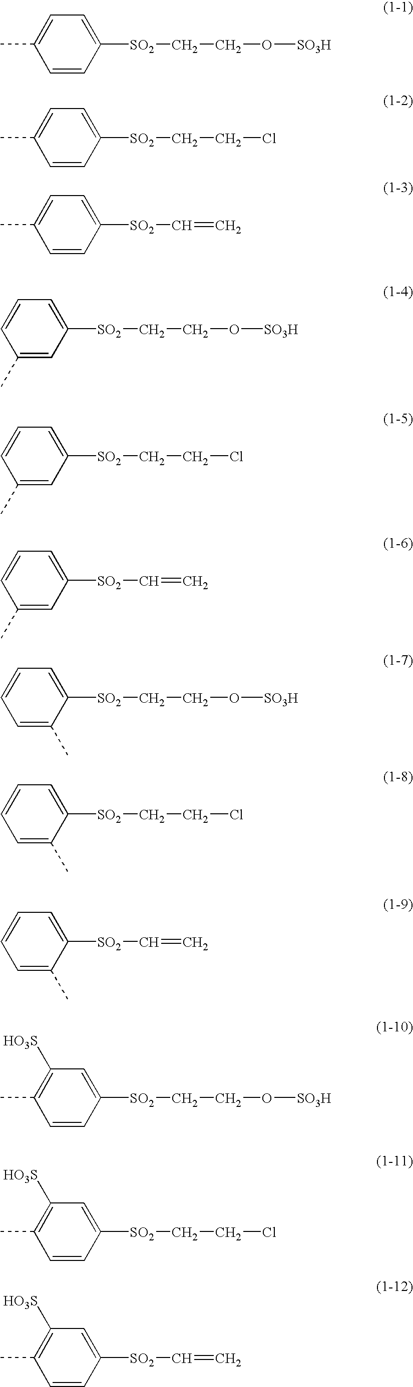

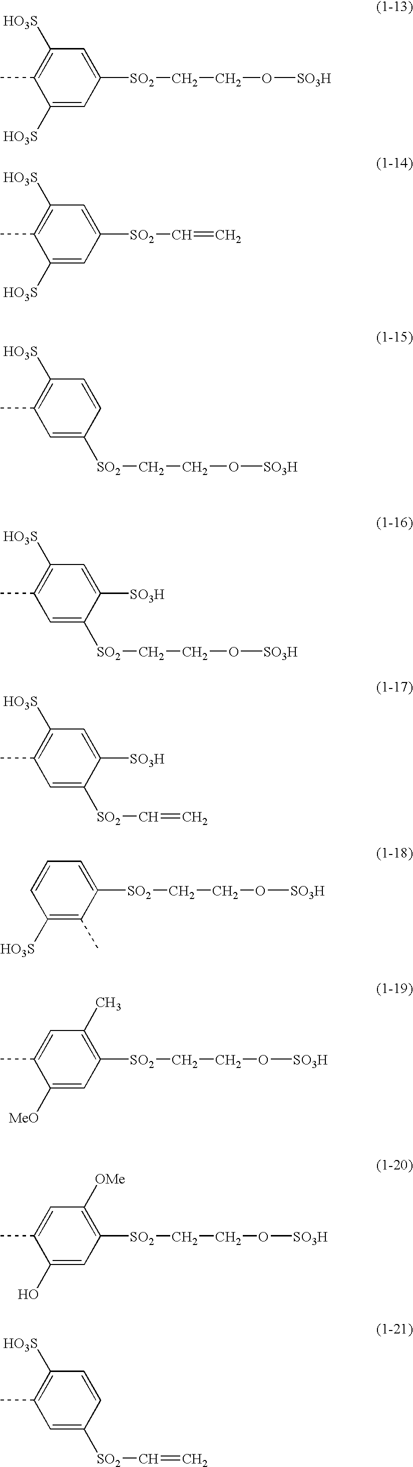

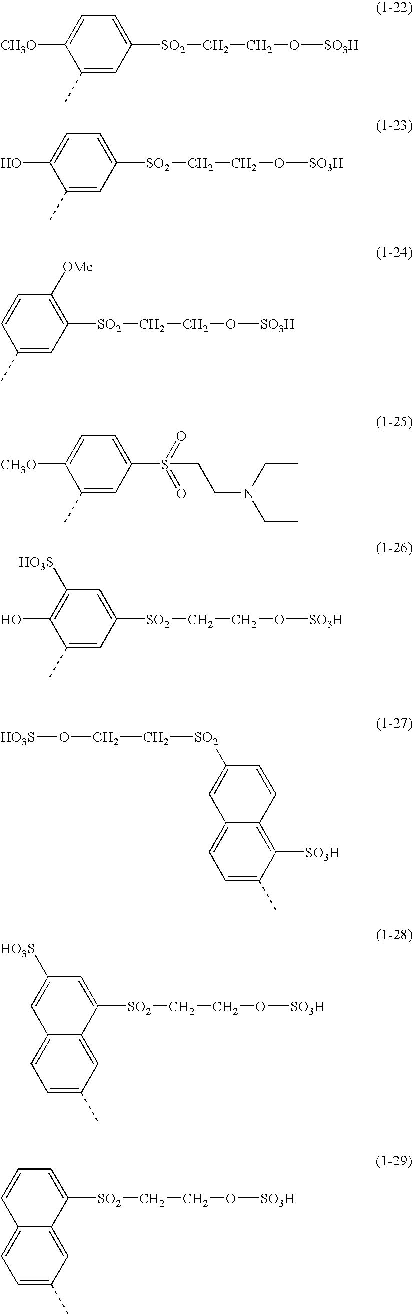

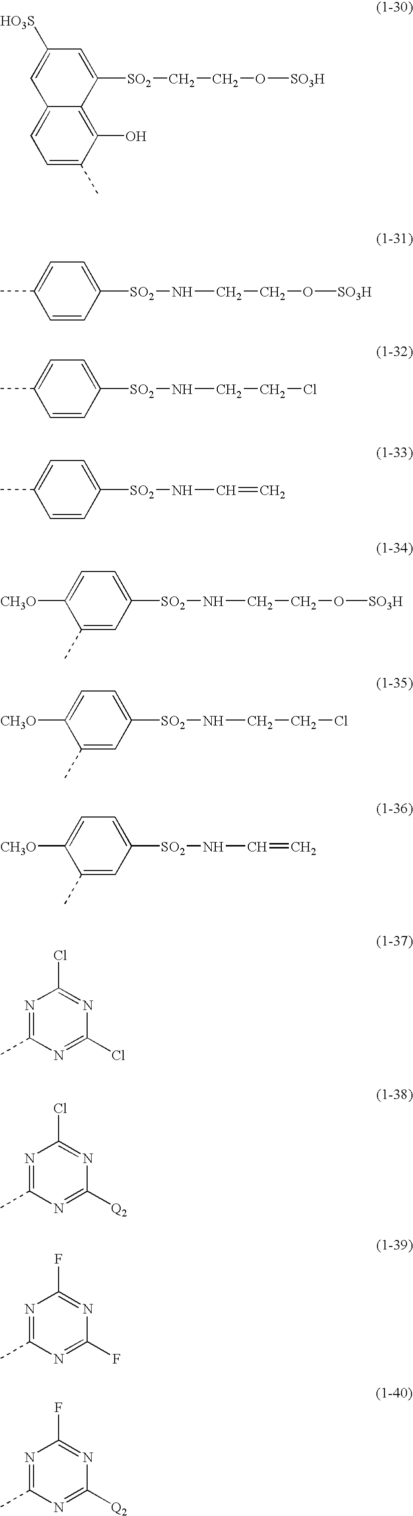

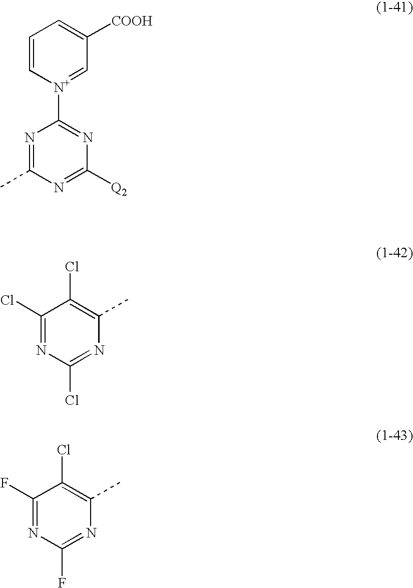

The present invention also preferably provides keratin-binding effector molecules in which the reactive dye (i) is coupled to the keratin-binding polypeptide (ii) indirectly via a linker molecule.

In the case of keratin-binding effector molecules preferred according to the invention, the dye or the reactive dye is coupled to the keratin-binding polypeptide via a linker molecule (iii) according to formula 9 or 10,

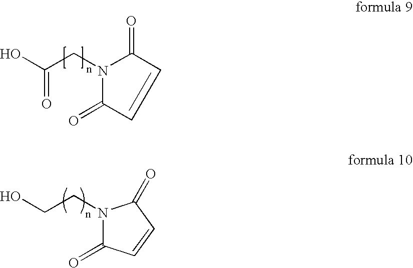

where “n” is an integer between 0 and 40.

The present invention further provides the use of the keratin-binding effector molecules described above in skin colorants, hair colorants or decorative cosmetics.

In addition, the present invention relates to skin colorants, nail colorants and/or hair colorants, preferably hair colorants, comprising at least one of the keratin-binding effector molecules according to the invention described above.

This invention further provides a method of coloring hair, skin or nails using keratin-binding effector molecules comprising (a) at least one keratin-binding polypeptide (ii) and (b) a dye or reactive dye (i).

In a particularly preferred embodiment of the method according to the invention, the above defined keratin-binding effector molecules according to the invention are used.

In a particularly preferred embodiment of the abovementioned method for coloring hair, skin or nails, it is a reversible method in which the keratin-binding effector molecule is displaced from the hair keratin, skin keratin or nail keratin in a displacement reaction by treatment with (i) a keratin-binding polypeptide or (ii) a keratin-containing solution, or the keratin-binding effector molecule is removed from skin, hair or nails by a solution with a high detergent content (e.g. SDS).

### DEFINITIONS

In connection with the description, “amino function-bearing effector molecule”, “amino functions” means amino groups which allow said amino function-bearing molecules to covalently link to other molecules via an amide bond. For the purposes of the present invention, “amino functions” are also those which can be converted chemically into amino functions. Here, the effector molecules according to the invention have at least one amino function. However, it is also possible to use effector molecules with two, three or more amino functions and/or secondary amino groups.

For the purposes of the present invention, “antibodies” are Proteins which humans and jaw-bearing vertebrates produce to predect against antigens (infection pathogens or biological material alien to the body). They are a central constituent of the immune system of higher eukaryotes and are secreted by a class of white blood corpuscles, the B cells. They occur in blood and in the extracellular liquid of tissue.

“Decorative cosmetics” means cosmetic auxiliaries which are not primarily used for the care, but for beautifying or improving the appearance of skin, hair and/or fingernails and toenails. Auxiliaries of this type are appropriately known to the person skilled in the art and comprise, for example, kohl pencils, mascara, eye shadows, tinted day creams, powders, concealing sticks, blusher, lipsticks, lipliner sticks, make-up, nail varnish, glamour gel etc. For the purposes of the present invention, they are particularly compositions suitable for coloring skin, nails and/or hair.

“Hair colorants” are compositions or preparations (i) coloring hair. For the purposes of the present invention, “hair colorants” are divided into direct colors and true hair colors. In the case of direct colors, one or more hair washes suffice to remove the color again (tints). The dye is only positioned on the surface of the hair substance, no chemical reaction takes place. For the hair itself, the process is without risk. The true (permanent) hair colors use oxidation coloring (see also “Prior art”). Hair colorants comprise, in a cosmetically compatible medium, suitable auxiliaries and additives which are familiar to the person skilled in the art and can be found in cosmetics handbooks, for example, Schrader, Grundlagen und Rezepturen der Kosmetika [Fundamentals and formulations of cosmetics], Hüthig Verlag, Heidelberg, 1989, ISBN 3-7785-1491-1, or Umbach, Kosmetik: Entwicklung, Herstellung und Anwendung kosmetischer Mittel [Cosmetics: development, manufacture and use of cosmetic compositions], 2nd extended edition, 1995, Georg Thieme Verlag, ISBN 3 13 712602 9.

For the purposes of the present invention, “effector molecule” means molecules which have a certain foreseeable effect, preferably a color-changing and/or care effect or a cosmetically decorative effect on skin, hair or nails. Preferably, the effector molecules are dyes as depicted, for example, in Tables 3, 5 and 6

For the purposes of the present invention, “keratin” means intermediate filaments constructed from rope-like Protein complexes. Intermediate filaments are constructed from many Proteins or the same type (monomers) which position themselves in parallel to give a tube-like structure. Intermediate filaments are bound to give relatively large bundles (tonofibrils). Intermediate filaments form the cytoskeleton of the cell with the micredubules and actin filaments. A distinction is made between five types of intermediate filaments: acidic and basic keratins, desmins, neurofilaments and lamins. Of specific preference for the purposes of the present invention are the acidic and basic keratins occurring in the epithelia (single or multiple cell layers which cover all external body surfaces of multicellular animal organisms). “Keratin” or “keratins” (also: horny substance, scleroProtein) means a Protein which is responsible for the stability and shape of the cells. This Protein is a constituent of mammal skin, hair and nails. The strength of keratin is increased through fiber formation: the individual amino acid chains form a right-handed alpha-helix, and every three of these helixes form a left-hand superhelix (=predofibrils). Eleven predofibrils combine to give a microfibril these combine in turn to give bundles and form macrofibrils which, for example, surround the cells of the hair.

“Keratin-binding polypeptide” means a polypeptide or a Protein which has the property of binding to keratin. Keratin-binding polypeptides are thus also intermediate filament-associated Proteins. These keratin-binding polypeptides have a binding affinity toward the keratin or the macrostructures consisting of keratin such as predofibrils, microfibrils or macrofibrils. In addition, keratin-binding polypeptides are understood as meaning those polypeptides which have a binding affinity to skin, hair and/or fingernails or toenails of mammals.

“Keratin-binding polypeptides” are also polypeptides which, within a mammal organism, have a biological function associated with the binding of keratin, keratin fibers, skin or hair. Keratin-binding polypeptides likewise means the binding motifs or Protein domains necessary for the actual binding to the keratin, the keratin fibers, skin, hair or nails. The binding of the keratin-binding polypeptide (ii) to keratin can be tested under the conditions described in Example 8, 9 and 10. Keratin-binding polypeptides are those polypeptides which, in the abovementioned quantitative keratin-binding tests, have about 10%, 20%, 30%, 40% or 50%, preferably 50%, 60%, 70%, 80% or 90%, particularly preferably 100%, 125%, 150%, very particularly preferably 200%, 300% or 400%, most preferably 500%, 600%, 700% or 1000% or more of the keratin-binding capacity of desmoplakin (SEQ ID No.: 2), preferably of the keratin-binding domain B of desmoplakin (SEQ ID No.: 4).

“Coupling” in connection with the binding of a linker molecule to an effector molecule or keratin-binding Protein means a covalent linking of said molecules.

“Cosmetically compatible medium” is to be understood in the wide sense and means substances suitable for the production of cosmetic or dermocosmetic preparations, and mixtures thereof. They are preferably Protein compatible media.

Upon contact with human and/or animal skin tissue or hair, “cosmetically compatible substances” lead to no irritations or damage and have no incompatibilities with other substances. In addition, these substances have a slight allergenic potential and are approved by state registration authorities for use in cosmetic preparations. These substances are familiar to the person skilled in the art and can be found, for example, in cosmetics handbooks, for example Schrader, Grundlagen und Rezepturen der Kosmetika [Fundamentals and formulations of cosmetics], Hüthig Verlag, Heidelberg, 1989, ISBN 3-7785-1491-1.

“Nucleic acid” or “nucleic acid molecule” means deoxyribonucleotides, ribonucleotides or polymers or hybrids thereof in single-strand or double-strand form, in sense or antisense orientation. The term nucleic acid or nucleic acid molecule can be used to describe a gene, DNA, cDNA, mRNA, oligonucleotide or polynucleotide.

“Nucleic acid sequence” means a successive and linked together sequence of deoxyribonucleotides or ribonucleotides of a nucleic acid molecule according to the definition given above, as can be ascertained using available DNA/RNA sequencing techniques, and depicted or shown in a list of abbreviations, letters or words which represent nucleotides.

For the purposes of the present invention, “polypeptide” means a macromolecule constructed from amino acid molecules in which the amino acids are linked together linearly via peptide bonds. A polypeptide can be made up of a few amino acids (about 10 to 100), but also includes Proteins. Proteins are generally constructed from at least 100 amino acids, but can also comprise several thousand amino acids. Preferably, polypeptides comprise at least 20, 30, 40 or 50, particularly preferably at least 60, 70, 80 or 90, very particularly preferably at least 100, 125, 150, 175 or 200, most preferably at least more than 200 amino acids, it being possible for the upper limit to be several thousand amino acids.

“Homology” or “identity” between two nucleic acid sequences is understood as meaning the identity of the nucleic acid sequence over the entire sequence length in question, which is calculated by comparison with the help of the program algorithm GAP (Wisconsin Package Version 10.0, University of Wisconsin, Genetics Computer Group (GCG), Madison, USA; Altschul et al. (1997) Nucleic Acids Res. 25:3389ff) with the following parameter settings:

| | |
|---|---|
| Gap Weight: 50 | Length Weight: 3 |
| Average Match: 10 | Average Mismatch: 0 |

By way of example, a sequence which has a homology of at least 80% based on nucleic acid with the sequence SEQ ID NO: 1 is understood as meaning a sequence which has a homology of at least 80% when compared with the sequence SEQ ID NO: 1 according to the shove program algorithm with the above set of parameters.

Homology between two polypeptides is understood as meaning the identity of the amino acid sequence over the entire sequence length in question, which is calculated by comparison with the help of the program algorithm GAP (Wisconsin Package Version 10.0, University of Wisconsin, Genetics Computer Group (GCG), Madison, USA) with the following parameter settings:

| | |
|---|---|
| Gap Weight: 8 | Length Weight: 2 |
| Average Match: 2.912 | Average Mismatch: −2.003 |

By way of example, a sequence which has a homology of at least 80% based on polypeptide with the sequence SEQ ID NO: 2 is understood as meaning a sequence which has a homology or at least 80% when compared with the sequence SEQ ID NO: 2 as cording to the above program algorithm with the above set of parameters.

“Hybridization conditions” is to be understood in the wide sense and means stringent or less stringent hybridization conditions depending on the application. Such hybridization conditions are described, inter alia, in Sambrook J, Fritsch E F, Maniatis T et al., in Molecular Cloning (A Laboratory Manual), 2nd edition, Cold Spring Harbor Laboratory Press, 1989, pages 9.31-9.57) or in Current Protocols in Molecular Biology, John Wiley & Sons, N.Y. (1989), 6.3.1-6.3.6. The person skilled in the art would choose hybridization conditions which would allow him to differentiate specific hybridizations from unspecific hybridizations. For example, the conditions during the washing step can be chosen from conditions with low stringency (with approximately 2×SSC at 50° C.) and those with high stringency (with approximately 0.2×SSC at 50° C., preferably at 65° C.) (20×SSC: 0.3M sodium citrate, 3M NaCl, pH 7.0). Moreover, the temperature during the washing step can be increased from low stringency conditions at room temperature, approximately 22° C., to higher stringency conditions, approximately 65° C. Both parameters, salt concentration and temperature, can be varied at the same time or individually, keeping the other parameter in each case constant. During the hybridization, it is also possible to use denaturing agents such as, for example, formamide or SDS. In the presence of 50% formamide, the hybridization is preferably carried out at 42° C. Some illustrative conditions for hybridization and washing step are given below:
1. Hybridization conditions can be chosen, for example, from the following conditions;
   a) 4×SSC at 65° C.,
   b) 6×SSC at 45° C.,
   c) 6×SSC, 100 μg/ml of denatured, fragmented fish sperm DNA at 68° C.,
   d) 6×SSC, 0.5% SDS, 100 μg/ml of denatured, salmon sperm DNA at 68° C.,
   e) 6×SSC, 0.5% SDS, 100 μg/ml of denatured, fragmented salmon sperm DNA,
      50% formamide at 42° C.,
   f) 50% formamide, 4×SSC at 42° C., or
   g) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C., or
   h) 2× or 4×SSC at 50° C. (low stringency condition),
   i) 30 to 40% formamide, 2× or 4×SSC at 42° C. (low stringency condition).

500 mN of sodium phosphate buffer pH 7.2, 7% SDS (g/V). 1 mM EDTA, 10 μg/ml single stranded DNA, 0.5% BSA (g/V) (Church and Gilbert, Genomic sequencing. Proc. Natl. Acad. Sci. U.S.A. 81: 1991. 1984)
2. Washing steps can be chosen, for example, from the following conditions:
   a) 0.015 M NaCl/0.0015 M sodium citrate/0.1% SDS at 50° C.
   b) 0.1×SSC at 65° C.
   c) 0.1×SSC, 0.5% SDS at 68° C.
   d) 0.1×SSC, 0.5% SDS, 50% formamide at 42° C.
   e) 0.2×SSC, 0.1% SDS at 42° C.
   f) 2×SSC at 65° C. (low stringency condition).

In one embodiment, the stringent hybridization conditions are chosen as follows:

A hybridization buffer is chosen which comprises formamide, NaCl and PEG 6000. The presence of formamide in the hybridization buffer destabilizes double stranded nucleic acid molecules, as a result of which the hybridization temperature can be reduced to 42° C. without lowering the stringency. The use of salt in the hybridization buffer increases the renaturation rate of a duplex, or the hybridization efficiency. Although PEG increases the viscosity of the solution, which has a negative effect on renaturation rates, as a result of the presence of the polymer in the solution, the concentration of the probe in the remaining medium is increased, which increases the hybridization rate. The composition of the buffer is as follows:

TABLE 1
Hybridization buffer
Hybridization buffer

250 mM sodium phosphate buffer pH 7.2
1 mM EDTA
7% SDS (g/v)
250 mM NaCl
10 μg/ml ssDNA
5% polyethylene glycol (PEG) 6000
40% formamide

The hybridizations are carried out overnight at 42° C. The filters are washed the next morning 3× with 2×SSC+0.1% SDS for about 10 min in each case.

In connection with the description “hydroxy function-bearing effector molecule”, “hydroxy function” means free OH groups or hydroxyl groups which enable these OH group-bearing molecules to covalently link to other molecules via an esterification reaction. For the purposes of the present invention, “hydroxy functions” are also those which can be converted chemically into OH functions (for example, derivatives such as methoxy, ethoxy). Here, the effector molecules according to the invention have at least one hydroxyl group. However, it is also possible to use effector molecules with two, three or more hydroxy functions.

“Coupling functionalities” are functional groups of a linker molecule which can enter into a covalent bond with functional groups of the effector molecule or of the keratin-binding Protein. Nonlimiting examples which may be mentioned are: hydroxy groups, carboxyl groups, thio groups and amino groups. “Coupling functionalities” or “Coupling functionality” and “anchor groups” or “anchor group” are used synonymously.

For the purposes of the present invention, “reactive dye” means dyes comprising at least one reactive anchor which can be coupled to a keratin-binding polypeptide via a covalent bond.

For the purposes of the present invention, “reactive anchor” means chemically functional groups or radicals by means of which a covalent bond between a carbon atom or phosphorus atom of the reactive dye molecule and an oxygen, nitrogen or sulfur atom of a hydroxy, amino or thiol group of another molecule is realized.

A “group Q which can be cleaved off under alkaline conditions” is understood as meaning radicals which are cleaved off under alkaline conditions, i.e. at a pH of 7 or above, preferably 7.5 or above, with elimination and formation of a vinylsulfone group. Examples of such groups are halogen, e.g. chloro, bromo or iodo, also —O—SO₃H, —S—SO₃H, dialkylamino, quaternary ammonium radicals, such as tri-C₁-C₄-alkylammonium, benzyldi-C₁-C₄-alkylammonium or N-bonded pyridinium, and radicals of the formulae R³S(O)₂—, R⁴S(O)₂—O—, R⁵C(O)—O—. Here, R³, R⁴ and R⁵, independently of one another, are alkyl, haloalkyl or optionally substituted phenyl, where R⁵ can also be hydrogen. Preferably, Q is a group —O—(CO)CH₃ and in particular is —O—SO₃H.

For the purposes of the present invention, “reversible coloring” means a change in the color of skin keratin, hair keratin or nail keratin achieved using the method according to the invention which can be reversed.

For the purposes of the present invention, “backtranslation” means the translation of a Protein sequence into a nucleic acid sequence coding for this Protein. The backtranslation is thus a process of decoding an amino acid sequence into the nucleic acid sequence corresponding to it. Customary methods are based on codon usage tables of individual types, which are produced by computer-aided sequence comparisons. Using the codon usage tables it is possible to determine the codons used most frequently for a certain amino acid for a specific type. Protein backtranslation can be carried out using computer algorithms which are known to the person skilled in the art and specifically generated for this purpose (Andrés Moreira and Alejandro Maass. TIP: Protein backtranslation aided by genetic algorithms. Bioinformatics, Volume 20, Number 13 Pp. 2148-2149 (2004); G Pesole, M Attimonelli, and S Liuni. A backtranslation method based on codon usage strategy. Nucleic Acids Res. 1988 Mar. 11; 16(5 Pt A): 1715 1728.).

### DETAILED DESCRIPTION OF THE INVENTION

The present invention provides keratin-binding effector molecules comprising (a) at least one reactive dye (i) and (b) at least one keratin-binding polypeptide (ii).

In a preferred embodiment, the specified keratin-binding effector molecules comprise at least one keratin-binding polypeptide (ii) which has a binding affinity to human skin keratin, hair keratin or nail keratin. Keratin-binding polypeptide domains suitable according to the invention are present, for example, in the polypeptide sequences of desmoplakins, plakophilins, plakoglobins, plectins, periplakins, envoplakins, trichohyalins, epiplakins or hair follicle Proteins.

Preferably, the keratin-binding polypeptide (ii) specified under (b) comprises
(c) at least one polypeptide with one of the sequences shown in SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170, or
(d) a polypeptide which is at least 40% identical to at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 109, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170 and is able to bind keratin.

Preferably, the specified keratin-binding effector molecules comprise at least one keratin-binding polypeptide (ii), which is encoded by a nucleic acid molecule, comprising at least one nucleic acid molecule chosen from the group consisting of:
c) nucleic acid molecule which encodes a polypeptide, comprising the sequence shown in SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170;
d) b) nucleic acid molecule which comprises at least one polynucleotide of the sequence shown in SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 167 or 169;
c) nucleic acid molecule which encodes a polypeptide according to the sequences SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170;
j) nucleic acid molecule with a nucleic acid sequence corresponding to at least one of the sequences according to SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 167 or 169 or a nucleic acid molecule derived therefrom by substitution, deletion or insertion which encodes a polypeptide which is at least 40% identical to at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 104, 166, 168 or 170 and is able to bind to keratin;
k) nucleic acid molecule which encodes a polypeptide which is recognized by a monoclonal antibody directed toward a polypeptide which is encoded by the nucleic acid molecules as in to (a) to (c);
l) nucleic acid molecule coding for a keratin-binding Protein which hybridizes under stringent conditions with a nucleic acid molecule as in (a) to (c);
m) nucleic acid molecule coding for a keratin-binding Protein which can be isolated from a DNA bank using a nucleic acid molecule as in (a) to (c) or part fragments thereof comprising at least 15 nt, preferably 20 nt, 30 nt, 50 nt, 100 nt, 200 nt or 500 nt as probe under stringent hybridization conditions, and
n) nucleic acid molecule which can be produced by backtranslation of one of the amino acid sequences shown in the sequences SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170.

In a preferred embodiment of the present invention, the said keratin-binding effector molecules comprise desmoplakins or part fragments thereof according to the sequences SEQ ID No.: 2, 42, 44, 46, 48, 146, 150, 153, 156, 157, 158, 160, 162, 164 or 166, and/or plakophillins or part sequences thereof according to the sequences SEQ ID No.: 18, 20, 26, 28, 32, 34, 36, 168, 170 and/or plakoglobins or part sequences thereof according to the sequences with the SEQ ID No.: 50, 52, 54, 56, 58, 60, 62, 64, 66, 68, 70, and/or the periplakin according to the sequence with the SEQ ID No.: 86, and/or envoplakins or part sequences thereof according to the sequences with the SEQ ID No.: 90, 92, 94, 96, 98, 102, 104, 105 and/or the sequences according to SEQ ID No.: 138 and 140 as keratin-binding polypeptides. Preferred keratin-binding domains are the desmoplakin polypeptides depicted in the sequences SEQ ID NOs: 4, 6, 8, 10, 12, 14, 146, 150, 153, 156, 157, 158, 160, 162, 164, 166, 168 or 170, and their functional equivalents. In a very particularly preferred embodiment of the present invention, the keratin-binding polypeptides depicted in the sequences SEQ ID No.: 156, 157, 158, 160, 162, 164, 168 and/or 170 are used in the method according to the invention. In an embodiment of the present invention which is preferred most of all, the keratin-binding Protein shown in the sequence SEQ ID No.: 168 is used. It goes without saying here that this Protein can be used either with or without the histidine anchors present in the SEQ ID No.: 168. Thus, the histidine anchor (or a purification/detection system to be used analogously) can also be present C-terminally. In practical use of said keratin-binding Proteins (e.g. in cosmetic preparations), a histidine anchor (or a purification/detection system to be used analogously) is not necessary. The use of said Proteins without additional amino acid sequences is thus preferred.

Likewise included according to the invention are “functional equivalents” of the specifically disclosed keratin-binding polypeptides (ii) and the use of these in the method according to the invention.

For the purposes of the present invention, “functional equivalents” or analogs of the specifically disclosed keratin-binding polypeptides (ii) are polypeptides different therefrom which also have the desired biological activity, such as, for example, keratin binding. Thus, for example, “functional equivalents” of keratin-binding polypeptides are understood as meaning those polypeptides which, under otherwise comparable conditions, in the quantitative keratin-binding tests described in the examples, have about 10%, 20%, 30%, 40% or 50%, preferably 60%, 70%, 80% or 90%, particularly preferably 100%, 125%, 150%, very particularly preferably 200%, 300% or 400%, most preferably 500%, 600%, 700% or 1000% or more of the keratin-binding capacity of the polypeptides shown under the SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170, preferably in SEQ ID No.: 2, 4, 6, 8, 10, 12, 14, 40, 42, 44, 46, 48, 146, 150, 153, 156, 157, 158, 160, 162, 164, 166, 168 or 170, particularly preferably 166 and 168, most preferably 168.

According to the invention, “functional equivalents” are understood in particular as meaning also muteins which have an amino acid other than that specifically given in at least one sequence position of the abovementioned amino acid sequences but nevertheless have one of the abovementioned biological activities. “Functional equivalents” thus include the muteins obtainable by a mutation where the specified changes can arise in any sequence position provided they lead to a mutein with the profile of properties according to the invention.

For the purposes of the present invention, “mutation” means the change in the nucleic acid sequence of a gene variant in a plasmid or in the genome of an organism. Mutations can arise, for example, as a result of errors during replication, or be caused by mutagens. The rate of spontaneous mutations in the cell genome of organisms is very low although a large number of biological, chemical or physical mutagens is known to the knowledgeable person skilled in the art.

Mutations include substitutions, insertions, deletions of one or more nucleic acid radicals. Substitutions are understood as meaning the replacement of individual nucleic acid bases, a distinction being made here between transitions (substitution of a purine base for a purine base or a pyrimidine base for a pyrimidine base) and transversions (substitution of a purine base for a pyrimidine base (or vice versa)).

Additions or insertions are understood as meaning the incorporation of additional nucleic acid radicals into the DNA, possibly resulting in shifts in the reading frame. With reading frame shifts of this typo, a distinction is made between “in frame” insertions/additions and “out of frame” insertions. In the case of “in frame” insertions/additions, the reading frame is retained and a polypeptide enlarged by the number of amino acids encoded by the inserted nucleic acids arises. In the case of “out of frame” insertions/additions, the original reading frame is lost and the formation of a complete and functioning polypeptide is no longer possible.

Deletions describe the loss of one or more base pairs, which likewise lead to “in frame” or “out of frame” shifts in the reading frame and the consequences associated therewith regard to the formation of an intact Protein.

The mutagenic agents (mutagens) which can be used for producing random or targeted mutations and the applicable methods and techniques are known to the person skilled in the art. Such methods and mutagens are described, for example, in A. M. van Harten [(1998), “Mutation breeding: theory and practical applications”, Cambridge University Press, Cambridge, UK], E Friedberg, G Walker, W Siede [(1995), “DNA Repair and Mutagenesis”, Blackwell Publishing], or K. Sankaranarayanan, J. M. Gentile, L. R. Ferguson [(2000) “Protocols in Mutagenesis”, Elsevier Health Sciences].

For introducing targeted mutations, customary molecular biological methods and processes such as, for example, the in vitro MutagenEse Kit, LA PCR in vitro Mutagenesis Kit (Takara Shuzo, Kyoto) or the QuikChange® Kit from Stratagene or PCR mutageneses using suitable primers can be used.

As already discussed above, there is a large number of chemical, physical and biological mutagens.

The mutagens listed below are given by way of example, but are non-limiting.

Chemical mutagens can be subdivided according to their mechanism of action. Thus, there are base analogs (e.g. 5-bromouracil, 2-aminopurine), mono- and bifunctional alkylating agents (e.g. monofunctional ones such as ethylmethylsulfonate, dimethyl sulfate, or bifunctional ones such as dichloroethyl sulfite, mitomycin, nitrosoguanidines-dialkylnitrosamines, N-nitrosoguanidine derivatives) or intercalating substances (e.g. acridines, ethidium bromide).

Thus, for example, in the keratin-binding effector molecules according to the invention, it is also possible for those polypeptides to be present which are obtained as a result of a mutation of a polypeptide according to the invention e.g. according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 and/or 170.

Examples of suitable amino acid substitutions are given in the table below:

TABLE 4
Suitable amino acid substitutions

| Original radical | Examples of substitution |
|---|---|
| Ala | Ser |
| Arg | Lys |
| Asn | Gln; His |
| Asp | Glu |
| Cys | Ser or Ala |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

It is known that in SEQ ID NO: 2, the serine naturally present at position 2849 can, for example, be replaced by glycine in order to avoid a phosphorylation at this position (Fontao L, Favre B, Riou S, Geerts D, Jaunin F, Saurat J H, Green K J, Sonnenberg A, Rorradori L., Interaction of the bullous pemphigoid antigen 1 (BP230) and desmoplakin with intermediate filaments is mediated by distinct sequences within their COOH terminus, Mol Biol Cell. 2003 May; 14(5):1978-92. Epub 2003 Jan. 26).

In the above sense, “functional equivalents” are also “precursors” of the described polypeptides, and “functional derivatives” and “salts” of the polypeptides.

Here, “precursors” are natural or synthetic precursors of the polypeptides with or without desired biological activity.

The expression “salts” is understood as meaning either salts of carboxyl groups or acid addition salts of amino groups of the Protein molecules according to the invention. Salts of carboxyl groups can be prepared in a manner known per se and include inorganic salts, such as, for example, sodium, calcium, ammonium, iron and zinc salts, and also salts with organic bases, such as, for example, amines such as triethylamine, arginine, lysine, piperidine and the like. Acid addition salts, such as, for example, salts with mineral acids, such as hydrochloric acid or sulfuric acid, and salts with organic acids, such as acetic acid and oxalic acid, are likewise provided by the invention.

“Functional equivalents” naturally also include polypeptides which are accessible from other organisms, and naturally occurring variants (alleles). For example, through sequence comparisons, areas of homologous sequence regions or preserved regions can be determined. Using these sequences, DNA databases (e.g. genomic or cDNA databases) can be inspected for equivalent enzymes using bioinformatic comparison programs. Suitable computer programs and databases which are accessible to the public are sufficiently known to the person skilled in the art.

These alignments of known Protein sequences can be carried out, for example, using a computer program such as Vector NTI 8 (version from 25 Sep. 2002) from InforMax Inc.

Furthermore, “functional equivalents” are fusion Proteins which have one of the abovementioned polypeptide sequences or functional equivalents derived therefrom and have at least one further heterologous sequence functionally different therefrom in functional N- or C-terminal linkage (i.e. without mutual essential functional impairment of the fusion Protein parts). Nonlimiting examples of such heterologous sequences are, for example, signal peptides or enzymes.

“Functional equivalents” included according to the invention are homologs to the specifically disclosed Proteins. These have at least 40%, 45% or 50%, preferably at least 55%, 60%, 65% or 70%, particularly preferably at least 75%, 80%, 85%, 90%, 91%, 92%, 93% or 94%, very particularly preferably at least 95% or 96% homology to one of the specifically disclosed amino acid sequences, calculated using the computer programs and computer algorithms disclosed in the definitions.

In the case of a possible Protein glycosylation, “functional equivalents” according to the invention include Proteins of the type referred to above in deglycosylated or glycosylated form, and also modified forms obtainable by changing the glycosylation pattern.

In the case of a possible Protein phosphorylation, “functional equivalents” according to the invention include Proteins of the type referred to above in dephosphorylated or phosphorylated form, and also modified forms obtainable by changing the phosphorylation pattern.

Homologs of the polypeptides according to the invention can be identified by screening combinatorial banks of mutants, such as, for example, shortening mutants. For example, a bank of Protein variants can be produced by combinatorial mutagenesis at nucleic acid level, such as, for example, by enzymatic ligation of a mixture of synthetic oligonucleotides. There is a large number of methods which can be used for producing banks of potential homologs from a degenerated oligonucleotide sequence. The chemical synthesis of a degenerated gene sequence can be carried out in an automatic DNA synthesis machine, and the synthetic gene can then be ligated into a suitable expression vector. The use of a degenerated set of genes makes it possible to provide all of the sequences in one mixture which encode the desired set of potential Protein sequences. Methods for synthesizing degenerated oligonucleotides are known to the person skilled in the art (e.g. Narang, S. A. (1983) Tetrahedron 39:3; Itakura et al. (1984) Annu. Rev. Biochem. 53:323; Itakura et al., (1984) Science 198.1056; Ike et al. (1983) Nucleic Acids Res. 11:477).

In the prior art, a number of techniques for the screening of gene products of combinatorial banks which have been produced by point mutations or shortening, and for the screening of cDNA banks for gene products with a selected property are known. The most often used techniques for screening large gene banks which are subjected to analysis with a high throughput include the cloning of the gene bank in replicable expression vectors, transforming the suitable cells with the resulting vector bank and expressing the combinatorial genes under conditions under which the detection of the desired activity facilitates the isolation of the vector which encodes the gene whose product has been detected. Recursive ensemble mutagenesis (REM), a technique which increases the frequency of functional mutants in the banks can be used in combination with the screening tests in order to identify homologs (Arkin and Yourvan (1992) PNAS 89:7811-7815; Dolgrave et al. (1993) Protein Engineering 6(3):327-331). The inspection of physically available cDNA or genomic DNA libraries of other organisms using the nucleic acid sequences described under SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 167 and/or 169, particularly preferably 165 and 167, most preferably 167, or parts thereof as probe is a method known to the person skilled in the art for identifying homologs in other ways. Here, the probes derived from the nucleic acid sequence according to SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 165, 167 and/or 169, particularly preferably 165 and 167, most preferably 167, have a length of at least 20 bp, preferably at least 50 bp, particularly preferably at least 100 bp, very particularly preferably at least 200 bp, most preferably at least 400 bp. The probe can also be one or more kilobases long, e.g. 1 Kb, 1.5 Kb or 3 Kb. For inspecting the libraries it may also be possible to use one of the sequences of complementary DNA strand described under SEQ ID No.: 1, 3, 6, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 165, 167 and/or 169, particularly preferably 165 and 167, most preferably 167, or a fragment thereof with a length between 20 bp and several kilobases. The hybridization conditions to be used are described above.

In the method according to the invention, it is also possible to use those DNA molecules which, under standard conditions, hybridize with the nucleic acid molecules described by SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 165, 167 and/or 169, particularly preferably 165 and 167, most preferably 167, and encoding keratin-binding polypeptides, which hybridize to these complementary nucleic acid molecules or parts of the above, and as complete sequences encode polypeptides which have the same properties as the polypeptides described under SEQ ID No.: 2, 4, 6, 8, 10, 12, 14, 16, 18, 20, 22, 24, 26, 28, 30, 32, 34, 36, 38, 40, 42, 44, 46, 48, 50, 52, 64, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170.

In a particularly advantageous embodiment of the invention, the keratin-binding effector molecules comprise, as keratin-binding polypeptides (ii), those polypeptides which comprise at least one of the polypeptide sequences as shown in SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 160, 108 or 170, with the proviso that the keratin binding of said polypeptides is at least 10%, 20%, 30%, 40% or 50%, preferably 60%, 70%, 80% or 90%, particularly preferably 100%, of the value which the corresponding polypeptide sequences as shown in SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170 have, measured in the test according to Example 9 or 10.

Preference is given to using keratin-binding polypeptides (ii) which have a highly specific affinity for the desired organism. Accordingly, for uses for human hair coloring, preference is given to using those keratin-binding polypeptides (ii) which have a particularly high affinity to human hair keratin.

However, it is also possible to use more than one keratin-binding polypeptide (ii) coupled to the effector molecule (i) according to the invention, for example a keratin-binding polypeptide (ii) which has a high binding affinity to human skin keratin can be combined with an effector molecule in combination with another keratin-binding polypeptide (ii) which has a high affinity to human hair keratin. It is also possible to use chimeric polypeptides which comprise two or more copies of the same (and also different) keratin-binding polypeptides (ii) or keratin-binding domains thereof. For example, it was thus possible to achieve particularly effective keratin binding.

Suitable keratin-binding polypeptides (ii) are known. For example, desmoplakins and plectins comprise keratin-binding domains (Fontao L, Favre B, Riou S, Geerts D, Jaunin F, Saurat J H, Green K J, Sonnenberg A, Borradori L., Interaction of the bullous pemphigoid antigen 1 (BP230) and desmoplakin with intermediate filaments is mediated by distinct sequences within their COOH terminus, Mol Biol Cell. 2003 May; 14(5):1978-92. Epub 2003 Jan. 26; Hopkinson S B, Jones J C., The N-terminus of the transmembrane Protein BP180 interacts with the N-terminal domain of BP230, thereby mediating keratin cytoskeleton anchorage to the cell surface at the site of the hemidesmosome, Mol Biol Cell. 2000 January; 11(1): 277-86).

Preferably, the keratin-binding effector molecules according to the invention are used in hair cosmetics, preferably hair coloring. They permit a high concentration and long actin time of care, predecting or color-changing effector molecules.

In a particularly preferred embodiment of the present invention, keratin-binding polypeptides are used which has a binding affinity to human skin keratin, hair keratin or nail keratin.

In a further preferred embodiment of the present invention, the specified keratin-binding effector molecules comprise at least one reactive dye (ii) which has at least one reactive anchor, chosen from the group consisting of

(a) a group of the formula 1 activatable under alkaline conditions,

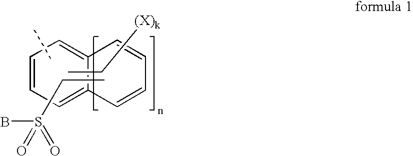

in which
[symbol] means the bond to the dye molecule (D),
X is an electron-withdrawing radical or is an alkyl, —O-alkyl, cyano, hydroxy, halogen radical or H,
k is 1, 2 or 3,
n is 0 or 1, and
B is a group —CH═CH₂ or a group —CH₂—CH₂-Q or —NH—CH₂—CH₂-Q or —NH—CH═CH₂, in which 0 is a group which can be cleaved off under alkaline conditions, and
   (b) an acrylamido anchor of formula 2,

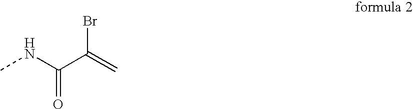

in which
[symbol] means the bond to the dye molecule (D), and
   (c) one of the compounds according to formulae 3, 4 or 5,

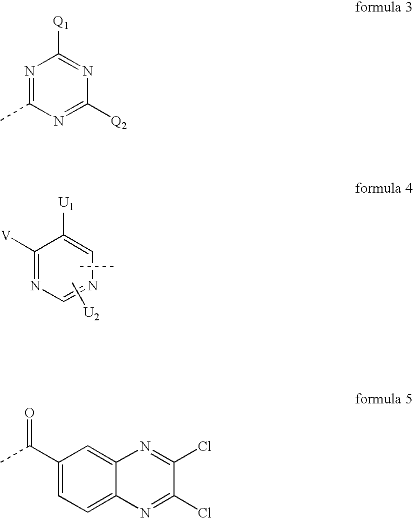

in which [symbol]
means the bond to the dye molecule (D),
V is fluorine or chlorine;
U₁, U₂, independently of one another, are fluoro, chloro or hydrogen; and
Q₁, Q₂, independently of one another, are chloro, fluoro, cyanamido, hydroxy, (C₁-C₆)-alkoxy, phenoxy, sulfophenoxy, mercapto, (C₁-C₆)-alkylmercapto, pyridino, carboxypyridino, carbamoylpyridino, or a group of the general formula (6) or (7),

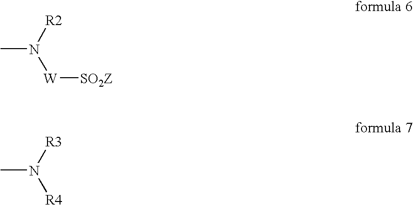

in which R² is hydrogen or (C₁-C₆)-alkyl, sulfo-(C₁-C₆)-alkyl, or phenyl, which is unsubstituted or substituted by (C₁-C₄)-alkyl, (C₁-C₄)-alkoxy, sulfo, halogen, carboxy, acetamido, ureido; R³ and R⁴, independently of one another, have one of the meanings of R², or are a group of the general formula (8),

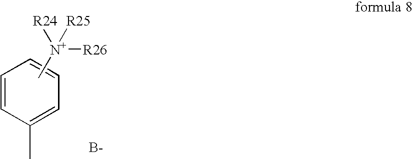

or form a cyclic ring system of the formula —(CH₂)ⱼ—, where j is 4 or 5, or alternatively —(CH₂)₂-E-(CH₂)₂—, where E is oxygen, sulfur, sulfo, —NR⁵— where R^{5′}=(C₁-C₆)-alkyl;
W is phenylene which is unsubstituted or substituted by 1 or 2 substituents, such as (C₁-C₄)-alkyl, (C₁-C₄)-alkoxy, carboxy, sulfo, chloro, bromo, or is (C₁-C₄)-alkylene-arylene or (C₂-C₆)-alkylene, which may be interrupted by oxygen, sulfur, sulfo, amino, carbonyl, carbonamido, or is phenylene-CONH-phenylene which is unsubstituted or substituted by (C₁-C₄)-alkyl, (C₁-C₄)-alkoxy, hydroxy, sulfo, carboxy, amido, ureido or halogen, or is naphthylene which is unsubstituted or substituted by one or two sulfo groups; and
Z is a group CH═CH₂ or a group CH₂—CH₂-Q or —NH—CH₂—CH₂-Q or —NH—CH═CH₂, in which Q is a group which can be cleaved off under alkaline conditions;
R²⁴, R²⁵ and R²⁶ are (C₁-C₄)-alkyl or (C₁-C₄)-hydroxyalkyl;
B— is the equivalent of an anion, such as hydrogensulfate, sulfate, fluoride, chloride, bromide, dihydrogenphosphate, hydrogenphosphate, phosphate, hydroxide or acetate.

Accordingly, the present invention relates, in a preferred embodiment, to a keratin-binding effector molecule which comprises, as effector molecule, at least one reactive dye (ii) which has at least one group of the formula I which is activatable under alkaline conditions

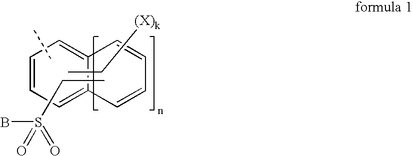

in which
[symbol] is the bond to the radical of the dye molecule (D);
X is an electron-withdrawing radical,
k is 1, 2 or 3,
n is 0 or 1, and
B is a group CH═CH₉ or a group CH₂—CH₂-Q or —NH—CH₂—CH₂-Q or —NH—CH═CH₂, in which Q is a group which can be cleaved off under alkaline conditions.

Here and below, the expression alkyl is usually a linear or branched hydrocarbon radical having 1 to 6 and preferably 1 to 4 carbon atoms (C₁-C₆- or C₁-C₄-alkyl), such as methyl, ethyl, propyl, isopropyl and the like. Haloalkyl is alkyl, as defined above, in which the hydrogen atoms are partly or completely replaced by halogen atoms, in particular by fluorine atoms, as in trifluoromethyl, trichloromethyl, pentafluoroethyl, and the like. Alkoxy is an alkyl radical bonded via an oxygen atom, as defined above. Optionally substituted phenyl means that the phenyl radical can have one or more, e.g. 1, 2, 3 or 4, substituents, which are chosen, for example, from halogen, alkyl, alkoxy, nitro, cyano, COOH, SO₃H and the like. Halogen is in particular fluorine, chlorine or bromine.

Electron-withdrawing radicals X are those which exert an -M and/or -I effect on the aromatic radical to which they are bonded. These include, for example, fluorine or chlorine, CN, NO₂, and groups of the formulae —C(O)—R¹ and S(O)₂R², in which R¹ and R², independently of one another, are OH, alkyl, haloalkyl, alkoxy or optionally substituted phenyl. If formula 1 has a plurality of groups (k>1), then the groups X may be identical or different.

Preferably, at least one of the groups X is a hydroxysulfonyl group (SO₃H).

The variable k is preferably 1 or 2, i.e. formula 1 has one or two electron-withdrawing radicals X. Preferably “n” in formula 1 is 0, i.e. formula 1 is derived from benzene. If “n” is 1, formula 1 is derived from naphthalene. In these cases, the group SO₂—B can be located on the same benzene ring as group X.

Furthermore, the invention relates to keratin-binding effector molecules which comprise a reactive dye which comprises a reactive anchor according to formula 1 and in which B in formula 1 is CH═CH₂, a group CH₂—CH₂—O—SO₃H or is CH₂—CH₂—Cl.

According to the invention, preference is given to the above described keratin-binding effector molecules in which the group of formula 1 which can be activated under alkaline conditions is bonded to the dye molecule via a group —NH—, —N═N—, —NH—C(O)—, —NH—SO₂— or —N(R)—, where R is alkyl (as defined above). Expediently, keratin-binding effector molecules according to the invention have 1, 2 or 3, preferably 1 or 2, of the abovementioned reactive dyes. The group according to formula 1 which can be activated under alkaline conditions can (but does not have to) be a constituent of the dye chromophore.

As a rule, the reactive dye has one or more, e.g. 1 to 10, in particular 2 to 8, functional groups per dye molecule which impart water solubility to the dye. These are generally anionic or acidic functional groups which, in an aqueous medium at a weakly acidic or alkaline pH, generally at a pH above 4, dissociate to form anionic groups. Examples of such groups are hydroxysulfonyl groups (—SO₃H), carboxyl groups (COOH) and hydroxysulfonyloxy groups (—O—SO₃H), and the anions of these groups. These anionic/acidic groups can be bonded to a group according to formula 1 and/or to other parts of the dye molecule. If these groups are present in the dye as anionic groups, it goes without saying that the dye also comprises the counterions required for neutralization. Suitable counterions are, in particular, alkali metal ions, specifically sodium, potassium and lithium ions, and ammonium ions, e.g. ammonium ions derived from mono-, di- or triethanolamine.

A particularly preferred subject matter of the invention is keratin-binding effector molecules which comprise dyes (“D”) chosen from the group of dyes of the phthalocyanine series, anthraquinone dyes, azo dyes, formazane dyes, dioxazine dyes, actidine dyes, xanthene dyes, polymethine dyes, stilbene dyes, sulfur dyes, triarylmethane dyes, benzopyran dyes, dibenzanthrone dyes and the metal complexes of these dyes.

Such dyes D are partly known from the prior art and can be found, for example, in the patent applications WO 04/18381, EP A 356 931, EP-A 559 617, EP-A 201 868, DE-A 195 23 245, DE-A 197 31 166, EP 745 640, EP-A 889 098, EP-A 1 097 971, EP-A 880 098, or are prepared analogously to known preparation processes for structurally similar dyes, as are known, for example, from the documents EP 602 562, EP-A 597 411, EP-A 592 105 or DE 43 196 74.

To prepare the reactive dyes comprising reactive anchor according to formula 1, an amino compound of formula 1b will generally be reacted with a dye precursor which has a nucleophilically displaceable group in a manner known per se. Examples of nucleophilically displaceable groups are halogen, in particular chlorine or bromine, which is bonded to an aromatic (as in halotriazine radicals) or in the form of a halosulfonyl group or a halocarbonyl group. Processes for this are known from the prior art cited here and can be used analogously for the preparation of the dyes D. Alternatively, the amino compound 1b can also firstly be diazotized and then coupled to a corresponding dye precursor. The reaction product obtained in the reaction of the amino compound 1b or its diazonium salt with the dye precursor may already be the dye D or for its part represent a precursor of the dye D which is further processed to give the dye D analogously to known processes.

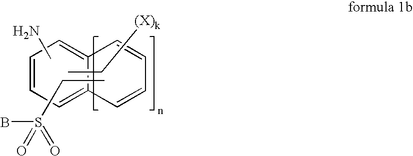

The radicals shown in formula 1b correspond to the definitions given in formula 1. It should be taken into consideration here that dyes can comprise inorganic salts and extenders as a result of the preparation. The content of such constituents, also referred to below as noncolored constituents, will generally not be more than 60% by weight and is often in the range from 10 to 50% by weight, based on the total weight of colored and noncolored constituents in the dye.

The chromophore systems of the dyes (D) can have different structures irrespective of whether they are reactive or nonreactive dyes. The chromophores (1 to 7) shown below are in each case compound specified by way of representation for a certain structural class. This list is not to be understood as restrictive. The person skilled in the art is of course aware that in principle all dyes belonging to these classes of substances can be bonded to a keratin-binding polypeptide directly as reactive dye or via a linker molecule. The dyes can be metal-free dyes, but also metal complexes, preferably transition metal complexes, in particular complexes of the transition metals of groups VI to X of the periodic table of the elements and, of these, in particular of Cu, Cr, Fe, Ni, Co and Mn. The molar ratio of transition metal to dye molecule in these metal complexes is usually in the range from 2:1 to 1:2. As a rule, complexation of the metal ions in these dyes takes place via depredonated hydroxyl groups, via amino groups, imino groups, nitrogen atoms which are incorporated into an aromatic π-electron system, or via azo groups.

### 1. Azo Dyes

Mono-, bis-, tris-, tetra- and polyazo dyes depending on how many azo bridges (—N═N—) the dyes comprise. The bond to the reactive anchor in 1a, 1b and 1c is of course also an azo bridge.

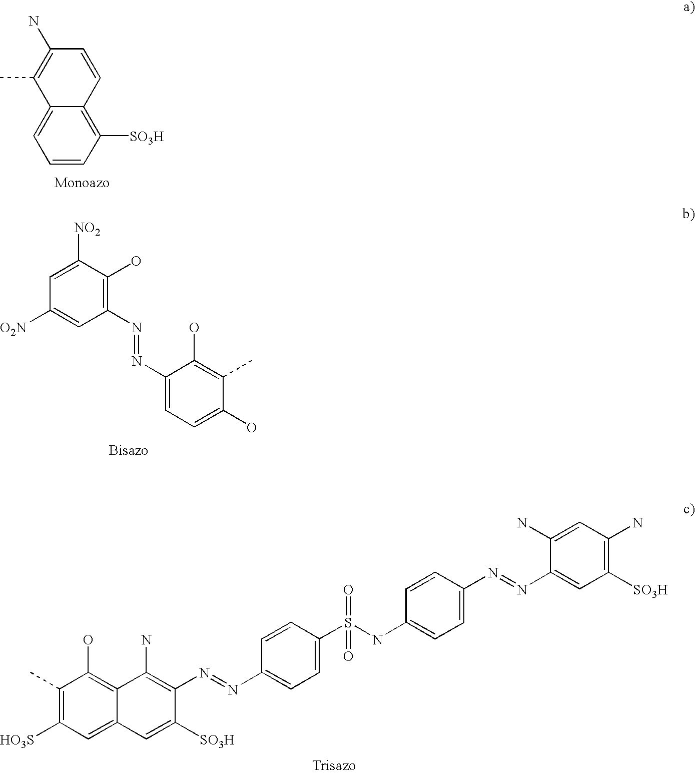

### 2. Anthraquinone Dyes

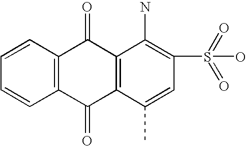

### 3. Triphendioxazine Dyes

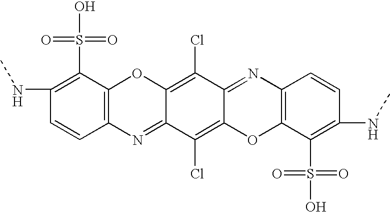

### 4. Phthalocyanine Dyes

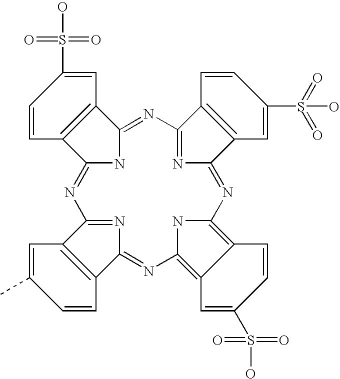

### 5. Benzopyran Dyes

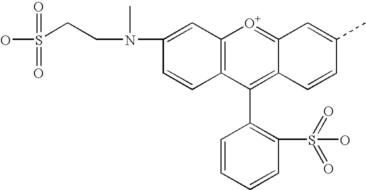

### 6. Dibenzanthrone Dyes

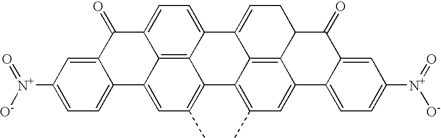

### 7. Formazane Dyes

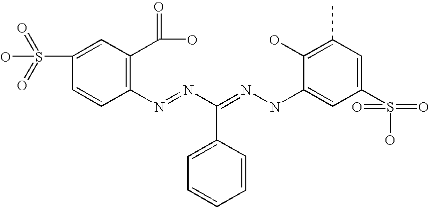

According to the invention, preference is also given to keratin-binding effector molecules which comprise a reactive dye (i) which comprises a reactive anchor which is chosen from the following radicals (1-1) to (1-43),

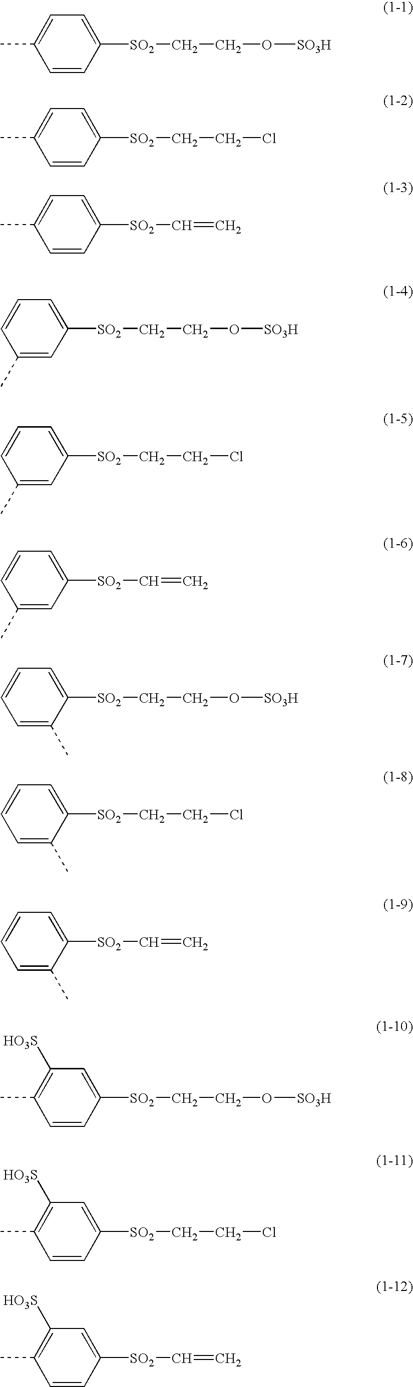

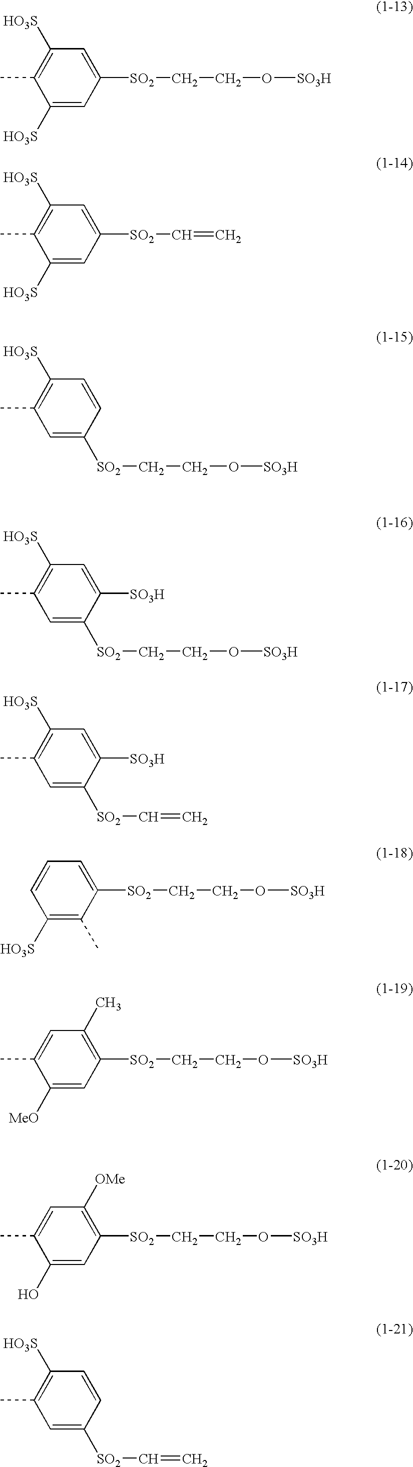

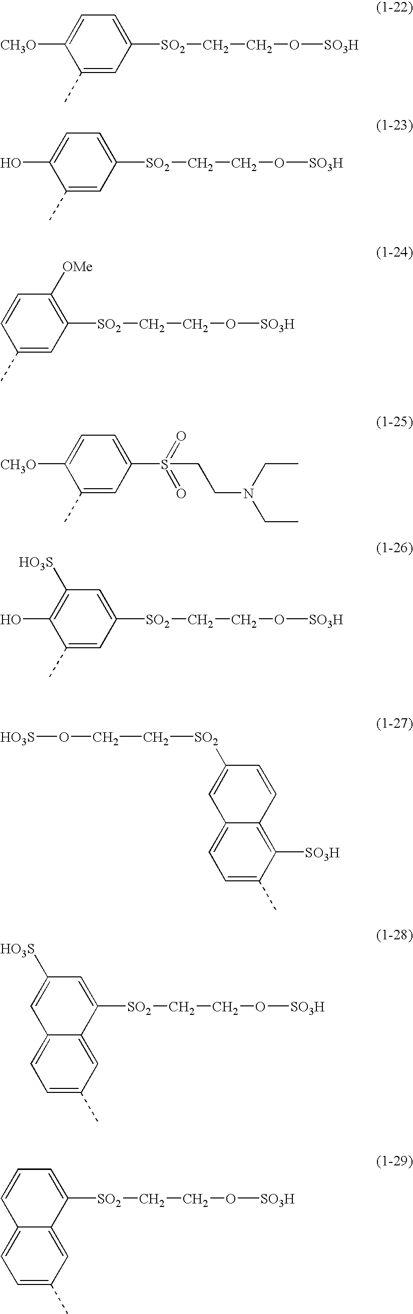

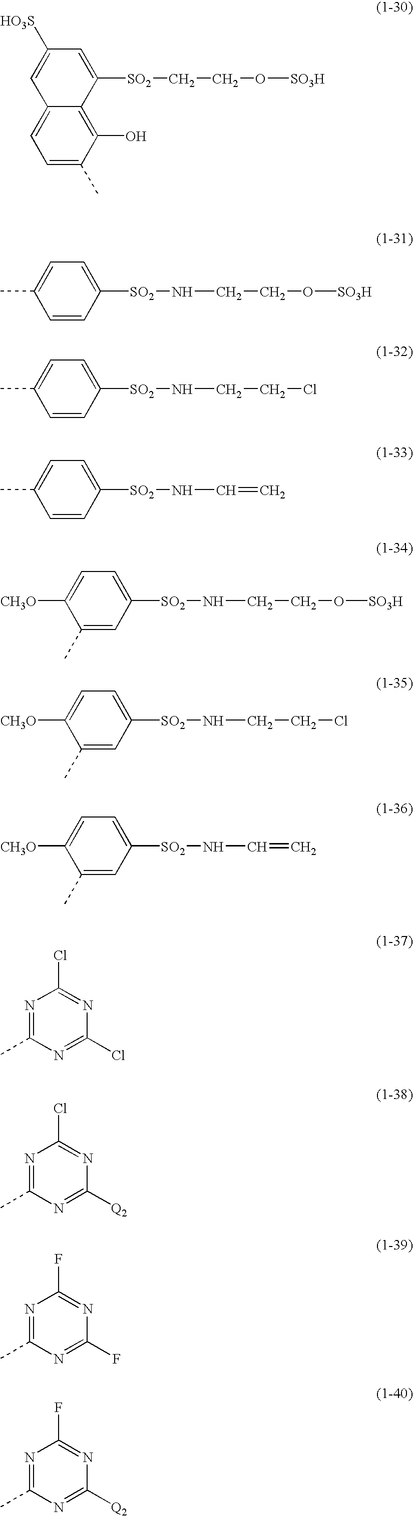

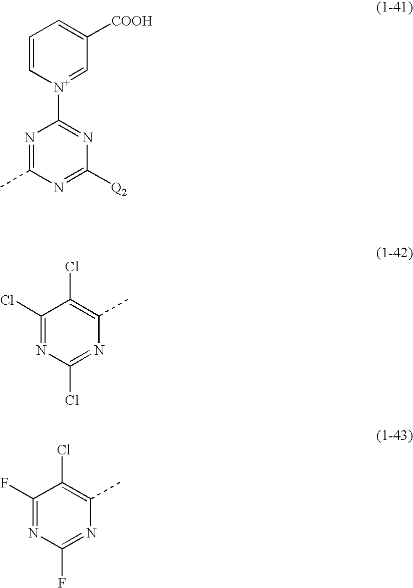

Particularly preferred reactive dyes comprise at least one reactive anchor according to formula 1, where this radical has at least one of the groups shown in the formulae (1-1) to (1-12) and (1-16), (1-17), preferably (1-1), (1-3), (1-4), (1-6), (1-7), (1-9), (1-10), (1-12), (1-16), (1-17), most preferably (1-1), (1-4), (1-7), (1-10), (1-17).

In the reaction of dyes (D) with a keratin-binding polypeptide, virtually physiological conditions should be maintained since the polypeptide or Protein otherwise untolds, resulting in a precipitation. This essentially means that no pure organic solvents can be used, and a pH close to neutral should be chosen. Furthermore, it is important that the reaction temperatures for the coupling do not exceed 45° C.

Accordingly, in theory it is possible to use all reactive dyes which can react under mild conditions with the SH groups of the keratin-binding polypeptides. Mild conditions are understood as meaning temperature, pH and concentration ranges in which the keratin-binding polypeptide retains or does not irreversibly lose its physical and chemical properties.

Suitable reactive dyes comprise at least one reactive anchor, but can also comprise two or more reactive anchors, where these may correspond to different reactive anchor types.

Dyes comprising vinylsulfone anchors (see formula 1) are usually prepared as sulfuric ester compounds. Activation (elimination to the active vinylsulfone form) of the sulfuric ester compounds takes place in situ during the coloring for textile and leather applications, i.e. the dyes do not need to be preeliminated to the vinylsulfone prior to use. The dyes can, if appropriate, also be isolated as vinylsulfone and only be reacted with the substrate at a later time.

As a result of the fact that with the keratin-binding polypeptides according to the invention or the keratin-binding domain (also referred to below as KBD), a pH close to neutral is chosen, and furthermore the reaction temperature for the coupling with dyes should not exceed 45° C., the dyes must be preeliminated to the active vinylsulfone form and only then be coupled with, for example, a KBD. The reaction with, for example, a KBD can take place directly after the elimination in the same liquor (without isolation of the dye in the vinylsulfone form), or the dye can also be isolated.

For the activation, a pH range from 5-14, in particular 7-12 and a temperature range from 0-80° C., in particular 15-50° C., preferably 25-45° C., is required. After the activation, a pH range from pH 5-9, in particular 7-8, and a temperature range from 20-50° C., in particular 25-45° C., is established, with the dye reacting rapidly (often within minutes) with the substrate (in this case with a keratin-binding polypeptide/KBD). Activation (elimination) of the sulfuric ester to the vinylsulfone proceeds according to the scheme shown in FIG. 1.

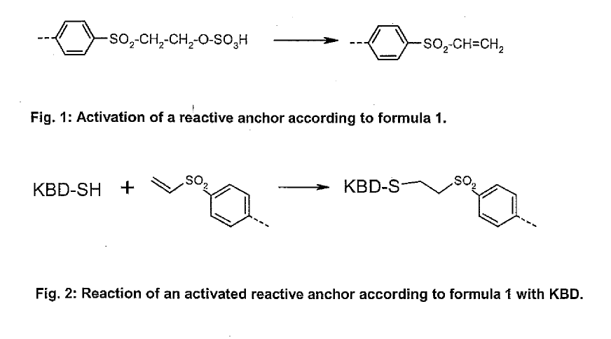

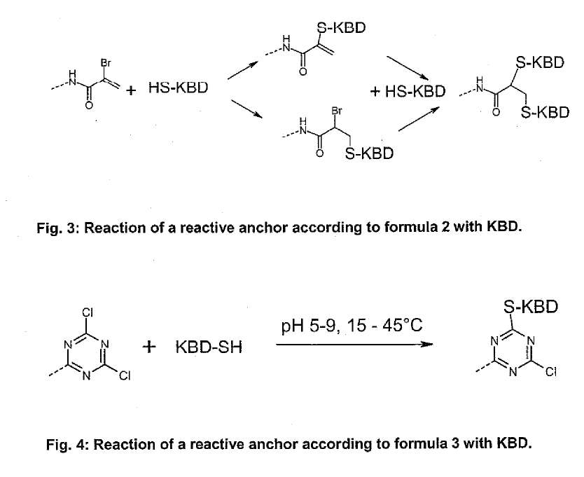

After the activation (elimination), the reaction shown in FIG. 2 takes place. Dyes with acrylamido anchor (formula 2) can be reacted directly, without preelimination (activation) with, for example, the KBD. The reaction conditions correspond to the conditions chosen for the activation of the vinylsulfone anchor. In the case, both addition and substitution can take place. (J. Soc. Dyers and Colourist, 1982, 98, 165-175) (see FIG. 3).

Dyes with a heteroatomatic anchor (formulae 3, 4, 5) can be reacted directly and without preelimination (activation) with, for example, the KBD (FIG. 4). The reaction conditions correspond to the conditions chosen for the vinylsulfone anchors. The reactive dyes used here can comprise 0-20% by weight, usually 0-10% by weight, often 0-5% by weight, of nonreactive secondary components. The secondary components, which have no reactive anchor, naturally do not react with the keratin-binding polypeptides. These can be removed during the synthesis or the coloring process. One option is to apply the keratin-binding polypeptides reacted with the reactive dye to hair and to wash out the nonbonded dye fractions from the hair.

Furthermore, there is the option to purify the keratin-binding polypeptides reacted with the reactive dye by, for example, column chromatography. It is also possible to carry out the reaction between keratin-binding polypeptides and the reactive dye in a type of “column reactor”. Here, the keratin-binding polypeptides could be coupled to a nickel affinity column, the dye bonded to the keratin-binding polypeptides and the unfixed dye radicals washed out directly. The keratin-binding polypeptide dye (keratin-binding effector molecule) could then be eluted from the column.

In a particularly preferred embodiment of the present invention, the keratin-binding effector molecules comprise dyes which are suitable for cosmetic purposes and are approved as such. Such dyes are listed, for example, in the publication “Kosmetische Färbemittel” [Cosmetic Colorants] from the Forbstoffkommission der Deutschen Forschungsgemeinschaft [Dyes Commission of the German Research Society], published by Verlag Chemie, Weinheim, 1984, or in the third completely revised edition from 1991.

Further dyes customarily used in coloring hair and which may be present in the keratin-binding effector molecules according to the invention are described, inter alia, in E. Sagarin, “Cosmetics, Science and Technology”, Interscience Publishers Inc., New York (1957), pages 503 ff., and H. Janistyn, “Handbuch der Kosmetika und Riechstoffe” [Handbook of cosmetics and fragrances], volume 3 (1973), pages 388 ff. and K. Schrader, “Grundlagen und Rezepturen der Kosmetika” [Fundamentals and formulations of cosmetics], 2nd edition (1989), pages 782-815.

According to the invention, preference is also given to keratin-binding effector molecules in which the reactive dye (i) is coupled to the keratin-binding polypeptide (ii) indirectly via a linker molecule.

The preparation of such a keratin-binding effector molecule can take place by coupling an effector molecule (i) carrying at least one hydroxy, amino or carboxyl function (e.g. chosen from the reactive dyes described above) onto one of the keratin-binding polypeptides (ii) described above using a linker molecule which has at least two coupling functionalities, and
a) in a first coupling step, firstly the effector molecule (i) is bonded to the linker molecule (iii), and
b) in a further coupling step, the reaction product from (a) is coupled to the keratin binding polypeptide (ii) via still free coupling functionality of the linker molecule (iii).

In a particularly preferred embodiment of the invention, the linker molecule (iii) has at least two different coupling functionalities, very particularly preferred are linker molecules (iii) which have a maleimide group.

The linker molecules (iii) used are particularly preferably maleimides carrying carboxylic acid groups according to the general formula 9,

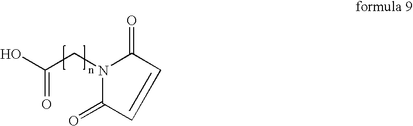

where “n” is an integer between 0 and 40 or 0-20, preferably between 0 and 15, particularly preferably between 0 and 10, very particularly preferably between 1 and 9, or between 2 and 8, or between 3 and 7, most preferably of all 5. The use of maleimidocoproic acid is the most preferred of all. In addition, the use of maleimidocaproic acid chloride is very particularly preferred.

In a further particularly preferred embodiment, the linker molecule (iii) has at least two different coupling functionalities and additionally a module which increases the hydrophilicity. This preferred linker molecule is depicted in formula 9b,

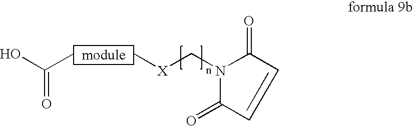

where “n” is an integer between 0 and 40 or 0 and 20, preferably between 0 and 15, particularly preferably between 0 and 10, very particularly preferably between 1 and 9, or between 2 and 8, or between 3 and 7, and X is the radicals O, S, N, CH₂, —O—C═O, O═C—O—, —NR, —NR—C═O, O═C—NR—, and R is H, C₁-C₁₂ branched or unbranched alkyl groups, such as methyl, ethyl, propyl, isopropyl, butyl, isobutyl, sec-butyl, tert-butyl, pentyl, isopentyl, neopentyl, tert-pentyl, hexyl, heptyl, octyl, nonyl, decyl, undecyl, dodecyl, or cycloalkyl, benzoyl, benzyl, C₆ to C₁₀ aryl groups, such as phenyl and naphthyl, heteroaryl, preferably H, methyl and ethyl, and
the “module” is an ethylene glycol or polyethylene glycol radical having 2 to 40, preferably 2 to 20, particularly preferably 2 to 10, repeat units, or an amino acid, preferably chosen from the group consisting of glycine, alanine, serine, threonine, glutamic acid, glutamine, aspartic acid, asparagine, arginine and cysteine, or a polypeptide having 2 to 40, preferably 2 to 20, particularly preferably 2 to 10, amino acids, where the amino acids are preferably polar amino acids, particularly preferably chosen from the group consisting of glycine, alanine, serine, threonine, glutamic acid, glutamine, aspartic acid, asparagine, arginine and cysteine, or a polyacrylic acid radical having 2-100, preferably 2-80, particularly preferably 2-50, most preferably 2-20, monomer units, or
for increasing the lipophilicity the “module” is an alkyl radical having 2-40 carbons or polyolefin radical having 2 to 40, preferably 2 to 20, particularly preferably 2 to 10, repeat units, or an amino acid, preferably chosen from the group consisting of glycine, valine, leucine, isoleucine, phenylalanine, tryptophan, proline, methionine, or a polypeptide having 2 to 40, preferably 2 to 20, particularly preferably 2 to 10, amino acids, where the amino acids are preferably nonpolar amino acids, particularly preferably chosen from the group consisting of glycine, valine, leucine, isoleucine, phenylalanine, tryptophan, proline, methionine, or a polyester, polyamide or polyurethane having 2-100, preferably 2-80, particularly preferably 2-50, most preferably 2-20 monomer units.

In a moreover preferred embodiment, the linker molecule is a molecule according to the general formula 9c,

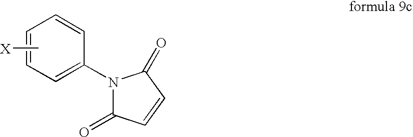

where X in the o, m or p position is COOH or R—COOH, and R is a C₁-C₁₂ linear alkyl group such as methyl, ethyl, propyl, isopropyl, butyl, isobutyl, sec-butyl, tert-butyl, pentyl, isopentyl, neopentyl, tert-pentyl, hexyl, heptyl, octyl, nonyl, decyl, undecyl, dodecyl, or a cyclic alkyl group such as a C₅-C₁₂-cycloalkyl radical, optionally substituted by one or more C₁-C₄-alkyl groups, or an o-, m- or p-oriented aryl, benzyl or benzoyl unit, preferably cyclohexyl, phenyl and naphthyl.

In a further preferred embodiment, R can also be the “module” described in formula 1b.

In a further embodiment of the present invention, the linker molecules (ii) shown by the general formula 10 are used,

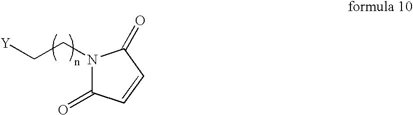

where “n” is an integer between 0 and 20, preferably between 0 and 15, particularly preferably between 1 and 10, very particularly preferably between 1 and 8, and Y is a hydroxy or amino group. Amino groups can be primary or secondary. The linker molecule (iii) is very particularly preferably a maleimidoalkanol. The maleimidoalkanols are preferably maleimidoethanol, most preferably of all maleimidopentanol.

In a further particularly preferred embodiment, the linker molecule (iii) according to formula 10 has at least two different coupling functionalities and additionally a module which increases the hydrophilicity or lipophilicity. This preferred linker molecule is shown in formula 10b,

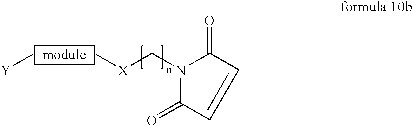

where “n” is an integer between 0 and 40 or 0 and 20, preferably between 0 and 15, particularly preferably between 0 and 10, very particularly preferably between 1 and 9, or between 2 and 8, or between 3 and 7, and X is the radicals O, S, N, CH₂, —O—C═O, O═C—O—, —NR, —NR—C═O, O═C—NR—, and R is H, C₁-C₁₂ branched or unbranched alkyl groups, such as methyl, ethyl, propyl, isopropyl butyl, isobutyl, sec-butyl, tert-butyl, pentyl, isopentyl, neopentyl, tert-pentyl, hexyl, heptyl, octyl, nonyl, decyl, undecyl, dodecyl, or cycloalkyl, benzoyl, benzyl, C₆ to C₁₀-aryl groups, such as phenyl and naphthyl, heteroaryl, preferably H, methyl and ethyl, and
the “module” is an ethylene glycol or polyethylene glycol radical having 2 to 40, preferably 2 to 20, particularly preferably 2 to 10, repeat units, or an amino acid, preferably chosen from the group consisting of glycine, alanine, serine, threonine, glutamic acid, glutamine, aspartic acid, asparagine, arginine and cysteine, or a polypeptide having 2 to 40, preferably 2 to 20, particularly preferably 2 to 10, amino acids, where the amino acids are preferably polar amino acids, particularly preferably chosen from the group consisting of glycine, alanine, serine, threonine, glutamic acid, glutamine, aspartic acid, asparagine, arginine and cysteine, or a polyacrylic acid radical having 2-100, preferably 2-80, particularly preferably 2-50, most preferably 2-20, monomer units, or
for increasing the lipophilicity the “module” is an alkyl radical having 2-40 carbon atoms or polyolefin radical having 2 to 40, preferably 2 to 20, particularly preferably 2 to 10, repeat units, or an amino acid, preferably chosen from the group consisting of glycine, valine, leucine, isoleucine, phenylalanine, tryptophan, proline, methionine, or a polypeptide having 2 to 40, preferably 2 to 20, particularly preferably 2 to 10, amino acids, where the amino acids are preferably nonpolar amino acids, particularly preferably chosen from the group consisting of glycine, valine, leucine, isoleucine, phenylalanine, tryptophan, proline, methionine, or a polyester, polyamide or polyurethane having 2-100, preferably 2-80, particularly preferably 2-50, most preferably 2-20 monomer units and Y is a functional group from hydroxy or amino groups.

In a further preferred embodiment, coupling of the linker molecule (iii) with the effector molecule (i) (e.g. reactive dye as described above) is a carbodiimide-, anhydride- or acid chloride-mediated esterification reaction or amide formation, where the use of the acid chloride of the linker molecule (iii) is particularly preferred. Carbodiimide-, anhydride- or acid chloride-mediated reaction means the activation of the carboxyl group of the linker molecule (iii) required for the formation of an ester or amide between linker molecule (iii) and effector molecule (i).

Carbodiimides to be mentioned are preferably dicyclohexylcarbodiimide (DCC), diisopropylcarbodiimide (DIC), N′-(3-dimethylaminopropyl)-N-ethylcarbodiimide hydrochloride (EDC), where the use of diisopropylcarbodiimide or EDC are particularly preferred. In addition, it is possible to carry out an activation with carbonyldiimidazole (CDI). These esterifications are carried out in the presence of 0.1-100 mol % of N,N-dimethylaminopyridine (DMAP), preferably 0.5-10%, particularly preferably 1-6%. The formation of amides can take place by reacting the compound activated with carbodiimide with the amine. Optionally, the amide formation can be carried out in the presence of additives, such as, for example, N-hydroxysuccinimide, pentafluorophenol or N-hydroxybenzotriazole. Such additives are known to the person skilled in the art. If active esters isolatable through these additives are obtained, the reactions of these isolated active esters with the effector molecules are also understood according to the invention as carbodiimide-mediated esterification.

The reaction of the linker molecule (iii) to give the anhydride takes place by general methods, as are known to the person skilled in the art. Preference is given to the use of mixed anhydrides, as are obtained, for example, by reaction with acetic anhydride, pivaloyl anhydride, acetyl chloride, pivaloyl chloride or chloroformic esters. Particular preference is given to pivaloyl anhydrides and to the anhydrides with carbonic acid. When using the acid chlorides, it is expedient to carry out the anhydride formation in the presence of a tertiary base, such as, for example, pyridine, triethylamine.

The coupling of the linker molecule (iii) with the effector molecule (i) described under (a) can preferably be carried out after the above-described activation of the linker molecule (iii) to give the anhydride in the presence of a base. Preferred bases to be mentioned are: aromatic and tertiary alkylamines, e.g. pyridine, triethylamine, tributylamine, trioctylamine, ethyldiisopropylamine etc. In a particularly preferred embodiment, the base used is triethylamine.

For the reaction of the linker molecule (iii) to the acid chloride, the chlorinating agents used are the customary chlorinating agents known to the person skilled in the art, for example thionyl chloride, phosphorus trichloride, phosphorus pentachloride, oxalyl chloride, phosgene, or phosphorus oxychloride. Very particular preference is given to the use of thionyl chloride (SOCl₂).

Using thionyl chloride, it is possible, for example, to convert maleimidocaproic acid into the acid chloride. Suitable solvents here are: aromatic and aliphatic hydrocarbons, e.g. benzene, toluene, xylenes, hexane, heptane, etc., halogenated hydrocarbons, e.g. methylene chloride, ethers, e.g. diethyl ether, THF etc., and an excess of the chlorinating agent itself. In a preferred embodiment, toluene is used.

The chlorination can be carried out with or without a catalyst. DMF is particularly preferred as catalyst for the chlorination.

Optionally, the reaction product from step (a) (referred to below as linker effector molecule (iv)) can be further purified to separate possible isomers of the reaction product. Here, all customary methods of purifying chemical substances can be used, e.g.: distillation, rectification, crystallization, extractions and chromatographic purification methods. Column chromatography is preferably carried out.

Besides the abovementioned reactive dyes, other dyes can also be coupled to a keratin-binding polypeptide by means of a linker and be used for the method according to the invention.

Here, particularly advantageous dyes are those specified in the list below. The Color Index Numbers (CIN) are taken from the Rowe Colour Index, 3rd edition, Society of Dyers and Colourists, Bradford, England, 1971.

TABLE 3
Advantageous dyes

| Chemical name or other name | CIN | Color |
|---|---|---|
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxynaphthalene | 12085 | red |
| Pigment Red 3 | 12120 | red |
| Ceres Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfodiethylamido-1′-phenylazo)-3-hydroxy-5″- | 12490 | red |
| chloro-2″,4″-dimethoxy-2-naphthanilide | | |
| Disperse Yellow 16 | 12700 | yellow |
| 1-(4-Sulfo-1-phenylazo)-4-aminobenzene-5-sulfonic acid | 13015 | yellow |
| 2,4-Dihydroxyazobenzene-4′-sulfonic acid | 14270 | orange |
| 2-(2,4-Dimethylphenylazo-5-sulfo)-1-hydroxynaphthalene-4-sulfonic acid | 14700 | red |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfo-4-chloro-5-carboxy-1-phenylazo)-2- | 15525 | red |
| hydroxynaphthalene | | |
| 1-(3-Methylphenylazo-4-sulfo)-2-hydroxynaphthalene | 15580 | red |
| 1-(4′,(8′)-Sulfonaphthylazo)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2-naphthylcarboxylic acid | 15850 | red |
| 1-(2-Sulfo-4-methyl-5-chloro-1-phenylazo)-2- | 15865 | red |
| hydroxynaphthalene-3-carboxylic acid | | |
| 1-(2-Sulfo-1-naphthylazo)-2-hydroxynaphthalene-3-carboxylic acid | 15880 | red |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic acid | 16185 | red |
| Acid orange 10 | 16230 | orange |
| 1-(4-Sulfo-1-naphthylazo)-2-naphthol-6,8-disulfonic acid | 16255 | red |
| 1-(4-Sulfo-1-naphthylazo)-2-naphthol-3,6,8-trisulfonic acid | 16290 | red |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5- | 19140 | yellow |
| hydroxypyrazolone-3-carboxylic acid | | |
| Pigment Yellow 16 | 20040 | yellow |
| 2,6-(4′-Sulfo-2″,4″-dimethyl)bisphenylazo)-1,3-dihydroxybenzene | 20170 | orange |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]-1-hydroxy- | 27755 | black |
| 7-aminonaphthalene-3,6-disulfonic acid | | |
| 4′-[(4″-Sulfo-1″-phenylazo)-7′-sulfo-1′-naphthylazo]-1-hydroxy-8- | 28440 | black |
| acetyl-aminonaphthalene-3,5-disulfonic acid | | |
| Direct orange 34, 39, 44, 46, 60 | 40215 | orange |
| Basic Violet 14 | 42510 | violet |
| Basic Violet 2 | 42520 | violet |
| 2-Hydroxy-3,6-disulfo-4,4′- | 44090 | green |
| bisdimethylaminonaphthofuchsonimmonium | | |
| 3-(2′-Methylphenylamino)-6-(2′-methyl-4′-sulfophenylamino)-9- | 45190 | violet |
| (2″-carboxyphenyl)xanthenium salt | | |
| Fluorescein | 45350:1 | yellow |
| Phenyl-2-oxyfluorone-2-carboxylic acid | 45350 | yellow |
| 4,5-Dibromofluorescein | 45370:1 | orange |
| 4,5-Dibromofluorescein | 45370 | orange |
| 2,4,5,7-Tetrabromofluorescein | 45380:1 | red |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410:1 | red |
| 3′,4′,5′,6′-Tetrachloro-2,4,5,7-tetrabromofluorescein | 45410 | red |
| 4,5-Diiodofluorescein | 45425:1 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| 1,2-Dioxyanthraquinone, calcium-aluminum complex | 58000 | red |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinone azine | 69800 | blue |
| Vat Blue 6; Pigment Blue 64 | 69825 | blue |
| Indigo | 73000 | blue |
| Indigo disulfonic acid | 73015 | blue |
| Quinacridone Violet 19 | 73900 | violet |
| Pigment Red 122 | 73915 | red |
| Keto and/or hydroxyl derivatives of caredane | 75135 | yellow |
| Guanine or pearlescent agent | 75170 | white |
| 1,7-Bis(4-hydroxy-3-methoxyphenyl)-1,6-heptadiene-3,5-dione | 75300 | yellow |
| Complex salt (Na, Al, Ca) of carminic acid | 75470 | red |
| 6,7-Dimethyl-9-(1′-D-ribityl)isoalloxazine, lactoflavin | | yellow |
| Capsanthin, capsorubin | | orange |
| Betanin | | red |
| Acid Red 195 | | red |

In a further preferred embodiment of the present invention, for the method according to the invention for coloring skin, hair or nails, keratin-binding effector molecules are used which comprise one of the dye molecules shown in Table 3 coupled via a linker described above.

The abovementioned dyes can also be used as effector molecules (i) to a skin- or nail-binding polypeptide sequence (ii) for the coloring of skin or nails e.g. in tattoos. Of particular suitability is the use of keratin-binding effector molecules comprising fluorescent dyes (e.g. the fluorescent dyes included in Table 3) to achieve a more healthy and luminous-looking skin shade and for optically lightening the skin (“skin whitening”) following application to the skin. The use of fluorescent pigments is described, for example, in U.S. Pat. No. 6,753,002. Fluorescent dyes for producing a healthier skin shade are described in “Filling the Fluorescent Palette, Cosmetics & Toiletries, 26-34, 121, No. 5, 2006”. Preference is given, for example, to fluorescent dyes from DayGlo. In addition, these keratin-binding effector molecules comprising fluorescent dyes can also be used for lightening hair and for producing special reflections or shimmers on the hair. This is described, for example in “Hair lightening by fluorescent dyes, Cosmetics & Toiletries, 56-57, 120, No. 7, 2005” and the specification US 2004/0258641 cited therein.

The binding of the reaction product arising from the above-described step (a) with the keratin-binding polypeptide (ii) takes place via the second still free anchor group of the linker molecule. For example, such an anchor group can be a thiol function, by means of which the linker can enter into a disulfide bond with a cysteine radical of the keratin-binding polypeptide (ii).

The use of tailored linkers allows the precise matching of the linking of the linker effector molecule to the keratin-binding polypeptide. Furthermore, it is possible as a result to link two or more effector molecules to a keratin-binding polypeptide (ii).

The linker used is governed by the functionality to be coupled. Of suitability are, for example, molecules which couple polypeptides (ii) to be keratin-bonded by means of sulfhydryl-reactive groups (e.g. maleimides, pyridyl disulfides, α-haloacetyls, vinylsulfones, sulfatoalkylsulfones (preferably sulfatoethylsulfones)).

Preference is given to a covalent linkage of the linker molecule (iii) with the keratin-binding polypeptide (ii). This can take place, for example, via the side chains of the keratin-binding polypeptide (ii), in particular via amino functions, hydroxy functions, carboxylate functions or thiol functions. Preference is given to a linkage via the amino functions of one or more lysine radicals, one or more thiol groups of cysteine radicals, one or more hydroxyl groups of serine, threonine or tyrosine radicals, one or more carboxyl groups of aspartic acid or glutamic acid radicals or via the N-terminal or C-terminal function of the keratin-binding polypeptide (ii). Apart from the amino acid functions occurring in the primary sequence of the keratin-binding polypeptide (ii), it is also possible to add amino acids with suitable functions (e.g. cysteines, lysines, aspartates, glutamates) to the sequence, or to substitute amino acids of the polypeptide sequence by such amino acid functions. Methods for the mutagenesis or manipulation of nucleic acid molecules are sufficiently known to the person skilled in the art. A few selected methods are described below.

Particular preference is given to the use of a linker effector molecule (iv) which has been prepared using the maleimidocaproic acid specified as being preferred for the method according to the invention. In the case of such a linker effector molecule (iv), the cysteine radicals present in the keratin-binding polypeptide are used for the coupling.

The success of the effector coupling can be monitored by means of three different tests:
(i) Ellmann test in which the number of free Cys-SH groups in the Protein can be determined before and after effector coupling. A considerable reduction in the free SH groups after coupling indicates good reaction progress (see Example 11).
(ii) Activity test in which the binding of the KBD-B (SEQ ID NO.: 166) with and without coupled effector to hair can be measured. Good reaction control should not reduce the activity of KBD-effector toward uncoupled KBD-B (SEQ ID No.: 166) (see Example 12)
(iii) Visible coloration of hair or skin as a result of the successful coupling of the dye onto the KBD-B (SEQ ID No.: 166) (see Example 14).

In a further embodiment according to the invention, the binding of the effector molecule takes place in such a way that they can be eliminated and released from the keratin-binding polypeptides (ii) in the sense of a “slow release” or “controlled release” as a result of the effect of endogenous enzymes (for example esterases, lipases or glucosidases) or as a result of the ambient conditions on the skin (e.g. moisture, acidic pH) over time. The keratin-binding polypeptides (ii) can thus be used as application system with which, through single or repeated application, small amounts of the free effector molecules on the skin can be achieved. In principle, it is known to the person skilled in the art that effectors can be released on the skin from their corresponding derivatives, for example from tocopherol acetate, ascorbyl palmitate or ascorbyl glucosides (exemplary literature: Redoulés, D. et al. J. Invest. Dermatol. 125, 2005, 270, Beijersbegen van Henegouwen, G. M. J. et al., J. Photochem. Photobiol. 29, 1995, 45.).

In a further preferred embodiment of the invention, for the method according to the invention, dyes carrying carboxyl, hydroxyl or amino groups are used. Here, the effector molecules used can have one or more carboxyl, hydroxyl or amino groups.

The present invention further provides the use of the above described keratin-binding effector molecules in cosmetic compositions suitable for coloring keratin fibers, preferably hair, skin or nails, particularly preferably human hair.

In another preferred embodiment, the use of the abovementioned keratin-binding effector molecules according to the invention is in compositions suitable for coloring hair in combination with cosmetically suitable auxiliaries and additives which are customarily used in hair colorants.

Here, the abovementioned dyes, preferably dyes approved for cosmetic purposes, can be used. These dyes are usually added in a concentration of from 0.001 to 1 percent by weight, preferably 0.01 to 0.9% by weight, particularly preferably 0.01 to 0.8% by weight or 0.01 to 0.7% by weight, very particularly preferably 0.01 to 0.6% by weight or 0.01 to 0.5% by weight, most preferably from 0.01 to 0.4% by weight or 0.01 to 0.3% by weight, based on the total weight of the composition. In a further embodiment, the compositions can comprise a keratin-binding effector molecule according to the invention in a concentration of from 1 to 10% by weight, preferably 2 to 8% by weight, 3 to 7% by weight, 4 to 6% by weight, based on the total weight of the composition. Moreover, the compositions can comprise a keratin-binding effector molecule according to the invention in a concentration of from 10 to 20% by weight, preferably 11 to 19% by weight, 12 to 18% by weight, 13 to 17% by weight, 14 to 16% by weight based on the total weight of the composition.

In order to ensure that the active ingredients of the hair-coloring compositions remain on the hair for a certain time following application and do not get into places where they are undesired, such as, for example, the face, the compositions should have a certain minimum viscosity. This viscosity is usually achieved through the use of thickeners, which are thus a further constituent of most hair colorants.

The thickeners used are usually crosslinked polyacrylic acids (e.g. Carbopol®), hydroxyethylcellulose, waxes and particularly mixtures of nonionic surfactants with a certain HLB value (hydrophobic lipophilic balance), anionic, cationic or nonionic association polymers. The use of ampholytic copolymers as thickeners for cosmetic compositions is known. Polymers are referred to as ampholytic or amphoteric if they have both anionogenic/anionic groups and cationogenic/cationic groups. Amphoteric polymers with an adequate number of dissociatable groups are water-soluble or water-dispersible and have found diverse uses in the pharmacy and cosmetics sector.

Suitable thickeners or polymers are described in the patent applications WO 00/039176, WO 04/058837, EP-A-0 982 021, WO 01/62809, WO 05/004821, DE 202 07 896 U1 and WO 02/000181.

To establish certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes, silicone resins or dimethicone copolyols (CTFA) and amino functional silicone compounds, such as amodimethicones (CTFA).

Propellants are the propellants customarily used for hair sprays or aerosol foams. Preference is given to mixtures of propane/butane, pentane, dimethyl ether, 1,1-difluoroethane (HFC-152 a), carbon dioxide, nitrogen or compressed air.

Emulsifiers which can be used are all emulsifiers customarily used in hair foams. Suitable emulsifiers may be nonionic, cationic or anionic or amphoteric. Examples of nonionic emulsifiers (INCI nomenclature) are laureths, e.g. laureth-4; ceteths, e.g. ceteth-1, polyethylene glycol cetyl ether, ceteareths, e.g. ceteareth-25, polyglycol fatty acid glycerides, hydroxylated lecithin, lactyl esters of fatty acids, alkyl polyglycosides.

Examples of cationic emulsifiers are cetyidimethyl-2-hydroxyethylammonium dihydrogenphosphate, cetyltrimonium chloride, cetyltrimonium bromide, cocotrimonium methyl sulfate, quaternium-1 to x (INCI).

Anionic emulsifiers can be chosen, for example, from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Gel formers which can be used are all gel formers customary in cosmetics. These include slightly crosslinked polyacrylic acid, for example Carbomer (INCI), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, polyquaternium-32 (and) paraffinum liquidum (INCI), sodium acrylate copolymers (and) paraffinum liquidum (and) PPG-1 trideceth-6, acrylamidopropyltrimonium chloride/acrylamide copolymers, steareth-10 allyl ether, acrylate copolymers, polyquaternium-37 (and) paraffinum liquidum (and) PPG-1 trideceth-6, polyquaternium 37 (and) propylene glycol dicaprate dicaprylate (and) PPG-1 trideceth-6, polyquaternium-7, polyquaternium-44.

In the shampoo formulations, all of the anionic, neutral, amphoteric or cationic surfactants customarily used in shampoos can be used.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Of suitability are, for example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, trmethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. In addition, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

In the shampoo formulations, in order to achieve certain effects, customary conditioning agents can be used in combination with the keratin-binding effector molecules according to the invention.

These include, for example, the abovementioned cationic polymers with the INCI name Polyquaternium, in particular copolymers of vinylpyrrolidone/N-vinylimidazolium salts (Luviquat FC, Luviquat&commat, HM, Luviquat MS, Luviquat Care), copolymers of N-vinylpyrrolidone/dimethylaminoethyl methacrylate, quaternized with diethyl sulfate (Luviquat D PQ 11), copolymers of N-vinylcaprolactam/N-vinylpyrrolidone/N-vinylimidazolium salts (Luviquat D Hold), cationic cellulose derivatives (Polyquaternium-4 and -10), acrylamide copolymers (Polyquaternium-7). In addition, Protein hydrolyzates can be used, and also conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and amino-functional silicone compounds, such as amodimethicones (CTFA). In addition, cationic guar derivatives, such as Guar Hydroxypropyltrimonium Chloride (INCI) can be used.

As a rule, the hair cosmetic or skin cosmetic preparation is used for application to the skin (topical) or hair. Topical preparations are understood here as meaning those preparations which are suitable for applying the active ingredients to the skin or the hair in a fine distribution. Of suitability for this purpose are, for example, aqueous and aqueous-alcoholic solutions, sprays, foams, foam aerosols, ointments, aqueous gels, emulsions of the O/W or W/O type, microemulsions or cosmetic stick preparations.

According to a preferred embodiment of the cosmetic composition according to the invention, the composition comprises a carrier. A preferred carrier is water, a gas, a water based liquid, an oil, a gel, an emulsion or microemulsion, a dispersion or a mixture thereof. The specified carriers exhibit good skin compatibility. Of particular advantage for topical preparations are aqueous gets, emulsions or microemulsions.

Emulsifiers which can be used are nonionogenic surfactants, zwitterionic surfactants, ampholytic surfactants or anionic emulsifiers. The emulsifiers may be present in the composition according to the invention in amounts of from 0.1 to 10% by weight, preferably 1 to 5% by weight, based on the composition.

The nonionogenic surfactant used may, for example, be a surfactant from at least one of the following groups:

addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;
C_{12/18}-fatty acid mono- and diesters of addition products of from 1 to 30 mol of ethylene oxide onto glycerol; glycerol mono- and diesters and sorbitan mono- and diesters of saturated and unsaturated fatty acids having 6 to 22 carbon atoms and ethylene oxide addition products thereof; alkyl mono- and oligoglycosides having 8 to 22 carbon atoms in the alkyl radical and ethoxylated analogs thereof; addition products of from 15 to 60 mol of ethylene oxide onto castor oil and/or hydrogenated castor oil; polyol and, in particular polyglycerol esters, such as, for example, polyglycerol polyricinoleate, polyglycerol poly-12-hydroxystearate or polyglycerol dimerate. Likewise suitable are mixtures of compounds from two or more of these classes of substances;
addition products of from 2 to 15 mol of ethylene oxide onto castor oil and/or hydrogenated castor oil;
partial esters based on linear, branched, unsaturated or saturated C_{6/22} fatty acids, ricinoleic acid, and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside), and polyglucosides (e.g. cellulose); mono-, di- and trialkyl phosphates, and mono-, di- and/or tri-PEG alkyl phosphates and salts thereof;
wool wax alcohols;
polysiloxane-polyalkyl polyether copolymers and corresponding derivatives;
mixed esters of pentaerythritol, fatty acids, citric acid and fatty alcohol as in German patent specification 1165574 and/or mixed esters of fatty acids having 6 to 22 carbon atoms, methylglucose and polyols, preferably glycerol or polyglycerol, and polyalkylene glycols.

In addition, zwitterionic surfactants can be used as emulsifiers. Zwitterionic surfactants is the term used to refer to those surface-active compounds which carry at least one quaternary ammonium group and at least one carboxylate group or a sulfonate group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazolines having in each case 8 to 18 carbon atoms in the alkyl or acyl group, and cocoacylaminoethylhydroxyethyl carboxymethylglycinate. Particular preference is given to the fatty acid amide derivative known under the CTFA name Cocamidopropyl Betaine.

Likewise suitable emulsifiers are ampholytic surfactants. Ampholytic surfactants are understood as meaning those surface-active compounds which, apart from a C_{8,18}-alkyl or -acyl group in the molecule, comprise at least one free amino group and at least one —COOH— or —SO₃H group, and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurtnes, N-alkylsarcosines, 2-alkylaminopropionic acids and alkylaminoacotic acids having in each case about 8 to 18 carbon atoms in the alkyl group.

Particularly preferred ampholytic surfactants are N-cocoalkylaminopropionate, cocoacylaminoethylaminopropionate and C_{12/18}-acylsarcosine. Besides the ampholytic emulsifiers, quaternary emulsifiers are also suitable, with those of the ester quat type, preferably methyl-quaternized difatty acid triethanolamine ester salts, being particularly preferred. Furthermore, anionic emulsifiers which may be used are alkyl ether sulfates, monoglyceride sulfates, fatty acid sulfates, sulfosuccinates and/or ether carboxylic acids.

Suitable oil bodies are Guerbet alcohols based on fatty alcohols having 6 to 18, preferably 8 to 10, carbon atoms, esters of linear C₆-C₂₂-fatty acids with linear C₅-C₂₂-fatty alcohols, esters of branched C₆-C₁₃-carboxylic acids with linear C₆-C₂₂-fatty alcohols, esters of linear C₆-C₂₂-fatty acids with branched alcohols, in particular 2-ethylhexanol, esters of linear and/or branched fatty acids with polyhydric alcohols (such as, for example, propylene glycol, dimerdiol or trimertriol) and/or Guerbet alcohols, triglycerides based on C₆-C₁₈-fatty acids, liquid mono-/di-, triglyceride mixtures based on C₆-C₁₈-fatty acids, esters of C₆-C₂₂-fatty alcohols and/or Guerbot alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear C₆-C₂₂-fatty alcohol carbonates, Guerbet carbonates, esters of benzoic acid with linear and/or branched C₆-C₂₂-alcohols (e.g. Finsolv® TN), dialkyl ethers, ring-opening products of epoxidized fatty acid esters with polyols, silicone oils and/or aliphatic or naphthenic hydrocarbons. Oil bodies which may be used are also silicone compounds, for example dimethylpolysiloxanes, methylphenylpolysiloxanes, cyclic silicones, and amino-, fatty-acid-, alcohol-, polyether-epoxy-, fluorine-, alkyl- and/or glycoside-modified silicone compounds, which may either be in the form of a liquid or in the form of a resin at room temperature. The oil bodies may be present in the compositions according to the invention in amounts of from 1 to 90% by weight, preferably 5 to 80% by weight, and in particular 10 to 50% by weight, based on the composition.

The list of specified ingredients which can be used together with the keratin-binding effector molecules according to the invention should of course not be regarded as being exhaustive or limiting. The ingredients can be used individually or in any combinations with one another.

In addition, the present invention relates to compositions suitable for coloring skin, nails and/or hair, preferably hair colorants, comprising at least one of the above described keratin-binding effector molecules according to the invention.

This invention further provides a method of coloring hair, skin and/or nails using the keratin-binding effector molecules according to the invention. In this method, preference is given to using keratin-binding effector molecules which comprise at least one of the abovementioned keratin-binding polypeptides (ii) and at least one dye or reactive dye according to Tables 3, 5 and 6.

Naturally, when producing the preparations suitable for coloring hair, keratin-binding effector molecules can also be mixed together with different dyes so that specific color nuances are achieved. Preparations with specific color nuances can be produced, for example, by
(a) mixing selected reactive dyes or dyes during the production of the keratin-binding effector molecule, or
(b) mixing individual keratin-binding effector molecules when creating the hair colorant.

In a particularly preferred embodiment of the method according to the invention, keratin-binding effector molecules are used comprising
(e) a keratin-binding polypeptide which comprises one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170, preferably in SEQ ID NO: 2, 4, 6, 8, 10, 12, 14, 40, 42, 44, 46, 48, 146, 150, 153, 156, 157, 158, 160, 162, 164, 166, 168 or 170, particularly preferably 166 and 168, most preferably 168, or correspond to a polypeptide which is at least 40%, 45% or 50%, preferably at least 55%, 60%, 65% or 70%, particularly preferably at least 75%, 80%, 85%, 90%, 91%, 92%, 93% or 94%, very particularly preferably at least 95% or 96%, identical to at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170, preferably in SEQ ID NO: 2, 4, 6, 8, 10, 12, 14, 40, 42, 44, 46, 48, 146, 150, 153, 156, 157, 158, 160, 162, 164, 166, 168 or 170, particularly preferably 166 and 168, most preferably 168, and is able to bind keratin,
(f) as linker molecule (iii), maleimidocaproic acid or a maleimidoalkanol, preferably maleimidopentanol, and
(g) an effector molecule (i) chosen from the dyes listed in Table 3, or
(h) a reactive dye chosen from Tables 6 or 7.

Preferably, the hair is colored by applying a preparation comprising the keratin-binding effector molecules according to the invention to the hair to be colored in an amount and time sufficient for producing the desired color change.

Here, a good coloring can be achieved with a suitable keratin-binding effector molecule after just a very short time (a few minutes) or at least within half an hour (see Example 20).

The person skilled in the art is aware that the amount and time required for the coloring depends on the length and number of hairs to be colored.

In a particularly preferred embodiment of the abovementioned method, it is a reversible coloring method in which the keratin-binding effector molecule can be removed from skin, hair or nails in a displacement reaction. For this, a rinse with keratin, for example, can be used, as a result of which the keratin-binding effector molecules are displaced from their existing bond to the keratin and are saturated with the keratin from the rinse. Alternatively, a rinse with a high fraction of detergent (e.g. SDS) for washing off is also possible. (see Example 20).

Sequences

| SEQ ID | Sequence | |
|---|---|---|
| NO.: | type | Sequence description |
| 1 | Nucleic acid | Homo sapiens Desmoplakin_Accession NM_004415 |
| 2 | Protein | Homo sapiens Desmoplakin_Accession NM 004415 |
| 3 | Nucleic acid | Homo sapiens Desmoplakin_Accession NM_004415 domain B |
| 4 | Protein | Homo sapiens Desmoplakin_Accession NM_004415 domain B |
| 5 | Nucleic acid | Homo sapiens Desmoplakin_Accession NM_004415 domain B-1 |
| 6 | Protein | Homo sapiens Desmoplakin_Accession NM_004415 domain B-1 |
| 7 | Nucleic acid | Homo sapiens Desmoplakin_Accession NM_004415 domain B-2 |
| 8 | Protein | Homo sapiens Desmoplakin_Accession NM_004415 domain B-2 |
| 9 | Nucleic acid | Homo sapiens Desmoplakin_Accession NM_004415 domain C |
| 10 | Protein | Homo sapiens Desmoplakin_Accession NM_004415 domain C |
| 11 | Nucleic acid | Homo sapiens Desmoplakin_Accession NM_004415 domain C-1 |
| 12 | Protein | Homo sapiens Desmoplakin_Accession NM_004415 domain C-1 |
| 13 | Nucleic acid | Homo sapiens Desmoplakin_Accession NM_004415 domain C-2 |
| 14 | Protein | Homo sapiens Desmoplakin_Accession NM_004415 domain C-2 |
| 15 | Nucleic acid | Homo sapiens_Filaggrin_Accession CAI19595 |
| 16 | Protein | Homo sapiens_Filaggrin_Accession CAI19596 |
| 17 | Nucleic acid | Homo sapiens plakophilin 1 Accession NM_001005337, transcript variant 1a |
| 18 | Protein | Homo sapiens plakophilin 1 Accession NM_001005337, transcript variant 1a |
| 19 | Nucleic acid | Homo sapiens plakophilin 1 Accession NM_000299, transcript variant 1b |
| 20 | Protein | Homo sapiens plakophilin 1 Accession NM_000299, transcript variant 1b |
| 21 | Nucleic acid | Mus musculus plakophilin 2 Accession NM_026163 NM_027894 |
| 22 | Protein | Mus musculus plakophilin 2 Accession NM_026163 NM_027895 |
| 23 | Nucleic acid | Mus musculus plakophilin 1 ACCESSION NM_019645 |
| 24 | Protein | Mus musculus plakophilin 1 ACCESSION NM_019646 |
| 25 | Nucleic acid | Bos taurus plakophilin 1 partial mRNA, Accession XM_868348 |
| 26 | Protein | Bos taurus plakophilin 1 partial mRNA, Accession XM_868349 |
| 27 | Nucleic acid | Canis familiaris similar to plakophilin 1 isoform 1a, Accession XM_851528 |
| 28 | Protein | Canis familiaris similar to plakophilin 1 isoform 1a, Accession XM_851529 |
| 29 | Nucleic acid | Danio rerio similar to Plakophilin 1 Accession XM_695832 |
| 30 | Protein | Danio rerio similar to Plakophilin 1 Accession XM_695833 |
| 31 | Nucleic acid | Rattus norvegicus similar to plakophilin 1, Accession XM_222666 |
| 32 | Protein | Rattus norvegicus similar to plakophilin 1, Accession XM_222667 |
| 33 | Nucleic acid | Pan troglodytes similar to Plakophilin 1, Accession XM_514091 |
| 34 | Protein | Pan troglodytes similar to Plakophilin 1, Accession XM_514092 |
| 35 | Nucleic acid | Gallus gallus similar to plakophilin 1, Accession XM_419240 |
| 36 | Protein | Gallus gallus similar to plakophilin 1, Accession XM_419241 |
| 37 | Nucleic acid | Xenopus laevis similar to plakophilin 4, Accession BI390496 |
| 38 | Protein | Xenopus laevis similar to plakophilin 4, Accession BI390497 |
| 39 | Nucleic acid | Homo sapiens desmoplakin, transcript variant 2, Accession NM_001008844 |
| 40 | Protein | Homo sapiens desmoplakin, transcript variant 2, Accession NM_001008845 |
| 41 | Nucleic acid | Mus musculus desmoplakin, Accession XM_621314 |
| 42 | Protein | Mus musculus desmoplakin, Accession XM_621315 |
| 43 | Nucleic acid | Rattus norvegicus similar to desmoplakin isoform II, Accession XM_225259 |
| 44 | Protein | Rattus norvegicus similar to desmoplakin isoform II, Accession XM_225260 |
| 45 | Nucleic acid | Pan troglodytes desmoplakin, Accession XM_518227 |
| 46 | Protein | Pan troglodytes desmoplakin, Accession XM_518228 |
| 47 | Nucleic acid | Gallus gallus similar to Desmoplakin, Accession XM_418957 |
| 48 | Protein | Gallus gallus similar to Desmoplakin, Accession XM_418958 |
| 49 | Nucleic acid | Homo sapiens junction plakoglobin (JUP), transcript variant 2, Accession |
| | | NM_021991 |
| 50 | Protein | Homo sapiens junction plakoglobin (JUP), transcript variant 2, Accession |
| | | NM_021992 |
| 51 | Nucleic acid | Mus musculus, plakoglobin; gamma-catenin, Accession NM_010593 |
| 52 | Protein | Mus musculus, plakoglobin; gamma-catenin, Accession NM_010594 |
| 53 | Nucleic acid | Rattus norvegicus gamma-catenin (plakoglobin), Accession NM_031047 |
| 54 | Protein | Rattus norvegicus gamma-catenin (plakoglobin), Accession NM_031048 |
| 55 | Nucleic acid | Danio rerio armadillo Protein family; plakoglobin, Accession NM_131177 |
| 56 | Protein | Danio rerio armadillo Protein family; plakoglobin, Accession NM_131178 |
| 57 | Nucleic acid | Xenopus tropicalis junction plakoglobin, Accession BC064717 |
| 58 | Protein | Xenopus tropicalis junction plakoglobin, Accession BC064718 |
| 59 | Nucleic acid | Canis familiaris similar to junction plakoglobin isoform 10, Accession |
| | | XM_856625 |
| 60 | Protein | Canis familiaris similar to junction plakoglobin isoform 10, Accession |
| | | XM_856626 |
| 61 | Nucleic acid | Xenopus laevis Jup Protein, Accession BC094116 |
| 62 | Protein | Xenopus laevis Jup Protein, Accession BC094117 |
| 63 | Nucleic acid | Bos taurus junction plakoglobin, Accession NM_001004024 |
| 64 | Protein | Bos taurus junction plakoglobin, Accession NM_001004025 |
| 65 | Nucleic acid | Sus scrofa plakoglobin, Accession NM_214323 |
| 66 | Protein | Sus scrofa plakoglobin, Accession NM_214324 |
| 67 | Nucleic acid | Danio rerio junction plakoglobin, Accession BC058305 |
| 68 | Protein | Danio rerio junction plakoglobin, Accession BC058306 |
| 69 | Nucleic acid | Saccharomyces cerevisiae, plakoglobin/armadillo/beta-catenin, Accession |
| | | AF005267 |
| 70 | Protein | Saccharomyces cerevisiae, plakoglobin/armadillo/beta-catenin, Accession |
| | | AF005268 |
| 71 | Nucleic acid | Homo sapiens plectin 1, intermediate filament binding Protein, Accession |
| | | NM_201380 |
| 72 | Protein | Homo sapiens plectin 1, intermediate filament binding Protein, Accession |
| | | NM_201381 |
| 73 | Nucleic acid | Mus musculus plectin 1 (Plec1), transcript variant 11, mRNA, Accession |
| | | NM_201394 XM |
| 74 | Protein | Mus musculus plectin 1 (Plec1), transcript variant 11, mRNA, Accession |
| | | NM_201394 XM |
| 75 | Nucleic acid | Bos taurus similar to plectin 1 isoform 1 (LOC510991), Accession XM_588232 |
| 76 | Protein | Bos taurus similar to plectin 1 isoform 1 (LOC510991), Accession XM_588233 |
| 77 | Nucleic acid | Canis familiaris similar to plectin 1 isoform, Accession XM_539204 |
| 78 | Protein | Canis familiaris similar to plectin 1 isoform, Accession XM_539205 |
| 79 | Nucleic acid | Trypanosoma cruzi, plectin-like Protein, Accession XM_809849 |
| 80 | Protein | Trypanosoma cruzi, plectin-like Protein, Accession XM_809850 |
| 81 | Nucleic acid | Rattus norvegicus plectin, Accession X59601 |
| 82 | Protein | Rattus norvegicus plectin, Accession X59602 |
| 83 | Nucleic acid | Cricetulus griseus plectin, Accession AF260753 |
| 84 | Protein | Cricetulus griseus plectin, Accession AF260754 |
| 85 | Nucleic acid | Homo sapiens periplakin, Accession NM_002705 |
| 86 | Protein | Homo sapiens periplakin, Accession NM_002706 |
| 87 | Nucleic acid | Mus musculus periplakin, Accession NM_008909 XM_358905 |
| 88 | Protein | Mus musculus periplakin, Accession NM_008909 XM_358906 |
| 89 | Nucleic acid | Homo sapiens envoplakin, Accession NM_001988 |
| 90 | Protein | Homo sapiens envoplakin, Accession NM_001989 |
| 91 | Nucleic acid | Mus musculus envoplakin, Accession NM_025276 XM_283024 |
| 92 | Protein | Mus musculus envoplakin, Accession NM_025276 XM_283025 |
| 93 | Nucleic acid | Bos taurus similar to Envoplakin, Accession XM_587641 |
| 94 | Protein | Bos taurus similar to Envoplakin, Accession XM_587642 |
| 95 | Nucleic acid | Canis familiaris similar to Envoplakin, Accession XM_540443 |
| 96 | Protein | Canis familiaris similar to Envoplakin, Accession XM_540444 |
| 97 | Nucleic acid | Danio rerio similar to Envoplakin, Accession XM_687958 |
| 98 | Protein | Danio rerio similar to Envoplakin, Accession XM_687959 |
| 99 | Nucleic acid | Rattus norvegicus, similar to envoplakin, db_xref GeneID: 303687 |
| 100 | Protein | Rattus norvegicus, similar to envoplakin, db_xref GeneID: 303688 |
| 101 | Nucleic acid | Pan troglodytes similar to Envoplakin, Accession XM_511692 |
| 102 | Protein | Pan troglodytes similar to Envoplakin, Accession XM_511693 |
| 103 | Nucleic acid | Human bullous pemphigoid antigen, Accession M63618 |
| 104 | Protein | Human bullous pemphigoid antigen, Accession M63619 |
| 105 | Nucleic acid | Mus musculus bullous pemphigoid antigen 1 (Bpag1), Accession AF396877 |
| 106 | Protein | Mus musculus bullous pemphigoid antigen 1 (Bpag1), Accession AF396878 |
| 107 | Nucleic acid | Mus musculus trichohyalin-like 1, Accession NM_027762 |
| 108 | Protein | Mus musculus trichohyalin-like 1, Accession NM_027763 |
| 109 | Nucleic acid | Bos taurus similar to trichohyalin-like 1, Accession XM_597026 |
| 110 | Protein | Bos taurus similar to trichohyalin-like 1, Accession XM_597027 |
| 111 | Nucleic acid | Homo sapiens trichohyalin-like 1, Accession NM_001008536 XM_060104 |
| 112 | Protein | Homo sapiens trichohyalin-like 1, Accession NM_001008536 XM_060105 |
| 113 | Nucleic acid | Strongylocentrotus purpuratus similar to Trichohyalin, Accession XM_793822 |
| 114 | Protein | Strongylocentrotus purpuratus similar to Trichohyalin, Accession XM_793823 |
| 115 | Nucleic acid | Trypanosoma cruzi trichohyalin, putative, Accession XM_809758 |
| 116 | Protein | Trypanosoma cruzi trichohyalin, putative, Accession XM_809759 |
| 117 | Nucleic acid | Giardia lamblia ATCC 50803 trichohyalin, Accession XM_765825 |
| 118 | Protein | Giardia lamblia ATCC 50803 trichohyalin, Accession XM_765826 |
| 119 | Nucleic acid | Aspergillus fumigatus Af293, trichohyalin, Accession XM_748643 |
| 120 | Protein | Aspergillus fumigatus Af293, trichohyalin, Accession XM_748644 |
| 121 | Nucleic acid | O. cuniculus trichohyalin, Accession Z19092 |
| 122 | Protein | O. cuniculus trichohyalin, Accession Z19093 |
| 123 | Nucleic acid | Pan troglodytes similar to Trichohyalin, Accession XM_526770 |
| 124 | Protein | Pan troglodytes similar to Trichohyalin, Accession XM_526771 |
| 125 | Nucleic acid | Human trichohyalin (TRHY), Accession L09190 |
| 126 | Protein | Human trichohyalin (TRHY), Accession L09191 |
| 127 | Nucleic acid | Mus musculus small proline-rich Protein 3, Accession NM_011478 |
| 128 | Protein | Mus musculus small proline-rich Protein 3, Accession NM_011479 |
| 129 | Nucleic acid | Homo sapiens small proline-rich Protein 2B (SPRR2B), Accession |
| | | NM_001017418 |
| 130 | Protein | Homo sapiens small proline-rich Protein 2B (SPRR2B), Accession |
| | | NM_001017419 |
| 131 | Nucleic acid | Mus musculus hair follicle Protein AHF, Accession XM_485271 |
| 132 | Protein | Mus musculus hair follicle Protein AHF, Accession XM_485272 |
| 133 | Nucleic acid | Homo sapiens epiplakin 1 (EPPK1), Accession NM_031308 XM_372063 |
| 134 | Protein | Homo sapiens epiplakin 1 (EPPK1), Accession NM_031308 XM_372064 |
| 135 | Nucleic acid | Mus musculus epiplakin 1, Accession NM_144848 NM_173025 |
| 136 | Protein | Mus musculus epiplakin 1, Accession NM_144848 NM_173026 |
| 137 | Nucleic acid | Mus musculus structural Protein FBF1, Accession AF241249 |
| 138 | Protein | Mus musculus structural Protein FBF1, Accession AF241250 |
| 139 | Nucleic acid | Streptococcus mutans spaP gene for antigen I/II, Accession X17390 |
| 140 | Protein | Streptococcus mutans spaP gene for antigen I/II, Accession X17391 |
| 141 | Nucleic acid | Sequence of the PCR primer Bag 43 |
| 142 | Nucleic acid | Sequence of the PCR primer Bag 44 |
| 143 | Nucleic acid | Sequence of the PCR primer Bag 53 |
| 144 | Nucleic acid | Sequence of the PCR primer Bag 51 |
| 145 | Nucleic acid | DNA fragment which has been amplified by means of the PCR primer Lib148 |
| | | (SEQ ID No.: 147) and Lib149 (SEQ ID No.: 148) |
| 146 | Protein | Translation product of the nucleic acid molecule SEQ ID No.: 145 |
| 147 | Nucleic acid | Sequence of the PCR primer Lib148 |
| 148 | Nucleic acid | Sequence of the PCR primer Lib149 |
| 149 | Nucleic acid | DNA fragment which has been amplified by means of the PCR primer Lib149 |
| | | (SEQ ID NO.: 148) and Lib150 (SEQ ID NO.: 151). |
| 150 | Protein | Translation product of the nucleic acid molecule SEQ ID No.: 149 |
| 151 | Nucleic acid | Sequence of the PCR primer Lib150 |
| 152 | Nucleic acid | DNA fragment which has been amplified by means of the PCR primer Lib151 |
| | | (SEQ ID No.: 156) and Lib152 (SEQ ID No.: 157) |
| 153 | Protein | Translation product of the nucleic acid molecule SEQ ID No.: 152 |
| 154 | Nucleic acid | Sequence of the PCR primer Lib151 |
| 155 | Nucleic acid | Sequence of the PCR primer Lib152 |
| 156 | Protein | KBD-B_3_Homo sapiens Desmoplakin_Accession NM_004415 domain B-3 |
| 157 | Protein | KBD-B_4 Homo sapiens Desmoplakin_Accession NM_004415 domain B-4 |
| 158 | Protein | KBD-B_5 Homo sapiens Desmoplakin_Accession NM_004415 domain B-5 |
| 159 | Nucleic acid | KBD-B_6 Homo sapiens Desmoplakin_Accession NM_004415 domain B-5 |
| 160 | Protein | KBD-B_6 Homo sapiens Desmoplakin_Accession NM_004415 domain B-5 |
| 161 | Nucleic acid | Homo sapiens trichoplein, BC004285 |
| 162 | Protein | Homo sapiens trichoplein, BC004285 |
| 163 | Nucleic acid | Homo sapiens Desmoplakin_Accession NM_004415 with nucleic acid exchanges |
| | | at positions around 2715, 8000 and 8000 compared to SEQ ID No.: ID 1 |
| 164 | Protein | Homo sapiens Desmoplakin_Accession NM_004415 with amino acid exchanges |
| | | at positions 905, 2687 and 2688 compared to SEQ ID No.: ID 2 |
| 165 | Nucleic acid | KBD-B_7 Homo sapiens Desmoplakin_Accession NM_004415 domain B-7 |
| 166 | Protein | KBD-B_7 Homo sapiens Desmoplakin_Accession NM_004415 domain B-7 |
| 167 | Nucleic acid | KBD-D with N-terminal histidine anchor, H. sapiens plakophilin 1a Accession |
| | | NM_001005337 |
| 168 | Protein | KBD-D with N-terminal histidine anchor, H. sapiens plakophilin 1a Accession |
| | | NP_001005337 |
| 169 | Nucleic acid | KBD-D amino acids 1-273 with C-terminal histidine anchor, H. sapiens plakophilin |
| | | 1a Accession NM_001005337 |
| 170 | Protein | BD-D amino acids 1-273 with C-terminal histidine anchor, H. sapiens plakophilin |
| | | 1a Accession NP_001005337 |
| 171 | Nucleic acid | Sequence of the PCR primer HRe6 |
| 172 | Nucleic acid | Sequence of the PCR primer HRe9 |
| 173 | Nucleic acid | Sequence of the PCR primer HRe7 |
| 174 | Nucleic acid | Sequence of the PCR primer HRe8 |
| 175 | Nucleic acid | Sequence of the PCR primer HRe26 |
| 176 | Nucleic acid | Sequence of the PCR primer HRe27 |

### EXPERIMENTAL EXAMPLES

The following examples are disclosed in order to illustrate preferred embodiments of the present invention. These examples are not to be regarded as being exhaustive or limiting the subject matter of the invention.

In the experimental description, the following abbreviations are used:

(2-amino-2-methylpropanol) AMP, (degrees Celsius) ° C., (ethylenediaminetetraacetic acid) EDTA, (1,1-difluoroethane) HFC 152, (international Nomenclature of Cosmetic Ingredients) INCI, (milliliters) ml, (minutes) min, (oil/water) O/W, (polyethylene glycol) PEG-25, (paraminobenzoic acid) PABA, (parts per million) ppm, (quantum satis) q.s., (vinylpyrrolidone) VP, (water/oil) W/O, (active ingredient) Al, (polyvinylpyrrolidone) PVP, keratin-binding domain (KBD), keratin-binding domain B of human desmoplakin (KBD-B), keratin-binding domain C of human desmoplakin (KBD-C).

### Example 1

### Expression Vectors and Production Strains

Various expression vectors were tested for the expression of the keratin-binding domains (KBD). For this, various promoters were used (e.g. IPTG-inducible, rhamnose-inducible, arabindose-inducible, methanol-inducible, constitutive promoters, etc.). Constructs were likewise tested in which the KBD were expressed as fusion Proteins (e.g. as fusion with thioredoxin, or eGFP, or YaaD [B. subtilis, SWISS-PROT: P37527, PDX1], etc.). Here, both the described KBD-B (keratin-binding domain B, SEQ ID No.: 4), and KBD-C (keratin-binding domain C, SEQ ID No.: 10), and the combination of the two domains KBD-BC were expressed using the various expression systems. The vector constructs mentioned are nonlimiting for the claim.

Given by way of representation as an example is the vector map of the IPTG-inducible vector pQE30-KBD-B (FIG. 1), of the methanol-inducible vectors pLib15 (FIG. 2) and pLib16 (FIG. 3), and of the inducible vector pLib19 (FIG. 4). The procedure for KBD-C may also be analogous to the described vector constructions and expressions.

For the expression of the KBD, various production hosts were used, such as, for example, E. coli strains (see Ex. 2; e.g. XL10-Gold [Stratagene], BL21-CodonPlus [Stratagene], and others). However, other bacterial production hosts, such as, for example, Bacillus megaterium or Bacillus subtilis, were also used. In the case of the KBD expression in B. megaterium, the procedure was carried out analogously to: Barg, H., Malten, M. & Jahn, D. (2005). Protein and vitamin production in Bacillus megaterium. In Methods in Biotechnology-Microbial Products and Biotransformations (Barredo, J.-L., ed, 205-224).

The fungal production strains used were Pichia pastoris (see Ex. 3; e.g. GS115 and KM71 [both from Invitrogen]; and others) and Aspergillus nidulans (see Ex. 4; e.g. RMS011 [Stringer, M A, Dean, R A, Sewall, T C, Timberlake, W E (1991) Rodletless, a new Aspergillus developmental mutant induced by direct gene activation. Genes Dev 5:1161-1171] und SRF200 [Karos, M, Fischer, R (1999) Molecular characterization of HymA, an evolutionarily highly conserved and highly expressed Protein of Aspergillus nidulans. Mol Genet Genomics 260:510-521], and others). However, it is also possible to use other fungal production hosts, such as, for example, Aspergillus niger (KBD expression analogous to EP 0635574A1 and/or WO 98/46772) for the KBD expression.

### Example 2

### KBD Expression in E. coli Strains with IPTG Inducible Promoters, e.g. by the Expression Plasmid pQE30-KBD-B

For the expression, various production hosts were used, such as, for example, various E. coli strains (e.g. XL10-Gold [Stratagene], BL21-CodonPlus [Stratagene], and others), Bacillus megaterium, Bacillus subtilis etc.

Described here—by way of representation as an example—is the cloning and expression of KBD-B by E. coli, transformed with pQE30-KBD-B:

Cloning of pQE30-KBD-B
Lambda-MaxiDNA (DNA-Lambda Maxi Kit, Qiagen) was prepared from a cDNA bank of human keratinocytes (BD Bioscience, Clontech, Human Keratinocyte cDNA, foreskin, primary culture in log phase, vector λgt11)
The PCR was carried out using the following oligonucleotides:

| | |
|---|---|
| Bag 43 | |
| (5′-GGTCAGTTACGTGCAGCTGAAGG-3′) | (SEQ ID No.: 141) |
| and | |
| Bag 44 | |
| (5′ GCTGAGGCTGCCGGATCG-3′) | (SEQ ID No.: 142) |

### 50 μl PCR Mixture:

| | | |
|---|---|---|
| 10x PCR buffer Pfu Ultra High Fidelity: | 5 | μl |
| Lambda DNA (744 ng/μl) | 1 | μl (1:30 dilution) |
| dNTP's.-Mix (10 mM) | 1 | μl |
| Oligo Bag 43 (192 ng/μl) | 0.5 | μl |
| Oligo Bag 44 (181 ng/μl) | 0.5 | μl |
| Pfu Ultra High Fidelity Polymerase | 1 | μl |
| H₂O | 41 | μl |

### Temperature Program:

$$30 \times \{ \begin{matrix}2 min & - 95 ° C . \\ 30 sec & - 95 ° C . \\ 30 sec & - Gradient 50 ° C , . -> 60 ° C . \\ 2 min 30 sec & - 72 ° C . \\ 10 min & - 72 ° C .\end{matrix}$$

The resulting PCR product about 1102 bp in size was cut out of an agarose gel and purified.
Using the purified PCR product as template, a 2nd PCR was then carried out:
Oligonucleotides used:

(SEQ ID No.: 143)

| | |
|---|---|
| Bag 53: | (5′-CGCGCCTCGAGCCACATACTGGTCTGC-3′) |
| and | |

(SEQ ID No.: 144)

| | |
|---|---|
| Bag 51 | (5′-GCTTAGCTGAGGCTGCCGGATCG-3′) |

### 50 μl PCR Mixture:

| | | |
|---|---|---|
| 10x PCR buffer TAQ: | 5 | μl |
| Template from above PCR | 3.5 | μl |
| dNTP's.-Mix (10 mM) | 1 | μl |
| Oligo Bag 53 (345 ng/μl) | 0.5 | μl |
| Oligo Bag 51 (157 ng/μl) | 0.5 | μl |
| TAQ Polymerase | 1 | μl |
| H₂O | 39 | μl |

### Temperature Program:

$$30 \times \{ \begin{matrix}2 min & - 95 ° C . \\ 30 sec & - 95 ° C . \\ 30 sec & - 58 ° C . \\ 3 min & - 72 ° C . \\ 10 min & - 72 ° C .\end{matrix}$$

The resulting PCR product about 1073 bp in size was cut out of an agarose gel, purified and cloned in the following vector: pCR2.1-TOPO (Invitrogen).
The resulting vector pCR2.1-TOPO+KBD-B (5027 bp) was then transformed, amplified in E. coli, then cleaved with XhoI and EcoRI and the resulting KBD-B fragment was cloned in pBAD/HisA (Invitrogen; likewise cleaved with XhoI and EcoRI).
The newly formed vector pBAD/HisA+KBD-B (5171 bp) was again cleaved with SacI and StuI and the resulting KBD-B fragment was cloned in pQE30 (Qiagen; cleaved with SacI and SmaI). The resulting expression vector pQE30-KBD-B (4321 bp; see also FIG. 1) was used for the following KBD-B expressions.

The KBD-B (SEQ ID No.: 4) expressed by the vector pQE30-KBD-B in E. coli additionally included, on the N-terminus, the amino acids MRGSHHHHHHGSACEL, and, on the C-terminus, the amino acids CVDLQPSLIS (SEQ ID No.: 166).

Expression of KBD-B by pQE30-KBD-B in E. coli
Precultures were inoculated from plate or glycerol culture with pQE30-KBD-B transformed E. coli strains (e.g. XL10-Gold [Stratagene]) Depending on the size of the main culture, inoculation with LB medium (about 1-100) was carried out in a tube or a small flask.
Antibiotics were used according to the strain used (for pQE30-KBD-B ampicillin 100 μg/ml).
Incubation was carried out at 250 rpm and 37° C.
The main culture was inoculated about 1:100 with preculture, main culture: LB medium or suitable minimal medium with the respective antibiotics. Incubation at 250 rpm and 37° C.
Induction was carried out with 1 mM IPTG above an OD (600 nm) of 0.5.
After induction for 4 h, the cells were centrifuged off.

In fermenters the procedure was analogous, although it was possible to carry out induction at much higher OD units and thus to considerably increase the cell and Protein yield.

### Example 3

### Intracellular and Secretory Expression of KBD by Means of Pichia pastoris Strains Using Methanol-Inducible Promoters, e.g. Through the Expression Plasmids pLib 15 and pLib 16 (Shaking Flask)

For the KBD expression, various Pichia pastoris strains were used, such as, for example, GS115 and KM71 (Pichia Expression Kit, Version M; Invitrogen Life Technologies).

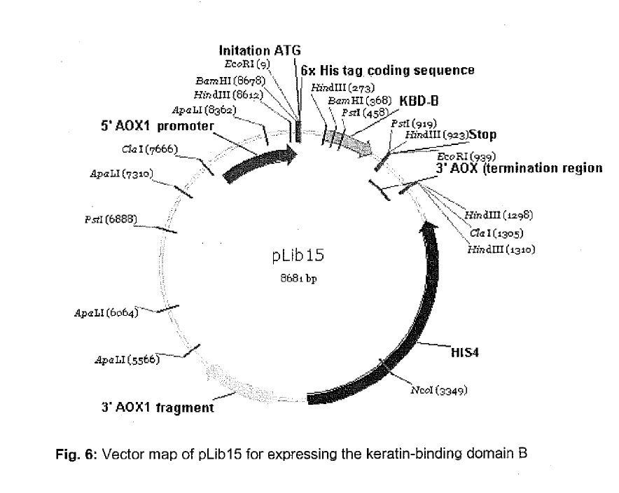

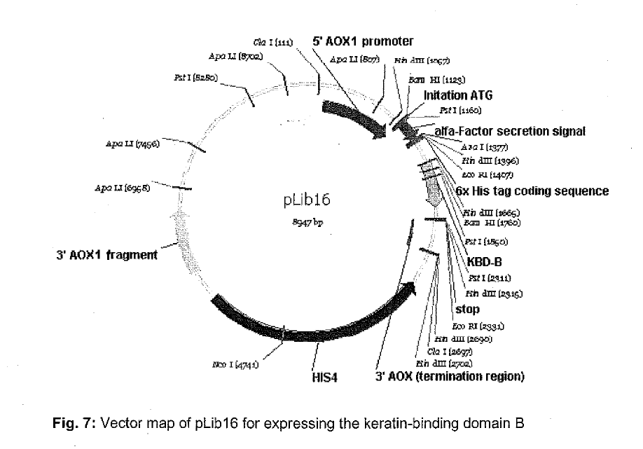

Described here is—by way of representation as an example—the expression of KBD-B by P. pastoris, transformed with pLib15 (intracellular expression, vector see FIG. 6) or pLib16 (secretory expression, vector see FIG. 7).

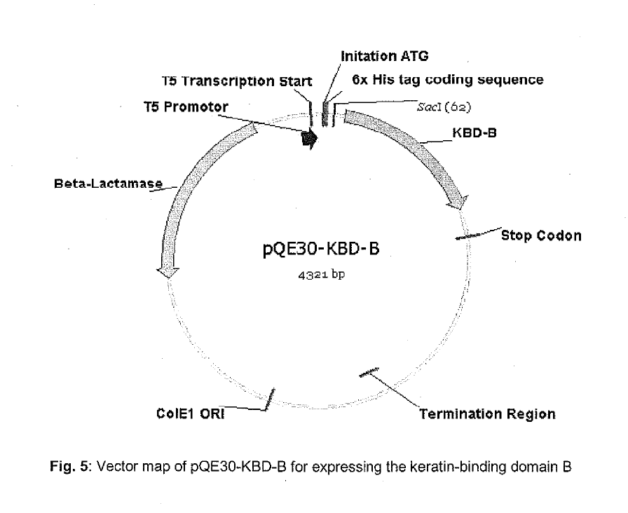

For the construction of pLib15, a KBD-B-encoding DNA fragment (SEQ ID No.: 145) 948 bp in size was amplified by means of PCR using the oligonucleotides Lib148
(5′-GCTAAGGAATTCACCATGCATCACCATCACCATCACGAGCCACATACTGGTC TGCT-3′ (SEQ ID No.: 147)) and Lib149
(5′-GCTGGAGAATTCTCAGCTAATTAAGCTTGGCTGCA-3′ (SEQ ID No.: 148)), and the vector pQE30-KBD-B (Example 2, FIG. 5) as templates. Here, EcoRI restriction sites were introduced at both ends of the PCR products.
For the construction of pLib16, a KBD-B-encoding DNA fragment (SEQ ID No.: 149) 942 bp in size was amplified by means of PCR using the oligonucleotides Lib149 (5′-GCTGGAGAATTCTCAGCTAATTAAGCTTGGCTGCA-3′ (SEQ ID No.: 148)) and Lib150 (5′-GCTAAGGAATTCCATCACCATCACCATCACGAGCCACATACTGGTCTGCT-3′ (SEQ ID No.: 151)) and the vector pQE30-KBD-B (Example 2, FIG. 5) as templates. Here, EcoRI restriction sites were introduced at both ends of the PCR products.
The PCR was carried out in 50 μl reaction mixtures which had the following composition:
1 μl plasmid-DNA pQE30-KBD-B
1 μl dNTP-Mix (each 10 mM; Eppendorf)
5 μl 10×PCR buffer+MgCl₂ (Roche)
1 μl Lib148 or Lib150 5′ primer (corresponds to 50 pmol)
1 μl Lib149 3′ primer (corresponds to 50 μmol)
5 U Pwo polymerase (Roche)
The PCR reactions were carried out under the following cycle conditions:
Step 1: 5 minutes at 95° C. (denaturation)
Step 2: 45 seconds at 95° C.
Step 3: 45 seconds at 50° C. (annealing)
Step 4: 2 minutes at 72° C. (elongation)
30 cycles of steps 2-4
Step 5: 10 minutes at 72° C. (post-elongation)
Step 6: 4° C. (pause)
The PCR product which was amplified with the oligonucleotides Lib148/Lib149 (SEQ ID No.: 145) was digested with EcoRI and ligated into the EcoRI-cleaved vector pPIC3.5 (Pichia Expression Kit, Version M, Invitrogen). The correct KBD-B amplification was checked by sequencing the vector pLib15 (FIG. 6) resulting from the ligation.
The PCR product which was amplified with the oligonucleotides Lib149/Lib150 (SEQ ID No.; 149) was digested with EcoRI and ligated into the EcoRI-cleaved vector pPIC9 (Pichia Expression Kit, Version M, Invitrogen). The correct KBD-B amplification was checked by sequencing the vector pLib16 (FIG. 7) resulting from the ligation.
Electrocompetent cells and spheroplasts of the P. pastoris strains were transformed with the circular and StuI-linearized vectors pLib15 and pLib16 according to the manufacturer's instructions (Pichia Expression Kit, Version M, Invitrogen).
The transformants were analyzed by means of PCR and Southern Blot using chromosomal DNA.
For the proculture, KBD-expressing P. pastoris transformants were inoculated from plate or glycerol culture. Depending on the size of the main culture, inoculation with MGY, BMG or BMGY medium (Pichia-Expression-Kit, Version M, Invitrogen) (about 1:100) was carried out in a tube or a small flask.
The culture was incubated at 250-300 rpm and 30° C. until OD₆₀₀=2-6.
The cells were harvested with 1500-3000×g for 5 min at room temperature
For the main culture, the harvested cell pellet was taken up at an OD₆₀₀=1 in methanol-comprising mM, BMM or BMMY medium (Pichia-Expression-Kit, Version M, Invitrogen) in order to induce the expression.
The main culture was incubated at 250-300 rpm and 30° C. for 1-96 h.
The induction was maintained every 24 h by adding 100% methanol at a methanol end concentration of 0.5%.
In the case of intracellular expression, the harvesting and disruption of the cells was carried out after the end of the main culture by means of a Menton-Gaulin.
In the case of secretory expression, the culture supernatant was collected and the KBD-B was purified from it directly.
The KBD-B expressed intracellularly in P. pastoris (SEQ ID No.: 145) (pLib15) included, besides the polypeptide sequence SEQ ID No.: 4, additionally, at the N-terminus, the amino acids MHHHHHH, and, at the C terminus, the amino acids GVDLQPSLIS.
The KBD-B expressed secretorily in P. pastoris (SEQ ID No.: 149) (pLib16) included, prior to processing, besides the polypeptide sequence SEQ ID No.: 4, additionally at the N-terminus the amino acids MRFPSIFTAVLFAASSALAAPVNTTTEDETAOIPAEAVIGYSDLPGDFDVAVLPFS NSTNNGLLFINTTIASIAAKEEGVSLEKREAEAYVEFHHHHHH, and, at the C-terminus, the amino acids GVDLQPSLIS.
The KBD-B processed and secreted by means of P. pastoris (SEQ ID No.: 149) (pLib16) included, besides the polypeptide sequence SEQ ID No.: 4, additionally at the N-terminus the amino acids YVEFHHHHHH, and at the C-terminus the amino acids GVDLQPSLIS.

### Example 4

### Expression of KBD by Means of Aspergillus nidulans Strains Using the Inducible alcA Promoter, e.g. Through the Expression Plasmid pLib 19 (Shaking Flask)

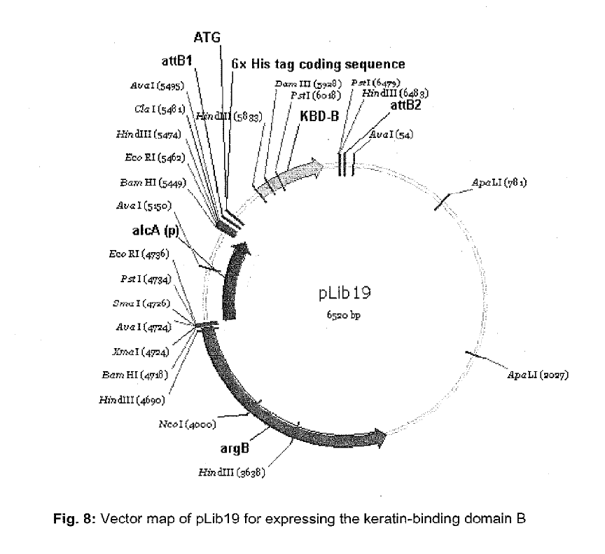

For the expression, A. nidulans wild type strains were used, such as, for example, RMS011 or SRF200. Described here is—by way of representation as an example—the expression of KBD-B by A. nidulans, transformed with pLib19 (FIG. 8).
For the construction of pLib19, a KBD-B-encoding DNA fragment 922 bp in size (SEQ ID No.: 152) was amplified by means of PCR using the oligonucleotides Lib151
(5′-CACCATGCATCACCATCACCATCACGAGCCACATACTGGTCTGCT-3′ (SEQ ID No. 154)) and Lib152 (5′-GCTAATTAAGCTTGGCTGCA-3′ (SEQ ID No.: 155)), and the vector pQE30-KBD-B (Example 2, FIG. 5) as template (using the abovementioned PCR conditions, with the annealing temperature of the PCR program of 53° C. being adapted to the Tm values of the primers Lib151 and Lib152). The PCR product was ligated into the vector pENTRID (pENTR™ Directional TOPO® Cloning Kit, Version E, Invitrogen). The correct KBD-B amplification was checked by sequencing.
The recombination of the KBD-B encoding DNA fragment was carried out into the vector pMT-OvE (Toews M W, Warmbold J, Konzack S, Rischitor P, Veith D, Vienken K, Vinuesa C, Wei H, Fischer R; Establishment of mRFP1 as a fluorescent marker in Aspergillus nidulans and construction of expression vectors for high-throughput Protein tagging using recombination in vitro (GATEWAY). (2004) Curr Genet. 45: 383-389) using the “Gateway® LR Clonase™ enzyme mix” (Invitrogen). This produced the vector pLib19 (FIG. 8).
Protoplasts of the A. nidulans wild type strains were transformed with the circular vector pLib19 (Yelton M M, Hamer J E, Timberlake W E; Transformation of Aspergillus nidulans by using a trpC plasmid., (1984) Proc Natl Acad Sci USA 81: 1479-1474). The transformants were analyzed by means of PCR and Southern Blot using chromosomal DNA.
For the preculture of KBD-B-expressing A. nidulans transformants, 100 ml of minimal medium (0.6% NaNO₃; 0.152% KH₂PO₄_{^{−}}, 0.052% KCl [pH 6.5]; 0.8% glucose; 0.05% MgSO₄; 1 ml trace element solution [1 g/l FeSO₄33 7H₂O; 8.8 g/l ZnSO₄×7H₂O; 0.4 g/l CuSO₄×5H₂O; 0.15 g/l MnSO₄×4H₂O; 0.1 g/l Na₂B₄O₇×10H₂O; 0.05 g/l (NH₄)₆Mo₇O₂₄×4H₂O], +strain-specific supplements) or 100 ml of complete medium (2% malt extract; 0.1% peptone; 2% glucose; +strain-specific supplements) were inoculated in 500 ml flasks with 10⁶-10⁷ spores and incubated for 16-24 h at 200-250 rpm and 37° C.
After the preculture, the fungal mycelium was harvested by filtration, washed with distilled water and transferred to flasks with 100-500 ml of fresh minimal medium. In this main culture medium, 0.1% fructose was used instead of glucose as the C-source. To induce the KBD expression, ethanol (1% final concentration) or glycerol (50 mM) or sodium acetate (50 mM) or ethylamine or threonine were additionally added to the medium. The additives mentioned for inducing the expression are not limiting for the claim. The main culture was incubated for a further 5-48 h at 200-250 rpm and 37° C.
After the end of the culture, the fungal mycelium was harvested with 1500-3000×g for 5 min at room temperature and disrupted by means of a Menton-Gaulin.
Besides the polypeptide sequence SEQ ID No. 4, the KBD-B expressed in A. nidulans (SEQ ID No.: 152) (pLib19) additionally included, at the N-terminus, the amino acids MHHHHHH, and, at the C-terminus, the amino acids GVDLQPSLISKGGRADPAFLYKVVMIRLLTKPERKLLEGGPGTQLLFPLVRVNCA LGVIMVIAVSCVKLLSAHNSTQHTSRKHKV.

### Example 5

### Cell Disruption and Inclusion Body Purification (pQE30-KBD-B)

Solubly expressed KBD could be used directly following cell disruption (e.g. by means of Menton-Gaulin) or be purified by means of chromatography (see Example 6). Insolubly expressed KBD (e.g. in inclusion bodies) was purified as follows:
The fermenter contents were centrifuged, the pellet was suspended in 20 mM phosphate buffer pH=7.5 and disrupted by means of a Menton-Gaulin.
The disrupted cells were centrifuged again (15 000 g), the pellet from this was treated with 20 mM phosphate, 500 mM NaCl and 8 M urea and so stirred. (Dissolution of the Inclusion Bodies)
The pH of the supernatant was adjusted to 7.5.
Centrifugation was then carried out again and the supernatant was applied to an Ni chelate Sepharose column and purified as described in Example 6.

### Example 6

### Purification of Keratin-Binding Domain B on Ni Chelate Sepharose

The KBD could be purified chromatographically through the attached His tag over an Ni column.

Column material: Ni-Sepharose High Performance
Amersham Biosciences order No.: 17-5268-02

The material was packed into a column (e.g. diameter 2.6 cm, height 10 cm) and equilibrated with buffer A+4% buffer B (corresponds to 20 mM imidazole).

The Protein extract (see e.g. cell disruption and inclusion body purification) was applied to the column at pH 7.5 using a Superloop (ÄKTA system) (flow about 5 ml/min).

Following application, washing was carried out with buffer A+20 mM imidazole. Elution was carried out with buffer B (500 mM imidazole in buffer A).

The eluate was collected in fractions using a fraction collector.

The eluate was then freed from salt (advantageous for samples which are to be concentrated). For this, the eluate was freed from salt, for example, over a Sephadex G25 medium column (Amersham). Then, for the concentration, for example an Amicon chamber (stirred ultrafiltration cell, Millipore) could.

### Buffer A:

20 mM sodium dihydrogenphosphate
500 mM NaCl (if desired, it is also possible to use buffer with lower NaCl concentrations)
8 M urea (urea does not need to be used if “active” KBD is chromatographed which has already been solubly expressed.
   Without urea, no subsequent renaturation of the Protein is required.)
pH=7.50

### Buffer B:

20 mM sodium dihydrogenphosphate
500 mM NaCl (if desired, it is also possible to use buffers with lower NaCl concentrations)
8 M urea
500 mM imidazole
pH=7.50

### Example 7

### Renaturation of Keratin-Binding Domain B

Insolubly expressed keratin-binding domain (e.g. from inclusion bodies) can be renatured and thus activated as follows:

### Method 1: Discontinuous Dialysis

6.5 ml of Cellytc IB (Sigma, order No. C5236) and 5 mM DTT were added to 6.5 ml of KBD-B inclusion bodies in 8 M urea (Ni chelate eluate, HiTrap). The solution to be renatured was then poured into a dialysis tube (Spectrum: Spectra Por MWCO: 12-14 kD).

Carry out dialysis for about 12 hours against 1 l 6 M urea solution at 4° C. with careful stirring.

500 ml of 25 mM Tris/HCl pH=7.50 were added and dialysis was carried out like this for 9 hours at 4° C. Subsequent addition of a further 250 ml of the Tris buffer (see above) and dialysis for a further 12 hours.

500 ml of 25 mM Tris/HCl pH=7.50 were then added again and dialysis was carried out like this for 9 hours at 4° C. Subsequent addition of a further 250 ml of the Tris buffer (see above) and dialysis for a further 12 hours.

500 ml of 25 mM Tris/HCl pH=7.50 were then added again and dialysis was carried out like this for 9 hours at 4° C. The dialysis tube containing the dialyzate was then placed into 2 l: 25 mM Tris+150 mM NaCl pH=7.50. Dialysis was then carried out again at 4° C. for 12 hours.

The contents of the dialysis tube were then removed.

### Method 2: Continuous Dialysis

20 ml of KBD-B inclusion bodies in 8 M urea (Ni chelate eluate, HiTrap) were treated with 10 ml of Cellytic IB (Sigma, order No. C5236) and 5 mM DTT. The solution was then poured into a dialysis chamber: Slide-A-Lyzer Dialyses Cassette PIERCE, MWCO: 10 kD. Order No.: 66830.

Dialysis was then carried out for about 1 hour against 1 l 6 M urea solution at 4° C.

Then, over a period of 48 h, 2 l of the following buffer were metered in continuously by means of a peristaltic pump: 25 mM Tris/HCl pH=7.5.

The dialysis tube containing the dialyzate was then added to 2 l of the end buffer:

25 mM Tris+150 mM NaCl pH=7.50 and dialysis was carried out for about 12 hours at 4° C.

The contents of the dialysis tube were then removed.

### Example 8

### Binding to Skin 1 (Qualitative)

A visual qualitative test was developed in order to examine whether KBD binds to skin.

### Solutions Used:

Blocking solution: Western Blocking Reagent 1921673 Roche (10× solution) diluted in TBS.

### TBS: 20 mM Tris; 150 mM NaCl pH 7.5

### TTBS: TBS+0.05% Tween20

The first step is the transfer of the outer keratin layer of the skin to a stable support. For this purpose, a transparent adhesive tape was firmly applied to depilated human skin and removed again. The test can be carried out directly on the transparent adhesive tape, or the adhering keratin layer can be transferred to a glass slide through renewed adhesion. Binding was demonstrated as follows:
For incubation with the various reagents, transfer to a Falcon vessel
If appropriate addition of ethanol for degreasing, removal of ethanol and drying of the slide
Incubation with blocking buffer for 1 h at room temperature
2× washing for 5 min with TTBS
1× washing for 5 min with TBS
Incubation with the KBD to be tested (coupled to tag—e.g. His₆, HA etc.) or control Protein in TBS/0.05% Tween 20 for 2-4 h at room temperature
Removal of the supernatant
3× washing with TBS
Incubation for 1 h at room temperature with monoclonal anti-polyHistidine (or specific KBD rabbit) antibodies, diluted 1:2000 in TBS+0.01% blocking
2× washing for 5 min with TTBS
1× washing for 5 min with TBS
Incubation for 1 h at room temperature with anti-mouse IgG alkaline-phosphatase conjugate, diluted 1:5000 in TBS+0.01% blocking
2× washing for 5 min with TTBS
1× washing for 5 min with TBS
Addition of phosphatase substrate (NBT-BCIP; Boehringer MA 1 tablet/40 ml of water 2.5 min; stop: with water)
Optical detection of the colored precipitate with the naked eye or using a microscope. A blue colored precipitate indicates that KBD has bound to the skin.

### Example 9

### Binding to Skin 2 (Quantitative)

A quantitative test was developed with which the hair/skin binding strength of the KBD can be compared with nonspecific Proteins.

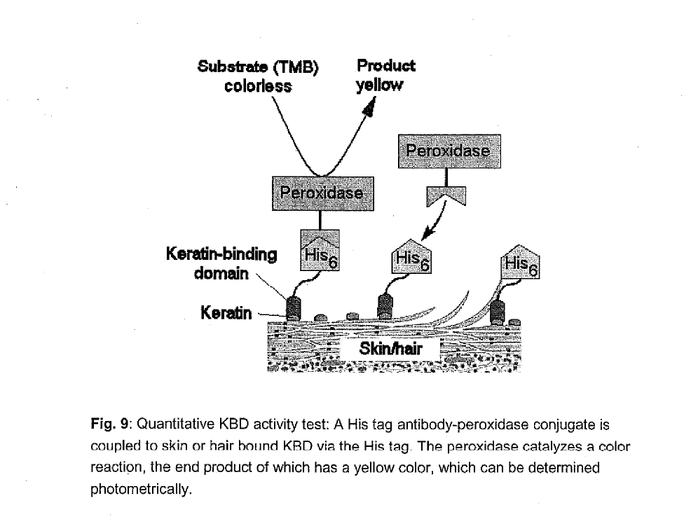

A 5 mm cork borer was used to bore a section out of a thawed dry piece of skin without hair (human or pig) (or in the case of a surface test a section of skin is inserted into a Falcon lid). The sample of skin was then converted to a thickness of 2-3 mm in order to remove any tissue present. The skin sample was then transferred to an Eppendorf vessel (Protein low-bind) in order to carry out the binding demonstration (see also FIG. 9; alternatively, the Episkin system [reconstituted human skin] from L'Oreal can also be used):
2× washing with PBS/0.05% Tween 20
Addition of 1 ml of 1% BSA in PBS and incubation for 1 h at room temperature, gentle swinging movements (900 rpm).
Removal of the supernatant
Addition of 100 μg of KBD in PBS with 0.05% Tween 20; incubation for 2 h at room temperature and gentle swinging movements (900 rpm).
Removal of the supernatant
3× washing with PBS/0.05% Tween 20
Incubation with 1 ml of monoclonal mouse anti-tag (His6 or HA or specific KBD) antibodies with peroxidase conjugate (1:2000 in PBS with 0.05% Tween 20) [Monoclonal AntipolyHistidine Peroxidase Conjugate, produced in mouse, lyophilized powder, Sigma] for 2-4 h at room temperature, gentle swinging movement (900 rpm)
3× washing with PBS/0.05% Tween 20
Addition of peroxidase substrate (1 ml/Eppendorf vessel; composition see below)
Allow reaction to run until blue coloration (about 90 seconds).
Stop the reaction with 100 μl of 2 M H₂SO₄.
The absorption was measured at 405 nm.

### Peroxidase Substrate (Prepare Shortly Beforehand):

0.1 ml TMB solution (42 mM TMB in DMSO)
+10 ml substrate buffer (0.1 M sodium acetate pH 4.9)
+14.7 μl H₂O₂ 3% strength

### Example 10

### Binding to Hair (Quantitative)

In order to be able to demonstrate the binding strength of KBD to hair also relative to other Proteins, a quantitative assay was developed (see also FIG. 9). In this test, hair was firstly incubated with KBD and excess KBD was washed off. An antibody-peroxidase conjugate was then coupled via the His tag of the KBD. Nonbound antibody-peroxidase conjugate was washed off again. The bound antibody-peroxidase conjugate [Monoclonal AntipolyHistidine Peroxidase Conjugate, produced in mouse, lyophilized powder, Sigma] can convert a colorless substrate (TMB) into a colored product, which can be measured photometrically at 405 nm. The intensity of the absorption indicates the amount of bound KBD or comparison Protein. The comparison Protein chosen was, for example, YaaD from B. subtilis, which likewise had—as is necessary for this test—a His tag for the detection. Instead of the His tag, other specific antibodies conjugated with peroxidase can also be used.

5 mg of hair (human) are cut into sections 5 mm in length and transferred to Eppendorf vessels (Protein low-bind) in order to carry out the binding demonstration:
Addition of 1 ml of ethanol for degreasing
Centrifugation, removal of ethanol and washing of the hair with H₂O
Addition of 1 ml of 1% BSA in PBS and incubation for 1 h at room temperature, gentle swinging movements.
Centrifugation, removal of the supernatant
Addition of the keratin-binding domains to be tested (coupled to tag—e.g. His₆, HA etc.) or control Protein in 1 ml of PBS/0.05% Tween 20; incubation for 16 h at 4° C. (or at least 2 h at room temperature) with gentle swinging movements.
Centrifugation, removal of the supernatant
3× washing with PBS/0.05% Tween 20
Incubation with 1 ml monoclonal mouse anti-tag (His6 or HA) antibodies with peroxidase conjugate (1:2000 in PBS/0.05% Tween 20) [Monoclonal AntipolyHistidine Peroxidase Conjugate, produced in mouse, lyophilized powder, Sigma] for 2-4 h at room temperature, gentle swinging movement
3× washing with PBS/0.05% Tween 20
Addition of peroxidase substrate (1 ml/Eppendorf vessel)
Allow reaction to run until blue coloration (about 2 minutes).
Stop the reaction with 100 μl of 2 M H₂SO₄.
The absorption is measured at 405 nm.

### Peroxidase Substrate (Prepare Shortly Beforehand):

0.1 ml TMB solution (42 mM TMB in DMSO)
+10 ml substrate buffer (0.1 M sodium acetate pH 4.9)
+14.7 μl H₂O₂ 3% strength
BSA=bovine serum albumin
PBS=phosphate buffered salt solution
Tween 20=polyoxyethylene sorbitan monolaureate, n about 20
TMB=3,5,3′,5′-tetramethylbenzidine

A binding test on hair carried out by way of example for KBD-B demonstrated considerable superiority of the binding of KBD-B (SEQ ID No.: 166) to hair compared with significantly poorer binding of the comparison Protein YaaD:

TABLE 4
Quantitative KBD activity test Hair: 1) buffer; 2) comparison Protein
YaaD; 3) KBD-B denatured; 4) KBD-B renatured. The table shows
the measured absorption values at 405 nm.

| | | |
|---|---|---|
| 1 | Buffer | A_{405 nm} = 0.000 |
| 2 | Comparison Protein YaaD | A_{405 nm} = 0.088 |
| 3 | KBD-B denatured | A_{405 nm} = 0.254 |
| 4 | KBD-B renatured | A_{405 nm} = 1.591 |

### Example 11

### Expression of KBD-D (SEQ ID No.: 167) by Means of Escherichia coli Strains Using the Expression Plasmid pRee024 with an IPTG Inducible Promoter (FIG. 8)

For the expression, the E. coli strain XL 10 Gold [Stratagene] was used.

Described here, by way of representation as an example, is the cloning of KBD-D (SEQ ID No.: 167) and the subsequent expression of the KBD-D Protein (SEQ ID No.:168) in E. coli, transformed with pRee024 (FIG. 8):

Cloning of pRee024:
Lambda-MaxiDNA (DNA-Lambda Maxi Kit, Qiagen) was prepared from a cDNA bank of human keratinocytes (BD Bioscience, Clontech, Human Keratinocyte cDNA, foreskin, primary culture in log phase, vector: λgt11).

The PCR for the amplification of the KBD-D gene was carried out in two steps. Firstly, the 5′ end and 3′ end were amplified independently. These fragments were the matrix for the amplification of the entire KBD-D gene.

The PCR for the amplification of the 5′ end was carried out as follows:

The primers had the following sequence:

(SEQ ID No.: 171)

| | |
|---|---|
| HRe6: | 5′-ATGAACCACTCGCCGCTCAAGACCGCCTTG-3′ |

(SEQ ID No.: 172)

| | |
|---|---|
| HRe9: | 5′-CGTTCCCGGTTCTCCTCAGGAGGCTGACTG-3′ |

### 100 μl PCR Mixture:

| | | |
|---|---|---|
| 10x PCR buffer Pfu Ultra High Fidelity: | 10 | μl |
| Lambda DNA (744 ng/μl) | 1 | μl (1:10 dilution) |
| dNTP's.-Mix (10 mM) | 10 | μl |
| HRe6 (196 ng/μl) | 1 | μl |
| HRe9 (201 ng/μl) | 1 | μl |
| Pfu Ultra High Fidelity Polymerase | 1 | μl |
| H₂O bidistilled | 76 | μl |

### Temperature Program:

$$30 \times \{ \begin{matrix}2 min & 95 ° C . \\ 30 sec & 95 ° C . \\ 30 sec & 63 ° C . \\ 1 min 30 sec & 72 ° C .\end{matrix}$$

A fragment approximately 1 kb in size was detected in the agarose gel. The reaction was purified and used below as 5′ end template for the amplification of the KBD-D gene.

The PCR for the amplification of the 3′ end was carried out as follows:

The primers had the following sequence:

(SEQ ID No.: 173)

| | |
|---|---|
| HReY: | 5′-TTAGAATCGGGAGGTGAAGTTCCTGAGGCT-3′ |

(SEQ ID No.: 174)

| | |
|---|---|
| HReB: | 5′-CACCACCAACAAGCTGGAGACCCGGAG-3′ |

### 100 μl PCR Mixture:

| | | |
|---|---|---|
| 10x PCR buffer Pfu Ultra High Fidelity: | 10 | μl |
| Lambda DNA (744 ng/μl) | 1 | μl (1:10 dilution) |
| dNTP's.-Mix (10 mM) | 10 | μl |
| HRe7 (201 ng/μl) | 1 | μl |
| HRe8 (209 ng/μl) | 1 | μl |
| Pfu Ultra High Fidelity Polymerase | 1 | μl |
| H₂O bidistilled | 76 | μl |

### Temperature Program:

$$30 \times \{ \begin{matrix}2 min & 95 ° C . \\ 30 sec & 95 ° C . \\ 30 sec & 63 ° C . \\ 1 min 30 sec & 72 ° C .\end{matrix}$$

A fragment approximately 1.2 kb in size was detected in the agarose gel. The reaction was purified and used below as 3′ end template for the amplification of the KBD-D gene.
For the amplification of the KBD-D gene, the 5′ end template and the 3′ end template were used as matrix. The PCR was carried out as follows;

### 100 μl PCR Mixture:

| | | |
|---|---|---|
| 10x PCR buffer Pfu Ultra High Fidelity: | 10 | μl |
| dNTP-mix (10 mM) | 10 | μl |
| H₂O bidistilled | 75 | μl |
| 5′ end template | 1 | μl |
| 3′ end template | 1 | μl |
| Pfu Ultra High Fidelity Polymerase | 1 | μl |
| H₂O | 76 | μl |

### Temperature Program:

$$10 \times \{ \begin{matrix}60 sec & 94 ° C . \\ 300 sec & 72 ° C .\end{matrix}$$

after the 10 cycles, 1 μl of primer HRe6 (196 μg/ml) and HRe7 (206 μg/ml) and 1 μl of Pfu Ultra High Fidelity Polymerase were added and the following temperature program was carried out with the reaction:

### Temperature Program:

$$30 \times \{ \begin{matrix}2 min & 95 ° C . \\ 30 sec & 95 ° C . \\ 30 sec & 63 ° C . \\ 1 min 30 sec & 72 ° C .\end{matrix}$$

Then, 1 μl of Taq polymerase was added and the mixture was incubated for 10 minutes at 72° C.
The resulting PCR product approximately 2150 bp in size was cut out of an agarose gel, purified and cloned in the following vector: pCR2.1-TOPO (Invitrogen).
The resulting vector pRee019 (6112 bp) was then transformed, amplified in E. coli, and the KBD-D gene was checked by a sequencing.

Subsequently, the KBD-D gene was cloned into the expression vector. For this, a further PCR was carried out with the vector pRee019 as template:

### Oligonucleotides Used:

HRe26:

(SEQ ID No.: 175)

5′-CTCGGTACCAACCACTCGCCGCTCAAGACCGCCTTGGCG-3′
HRe27:

(SEQ ID No.: 176)

5′-ATTAAGCTTTTAGAATCGGGAGGTGAAGTTCCTGAGGCT-3′

### 100 μl PCR Mixture:

| | | |
|---|---|---|
| 10x PCR buffer Pfu Ultra High Fidelity: | 10 | μl |
| pRee019 (25 ng/μl) | 1 | μl |
| dNTP's.-Mix (10 mM) | 10 | μl |
| HRe26 (287 ng/μl) | 1 | μl |
| HRe27 (354 ng/μl) | 1 | μl |
| Pfu Ultra High Fidelity Polymerase | 1 | μl |
| H₂O bidistilled | 76 | μl |

### Temperature Program:

$$30 \times \{ \begin{matrix}2 min & 95 ° C . \\ 30 sec & 95 ° C . \\ 30 sec & 79 ° C . \\ 1 min 30 sec & 72 ° C .\end{matrix}$$

A fragment approximately 2.2 kb in size was detected in the agarose gel. The reaction was purified and then cut with the restriction endonucleases KpnI and HindIII; the resulting fragment was cloned into the expression vector. This gave the vector pRee024, which was used subsequently for the KBD-D expression.
   Expression of KBD-D (SEQ ID No.: 167) by pRee024 in E. coli
Precultures were inoculated from plate or glycerol culture with pRee024 transformed E. coli strains (e.g. TG10). Depending on the size of the main culture, inoculation with LB medium (about 1:100) was carried out in a tube or a small flask.
Antibiotics were used according to the strain used (for E. coli transformed with pRee024 TG10 ampicillin 100 μg/ml).
Incubation was carried out at 250 rpm and 37° C.
The main culture was inoculated about 1:100 with preculture, main culture: LB medium or suitable minimal medium with the respective antibiotics. Incubation at 250 rpm and 37° C.
Induction was carried out with 1 mM IPTG above an OD₅₇₈ₙₘ of 1. The incubation temperature was then lowered to room temperature (about 20° C.). The cells were centrifuged off 2 hours after induction. (See FIG. 9)

### Example 12

### Cell Disruption and Inclusion Body Purification (pRee024)

Insolubly expressed KBD-D (SEQ ID No.:168) (e.g. in inclusion bodies) was purified as follows:

The cell sediment from Example 2 was resuspended in 20 mM phosphate buffer with 100 mM NaCl pH=7.5 and disrupted by ultrasound treatment.

The disrupted cells were centrifuged again (4° C., 12 000 g, 20 minutes). The supernatant was discarded. The sediment was dissolved in buffer A (10 mM NaH₂PO₄, 2 mM KH₂PO₄, 100 mM NaCl, 8 M urea, 5 mM DTT). The mixture was then centrifuged again and the supernatant was applied to an Ni chelate Sepharose. Following application, washing was carried out with buffer A and 20 mM imidazole. Elution from the column was carried out with buffer B (10 mM NaH₂PO₄, 2 mM KH₂PO₄, 100 mM NaCl, 8 M urea, 5 mM DTT, 500 mM imidazole). The eluate was collected in fractions and analyzed by means of SDS-PAGE. Fractions which comprised purified KBD-D were renatured as described in Example 13.

### Example 13

### Renaturation of Keratin-Binding Domain D (SEQ ID No.:168)

Insolubly expressed keratin-binding domain D (e.g. from inclusion bodies) could be renatured by dialysis and thus activated. The procedure was as follows:

The fractions from Example 12 which comprised purified KBD-D were poured into a dialysis tube (MWCO 12-14 KD).

Dialysis was then carried out for about 1 hour against 1 l 8 M urea solution.

Then, over a period of 12 hours, 2 l of deionized water were metered in continuously by means of a peristaltic pump.

The contents of the dialysis tube were then removed. The KBD-D activated in this way was used for the following activity tests.

### Example 14

### Qualitative Binding to Skin

A visual qualitative test was used in order to examine whether the KBD-D (SEQ ID No.:168) binds to skin.

### Solutions Used:

Blocking solution: Western Blocking Reagent 1921673 Roche (10× solution) diluted in TBS

### TBS: 20 mM Tris; 150 mM NaCl pH 7.5

### TTBS: TBS+0.05% Tween 20

The first step is the transfer of the outer keratin layer of the skin to a stable support. For this purpose, a transparent adhesive tape was firmly applied to depilated human skin and removed again. The test can be carried out directly on the transparent adhesive tape, or the adhering keratin layer can be transferred to a glass slide through renewed adhesion. Binding was demonstrated as follows:
for incubation with the various reagents, transfer to a Falcon vessel
   if appropriate, addition of ethanol for degreasing, removal of ethanol and drying of the slide
incubation with blocking buffer for 1 h at room temperature
2× washing for 5 min with TTBS
1× washing for 5 min with TBS
incubation with the KBD to be tested (coupled to tag—e.g. His₆, HA etc.) in TBS/0.05% Tween 20 for 2-4 h at room temperature
removal of the supernatant
3× washing with TBS
incubation for 1 h at room temperature with monoclonal mouse anti-tag (His6 or HA) antibodies with peroxidase conjugate (1:2000 in TBS+0.01% blocking) [Monoclonal AntipolyHistidine Peroxidase Conjugate, produced in mouse, lyophilized powder, Sigma]
2× washing for 5 min with TTBS
1× washing for 5 min with TBS
addition of phosphatase substrate (NBT-BCIP, Boehringer MA 1 tablet/40 ml of water 2.5 min; stop: with water)
optical detection of the colored precipitate with the naked eye or using a microscope.

A blue colored precipitate, being a reaction of the antipolyhistidine-AP conjugate interacting with the KBD-D, was visible on the transparent adhesive tape treated with KBD-D. As negative control, a transparent adhesive tape was treated only with buffer. No significant blue coloration could be seen here. These results show that KBD-D has bound to the skin keratin on the transparent adhesive tape.

### Example 15

### Quantitative Binding to Skin and Hair

In order to investigate the binding strength of the KBD-D (SEQ ID No.:168) to skin and hair compared to the KBD-B (SEQ ID No.:166), a quantitative test was carried out. In this test, firstly hair was incubated with KBD-B or KBD-D and excess KBD-B or -D was washed off. An antibody-peroxidase conjugate was then coupled via the His/tag of the KBD-B or -D. Nonbound antibody-peroxidase conjugate was washed off again. The bound antibody-peroxidase conjugate can convert a colorless substrate (TMB) into a colored product, which was measured photometrically at 405 nm. The intensity of the absorption indicates the amount of bound KBD-B or -D.

The test for binding to skin was carried out with human keratinocytes in microtiter plates as follows.
2× washing with PBS/0.05% Tween 20
addition of 1 ml of 1% BSA in PBS and incubation for 1 h at room temperature, gentle swinging movements (900 rpm).
removal of the supernatant
addition of 100 μg of KBD in PBS with 0.05% Tween 20, incubation for 2 h at room temperature and gentle swinging movements (900 rpm).
removal of the supernatant
3× washing with PBS/0.05% Tween 20
incubation with 1 ml of monoclonal mouse anti-tag-His6 antibodies for 2-4 h at room temperature, gentle swinging movement (900 rpm)
3× washing with PBS/0.05% Tween 20
addition of peroxidase substrate (1 ml/Eppendorf vessel; composition see below) reaction until a blue coloration (about 90 seconds).
reaction stopped with 100 μl of 2 M H₂SO₄.
the absorption was measured at 405 nm.

### Peroxidase Substrate (Prepared Shortly Beforehand)

0.1 ml TMB solution (42 mM TMB in DMSO)
+10 ml substrate buffer (0.1 M sodium acetate pH 4.9)
+14.7 μl H₂O₂ 3% strength

In order to characterize the hair binding of the KBD-D compared to the KBD B, the following binding assay was carried out:

5 mg of hair (human) were cut into sections 5 mm in length and transferred to Eppendorf vessels (Protein low-bind).
addition of 1 ml of ethanol for degreasing
centrifugation, removal of ethanol and washing of the hair with H₂O
centrifugation, removal of the supernatant
addition of the keratin-binding domains to be tested (coupled to tag—e.g. His₆, HA etc.) in 1 ml of PBS/0.05% Tween 20; incubation for 2 h at room temperature with gentle swinging movements
centrifugation, removal of the supernatant
3× washing with PBS/0.05% Tween 20
incubation with 1 ml monoclonal mouse anti-tag-(His6 or HA) antibodies with peroxidase conjugate (1:2000 in PBS/0.05% Tween 20) [Monoclonal AntipolyHistidine Peroxidase Conjugate, produced in mouse, lyophilized powder, Sigma] for 2-4 h at room temperature, gentle swinging movement
3× washing with PBS/0.05% Tween 20
addition of peroxidase substrate (1 ml/Eppendorf vessel)
allow reaction to proceed until blue coloration (90 seconds).
stop the reaction with 100 μl of 2 M H₂SO₄.

The absorption was measured at 405 nm

### Peroxidase Substrate (Prepared Shortly Beforehand):

0.1 ml TMB solution (42 mM TMB in DMSO)
+10 ml substrate buffer (0.1 M sodium acetate pH 4.9)
+14.7 pt H₂O₂ 3% strength
BSA=bovine serum albumin
PBS=phosphate buffered salt solution
Tween 20=polyoxyethylene sorbitan monolaureate, n about 20
TMB=3,5,3′,5′-tetramethylbenzidine

TABLE 5a
Quantitative binding of KBD-D or KBD-B to skin. The absorption
values listed are values standardized to the surface (of skin or hair)

| Keratin-binding domain | Absorption at 405 nm |
|---|---|
| KBD-D to skin | 3.69 |
| KBD-D to skin after 10% strength SDS | 3.15 |
| treatment | |
| KBD-B to skin | 0.93 |
| KBD-B to skin after 10% strength SDS | 0.185 |
| treatment | |

TABLE 5b
Quantitative binding of KBD-D to hair. The absorption values listed
are values standardized to the surface

| Keratin-binding domain | Absorption at 405 nm |
|---|---|
| KBD-D to hair | 0.88 |
| KBD-D to hair after 10% strength SDS | 0.62 |
| treatment | |

TABLE 5C
Quantitative binding of KBD-D and KBD-B to skin and hair
after 10% strength SDS treatment in % relative to
the KBD-D and KBD-B untreated hair or skin.

| | Relative absorption |
|---|---|
| | loss after 10% strength |
| Keratin-binding domain | SDS treatment in % |
| KBD-D to skin after 10% strength SDS | 15 |
| treatment | |
| KBD-B to skin after 10% strength SDS | 80 |
| treatment | |
| KBD-D to hair after 10% strength SDS | 30 |
| treatment | |
| KBD-B to hair after 10% strength SDS | 86 |
| treatment | |

These results show that the Protein KBD-D can bind to hair and more strongly to skin (see Tab. 5). In contrast to the KBD-B (SEQ ID No.: 166), the binding of the KBD-D (SEQ ID No.: 168) is only more weakly influenced by a washing with an up to 10% strength SDS solution (see Tab. 5a).

### Example 16

### Testing the Coupling Success (Ellmann's Test)

The success of the effector coupling was monitored via three different tests:
(i) Ellmann's test in which the number of free Cys-SH groups in the Protein before and after the effector coupling can be determined. Here, a considerable reduction in the free SH groups after coupling indicates good reaction progress.
(ii) Activity test in which the binding of the KBD-B (SEQ ID No.: 166) with and without coupled effector to hair can be measured. Good reaction control should not reduce the activity of KBD-effector compared with uncoupled KBD-B (SEQ ID No.: 166) (see Example 17).
(iii) Visible coloring of hair or skin as a result of the successful coupling of the dye to the KBD-R (SEQ ID No.: 166) (see Example 20).

Regarding (iii): The success of the effector coupling was tested as follows using Ellmann's test:

### Materials Required:

Ellmann's reagent: 5,5′-dithiobis(2-nitrobenzoic acid) (DTNB); 4 mg/1 ml in 0.1 M Na phosphate buffer
0.1 M Na phosphate buffer pH 8.0
Cysteine solution (26.3 mg of cysteine hydrochloride monohydrate/100 ml Na phosphate buffer). The solutions were and must only be prepared shortly prior to use.

1. In each case 25 μl, 50 μl, 100 μl, 150 μl, 200 μl and 250 μl of cysteine solution were pipetted into test tubes (13×100 mm) for a calibration curve. The Protein samples to be determined were poured into separate test tubes (volume <=250 μl). Of the KBD to be tested, at least an amount of 1 mg per reaction mixture was dispensed. In the case of the test tubes, the total volume was then adjusted in each case to 250 μl with Na phosphate buffer. If the volume of 250 μl of sample was exceeded (on account of the required 1 mg of KBD), this was taken into consideration when topping up in point 2 with 2.5 ml of Na phosphate buffer.

2. Addition of in each case 50 μl of Ellmann's reagent and 2.5 ml of Na phosphate buffer. Briefly mix and incubate for 15 min at RT.

3. Measure the absorption at 412 nm.

4. Construct the calibration curves, plot and read off the values of the Protein samples to be determined.

Evaluating the Ellmann test after coupling reactions with various effectors shows that ⅔ of the free thiol groups can be coupled to activated reactive dyes (see Example 19) or dye with maleimidocaproic acid linker if the mixing ratio of KBD-B:dye is 1:2.

### Example 17

### Hair-Binding Activity of KBD-B-Dye

In order to test whether KBD-B (SEQ ID No.: 166) also binds with coupled dye to hair, a quantitative binding assay was carried out (see FIG. 9): in this test, hair was firstly incubated with KBD-B-dye and nonbound KBD-B-dye was washed off. A peroxidase was then coupled via the His tag of the KBD-B. Nonbound peroxidase was washed off again. The bound peroxidase can convert a colorless substrate (TMB) into a colored product, which was measured photometrically at 405 nm. The intensity of the absorption indicates the amount of bonded KBD-B-panthenol. As comparison sample, KBD-B without dye was chosen. For the exact procedure, see Example 10.

### Example 18

### Activation of the Vinylsulfone Reactive Anchors Present in the Reactive Dyes

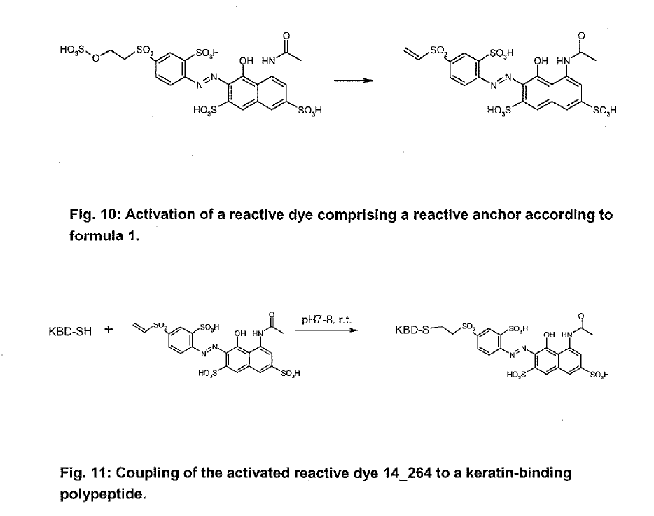

0.075 mol of dye (14-264, see Table 6) was dissolved in 800 ml of water and the pH was adjusted to 10.5 by adding 25% strength by weight NaOH solution. The reaction solution was stirred for 15 minutes at room temperature, during which the pH was kept at 10.5 until it remained stable. The pH was then adjusted to 4.5 by adding 21% strength by weight HCl solution and the reaction mixture was evaporated under reduced pressure (see FIG. 10).

### Example 19

### Coupling of Reactive Dyes to KBD-B (SEQ ID No.: 166)

During the reaction of dyes with the KBD-B, virtually physiological conditions should be maintained since the Protein otherwise unfolds, resulting in precipitation. This means essentially that no pure organic solvents can be used, and a pH close to neutral should be chosen. Furthermore, it is important that the reaction temperature for the coupling should not exceed 45° C.

For the coupling of reactive dyes, cysteines were used in the KBD (SEQ ID No.: 166). Thus, KBD-B (SEQ ID No.: 166) has four cysteines. Of these, two cysteines are inside the structure and are not accessible for the coupling of an effector (recognizable from the crystal structure). The two remaining cysteines close to the N terminus (amino acid positions 14 and 83; see sequence KBD-B (SEQ ID No.: 166) are accessible for an effector coupling.

The reactive dye capable of coupling was coupled to the KBD-B (SEQ ID No.: 166) via at least one of the two free SH groups of a cysteine. Ideally, the reaction between KBD-B and activated dye therefore takes place in the molar ratio 1:2.

### Example 19a

### Coupling of Reactive Dyes to KBD-D (SEQ ID No.: 168)

For coupling the activated dye, analogously to the KBD-B, cysteines can also be used in the KBD-D (SEQ ID No.: 168). Thus, KBD-D (SEQ ID No.: 168) has 24 cysteines. In addition, cysteine radicals capable of coupling can be introduced in a targeted manner by directed mutagenesis.

The coupling of the dye to the KBD-D (SEQ ID No.: 168) can thus take place analogously to the method described in Example 19 and the dyes (14-1 to 14-366) take place as described under 19 b) for the KBD-B using the KBD-D (see Ex. 65). The dyes 14_{—}153, 14_{—}154, 14_{—}155, 14_{—}156, 14_{—}181, 14_{—}182, 14_{—}183, 14_{—}276 and 14_{—}289 are already in the vinylsulfone form, i.e. these do not need to be activated (preeliminated), but can be reacted directly with the KBD-D (corresponding to FIG. 11). The KBD-D reactive dye obtained in this way could be used analogously to the KBD-D reactive dye from Ex. 65 in the cosmetic formulations according to Examples 66-108.

### Example 19b)

### Coupling of KBD-B (SEQ ID No.: 166) to

### a) Vinylsulfone Dyes (Formula 1):

The reaction between KBD-B and activated dyes takes place in the molar ratio 1:2 since the KBD-B has two free cysteines for effector coupling. The vinylsulfone dyes chosen for the coupling are usually synthesized in the sulfuric ester form and therefore have to be activated prior to the reaction with KBD-B to the vinylsulfone form because the dyes can only react with nucleophilic groups in their vinylsulfone form. Activation takes place under alkaline conditions, the dye in aqueous solution being adjusted to pH 11 at room temperature for 2 minutes. Then, prior to adding the Protein, the pH is adjusted to 7-8 with hydrochloric acid (see also Example 18).

The reaction solution is gently shaken for 30 min at 30° C., pH 7-8. During this, activated vinylsulfone dyes (e.g. 14-264, see Table 6) react with the thiol groups of the free cysteins of the KBD (see FIG. 2).

The following dyes (14_{—}1 to 14-291) can be reacted analogously with the KBD. The dyes 14_{—}153, 14_{—}154, 14_{—}155, 14_{—}156, 14_{—}181, 14_{—}182, 14_{—}183, 14_{—}276 and 14_{—}289 are already in the vinylsulfone form, i.e. these do not need to be activated (preeliminated), but can be reacted directly with the KBD (corresponding to FIG. 11). The color of the respective product is given.

TABLE 6

| | Structure | Color |
|---|---|---|
| 14_1 | 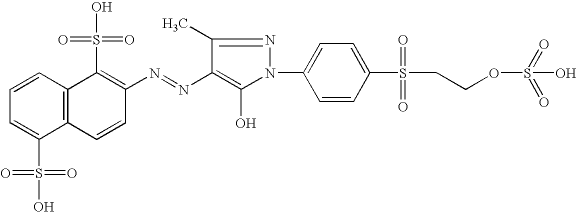 | yellow |
| 14_2 | 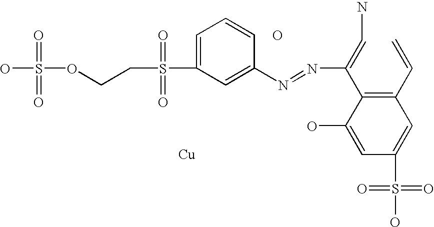 | black |
| | 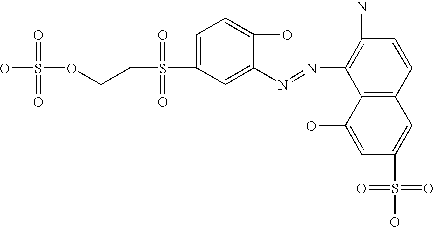 | |
| 14_3 | 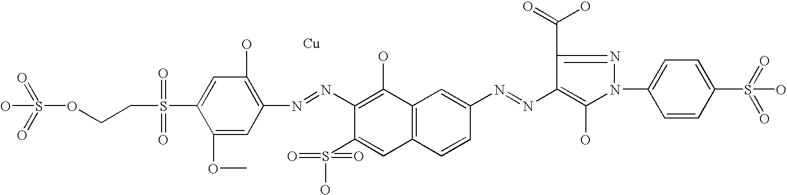 | black |
| 14_4 | 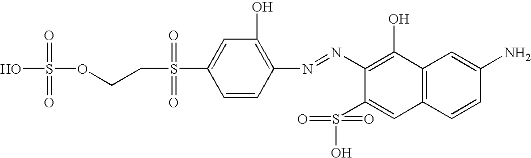 | black |
| | 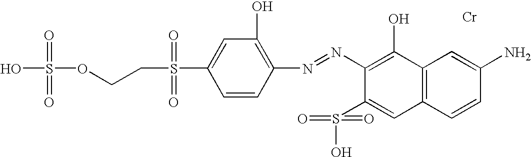 | |
| 14_5 | 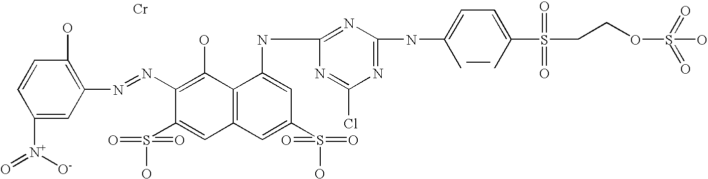 | black |
| | 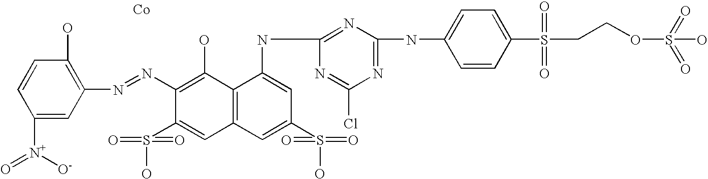 | |
| 14_6 | 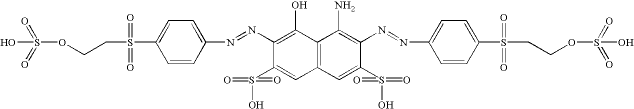 | blue |
| 14_7 | 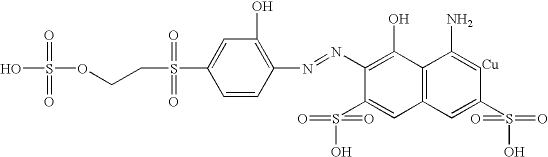 | blue |
| 14_8 | 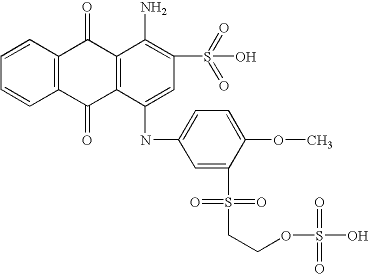 | blue |
| 14_9 | 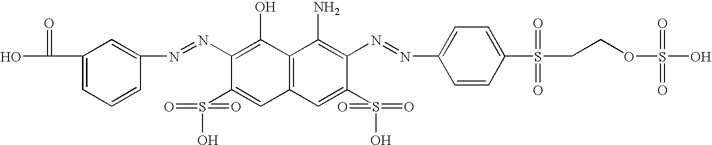 | blue |
| 14_10 | 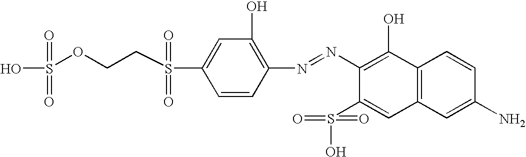 | blue |
| | 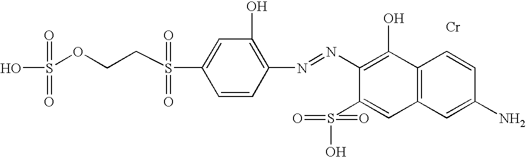 | |
| 14_11 | 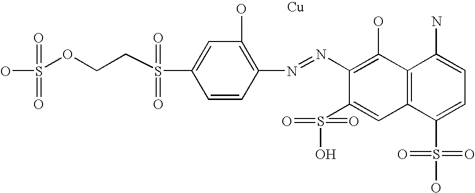 | blue |
| 14_12 | 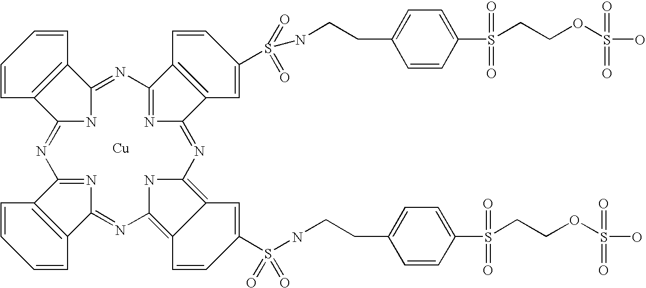 | blue |
| 14_13 | 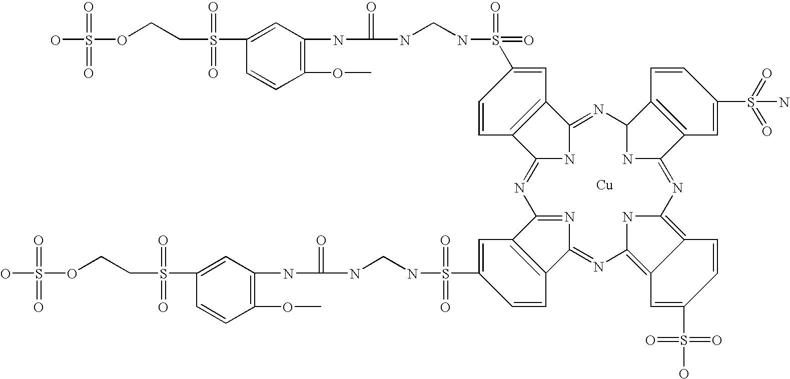 | blue |
| 14_14 | 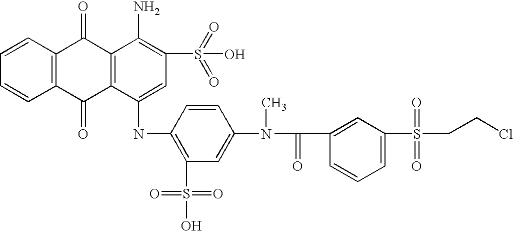 | blue |
| 14_15 | 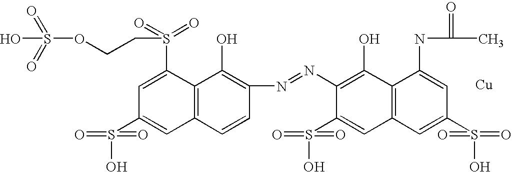 | blue |
| 14_16 | 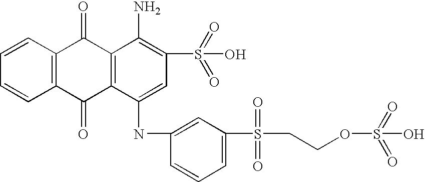 | blue |
| 14_17 | 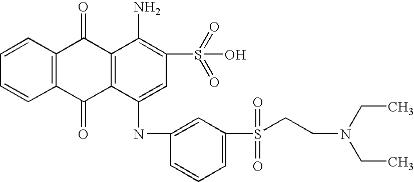 | blue |
| 14_18 | 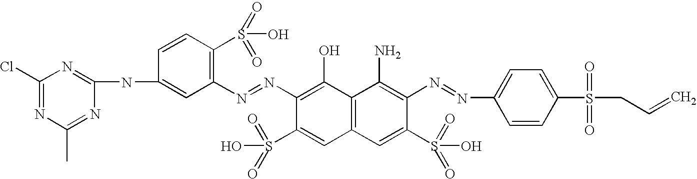 | blue |
| 14_19 | 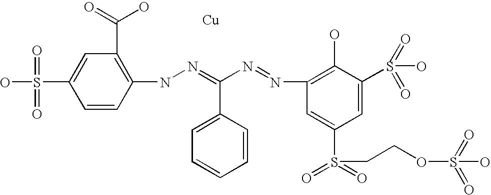 | blue |
| 14_20 | 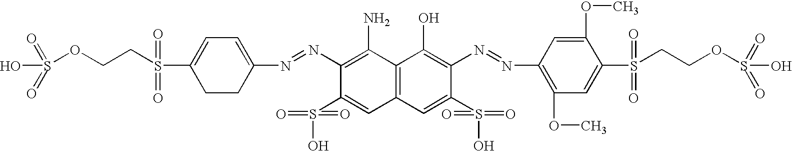 | blue |
| 14_21 | 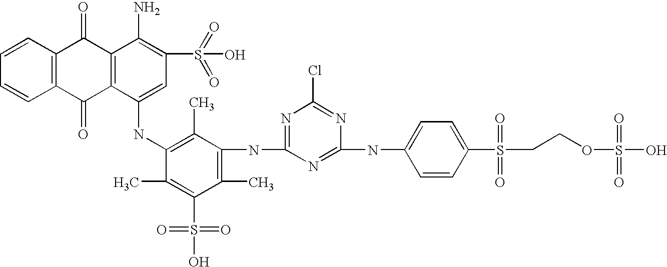 | blue |
| 14_22 | 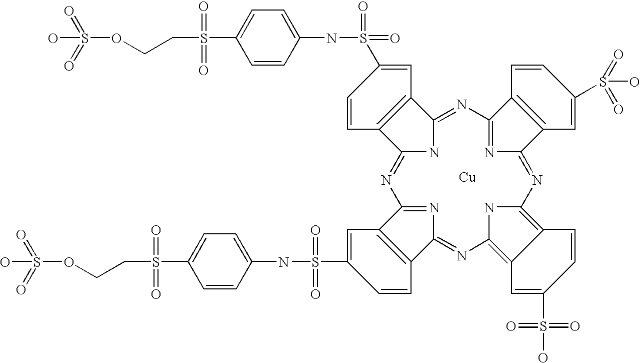 | blue |
| 14_23 | 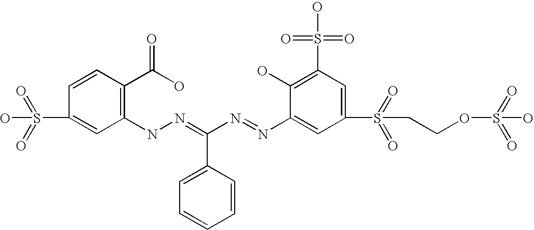 | blue |
| 14_24 | 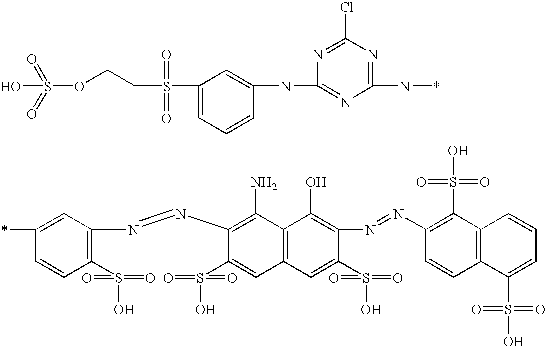 | blue |
| 14_25 | 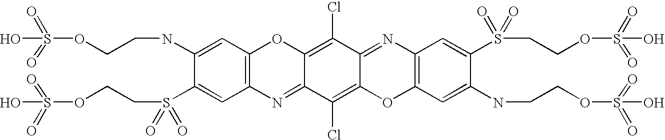 | blue |
| 14_26 | 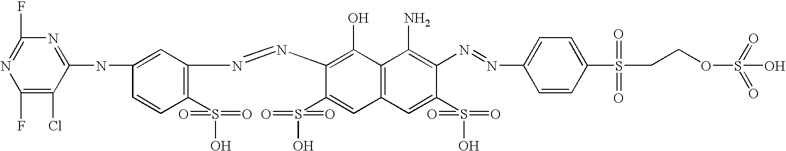 | blue |
| 14_267 | 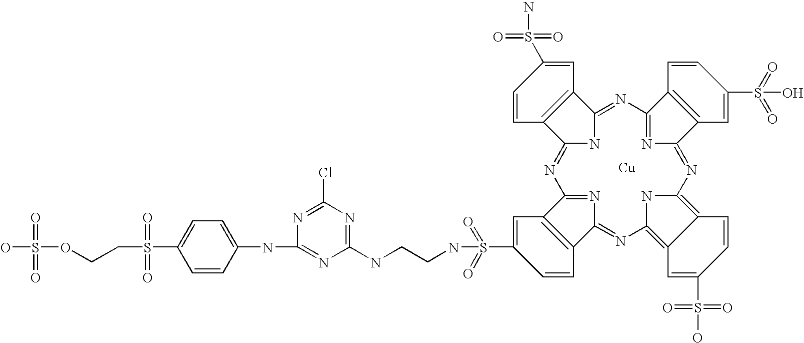 | blue |
| 14_28 | 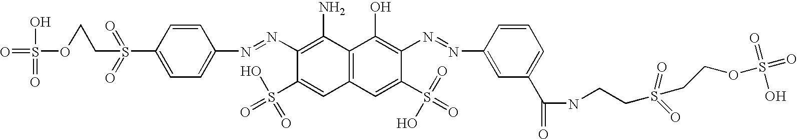 | blue |
| 14_29 | 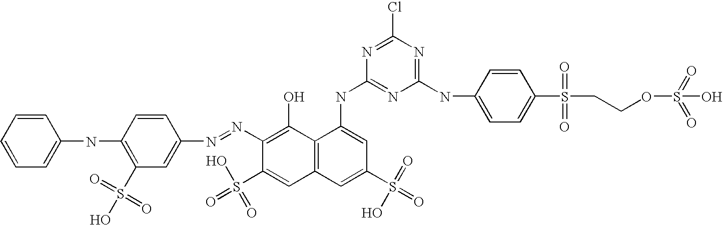 | blue |
| 14_30 | 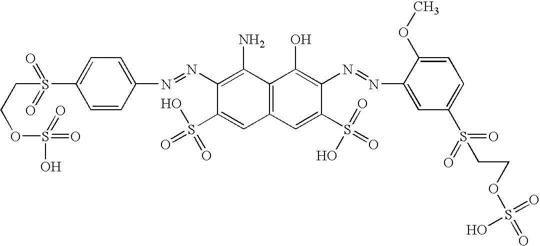 | blue |
| 14_31 | 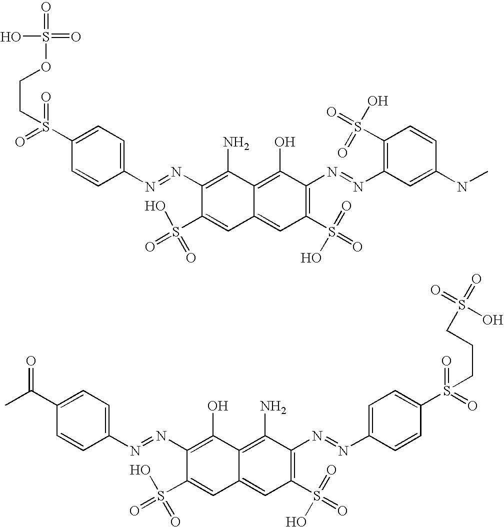 | blue |
| 14_32 | 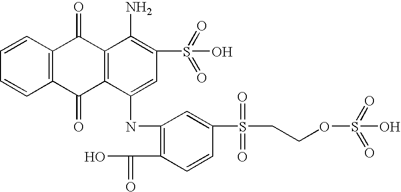 | blue |
| 14_33 | 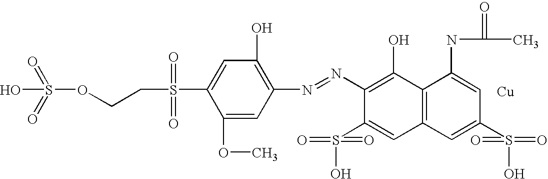 | blue |
| 14_34 | 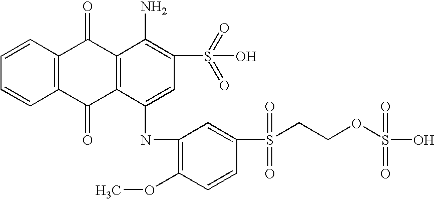 | blue |
| 14_35 | 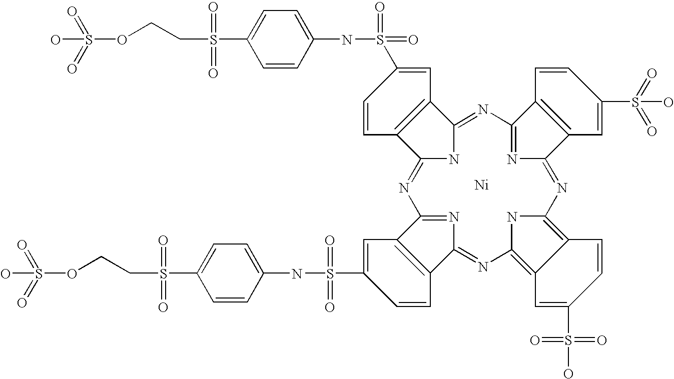 | green |
| 14_36 | 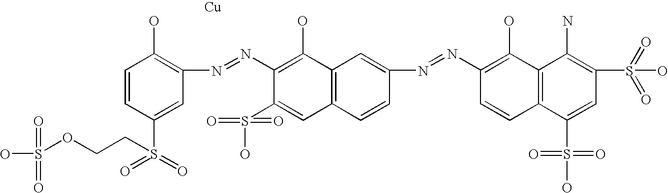 | blue |
| 14_37 | 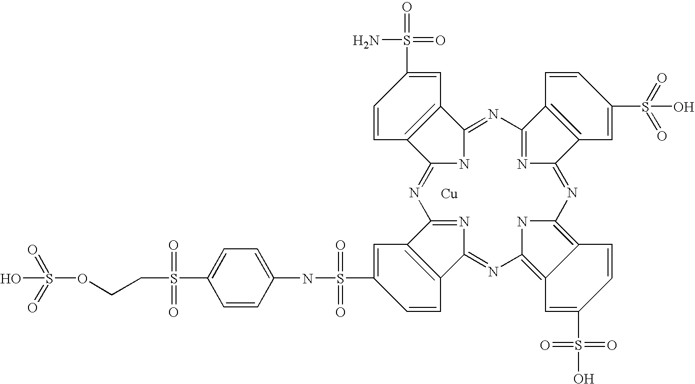 | blue |
| 14_38 | 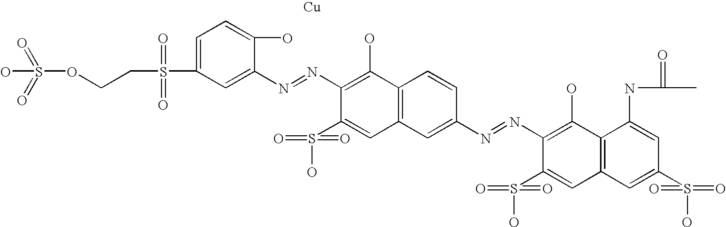 | blue |
| 14_39 | 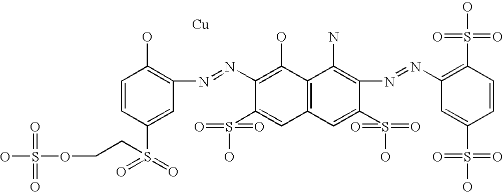 | blue |
| 14_40 | 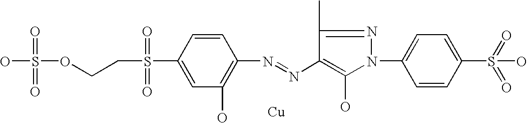 | brown |
| 14_41 | 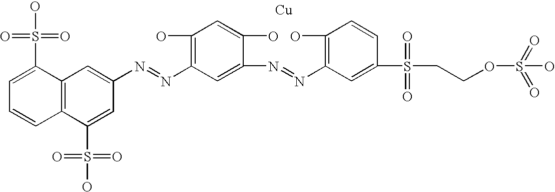 | brown |
| 14_42 | 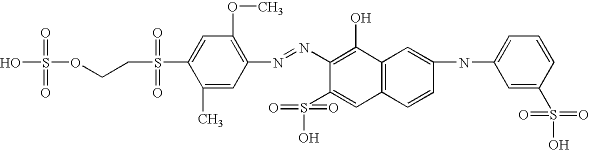 | brown |
| 14_43 | 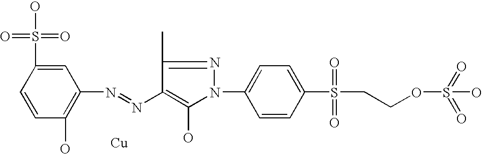 | brown |
| 14_44 | 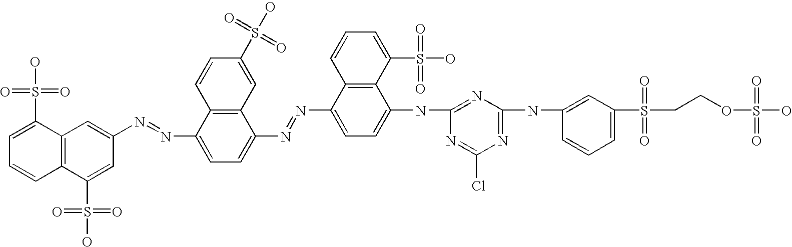 | brown |
| 14_45 | 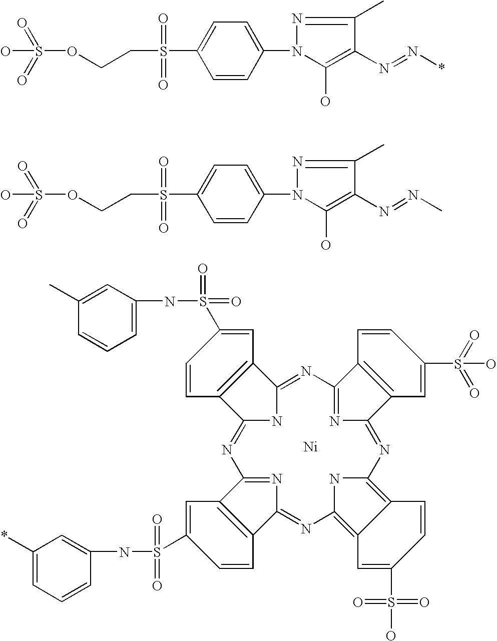 | green |
| 14_46 | 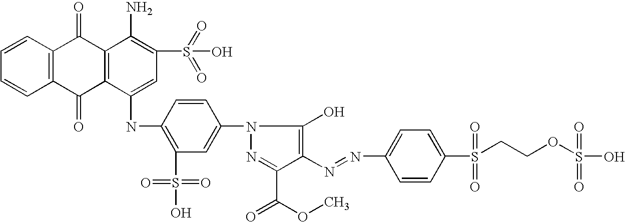 | green |
| 14_47 | 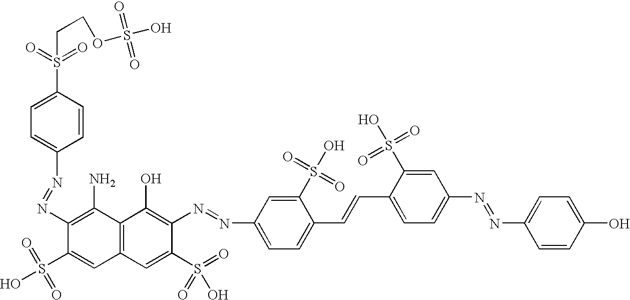 | green |
| 14_48 | 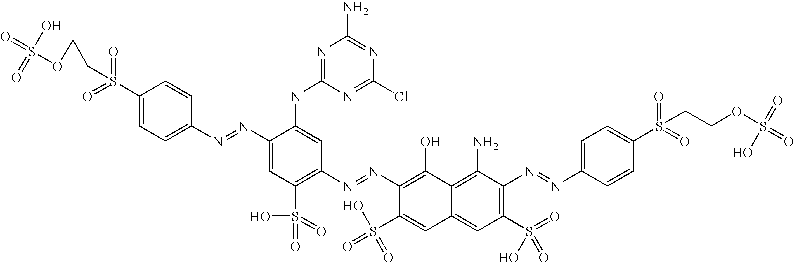 | green |
| 14_49 | 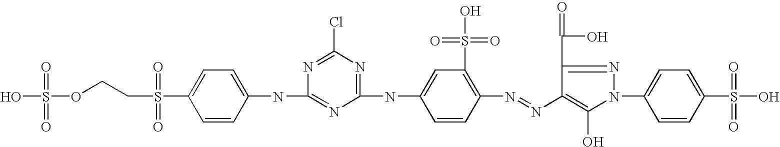 | orange |
| 14_50 | 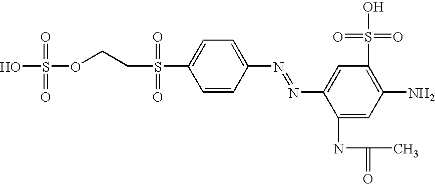 | orange |
| 14_51 | 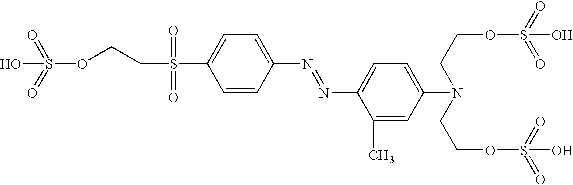 | orange |
| 14_52 | 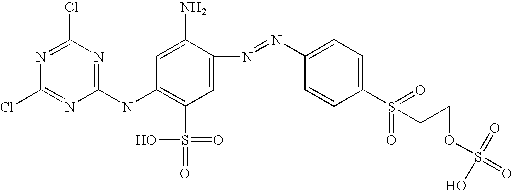 | orange |
| 14_53 | 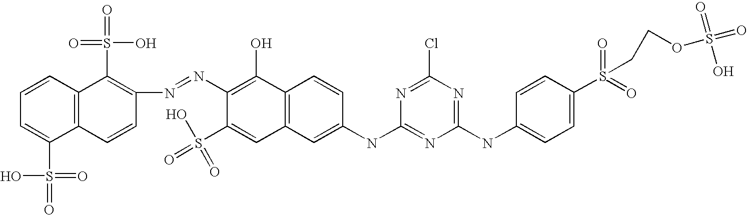 | orange |
| 14_54 | 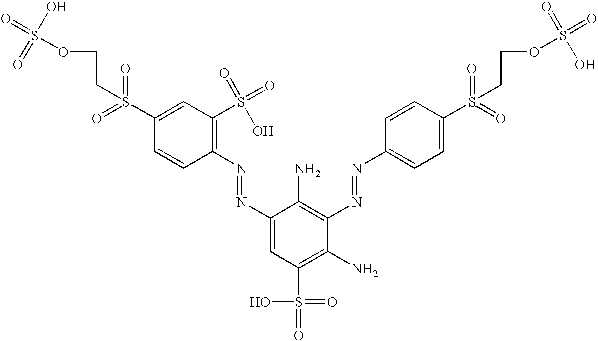 | orange |
| 14_55 | 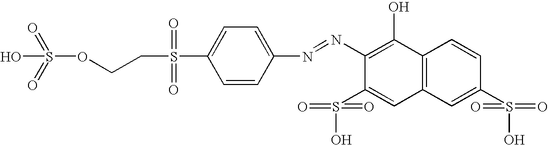 | orange |
| 14_56 | 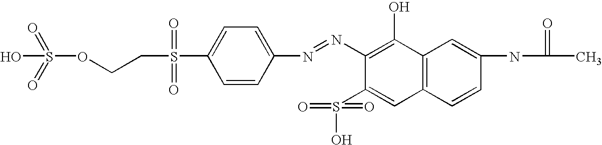 | orange |
| 14_57 | 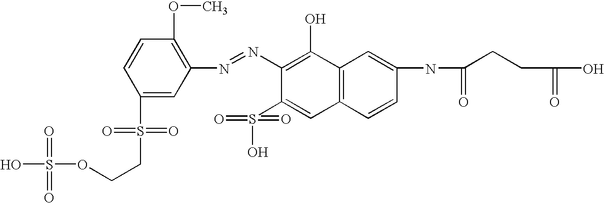 | orange |
| 14_58 | 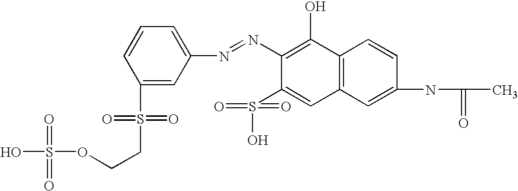 | orange |
| 14_59 | 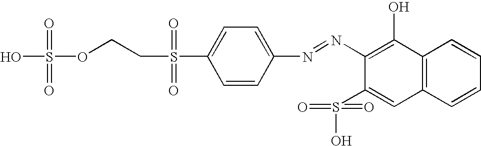 | orange |
| 14_60 | 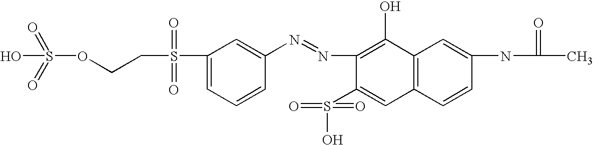 | orange |
| 14_61 | 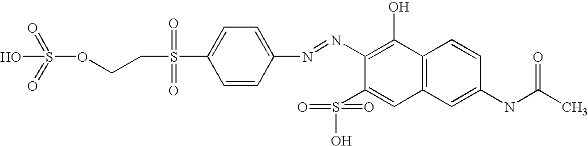 | orange |
| 14_62 | 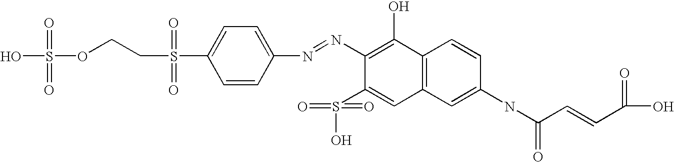 | orange |
| 14_63 | 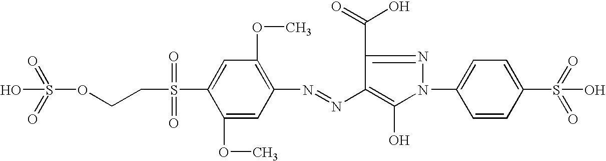 | orange |
| 14_64 | 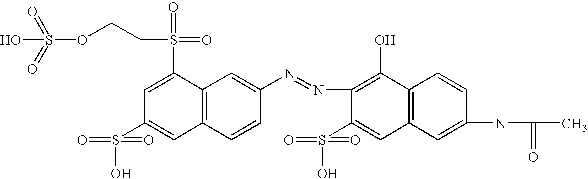 | orange |
| 14_65 | 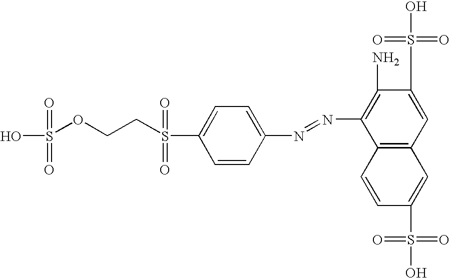 | orange |
| 14_66 | 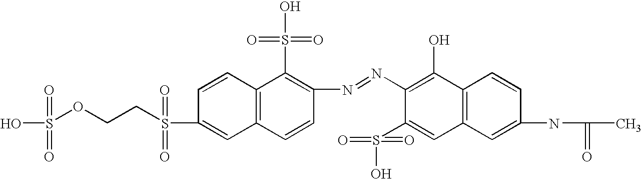 | orange |
| 14_67 | 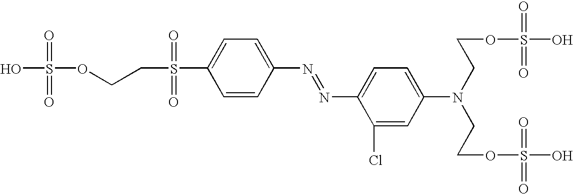 | orange |
| 14_68 | 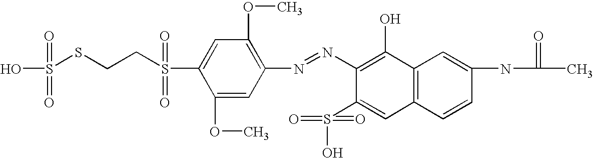 | red |
| 14_69 | 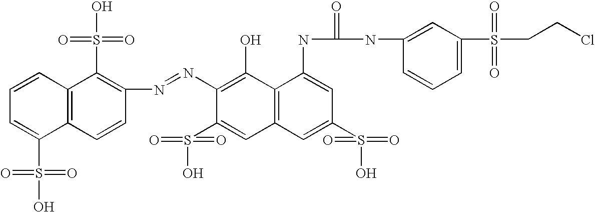 | red |
| 14_70 | 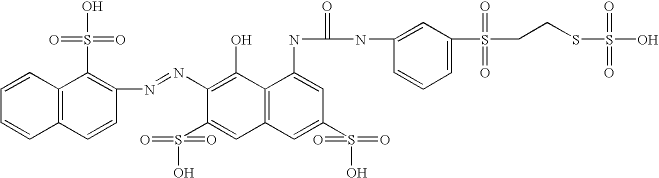 | red |
| 14_71 | 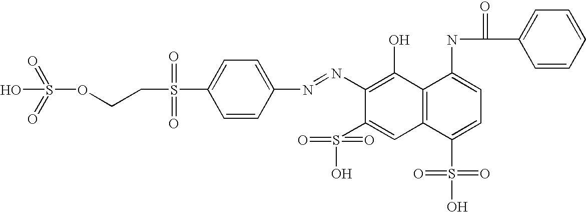 | red |
| 14_72 | 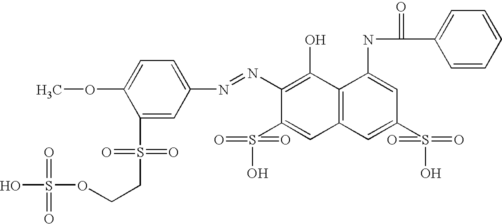 | red |
| 14_73 | 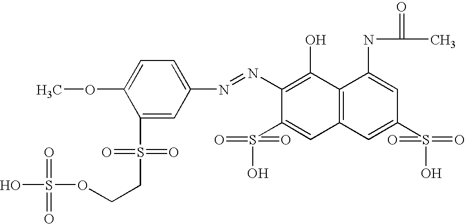 | red |
| 14_74 | 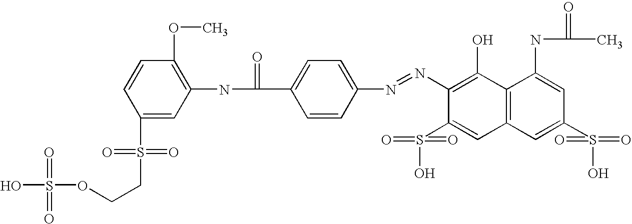 | red |
| 14_75 | 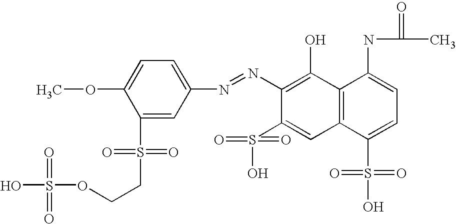 | red |
| 14_76 | 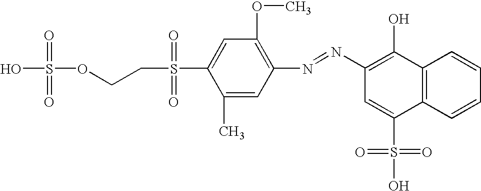 | red |
| 14_77 | 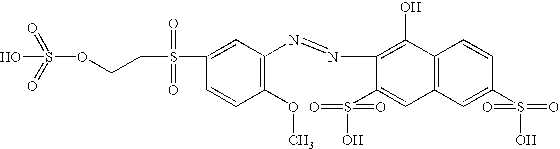 | red |
| 14_78 | 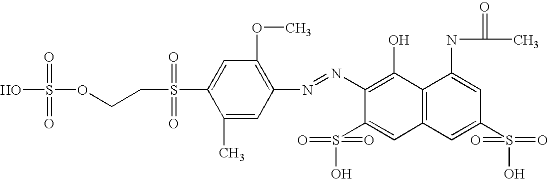 | red |
| 14_79 | 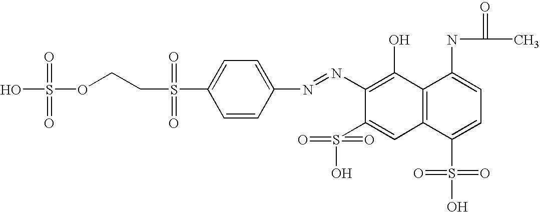 | red |
| 14_80 | 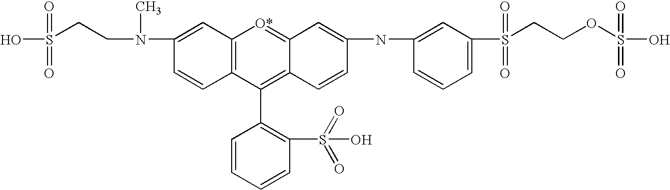 | red |
| 14_81 | 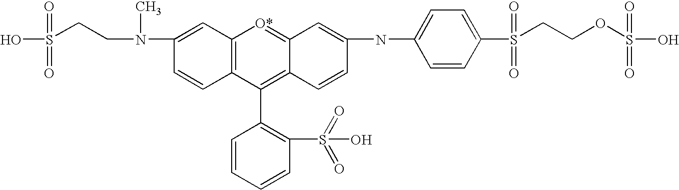 | red |
| 14_82 | 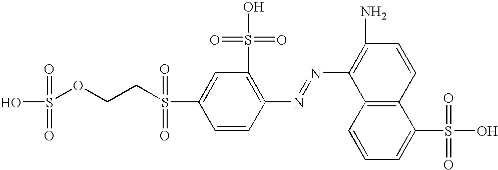 | red |
| 14_83 | 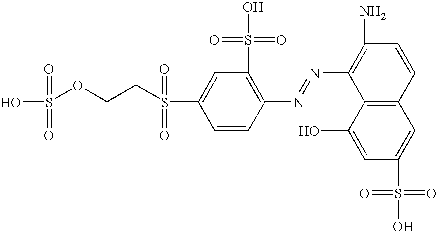 | red |
| 14_84 | 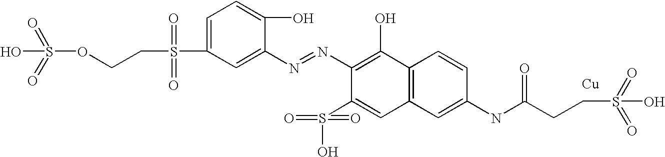 | red |
| 14_85 | 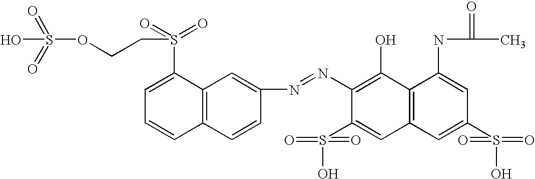 | red |
| 14_86 | 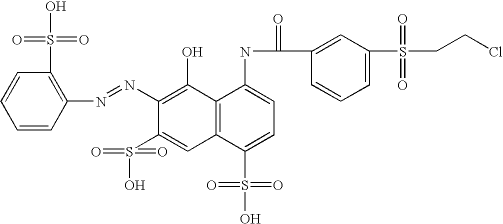 | red |
| 14_87 | 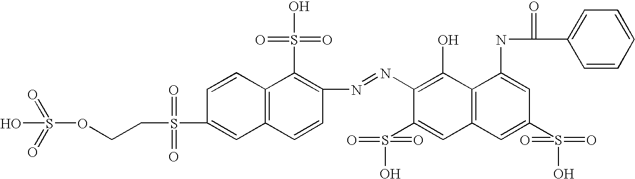 | red |
| 14_88 | 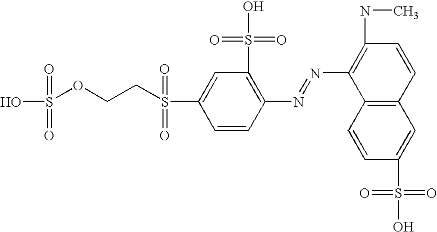 | red |
| 14_89 | 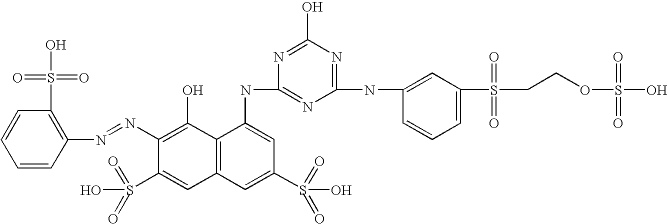 | red |
| 14_90 | 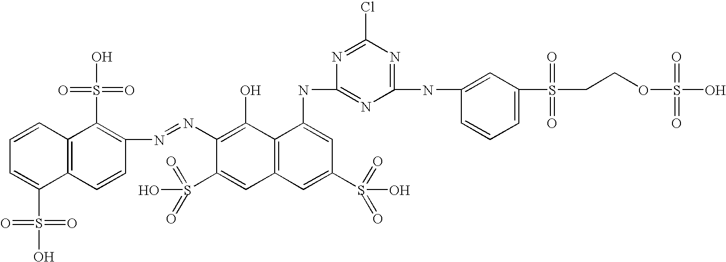 | red |
| 14_91 | 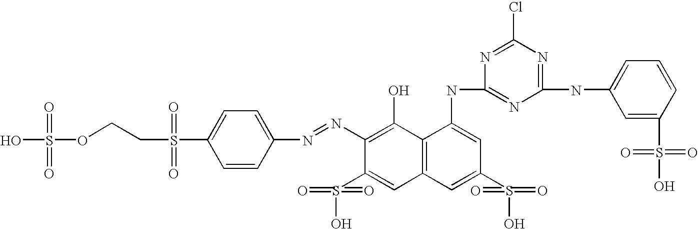 | red |
| 14_92 | 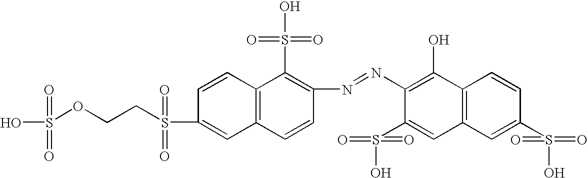 | red |
| 14_93 | 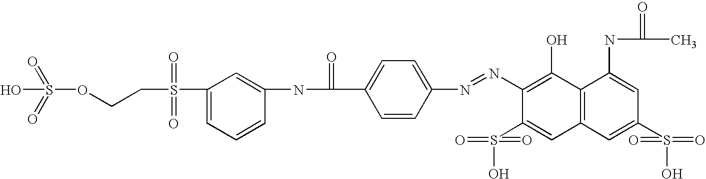 | red |
| 14_94 | 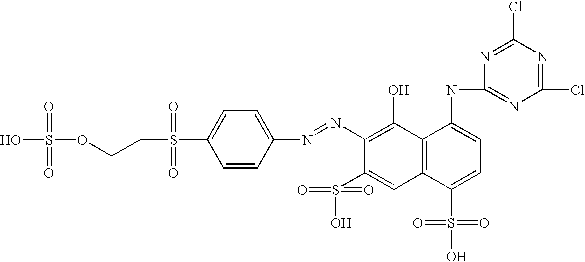 | red |
| 14_95 | 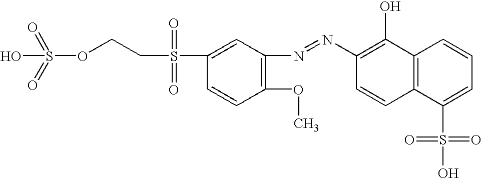 | red |
| 14_96 | 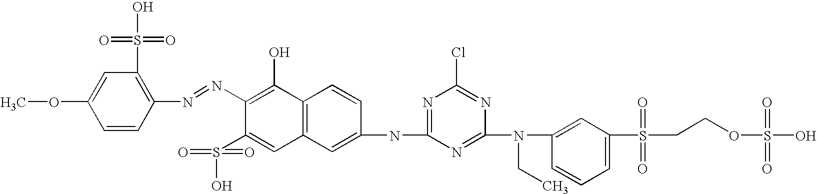 | red |
| 14_97 | 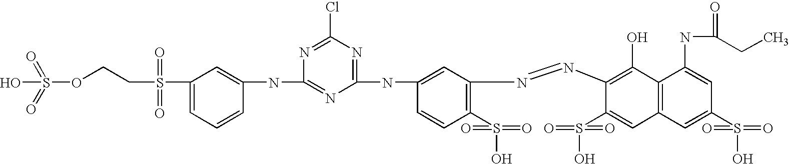 | red |
| 14_98 | 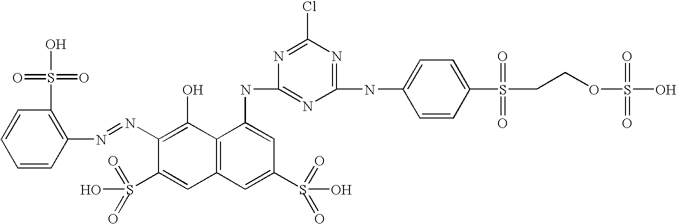 | red |
| 14_99 | 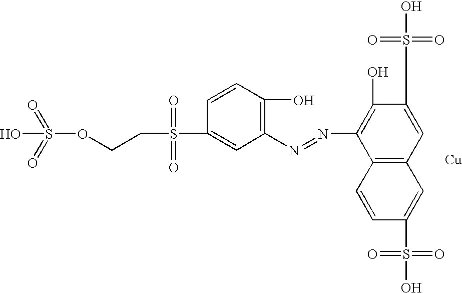 | red |
| 14_100 | 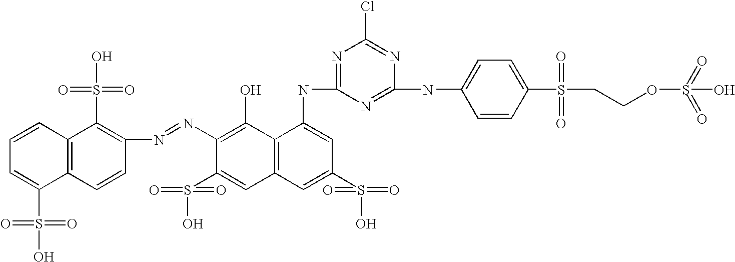 | red |
| 14_101 | 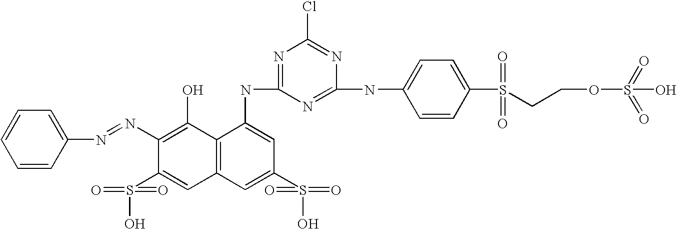 | red |
| 14_102 | 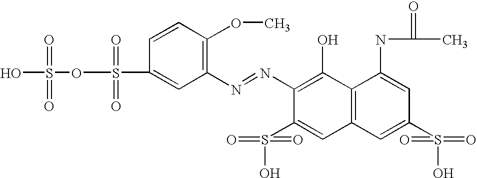 | red |
| 14_103 | 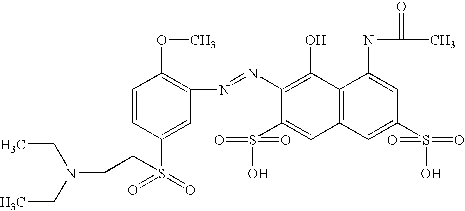 | red |
| 14_104 | 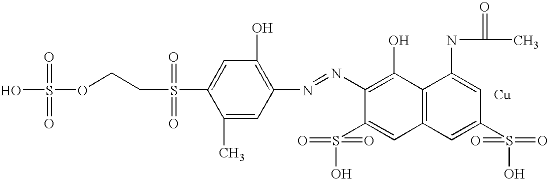 | violet |
| 14_105 | 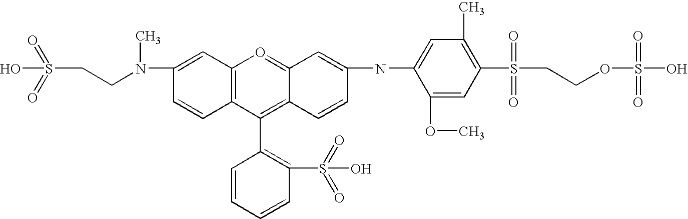 | violet |
| 14_106 | 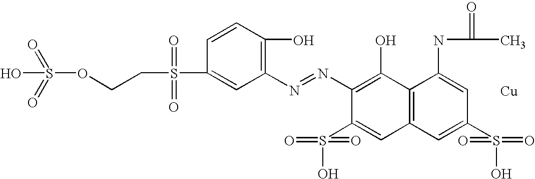 | violet |
| 14_107 | 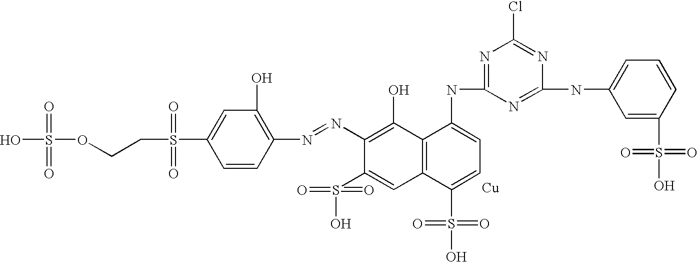 | violet |
| 14_108 | 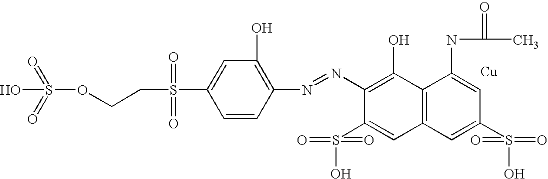 | violet |
| 14_109 | 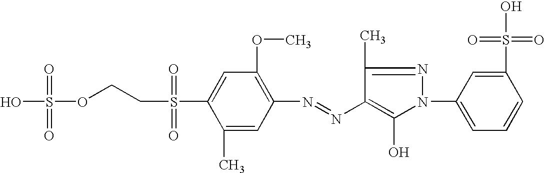 | yellow |
| 14_110 | 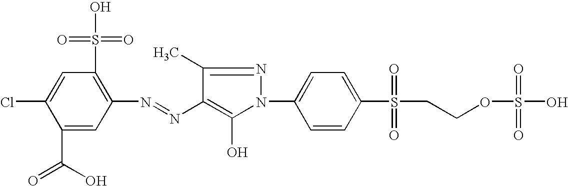 | yellow |
| 14_111 | 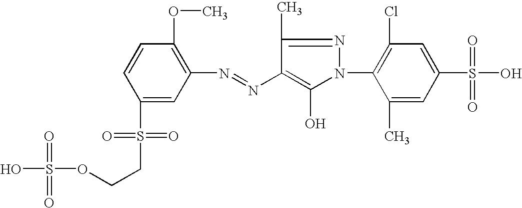 | yellow |
| 14_112 | 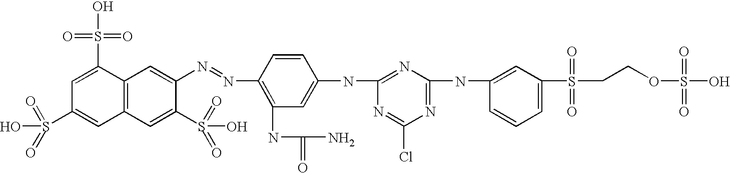 | yellow |
| 14_113 | 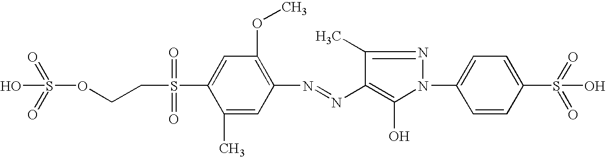 | yellow |
| 14_114 | 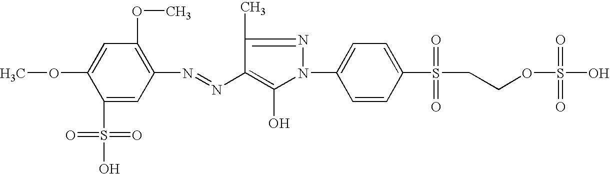 | yellow |
| 14_115 | 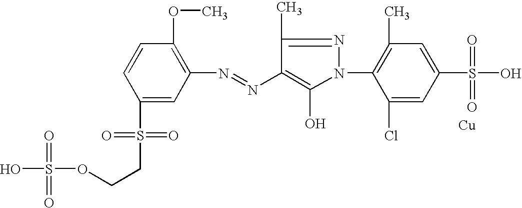 | yellow |
| 14_116 | 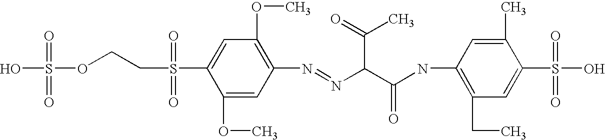 | yellow |
| 14_117 | 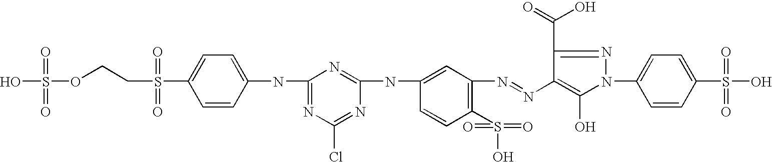 | yellow |
| 14_118 | 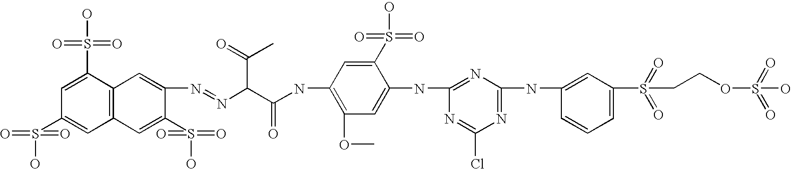 | yellow |
| 14_119 | 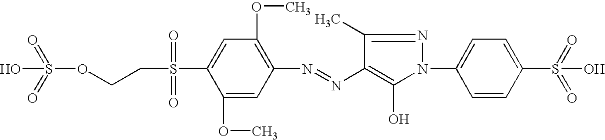 | yellow |
| 14_120 | 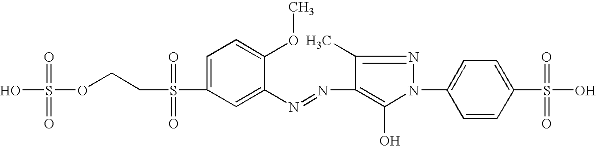 | yellow |
| 14_121 | 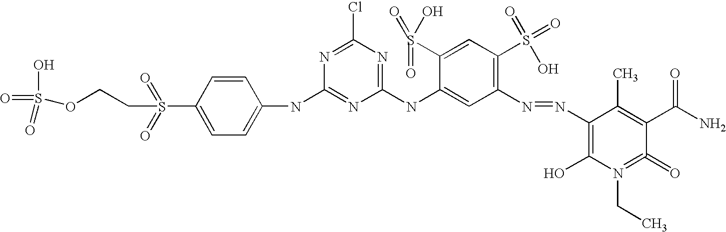 | yellow |
| 14_122 | 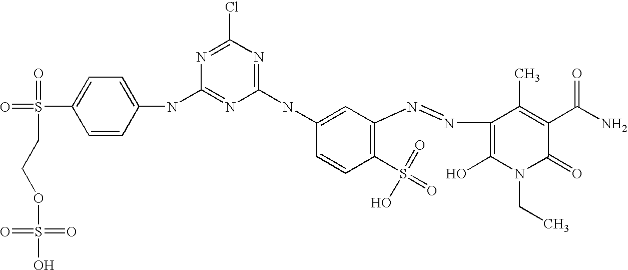 | yellow |
| 14_123 | 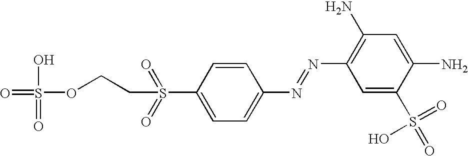 | yellow |
| 14_124 | 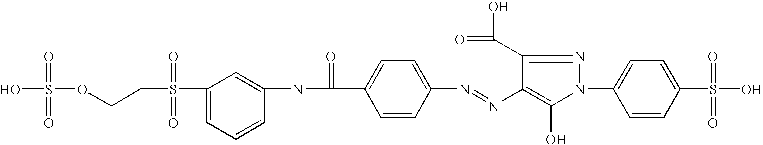 | yellow |
| 14_125 | 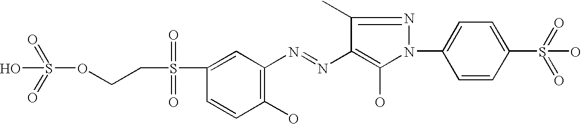 | yellow |
| 14_126 | 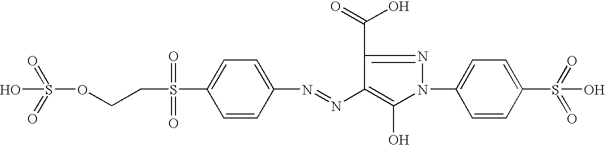 | yellow |
| 14_127 | 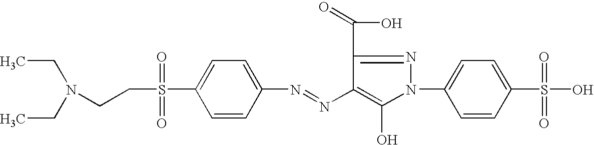 | yellow |
| 14_128 | 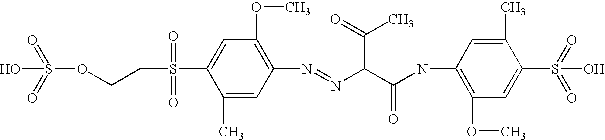 | yellow |
| 14_129 | 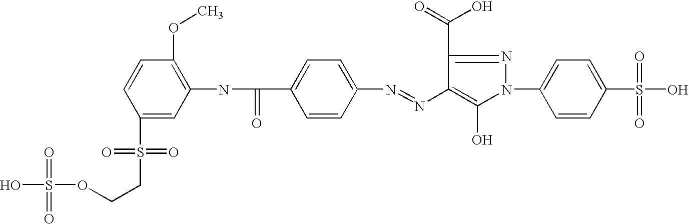 | yellow |
| 14_130 | 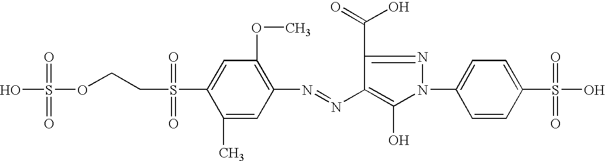 | yellow |
| 14_131 | 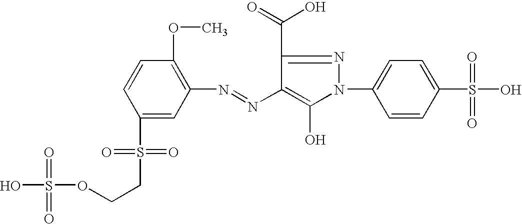 | yellow |
| 14_132 | 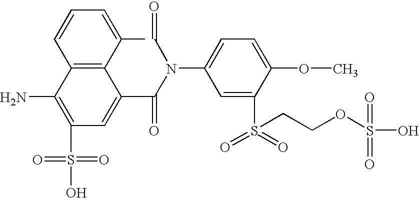 | yellow |
| 14_133 | 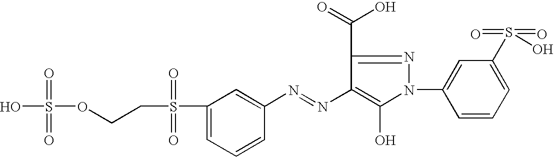 | yellow |
| 14_134 | 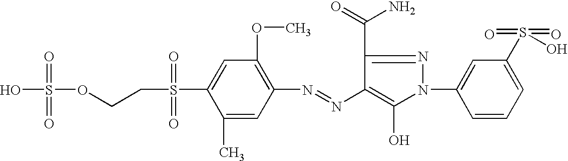 | yellow |
| 14_135 | 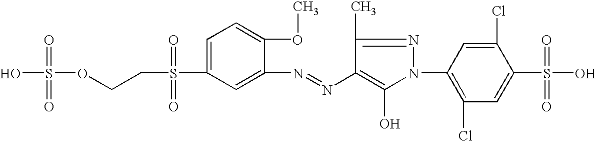 | yellow |
| 14_136 | 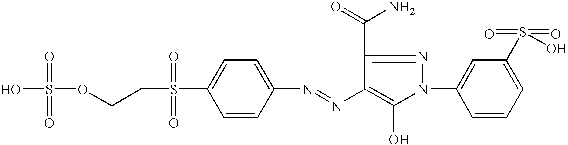 | yellow |
| 14_137 | 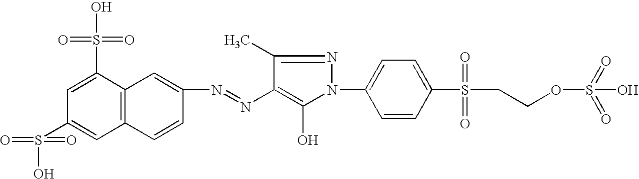 | yellow |
| 14_138 | 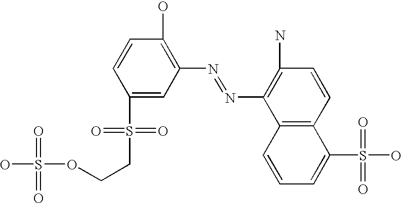 | black |
| | 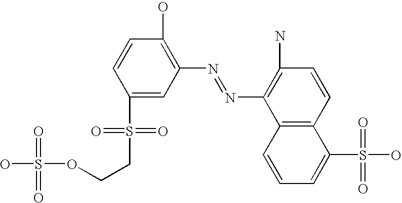 | |
| 14_139 | 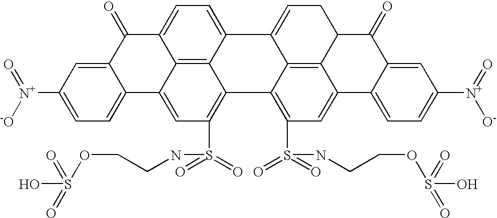 | black |
| 14_140 | 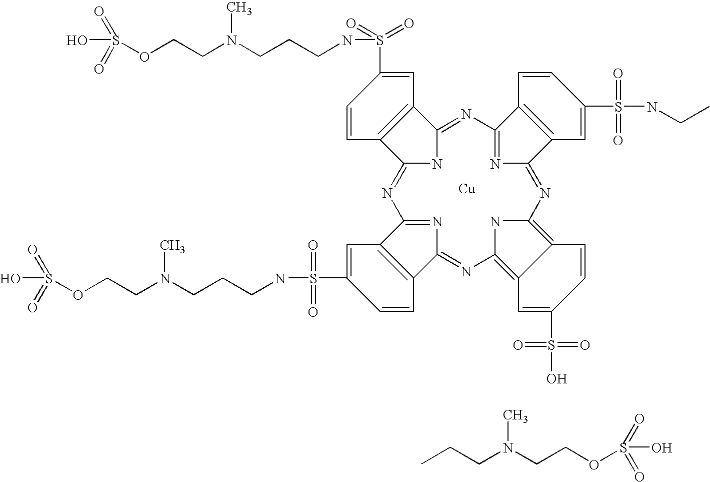 | blue |
| 14_141 | 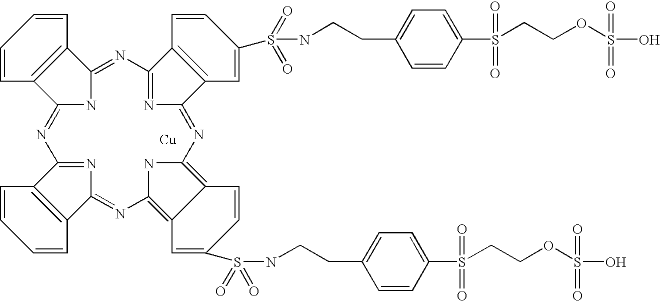 | blue |
| 14_142 | 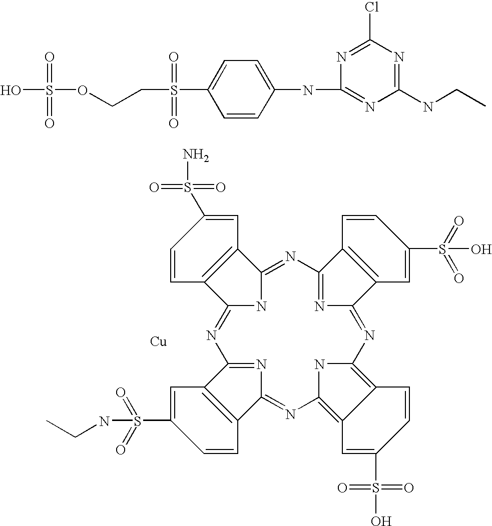 | blue |
| 14_143 | 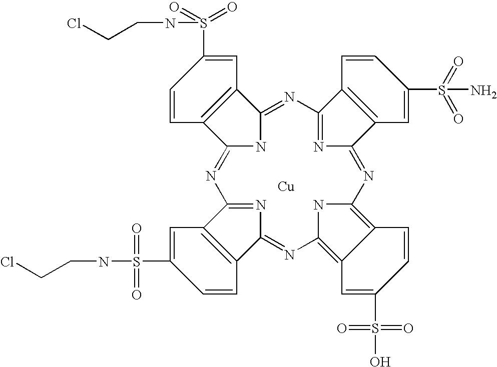 | blue |
| 14_144 | 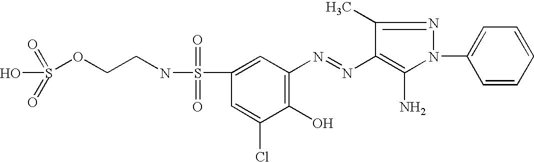 | brown |
| 14_145 | 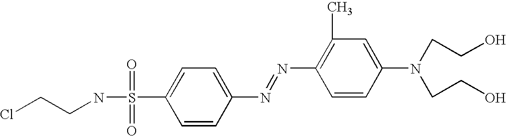 | orange |
| 14_146 | 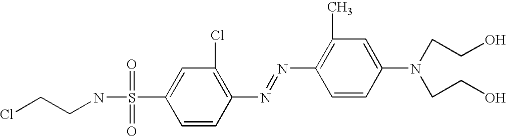 | red |
| 14_147 | 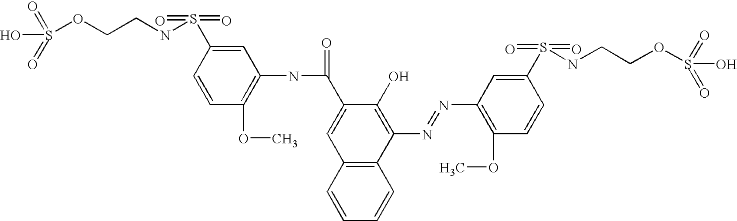 | red |
| 14_148 | 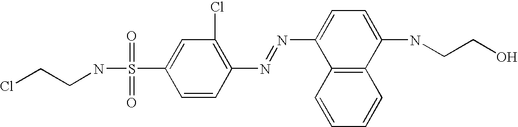 | red |
| 14_149 | 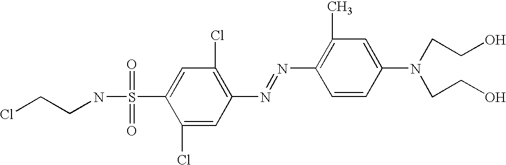 | red |
| 14_150 | 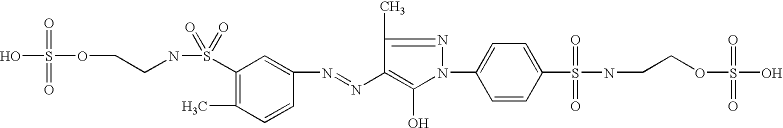 | yellow |
| 14_151 | 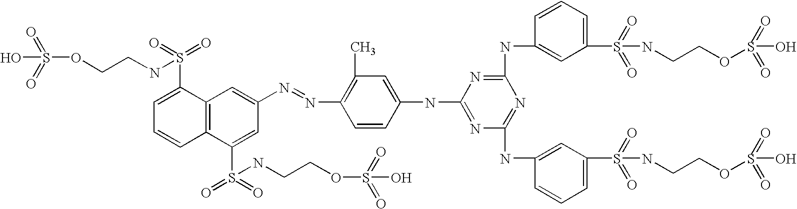 | yellow |
| 14_152 | 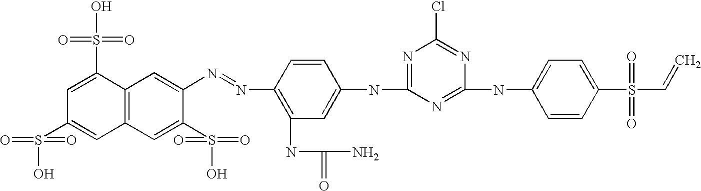 | yellow |
| 14_153 | 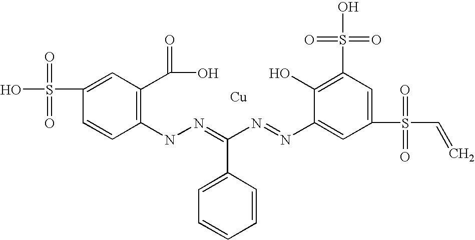 | blue |
| 14_154 | 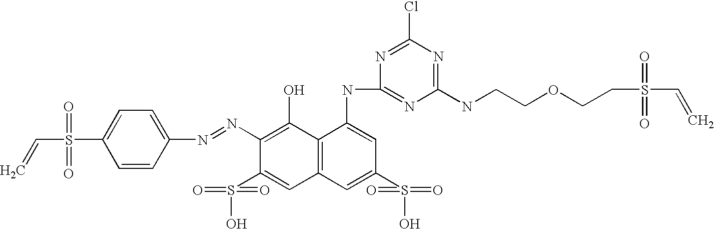 | red |
| 14_155 | 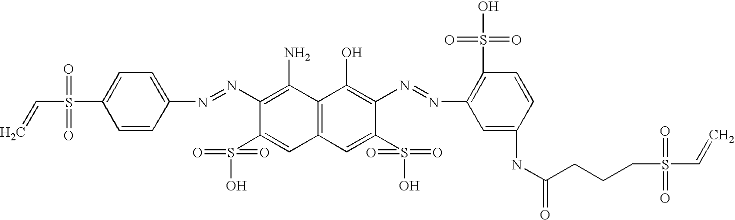 | blue |
| 14_156 | 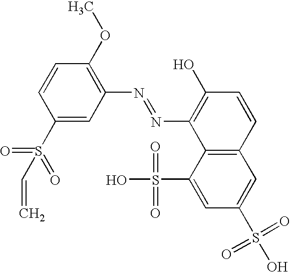 | orange |
| 14_157 | 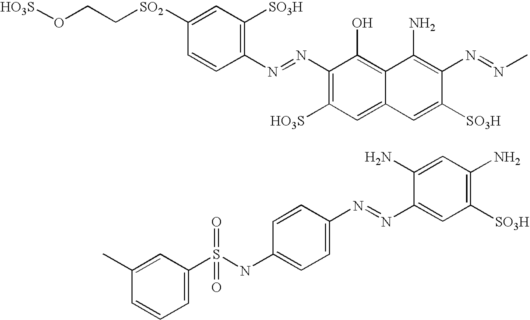 | black |
| 14_158 | 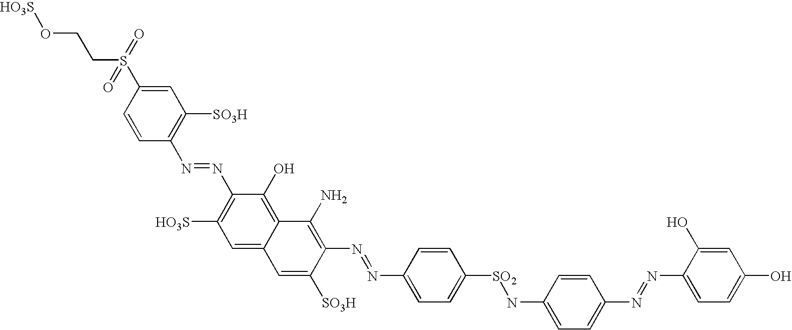 | darkgreen |
| 14_159 | 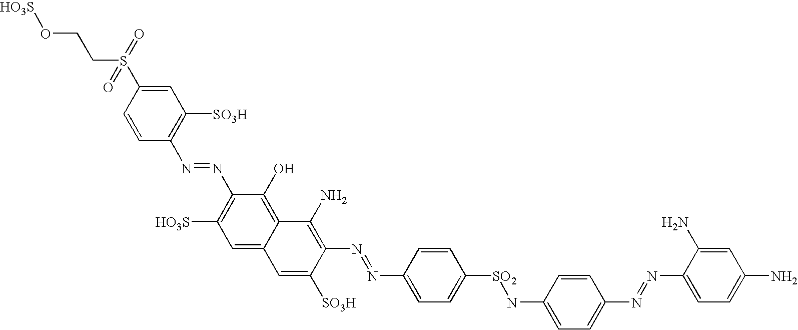 | black |
| 14_160 | 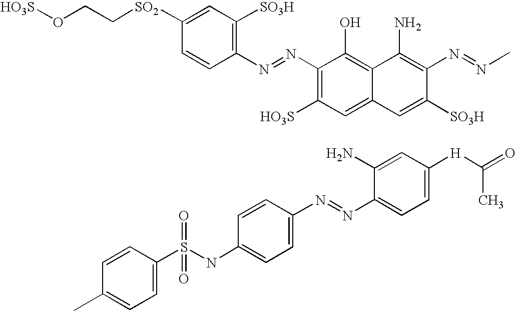 | green |
| 14_161 | 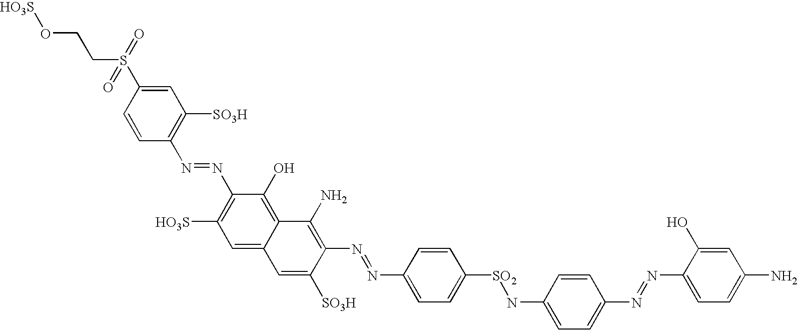 | green |
| 14_162 | 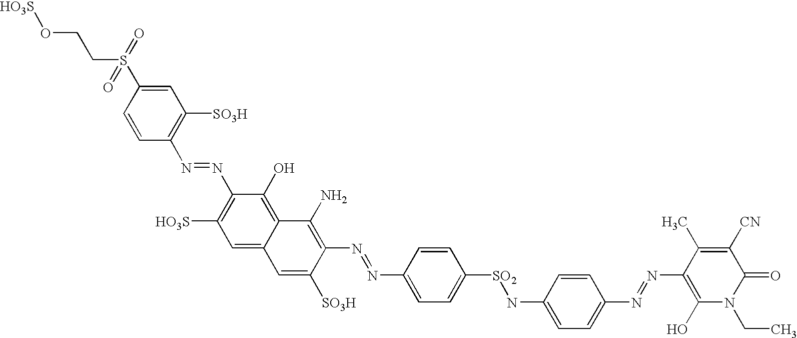 | green |
| 14_163 | 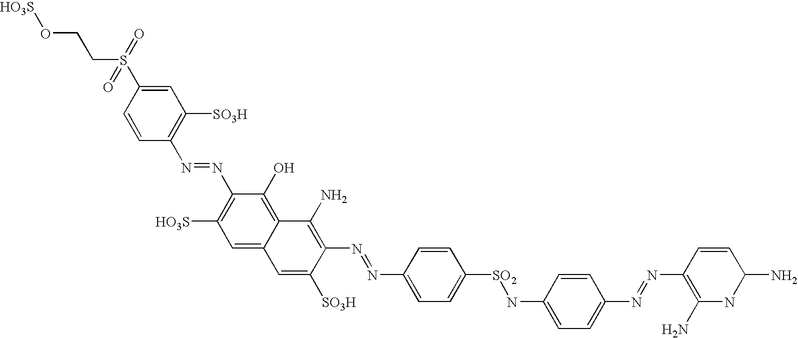 | green |
| 14_164 | 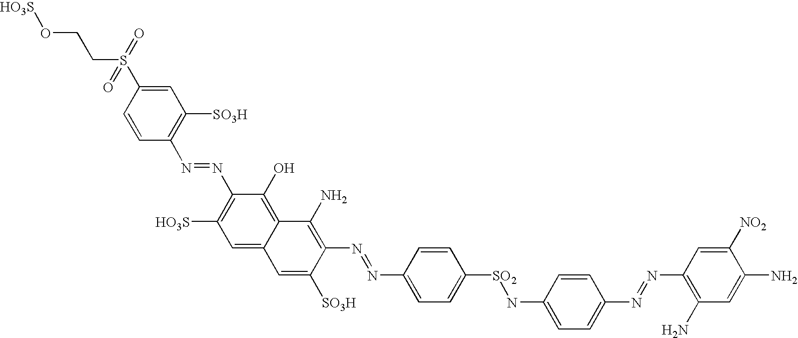 | green |
| 14_165 | 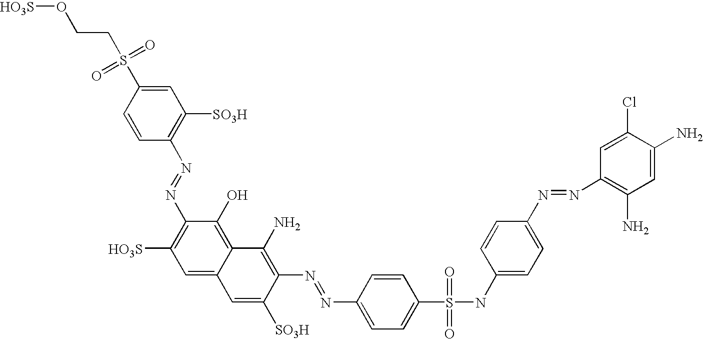 | black |
| 14_166 | 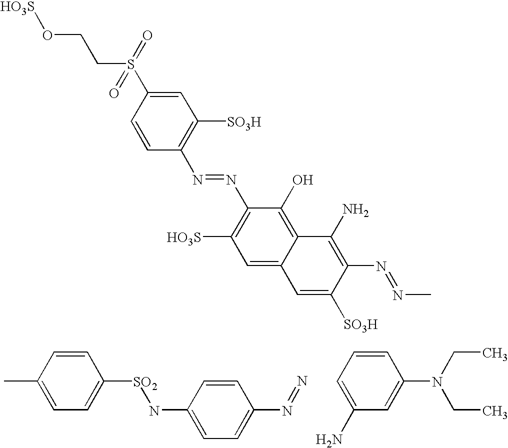 | black |
| 14_167 | 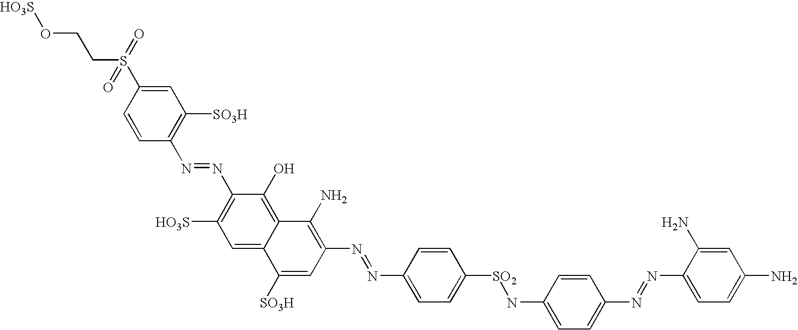 | black |
| 14_168 | 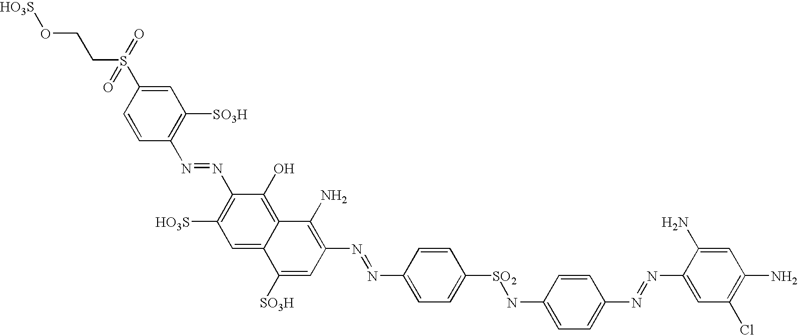 | black |
| 14_169 | 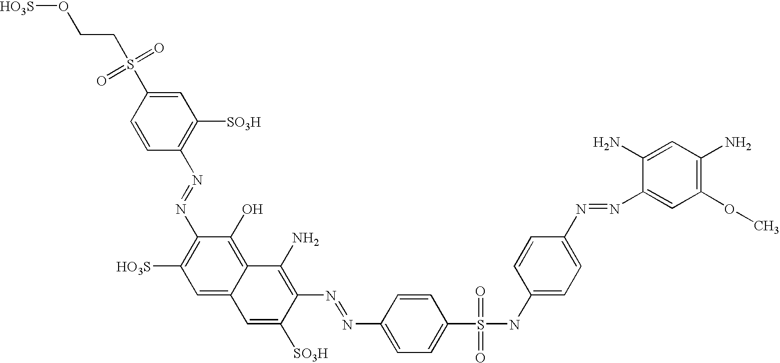 | black |
| 14_170 | 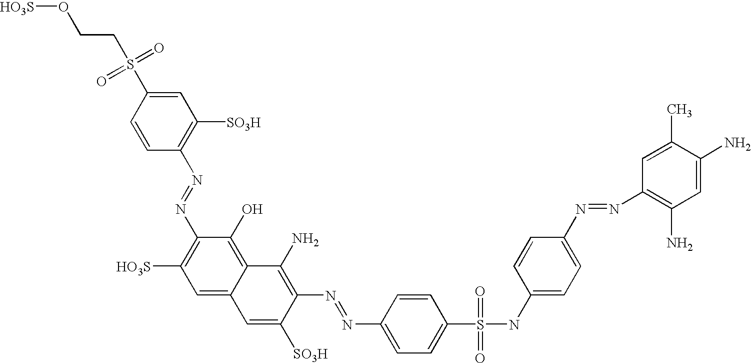 | black |
| 14_171 | 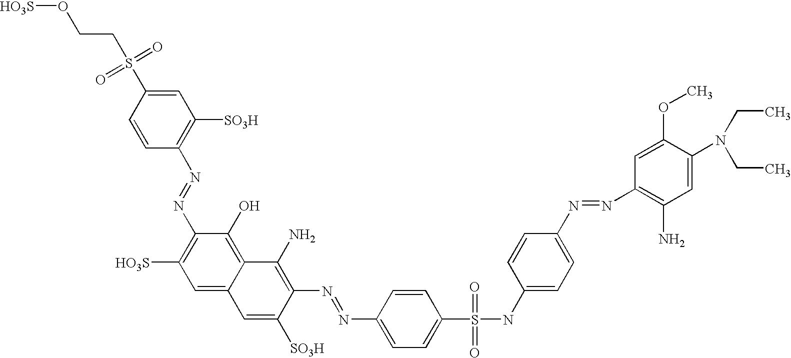 | black |
| 14_172 | 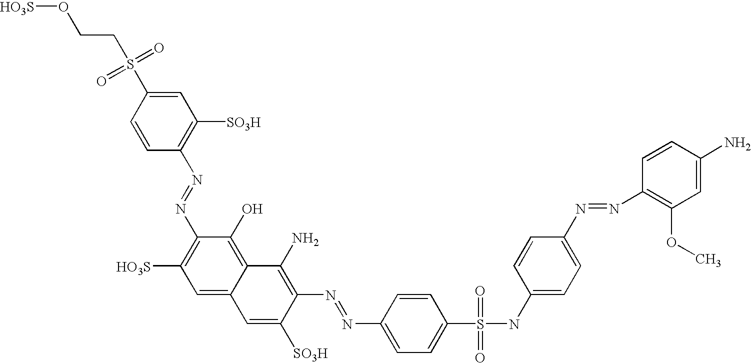 | black |
| 14_173 | 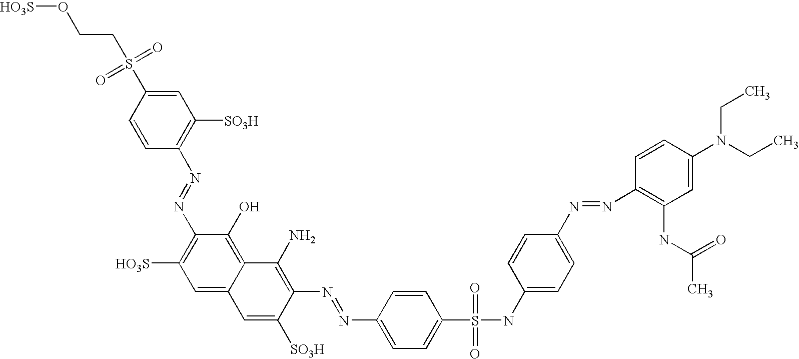 | black |
| 14_174 | 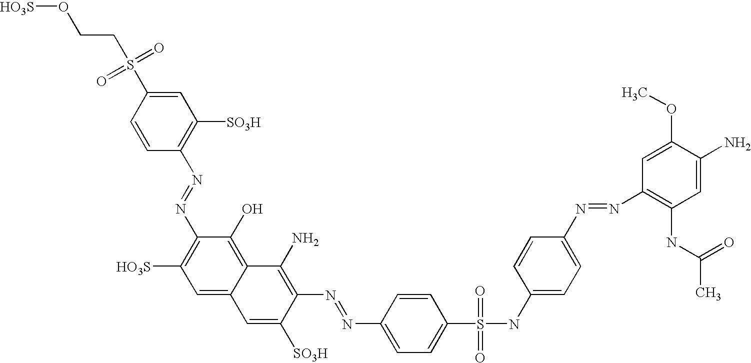 | black |
| 14_175 | 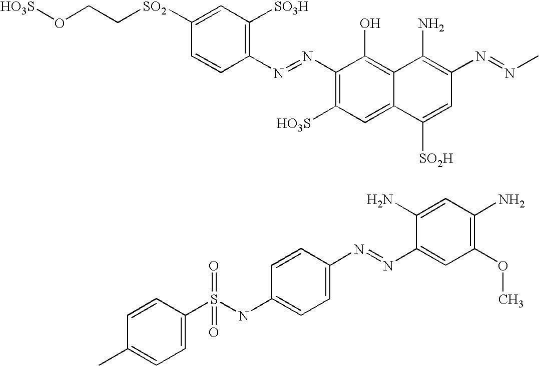 | black |
| 14_176 | 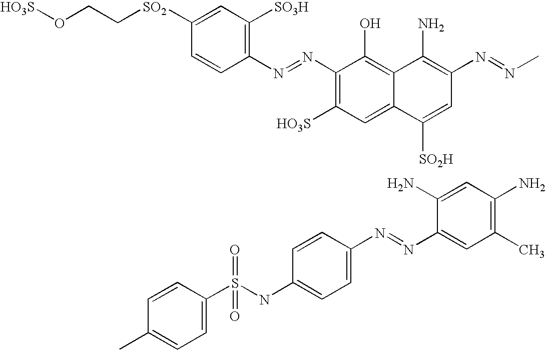 | black |
| 14_177 | 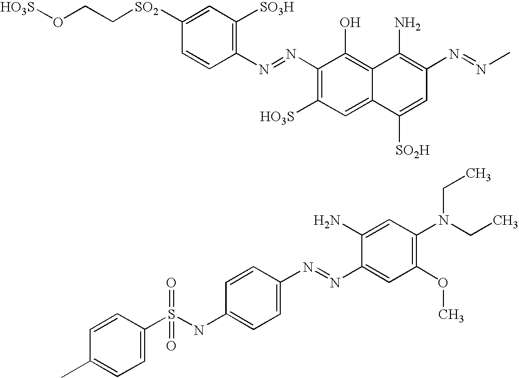 | violet |
| 14_178 | 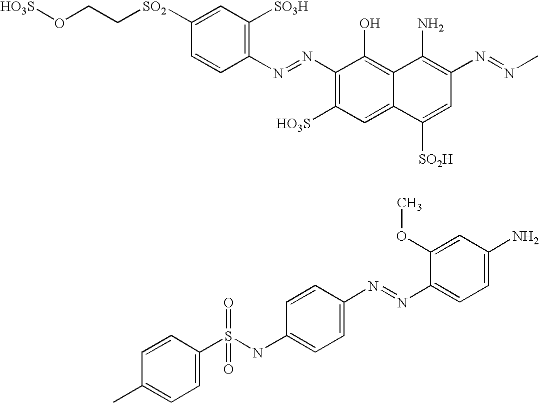 | black |
| 14_179 | 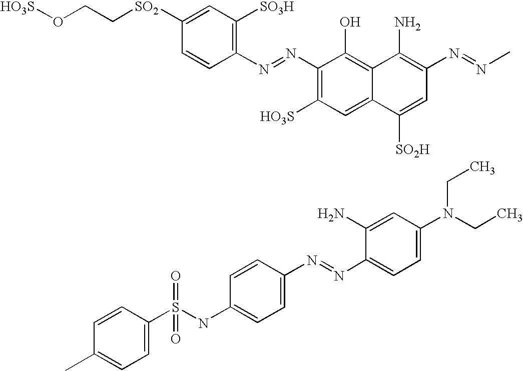 | black |
| 14_180 | 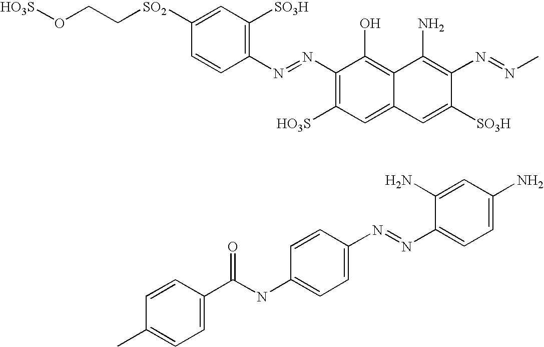 | black |
| 14_181 | 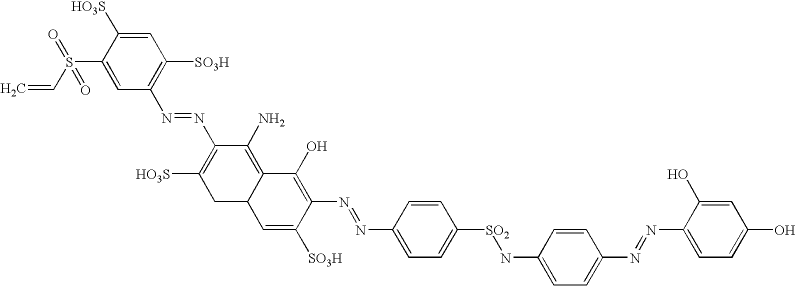 | darkgreen |
| 14_182 | 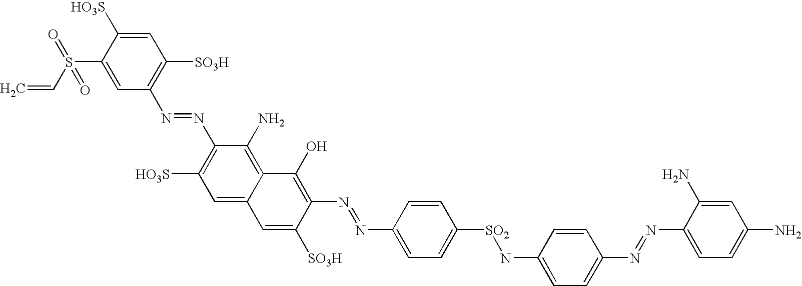 | black |
| 14_183 | 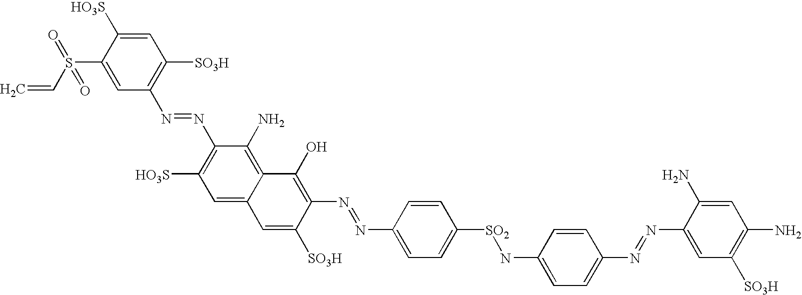 | black |
| 14_184 | 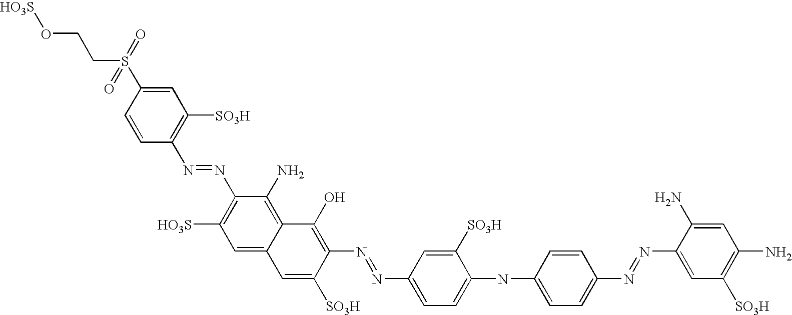 | darkblue |
| 14_185 | 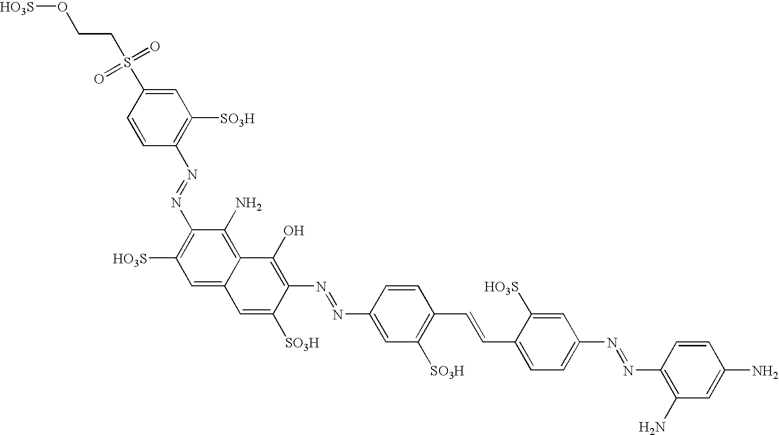 | blue |
| 14_186 | 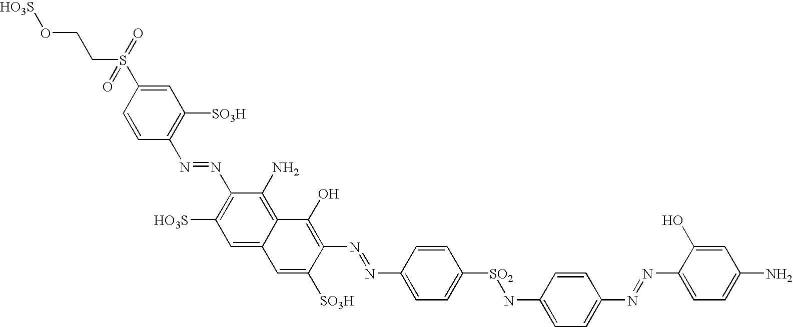 | darkgreen |
| 14_187 | 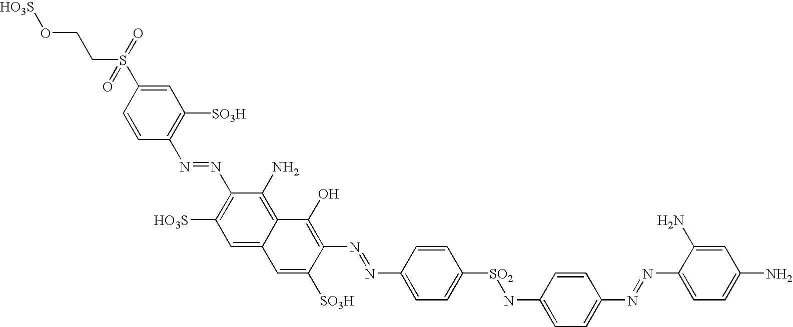 | black |
| 14_188 | 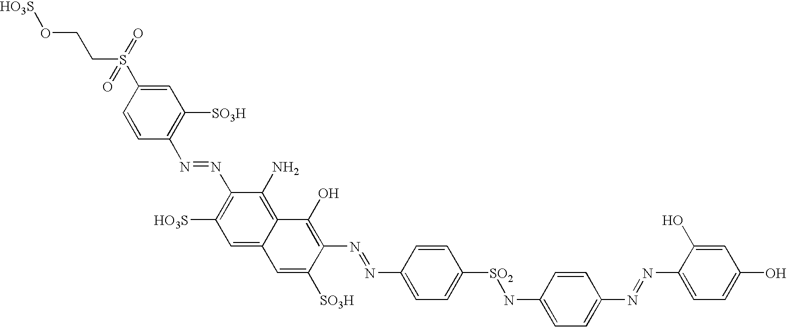 | darkgreen |
| 14_189 | 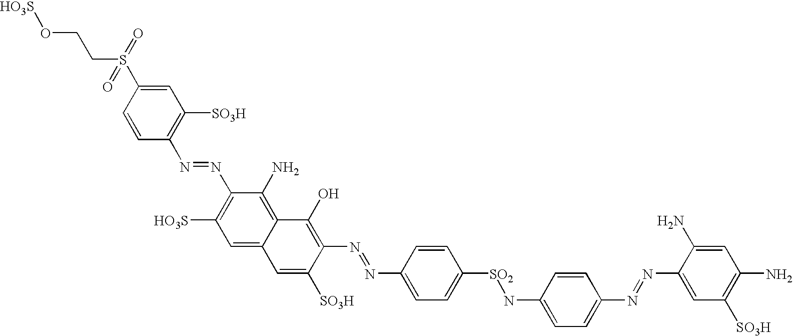 | black |
| 14_190 | 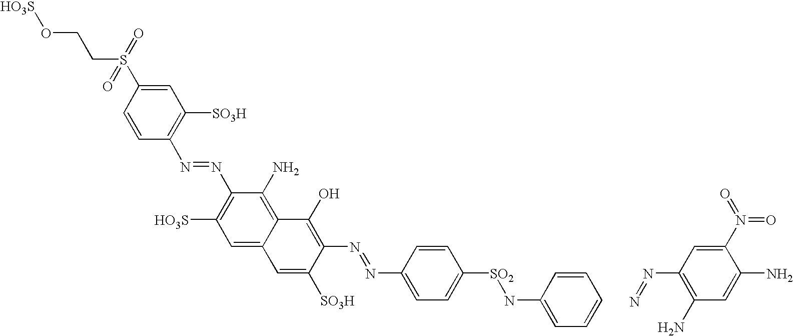 | green |
| 14_191 | 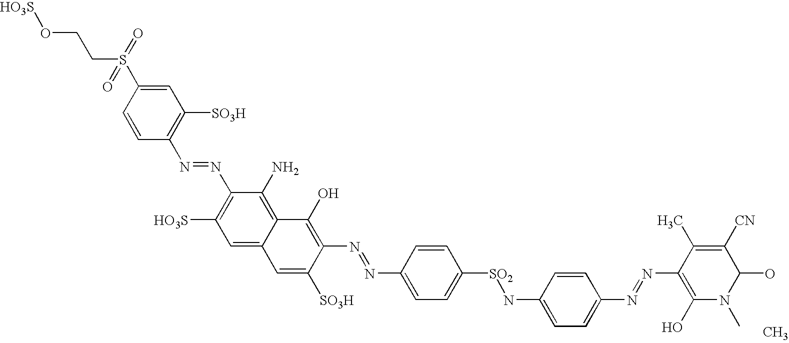 | green |
| 14_192 | 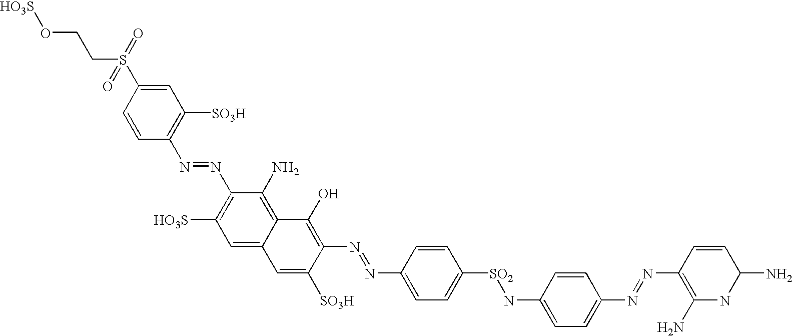 | green |
| 14_193 | 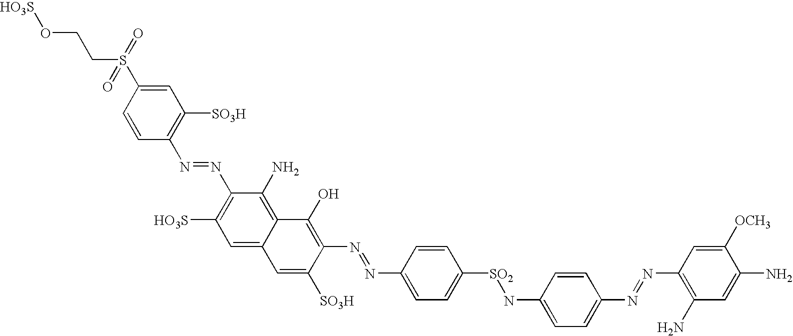 | black |
| 14_194 | 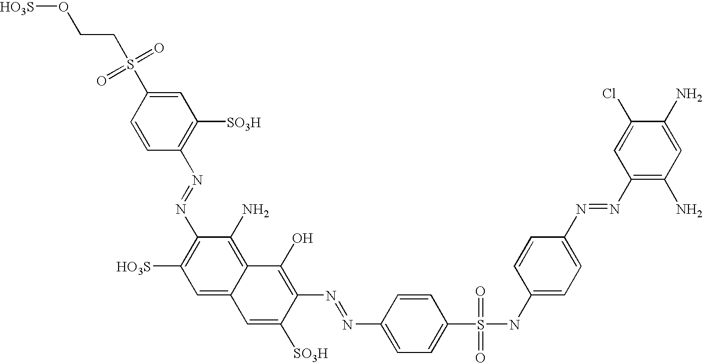 | black |
| 14_195 | 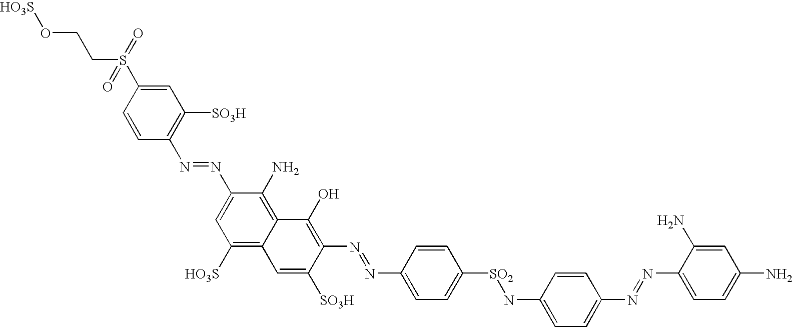 | black |
| 14_196 | 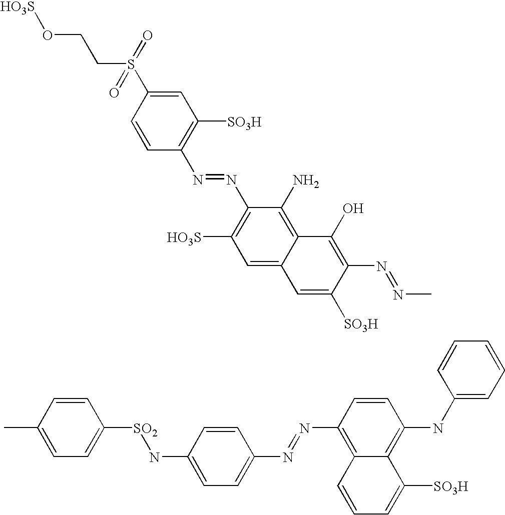 | black |
| 14_197 | 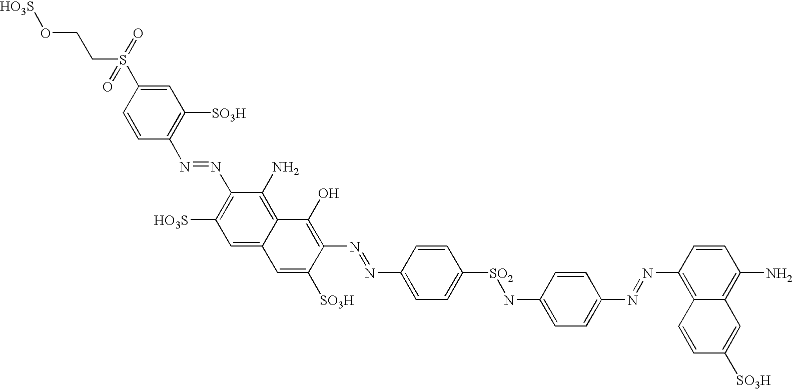 | black |
| 14_198 | 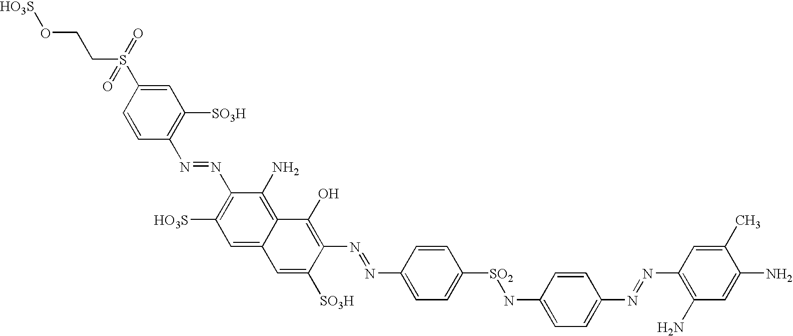 | black |
| 14_199 | 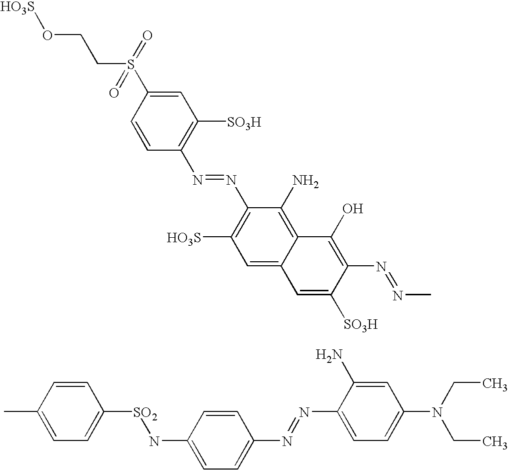 | black |
| 14_200 | 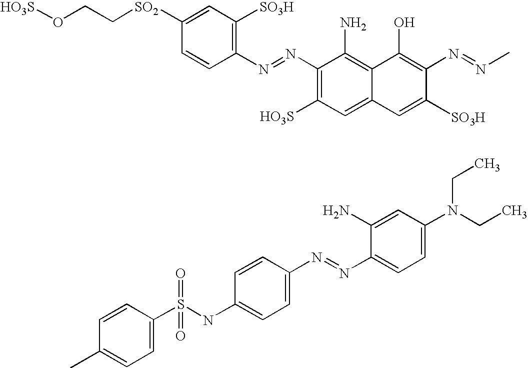 | black |
| 14_201 | 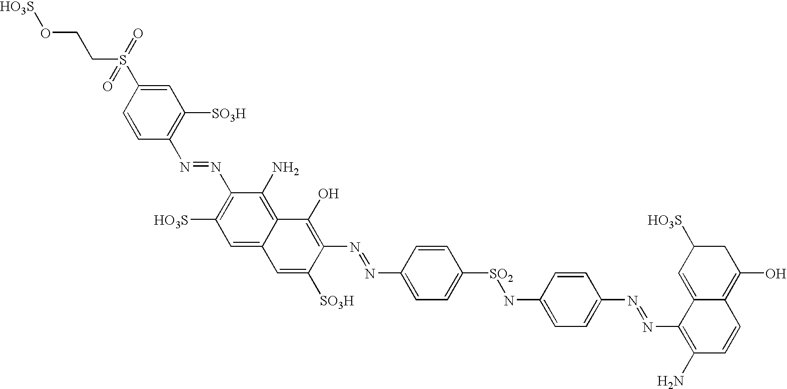 | black |
| 14_202 | 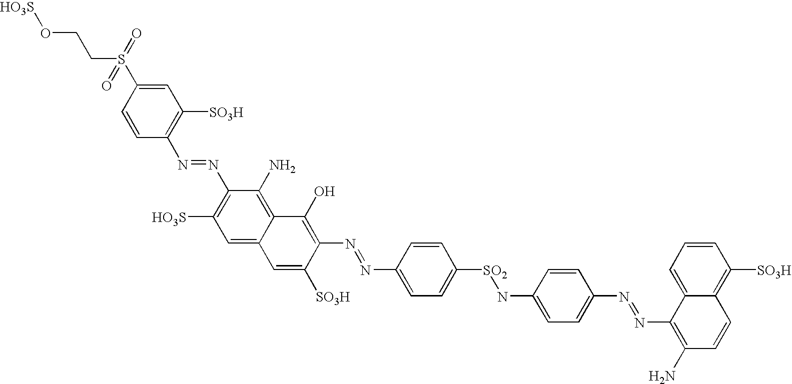 | black |
| 14_203 | 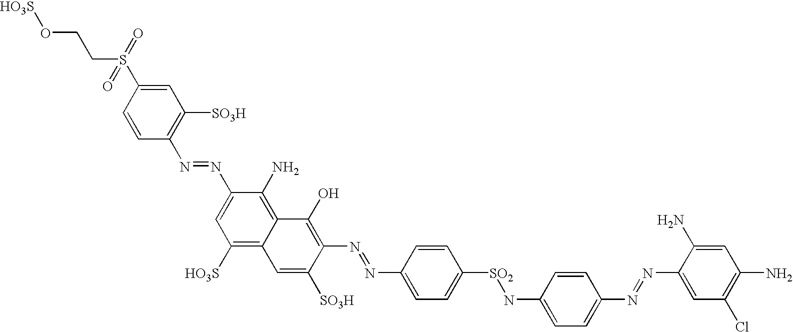 | black |
| 14_204 | 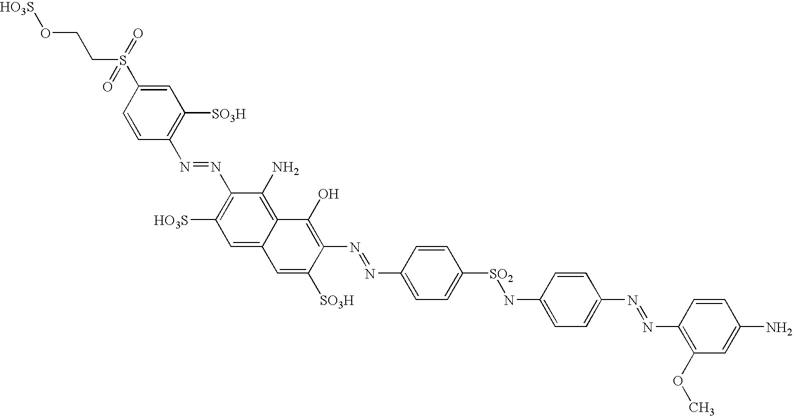 | black |
| 14_205 | 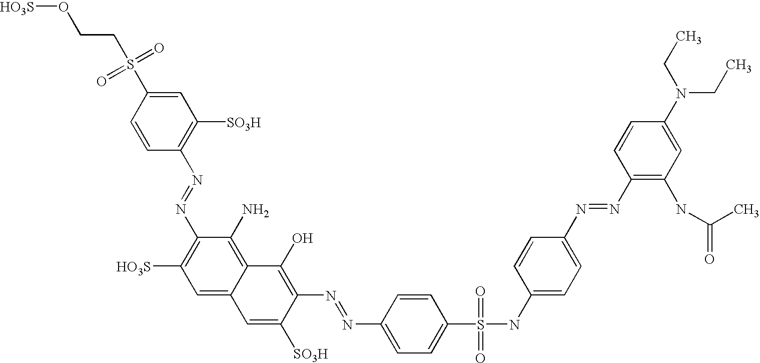 | black |
| 14_206 | 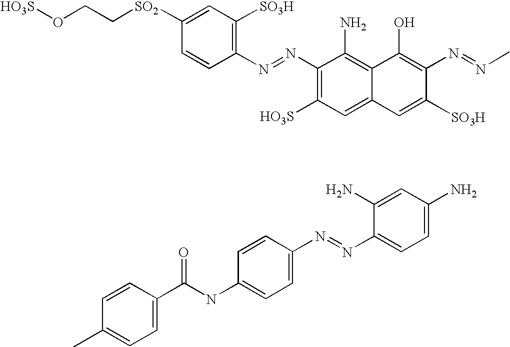 | black |
| 14_207 | 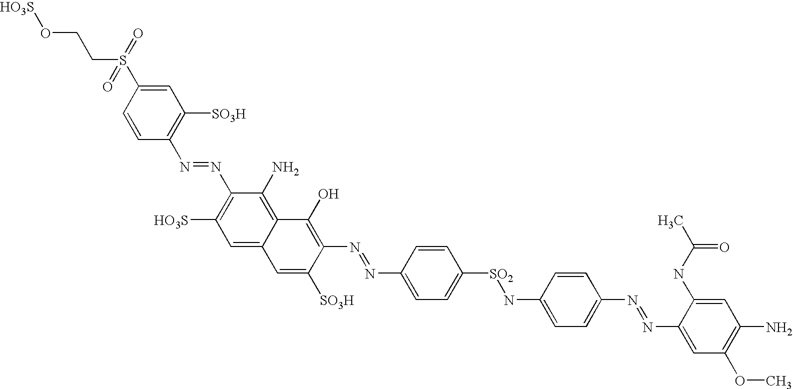 | black |
| 14_208 | 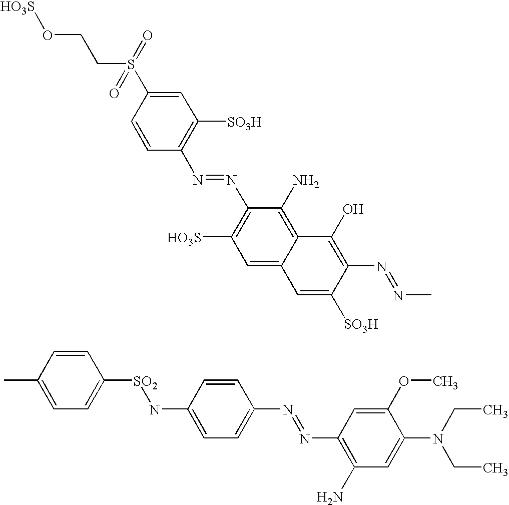 | black |
| 14_209 | 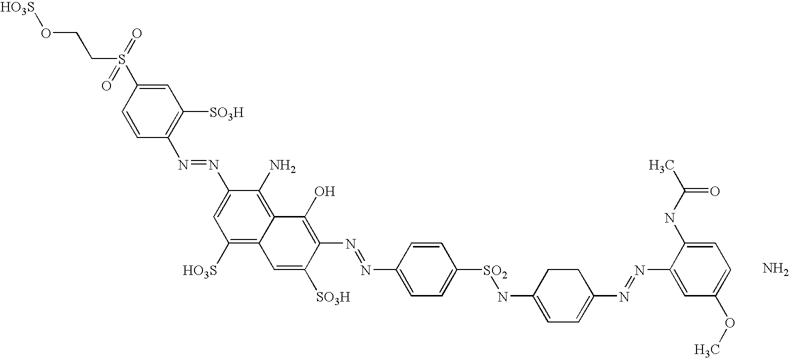 | black |
| 14_210 | 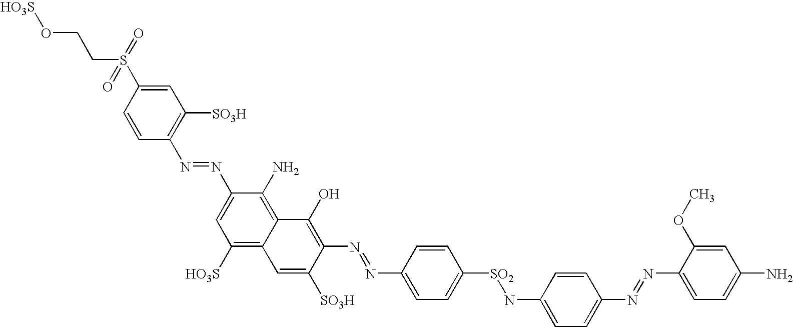 | black |
| 14_211 | 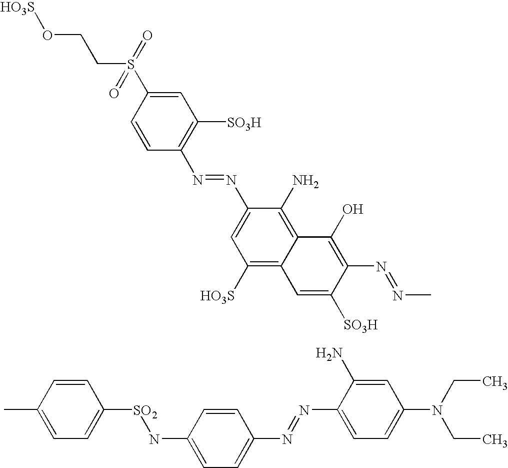 | black-violet |
| 14_212 | 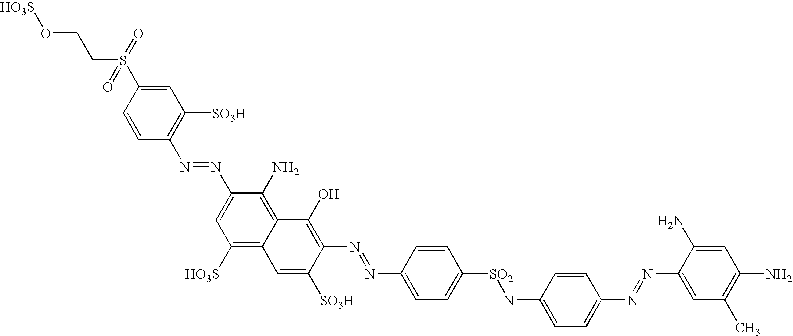 | black |
| 14_213 | 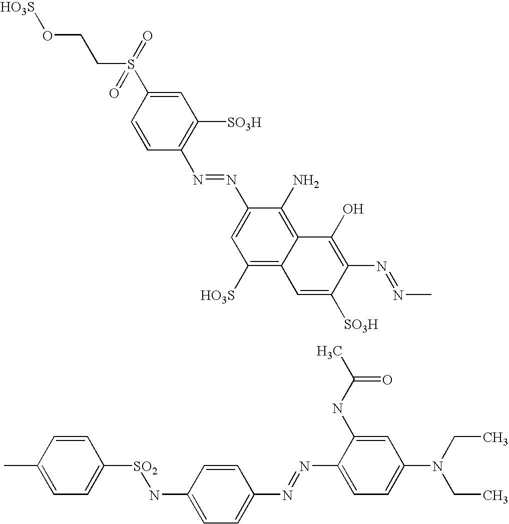 | black |
| 14_214 | 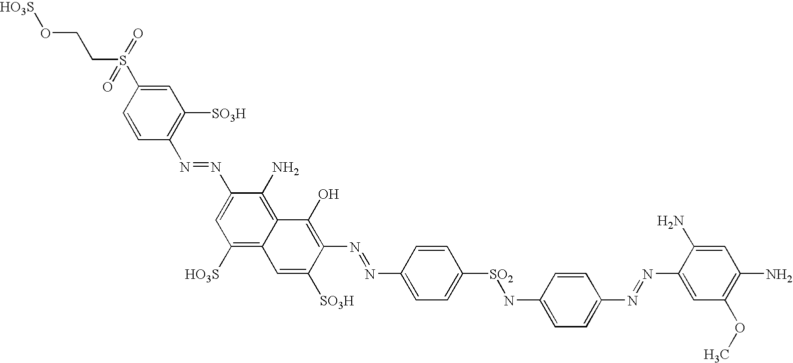 | black |
| 14_215 | 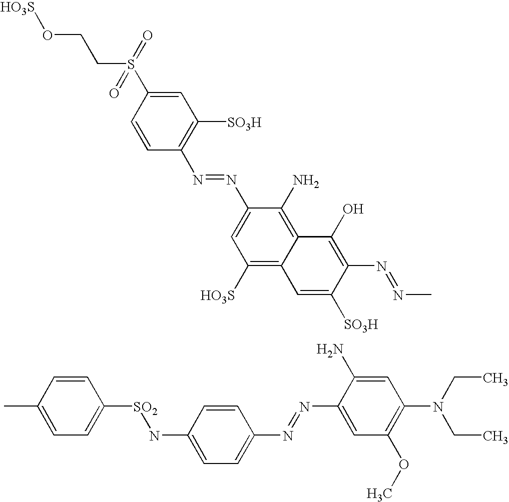 | black-violet |
| 14_216 | 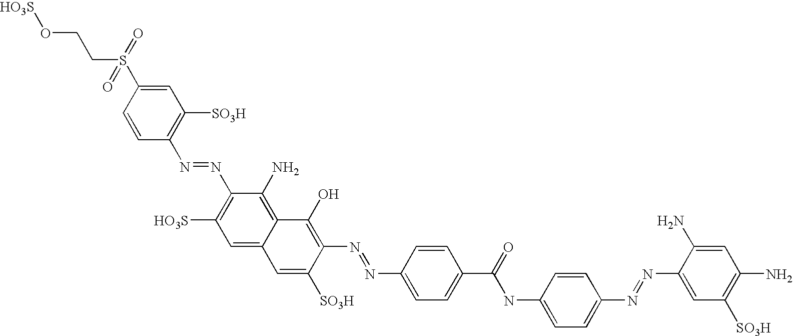 | black |
| 14_217 | 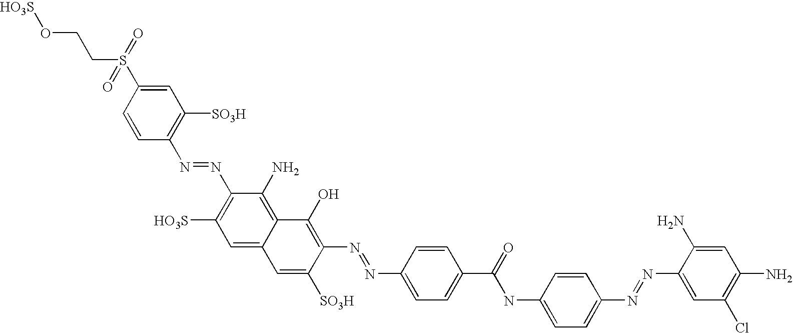 | black |
| 14_218 | 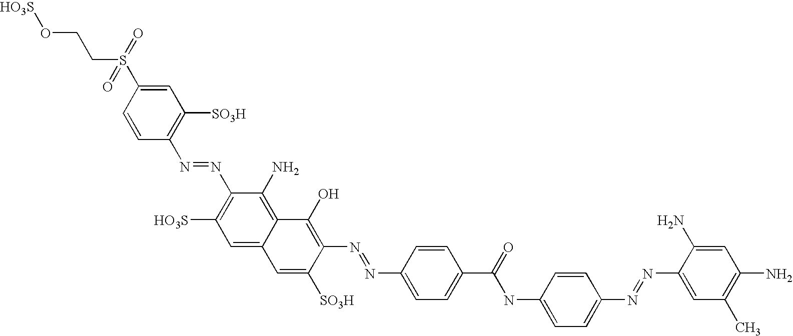 | black |
| 14_219 | 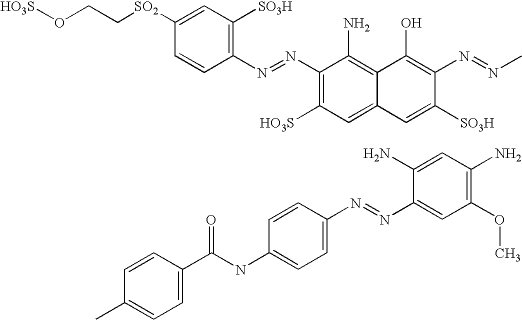 | black |
| 14_220 | 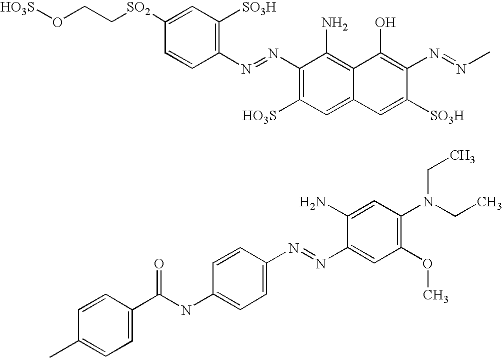 | black |
| 14_221 | 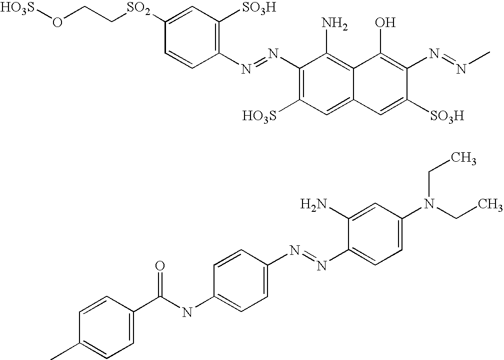 | black |
| 14_222 | 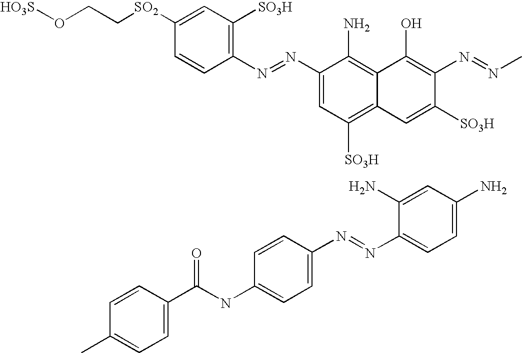 | black |
| 14_223 | 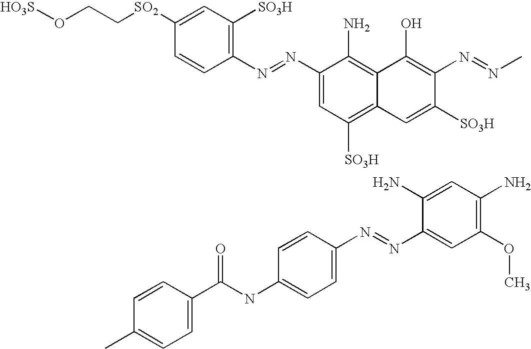 | black |
| 14_224 | 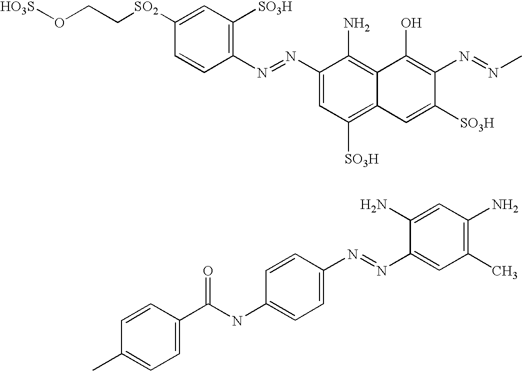 | black |
| 14_225 | 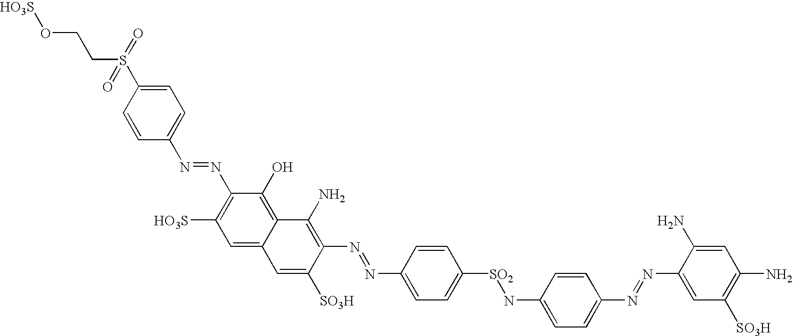 | black |
| 14_226 | 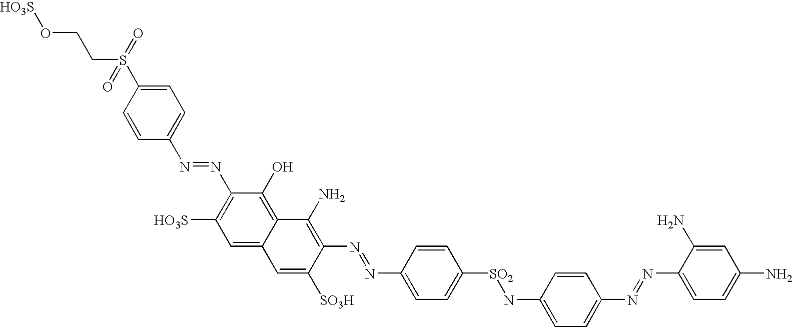 | black |
| 14_227 | 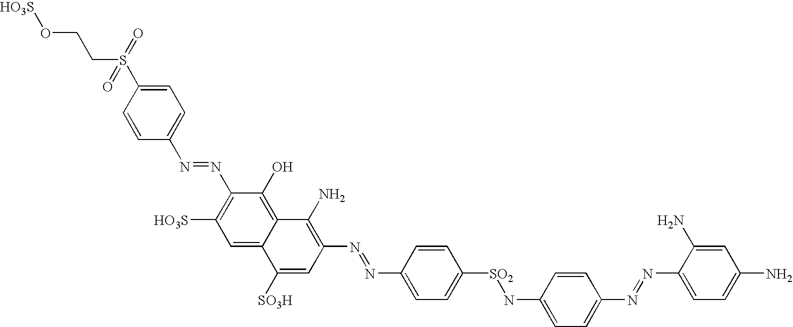 | black |
| 14_228 | 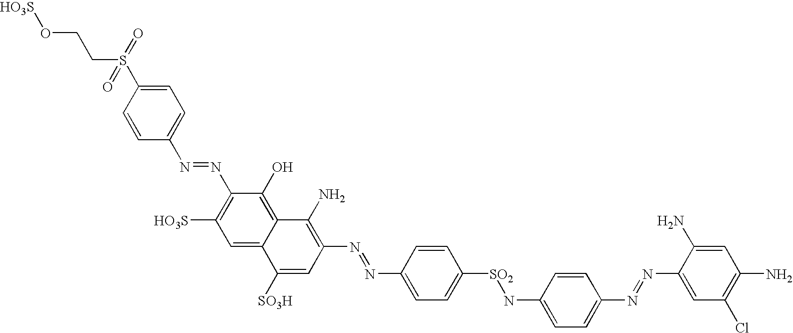 | black |
| 14_229 | 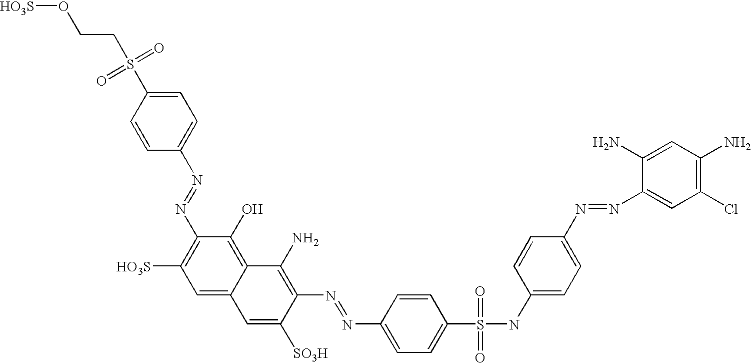 | black |
| 14_230 | 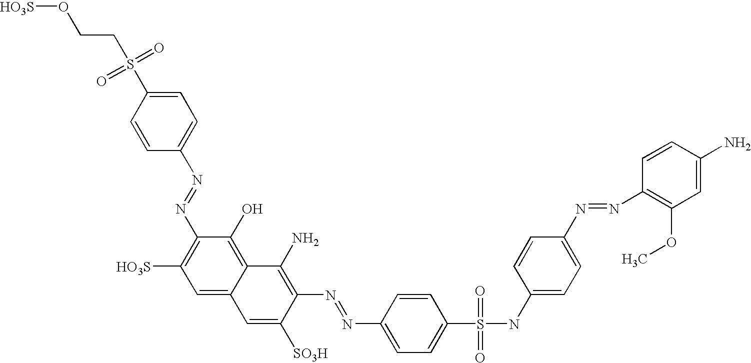 | black |
| 14_231 | 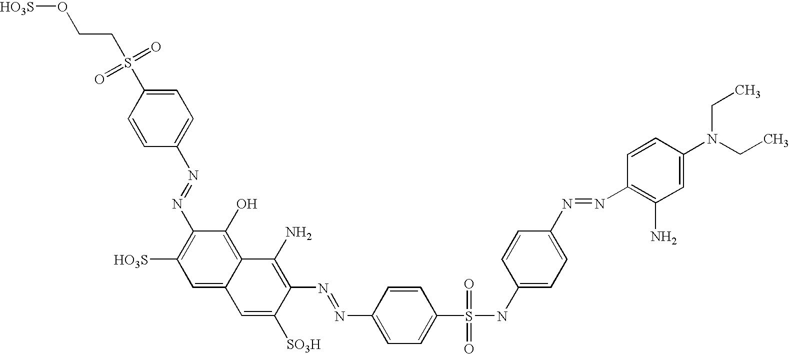 | black |
| 14_232 | 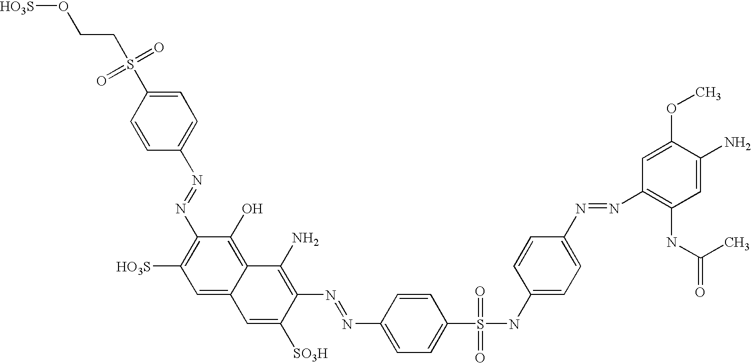 | black |
| 14_233 | 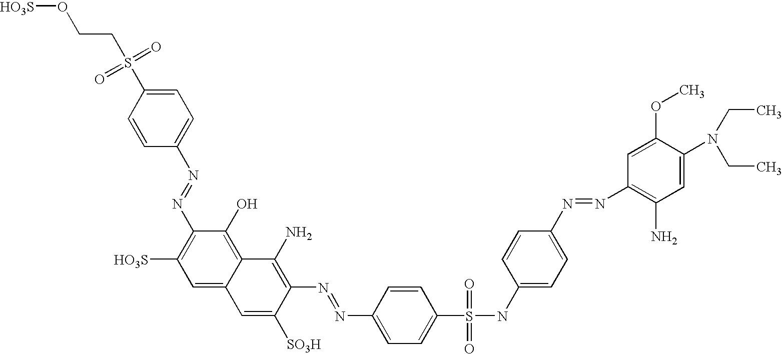 | black |
| 14_234 | 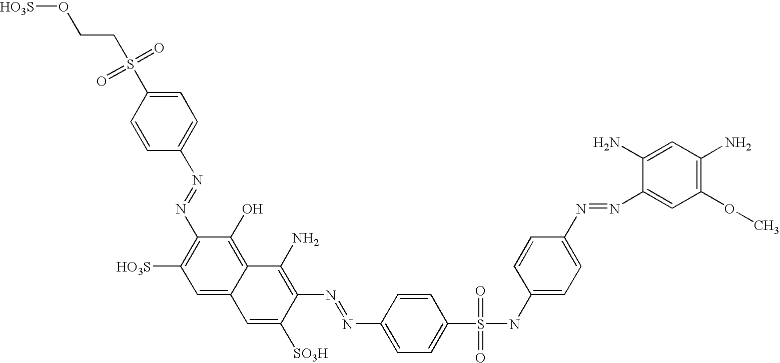 | black |
| 14_235 | 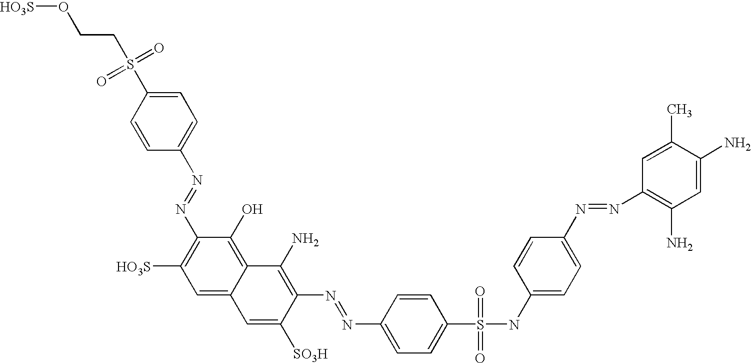 | black |
| 14_236 | 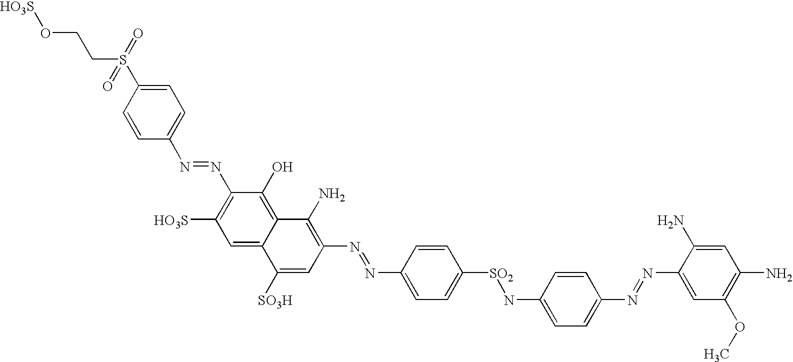 | black |
| 14_237 | 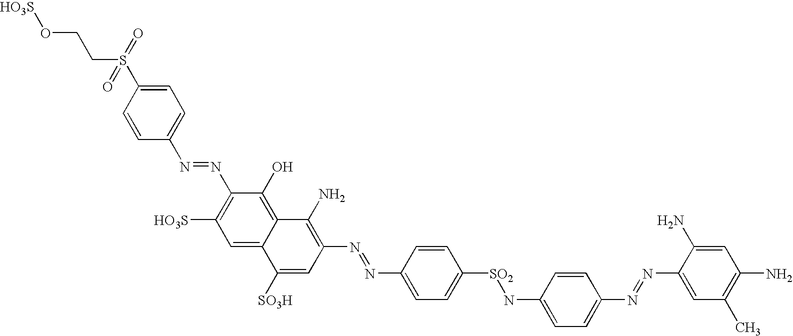 | black |
| 14_238 | 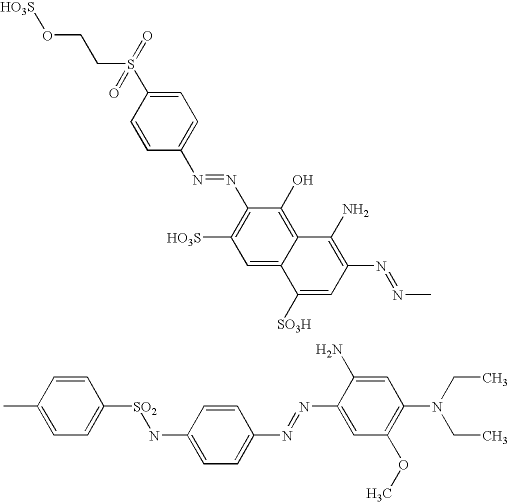 | violet |
| 14_239 | 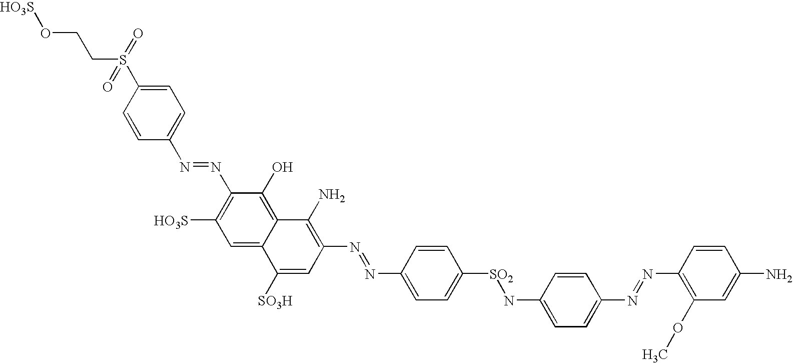 | black |
| 14_240 | 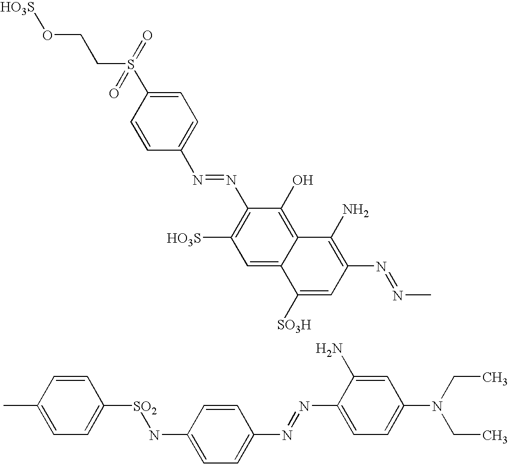 | violet |
| 14_241 | 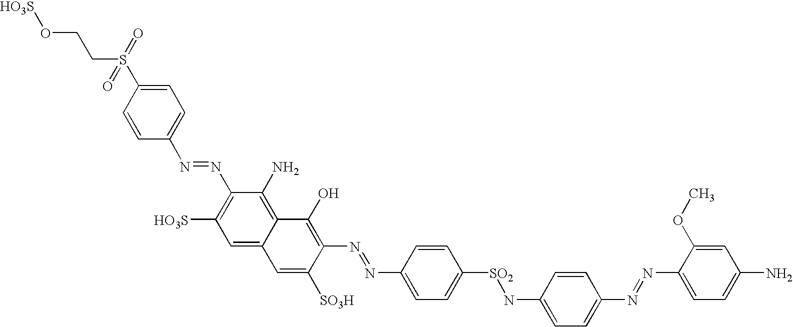 | black |
| 14_242 | 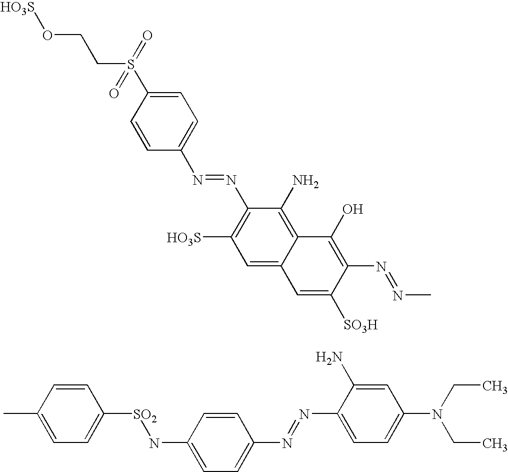 | black |
| 14_243 | 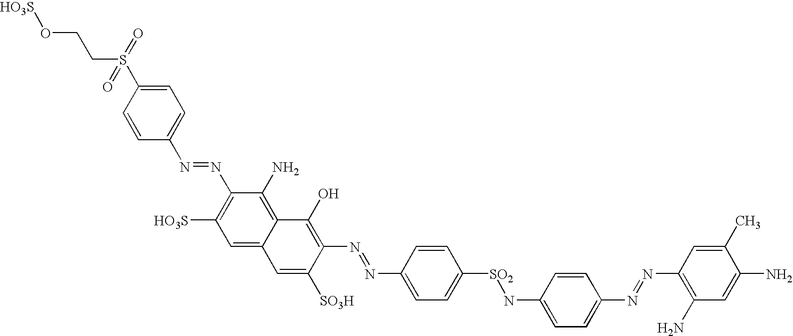 | black |
| 14_244 | 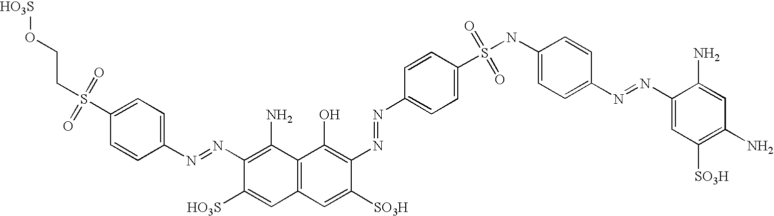 | black |
| 14_245 | 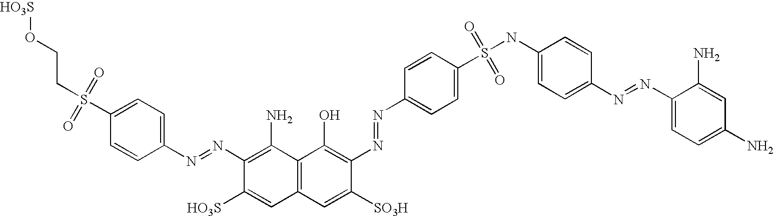 | black |
| 14_246 | 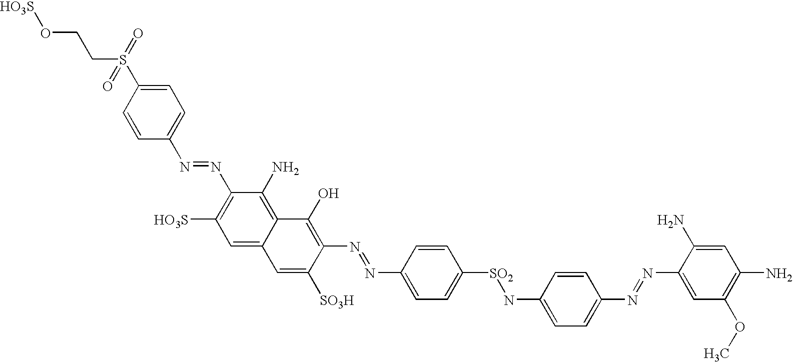 | black |
| 14_247 | 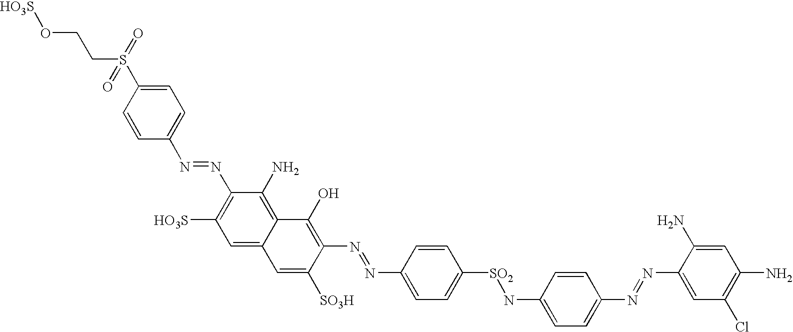 | black |
| 14_248 | 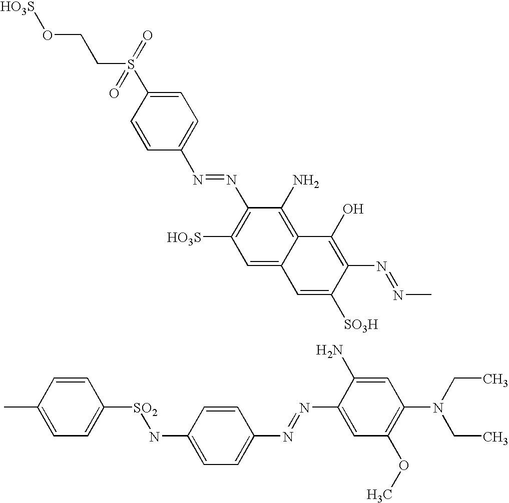 | black |
| 14_249 | 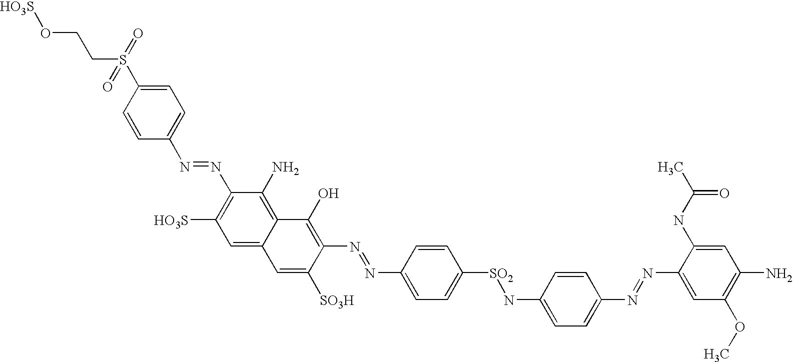 | black |
| 14_250 | 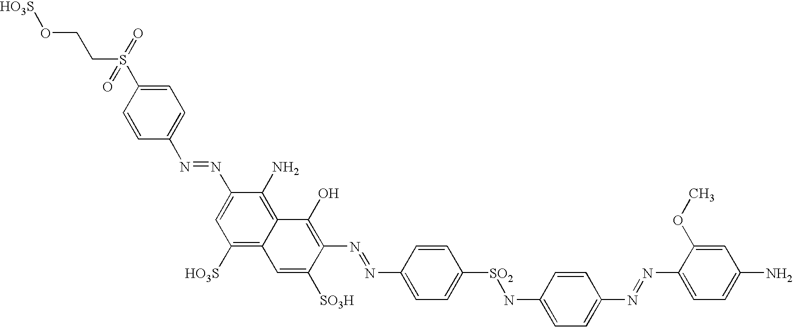 | black |
| 14_251 | 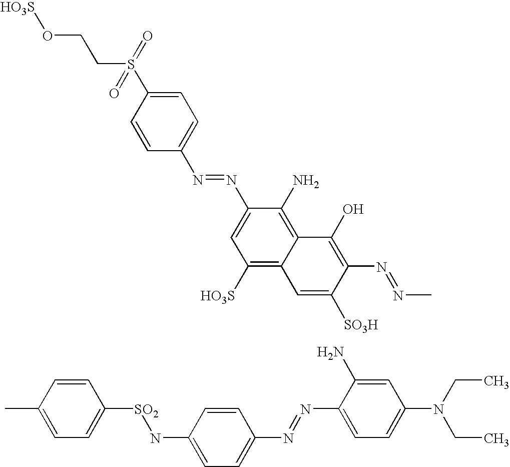 | black |
| 14_252 | 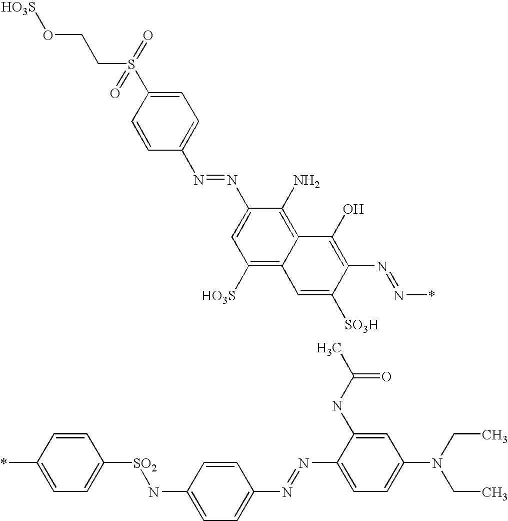 | black |
| 14_253 | 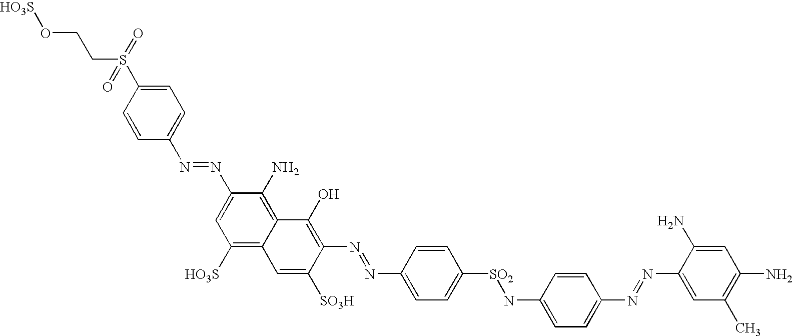 | black |
| 14_254 | 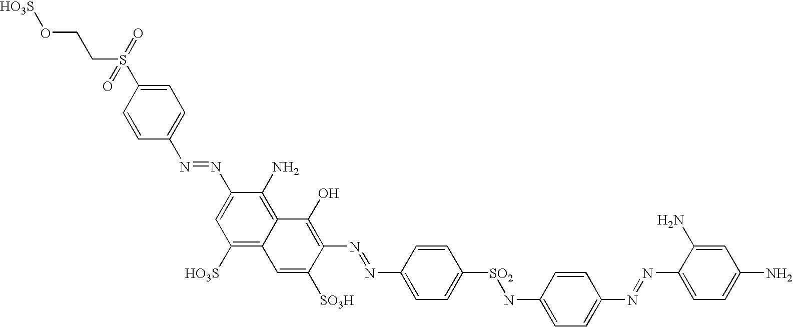 | black |
| 14_255 | 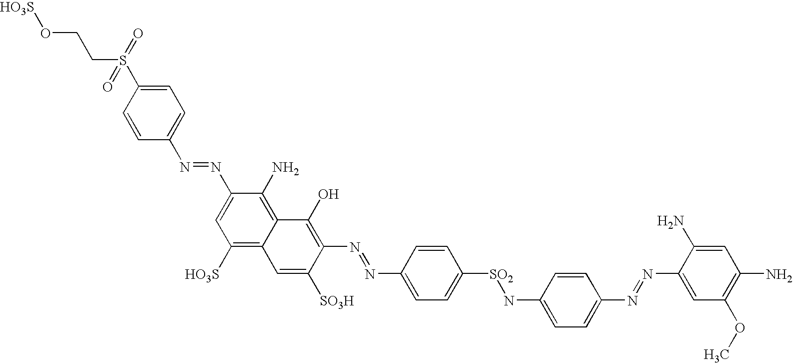 | black |
| 14_256 | 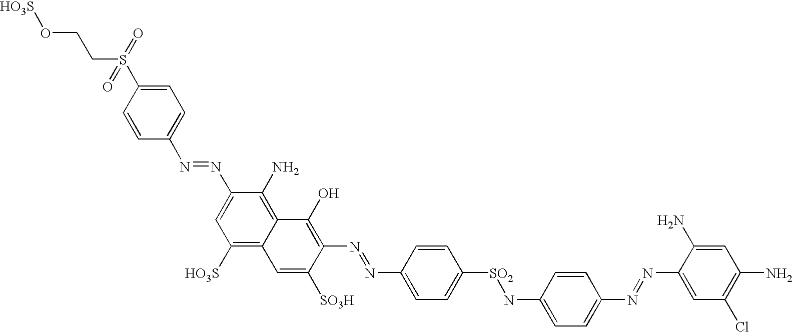 | black |
| 14_257 | 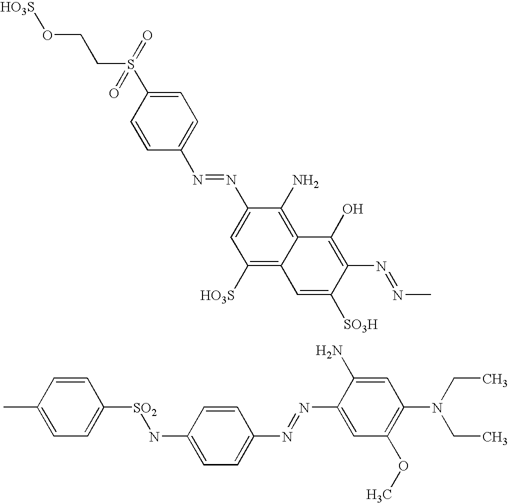 | black |
| 14_258 | 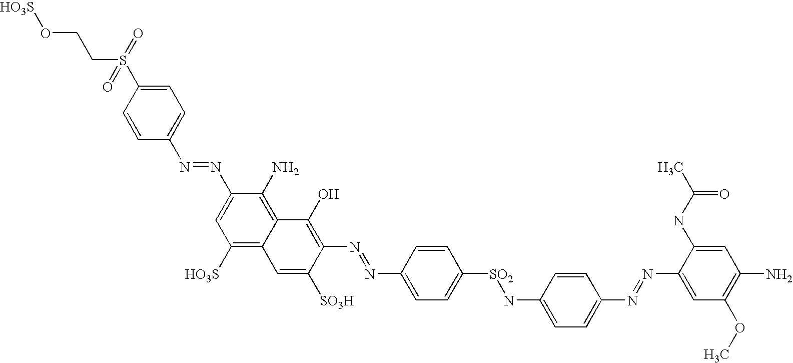 | black |
| 14_259 | 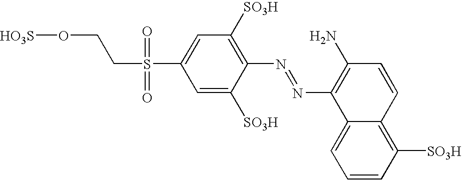 | beige |
| 14_260 | 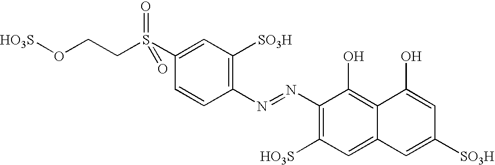 | violet |
| 14_261 | 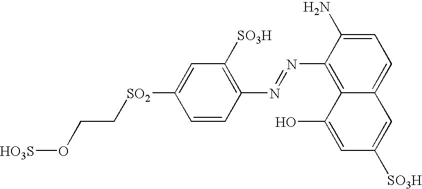 | red |
| 14_262 | 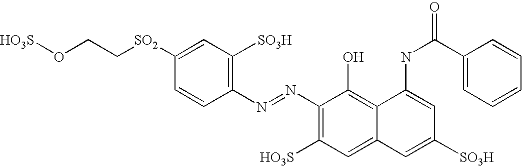 | red |
| 14_263 | 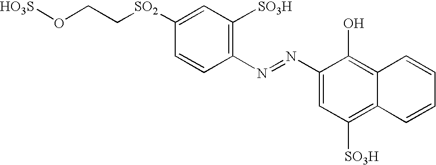 | red |
| 14_264 | 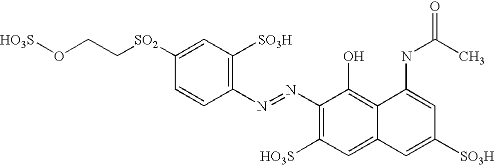 | red |
| 14_265 | 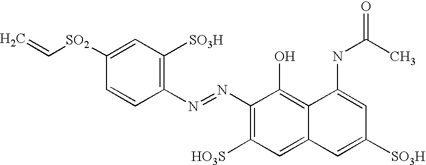 | red |
| 14_266 | 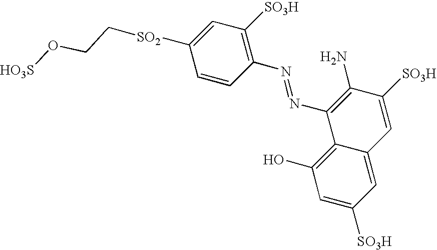 | fuchsia |
| 14_267 | 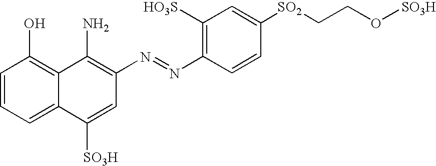 | violet |
| 14_268 | 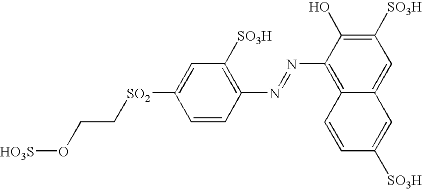 | orange |
| 14_269 | 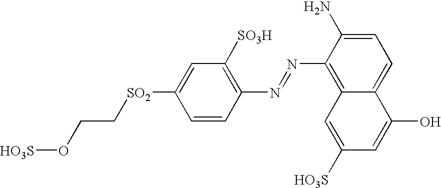 | red |
| 14_270 | 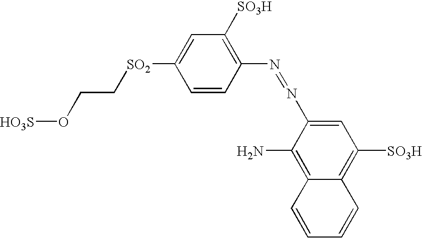 | red |
| 14_271 | 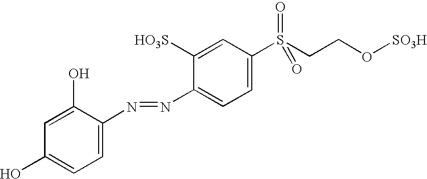 | yellow |
| 14_272 | 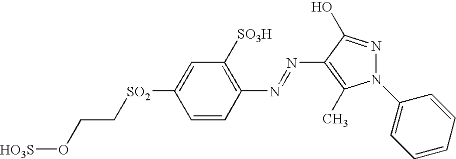 | yellow |
| 14_273 | 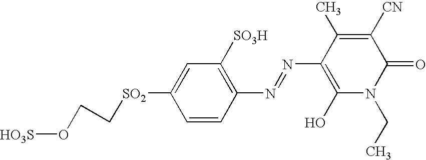 | yellow |
| 14_274 | 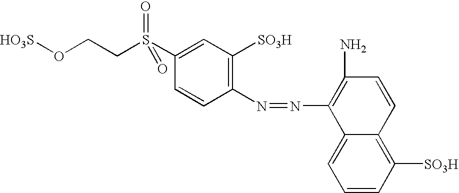 | red |
| 14_275 | 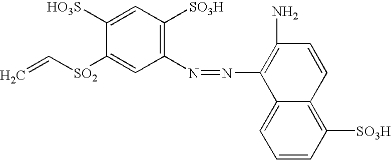 | red |
| 14_276 | 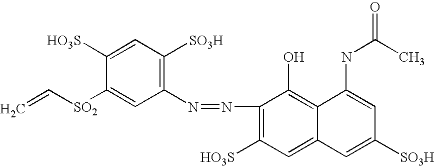 | red |
| 14_277 | 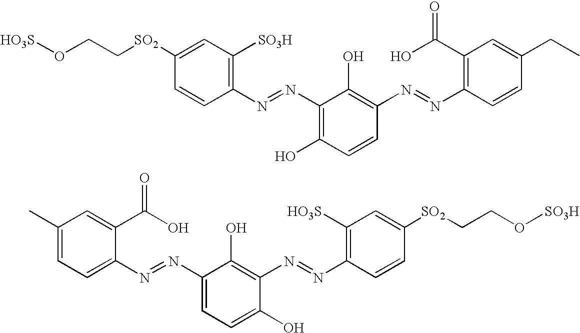 | brown |
| 14_278 | 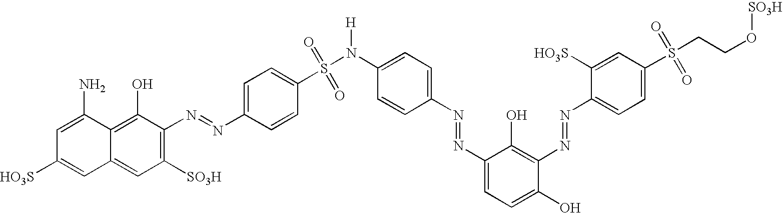 | rust-red |
| 14_279 | 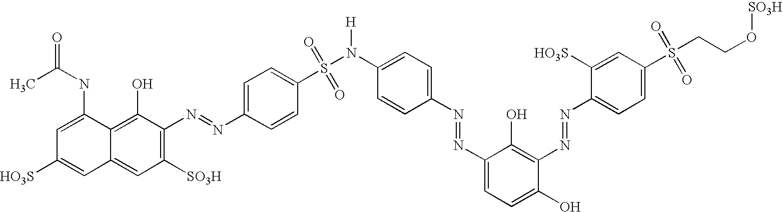 | red-brown |
| 14_280 | 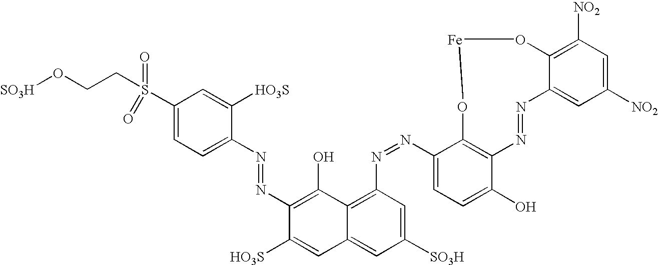 | brown |
| 14_281 | 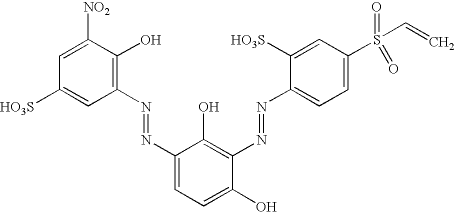 | brown |
| 14_282 | 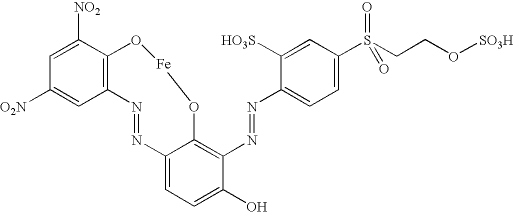 | brown |
| 14_283 | 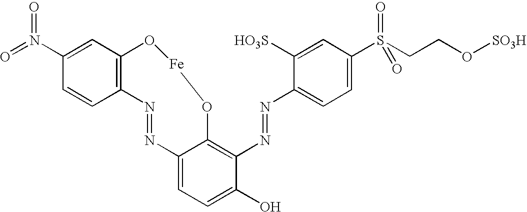 | red-brown |
| 14_284 | 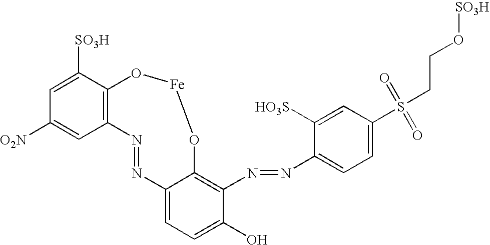 | brown |
| 14_285 | 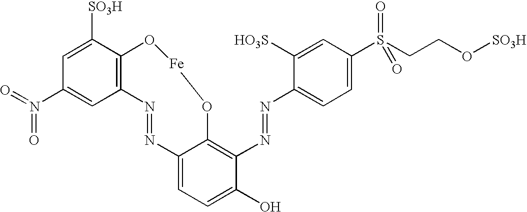 | olivebrown |
| 14_286 | 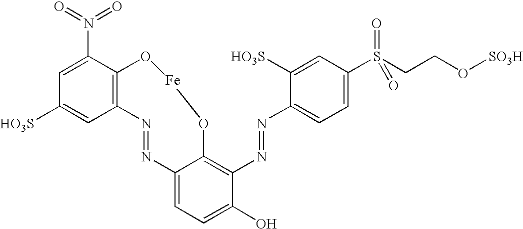 | red-brown |
| 14_287 | 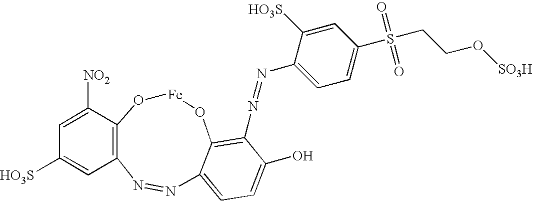 | red-brown |
| 14_288 | 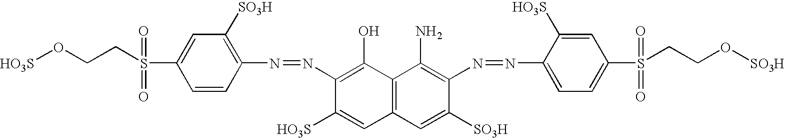 | blue |
| 14_289 | 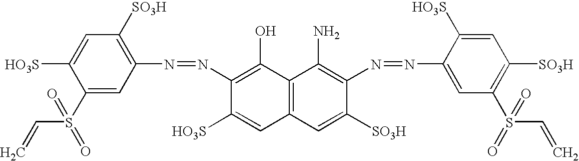 | blue |
| 14_290 | 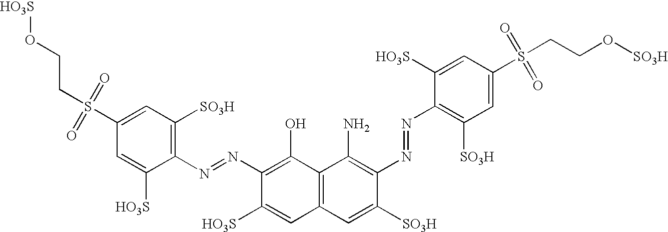 | blackberry |
| 14_291 | 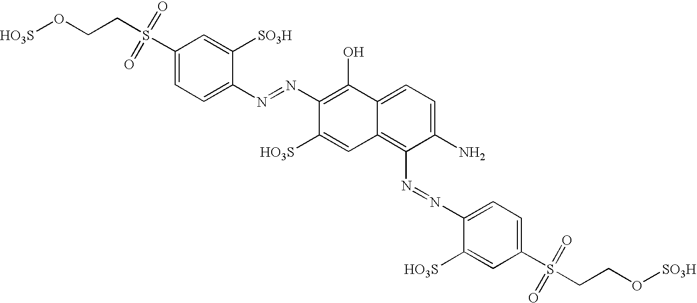 | red |

b) Dyes of formulae 3, 4, 5:

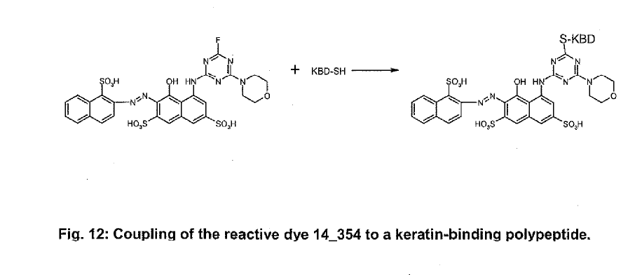

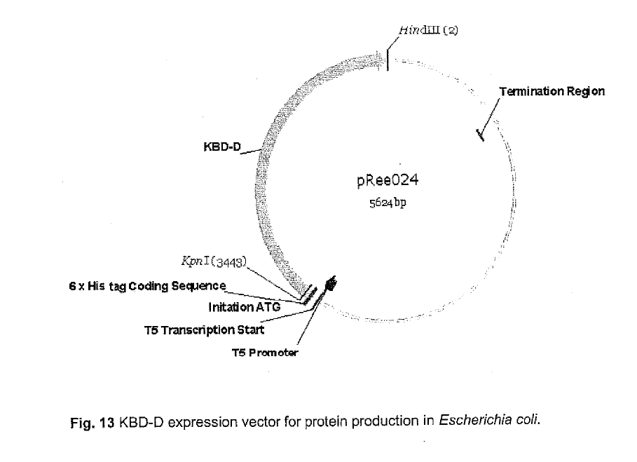

Alternatively, dyes (e.g. dye 14_{—}354, FIG. 12) which have a substitutable halogen atom in their reactive anchor can also be used. These dyes can be reacted without activation with the SH groups of the KBD-B. The reaction between KBD and dye 14_{—}354 takes place in the molar ratio 1:2 since the KBD-B has two free cysteines for the effector coupling. The reaction solution is gently shaken for 30 min at 30° C., pH 8-9.

The following dyes (14_{—}292 to 14_{—}366) with KBD-B (SEQ ID No.: 166) or KBD-D (SEQ ID No.: 168) can be reacted analogously. The color of the respective product is given.

TABLE 7

| | Structure | Color |
|---|---|---|
| 14_292 | 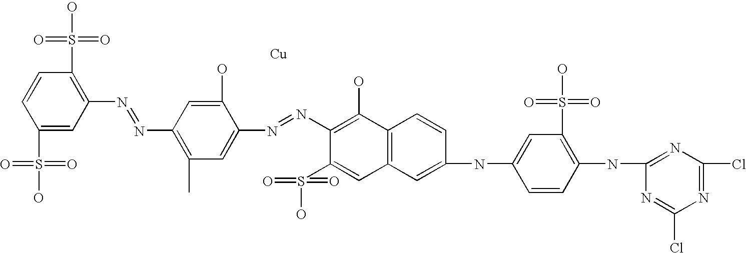 | black |
| 14_293 | 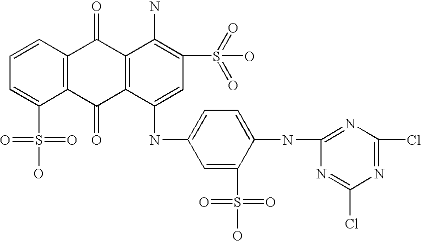 | blue |
| 14_294 | 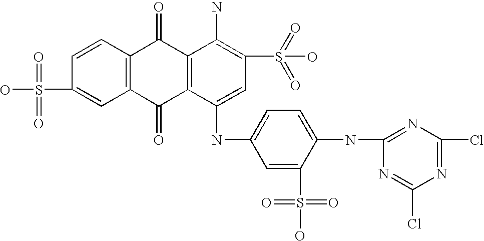 | blue |
| 14_295 | 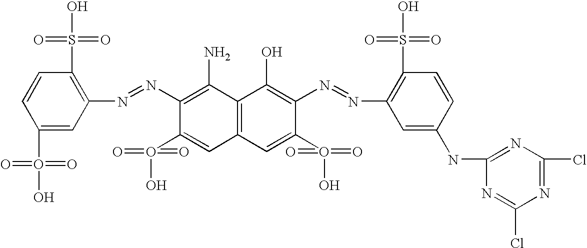 | blue |
| 14_296 | 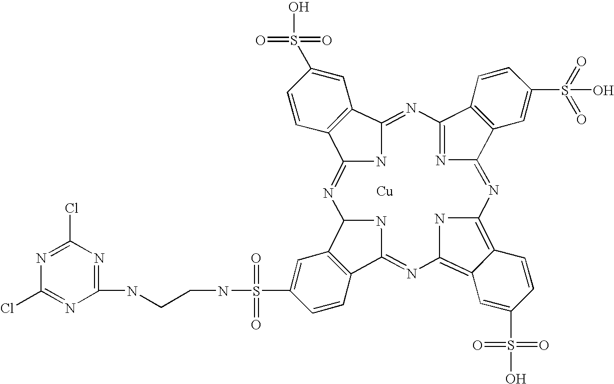 | blue |
| 14_297 | 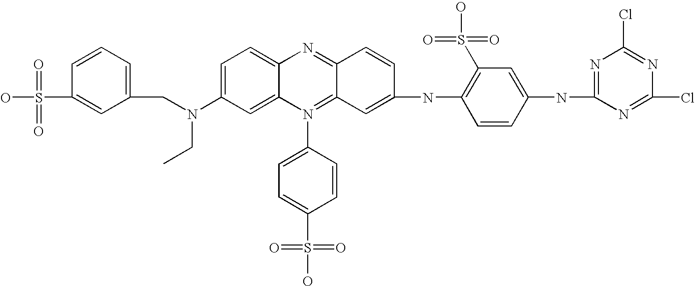 | blue |
| 14_298 | 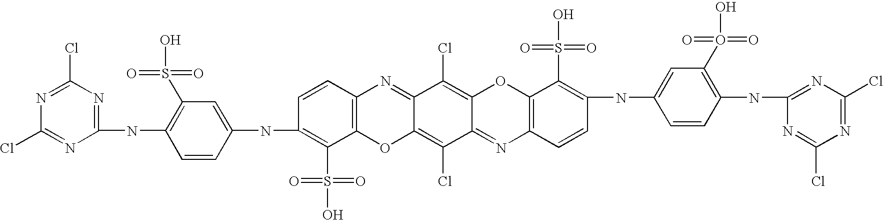 | blue |
| 14_299 | 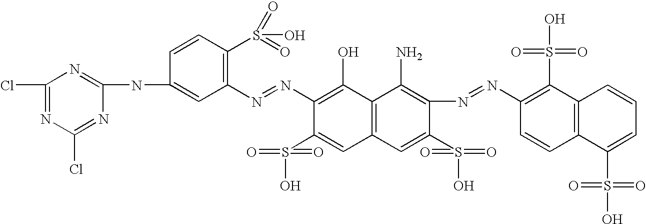 | blue |
| 14_300 | 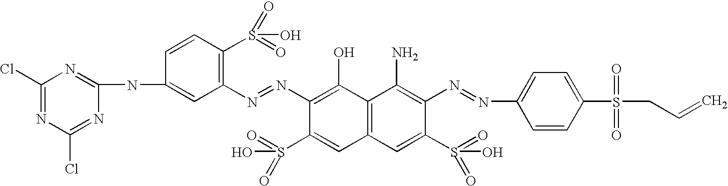 | blue |
| 14_301 | 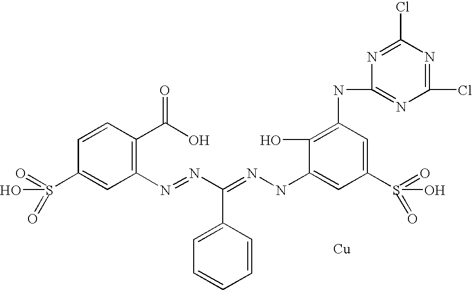 | blue |
| 14_302 | 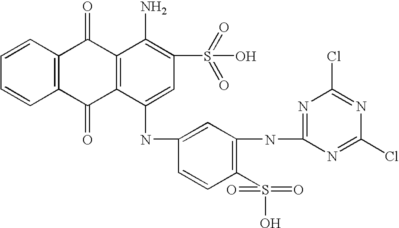 | blue |
| 14_303 | 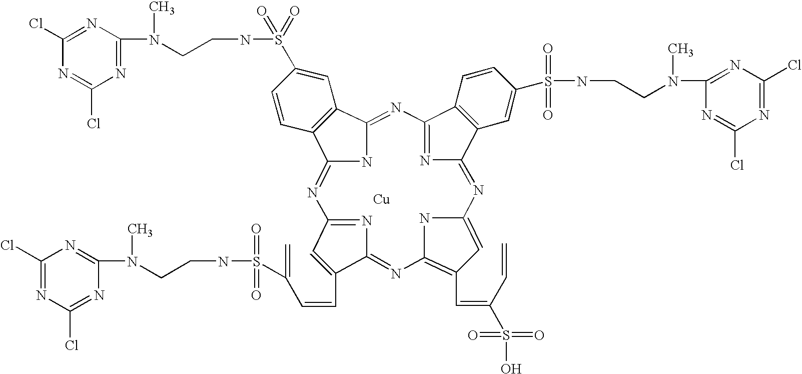 | blue |
| 14_304 | 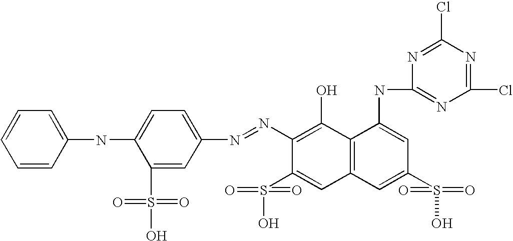 | blue |
| 14_305 | 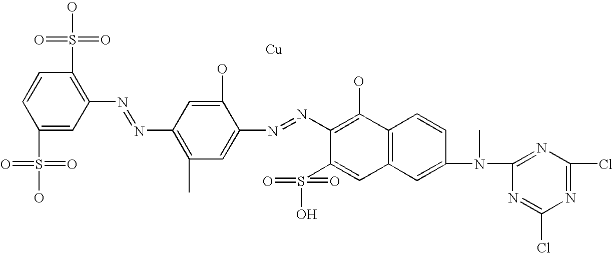 | blue |
| 14_306 | 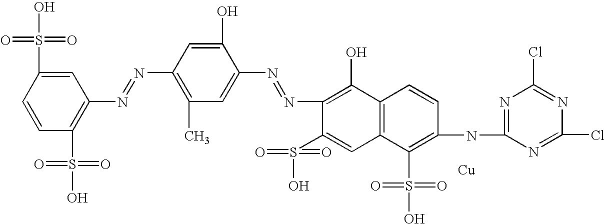 | blue |
| 14_307 | 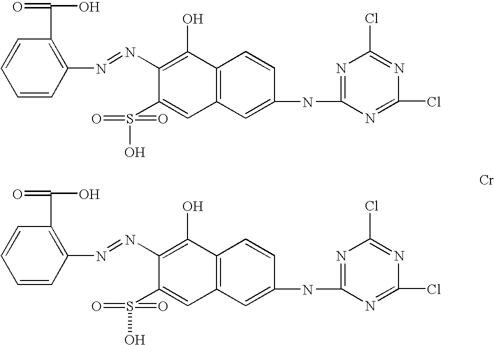 | brown |
| 14_308 | 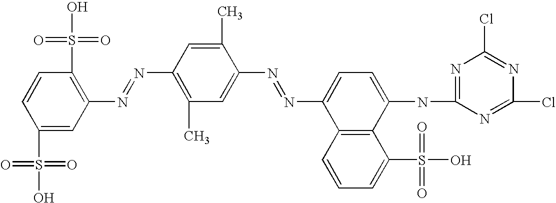 | brown |
| 14_309 | 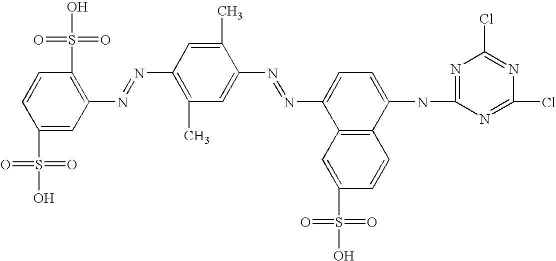 | brown |
| 14_310 | 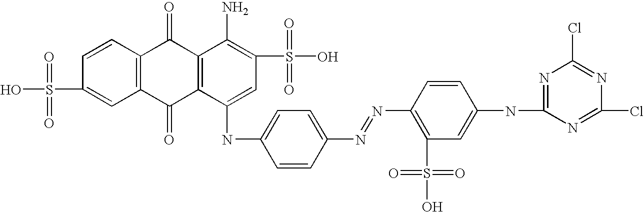 | green |
| 14_311 | 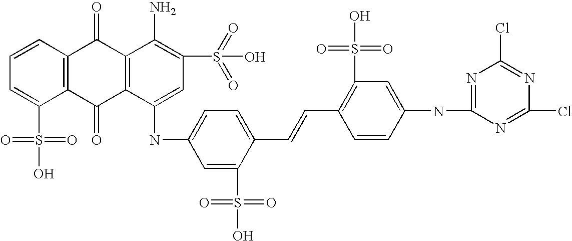 | green |
| 14_312 | 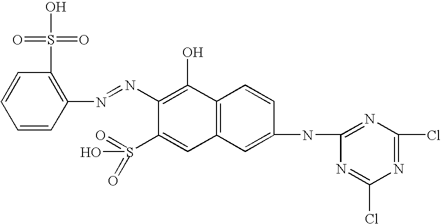 | orange |
| 14_313 | 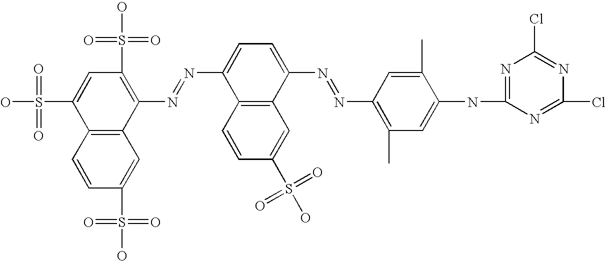 | orange |
| 14_314 | 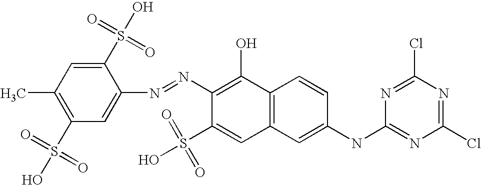 | orange |
| 14_315 | 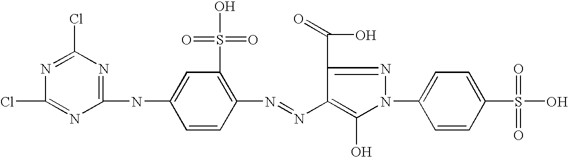 | orange |
| 14_316 | 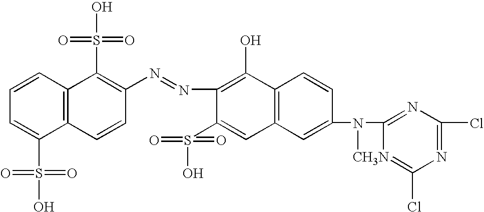 | orange |
| 14_317 | 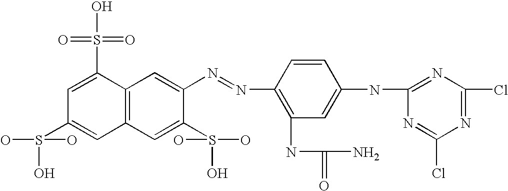 | orange |
| 14_318 | 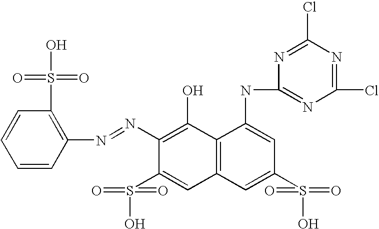 | red |
| 14_319 | 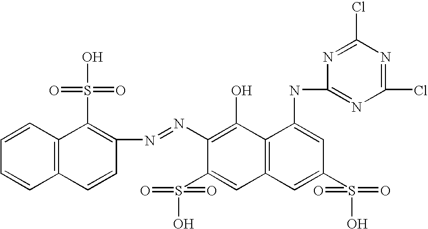 | red |
| 14_320 | 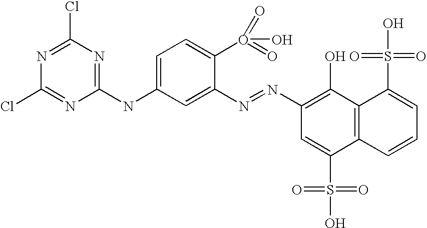 | red |
| 14_321 | 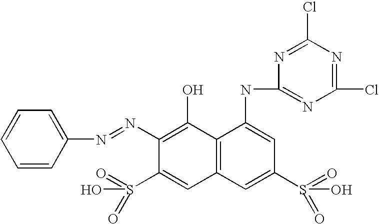 | red |
| 14_322 | 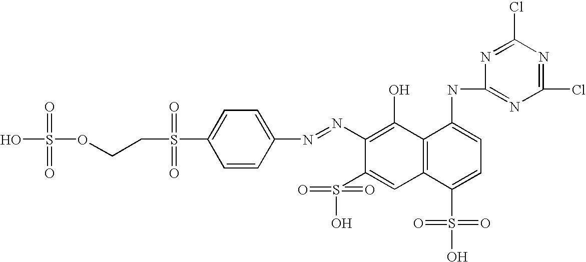 | red |
| 14_323 | 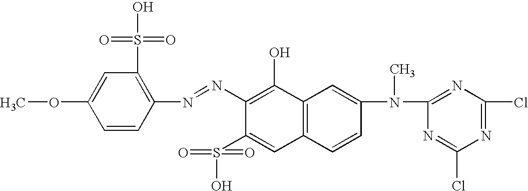 | red |
| 14_324 | 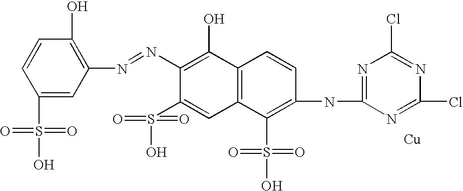 | red |
| 14_325 | 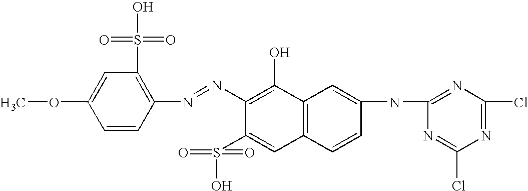 | red |
| 14_326 | 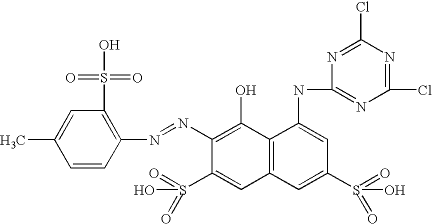 | red |
| 14_327 | 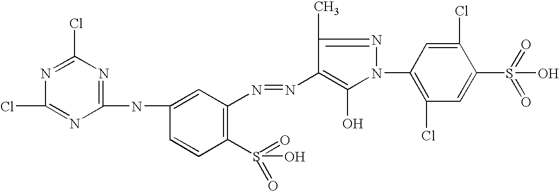 | yellow |
| 14_328 | 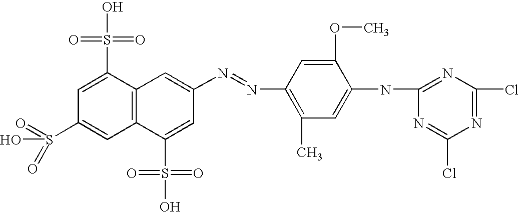 | yellow |
| 14_329 | 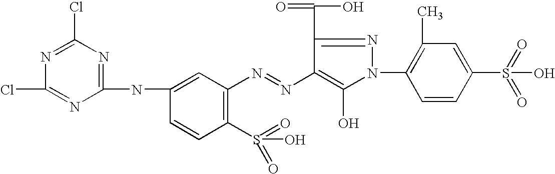 | yellow |
| 14_330 | 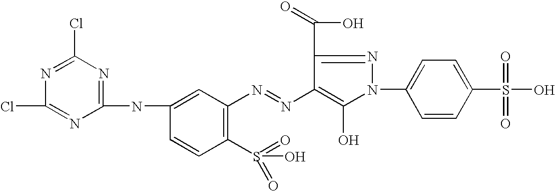 | yellow |
| 14_331 | 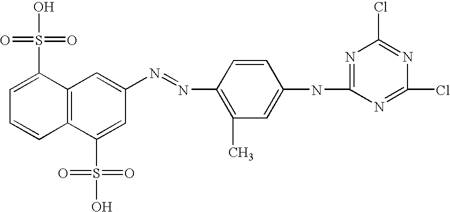 | yellow |
| 14_332 | 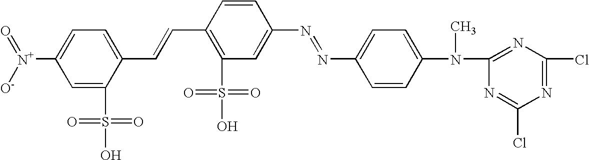 | yellow |
| 14_333 | 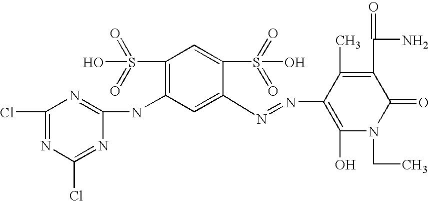 | yellow |
| 14_334 | 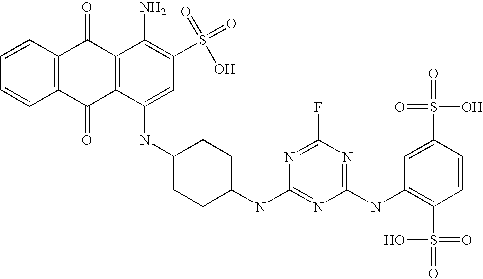 | blue |
| 14_335 | 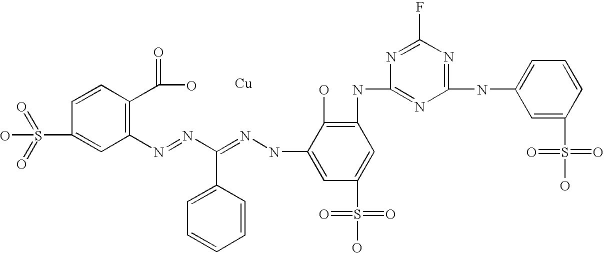 | blue |
| 14_336 | 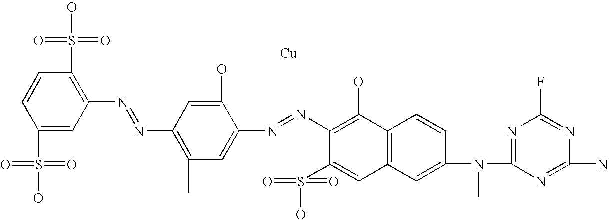 | blue |
| 14_337 | 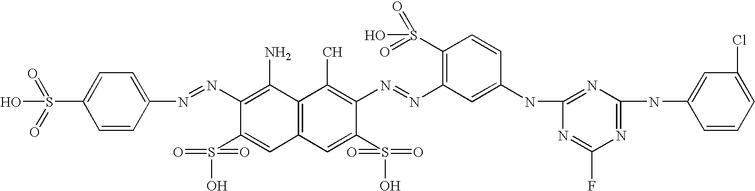 | blue |
| 14_338 | 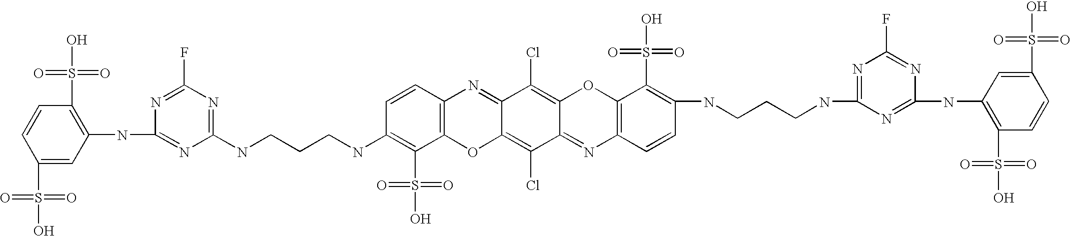 | blue |
| 14_339 | 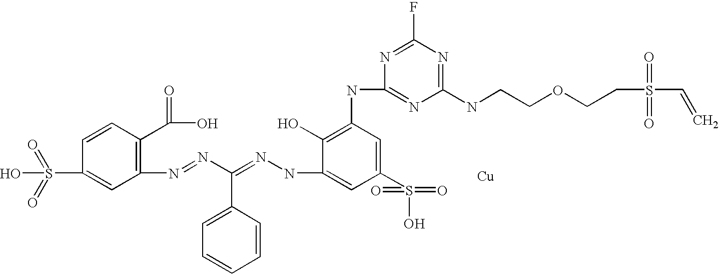 | blue |
| 14_340 | 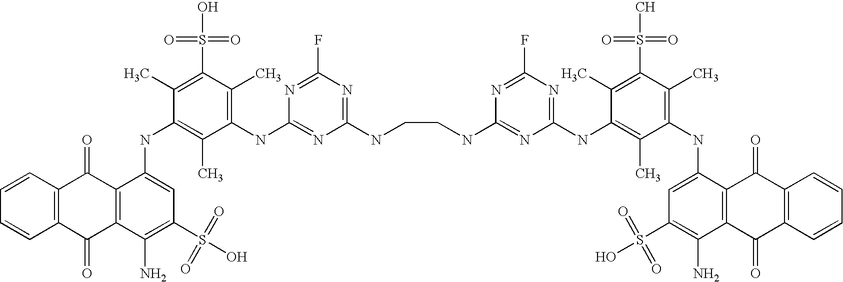 | blue |
| 14_341 | 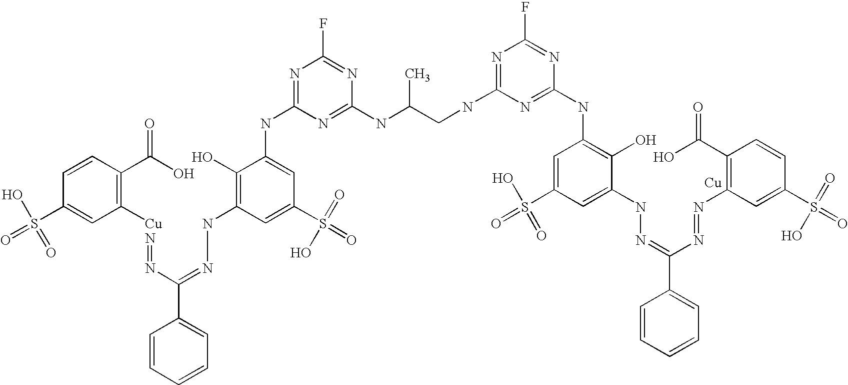 | blue |
| 14_342 | 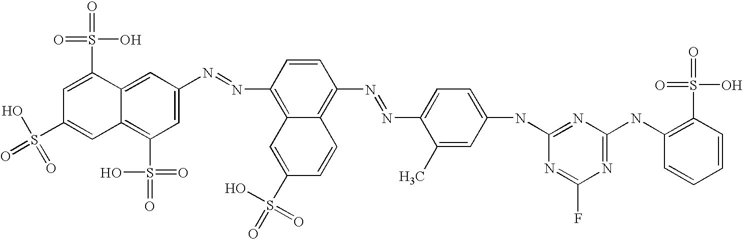 | brown |
| 14_343 | 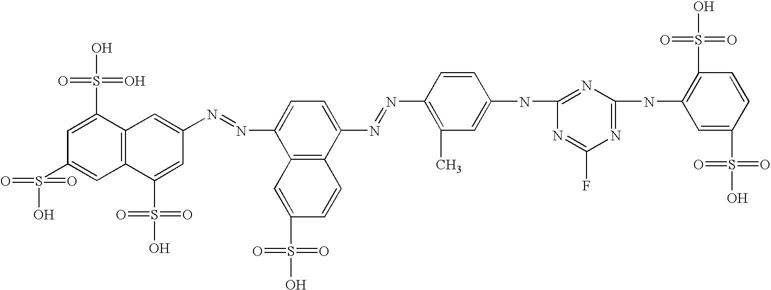 | brown |
| 14_344 | 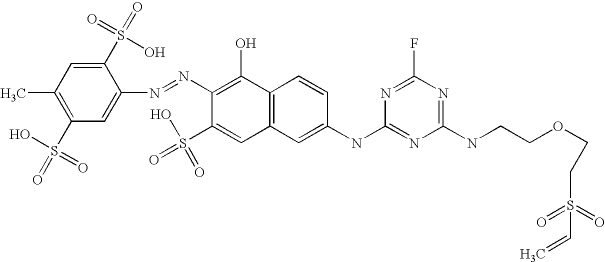 | orange |
| 14_345 | 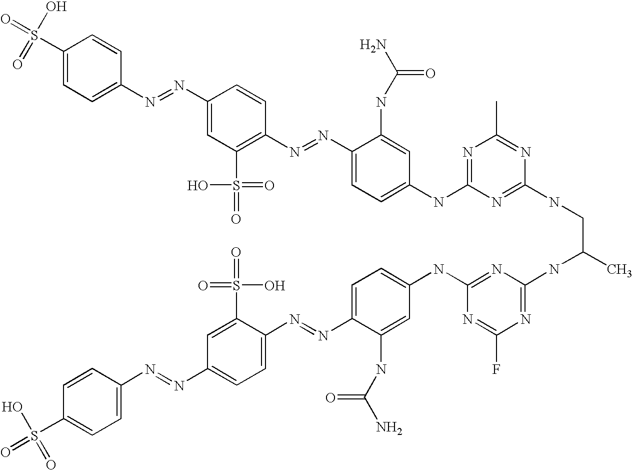 | orange |
| 14_346 | 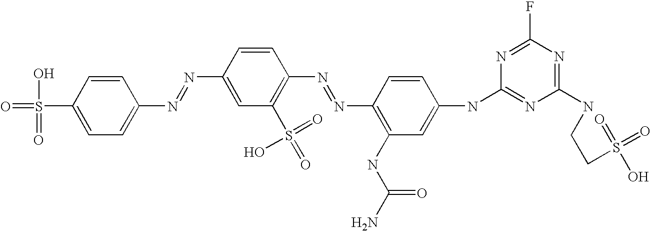 | orange |
| 14_347 | 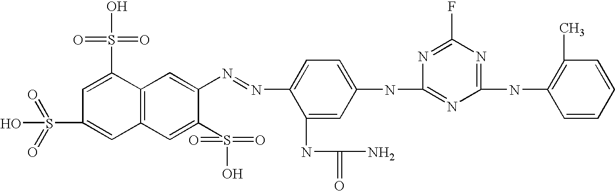 | orange |
| 14_348 | 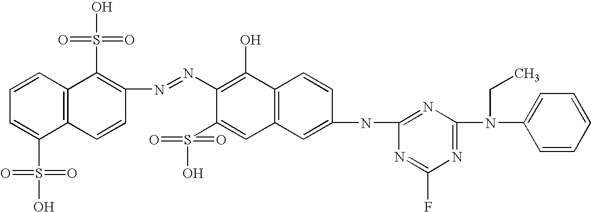 | orange |
| 14_349 | 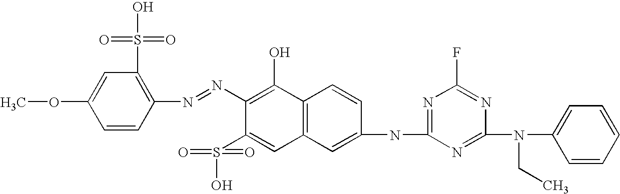 | red |
| 14_350 | 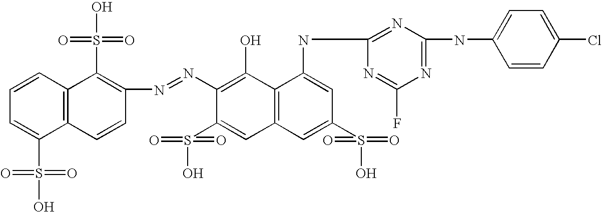 | red |
| 14_351 | 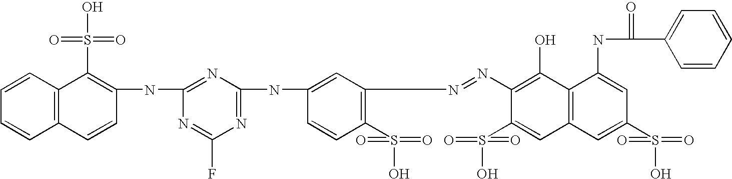 | red |
| 14_352 | 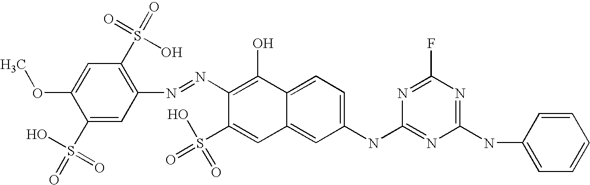 | red |
| 14_353 | 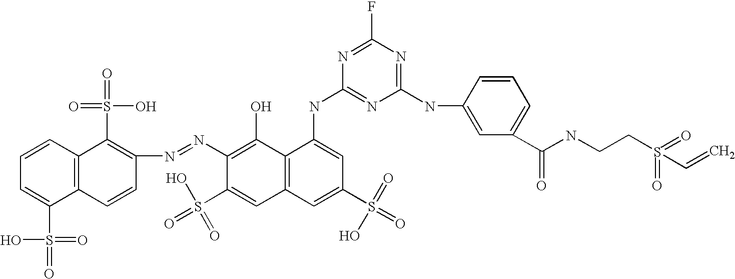 | red |
| 14_354 | 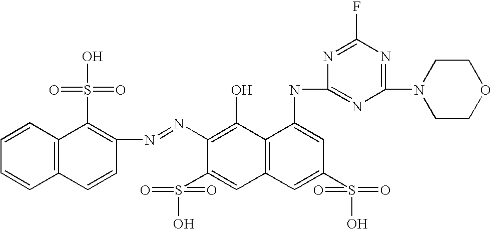 | red |
| 14_355 | 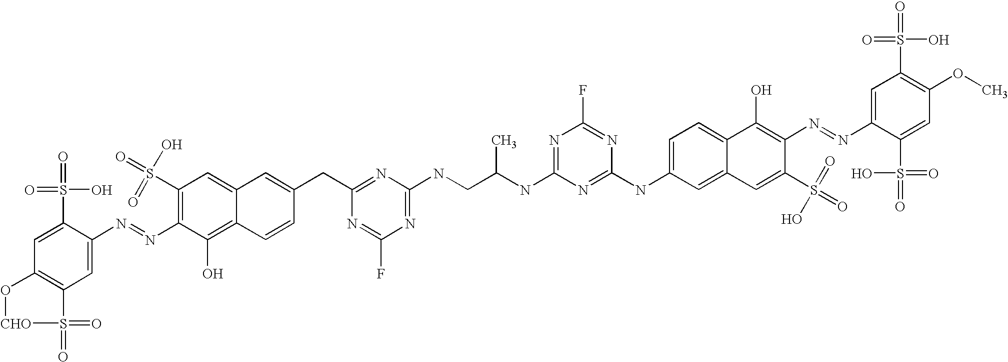 | red |
| 14_356 | 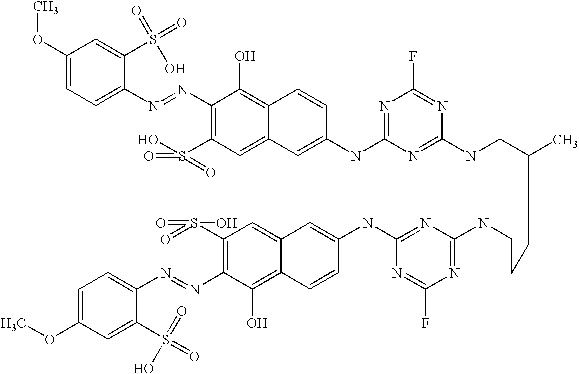 | red |
| 14_357 | 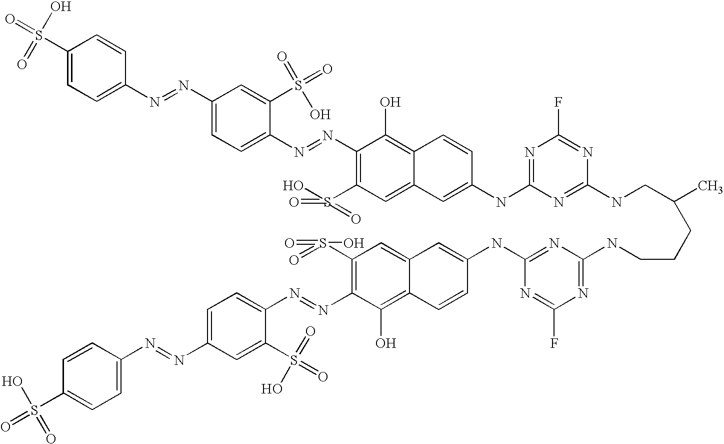 | red |
| 14_358 | 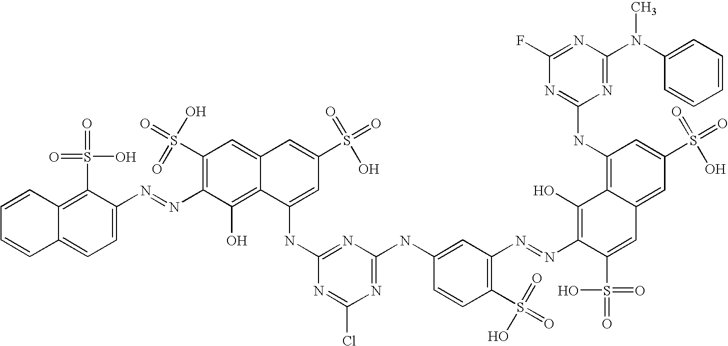 | red |
| 14_359 | 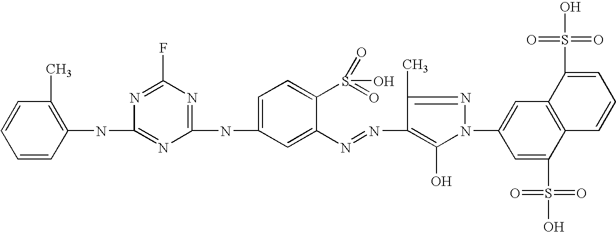 | yellow |
| 14_360 | 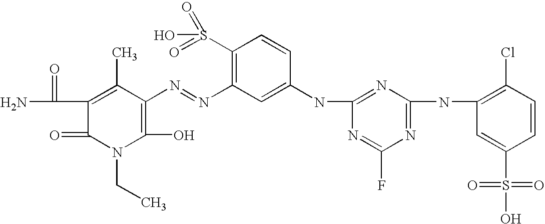 | yellow |
| 14_361 | 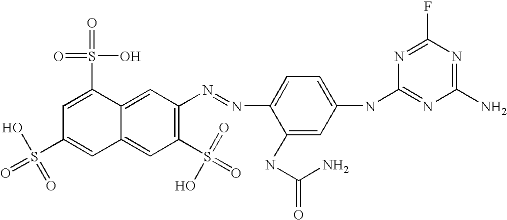 | yellow |
| 14_362 | 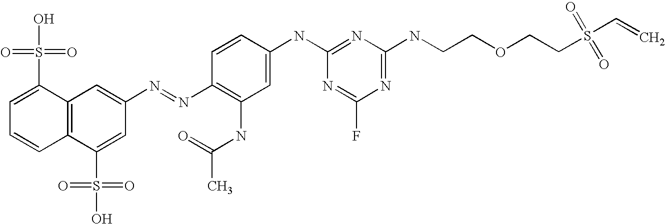 | yellow |
| 14_363 | 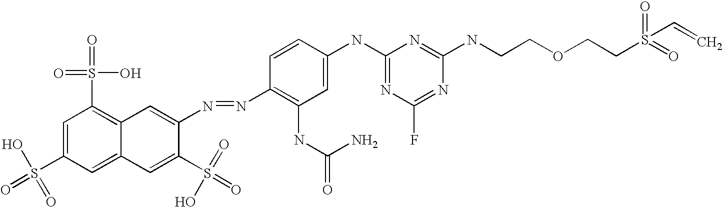 | yellow |
| 14_364 | 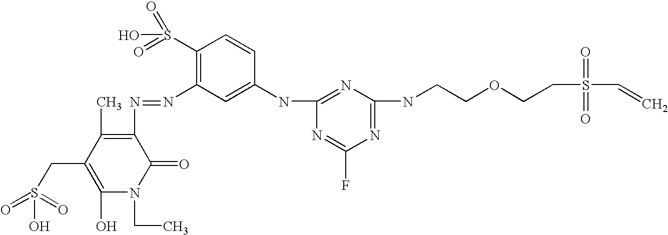 | yellow |
| 14_365 | 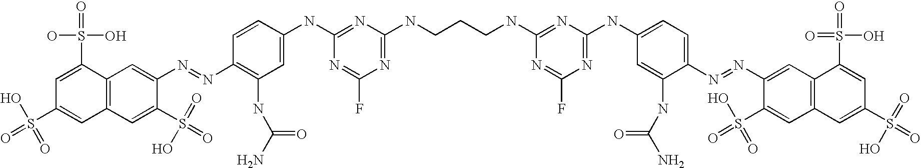 | yellow |
| 14_366 | 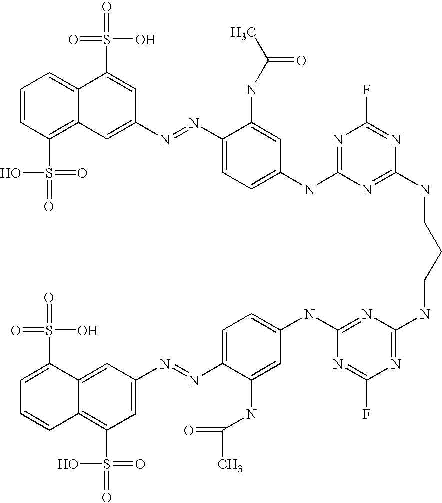 | yellow |

After the reactions described under a) and b), a molecule with free SH groups (e.g. cysteine) can be added which deactivates unreacted dye. Should the dyes used here not be purified after the synthesis, they comprise both reactive and unreactive secondary components. The secondary components which do not have a reactive anchor, or onto whose reactive anchor e.g. free cysteine has been bonded after the actual KBD coupling reaction do not react with the KBD-B molecules.

Such secondary components can be removed after the coupling reaction or e.g. during the hair-coloring process. One option consists in purifying the KBD-B reacted with the dye, e.g. by column chromatography. It is also possible to carry out the reaction between KBD-B and the reactive dye in a type of “column reactor”. In this connection, the KBD-B could be coupled to a nickel affinity column, the dye bonded to KBD-B and the unfixed dye radicals be washed out directly. The KBD-B-dye can then be eluted from the column. Another option is to apply to hair the KBD-B reacted with the dye together with the nonreactive secondary components, and to wash out the nonbonded dye fractions from the hair. For this, a 15% strength Tween 20 solution in water, for example, is suitable.

### Example 20

### Hair Coloring with KBD-B-Dye Couplings

Firstly, a red reactive dye 14_{—}264 was coupled to KBD-B (SEQ ID No.: 166) according to the method described above. In order to show that the binding of the KBD-B-dye coupling to hair is mediated by the protein and not by the free dye itself, the same dye was treated in each case only with cysteine but without KBD-B. Then, KBD-B-14_{—}264 and 14_{—}264 were each placed onto 5 mg of hair, which was briefly incubated, and nonbound KBD-B-dye or pure dye was washed off using a 15% strength Tween 20 solution. The result clearly shows that the binding to the hair is mediated by the KBD-B and coloring of the hair has taken place.

If, on the other hand, it is desired to decolor the hair again, a wash with a SDS fraction of 15%, or a treatment with a keratin-containing solution is suitable.

Dermocosmetic preparations according to the invention are described below, comprising the keratin-binding effector molecule prepared according to Example 19 (keratin-binding domain according to SEQ ID No.: ID 166 coupled with the dye 14_{—}264). Said keratin-binding effector molecule is referred to in the examples below as keratin-binding domain-reactive dye 14_{—}264. It will be appreciated by the person skilled in the art that all other dyes described in Tables 6 and 7 can be coupled with the KBD according to Example 19, 19 a or 19 b and be used in the preparations given below.

### Example 21

### Shampoo (Base Formulation without Keratin-Binding Domain-Reactive Dye)

| | % | Ingredient (INCI) |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 1.25 | Polyquaternium-16 |
| | 2.00 | Laureth-3 |
| | q.s. | Citric Acid |
| | 57.75 | Aqua dem. |
| B | 3.00 | PEG-150 Distearate |

Preparation: Weigh in the components of phase A and dissolve. Adjust the pH to 6-7. Add phase B and heat to about 50° C. Cool to room tern perature with stirring.

The content of keratin-binding domain-reactive dye 14_{—}264 in the examples below refers to 100% keratin-binding domain-reactive dye 14_{—}264. The active ingredient according to the invention can be used either in pure form or as an aqueous solution. In the case of the aqueous solution, the content of water dem. in the respective formulation must be adapted.

### Example 22

### Hair-Coloring Shampoo

| | |
|---|---|
| % | Ingredient (INCI) |

Variant 1

| | | |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 1.25 | Polyquaternium-16 |
| | 2.00 | Laureth-3 |
| | q.s. | Citric Acid |
| | 1.00 | Keratin-binding domain-reactive dye 14_264 |
| | 57.76 | Aqua dem. |
| B | 3.00 | PEG-150 Distearate |

Variant 2

| | | |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 1.25 | Polyquaternium-16 |
| | 2.00 | Laureth-3 |
| | q.s. | Citric Acid |
| | 5.00 | Keratin-binding domain-reactive dye 14_264 |
| | 53.75 | Aqua dem. |
| B | 3.00 | PEG-150 Distearate |

Preparation: Weigh in the components of phase A and dissolve. Adjust the pH to 6-7 Add phase B and heat to about 40° C. Cool rapidly to room temperature with stirring.

### Example 23

### Color Styling Mousse

| | |
|---|---|
| % | Ingredient (INCI) |

Variant 1

| | | |
|---|---|---|
| A | 2.00 | Cocotrimonium Methosulfate |
| | q.s. | Perfume oil |
| B | 6.70 | Acrylates Copolymer |
| | 0.60 | Aminomethyl Propanol |
| | 0.20 | Ceteareth-25 |
| | 0.20 | Panthenol |
| | 0.20 | Hydroxyethylcellulose |
| | 10.00 | Alcohol |
| | 69.97 | Aqua dem. |
| | 1.0 | Keratin-binding domain-reactive dye 14_264 |
| C | 10.00 | Propane/Butane |

Variant 2

| | | |
|---|---|---|
| A | 2.00 | Cocotrimonium Methosulfate |
| | q.s. | Perfume oil |
| B | 6.70 | Acrylates Copolymer |
| | 0.60 | Aminomethyl Propanol |
| | 0.20 | Ceteareth-25 |
| | 0.20 | Panthenol |
| | 0.20 | Hydroxyethylcellulose |
| | 10.00 | Alcohol |
| | 69.97 | Aqua dem. |
| | 0.5 | Keratin-binding domain-reactive dye 14_264 |
| C | 10.00 | Propane/Butane |

Preparation: Mix the components of phase A. Weigh in phase B and dissolve to give a clear solution, then stir into phase A. Bottle with phase C.

### Example 24

### Color Styling Mousse

| | |
|---|---|
| % | Ingredient (INCI) |

Variant 1

| | | |
|---|---|---|
| A | 2.00 | Cocotrimonium Methosulfate |
| | q.s. | Perfume oil |
| B | 7.00 | Polyquaternium-46 |
| | 2.00 | Polyquaternium-11 |
| | 78.87 | Aqua dem. |
| | 2.0 | Keratin-binding domain-reactive dye 14_264 |
| C | 10.00 | Propane/Butane |

Variant 2

| | | |
|---|---|---|
| A | 2.00 | Cocotrimonium Methosulfate |
| | q.s. | Perfume oil |
| B | 7.00 | Polyquaternium-46 |
| | 2.00 | Polyquaternium-11 |
| | 78.87 | Aqua dem. |
| | 1.0 | Keratin-binding domain-reactive dye 14_264 |
| C | 10.00 | Propane/Butane |

Preparation: Mix the components of phase A. Weigh in phase B and dissolve to give a clear solution, then stir into phase A. Bottle with phase C.

### Example 25

Two tresses of blond unbleached hair: European natural hair, color 9/0, natural blond (2 g) are treated with 0.5 g of the hair-coloring shampoo Example 22/variant 1 and the hair-coloring shampoo is left on the hair for 15 min. The hair tresses are then washed with water and cleansed using shampoo Example 21 and dried. The hair is completely red in color. Blond hair can no longer be detected. One tress is retained as comparison.

The second tress is then washed five times with shampoo Example 21 and dried. After the fifth wash, the tress is compared with the retained sample. The tress washed five times is still completely red in color. The two tresses have the same red shade. Visually, no difference in the color depth can be seen.

### Example 26

Two tresses of white hair: selected, white European natural hair (2 g) are treated with 0.5 g of the hair coloring shampoo Example 22/variant 1 and the hair-coloring shampoo is left on the hair for 15 min. The hair tresses are then washed with water and cleansed using shampoo Example 21 and dried. The hair is completely red in color. White hair can no longer be detected. One tress is retained as comparison.

The second tress is then washed five times with shampoo Example 21 and dried. After the fifth wash, the tress is compared with the retained sample. The tress washed five times is still completely red in color. The two tresses have the same red shade. Visually, no difference in color depth can be seen.

### Example 27

Two tresses of blond nonbleached hair: European natural hair, color 9/0, natural blond (2 g) are treated with 0.5 g of the color styling mousse Example 23/variant 2 and the color styling mousse is left on the hair for 15 min. The hair tresses are then washed with water and cleansed using shampoo Example 21 and dried. The hair is completely red in color. Blond hair can no longer be detected. One tress is retained as comparison.

The second tress is then washed five times with shampoo Example 21 and dried. After the fifth wash, the tress is compared with the retained sample. The tress washed five times is still completely red in color. The two tresses have the same red shade. Visually, no difference in the color depth can be seen.

### Example 28

Two tresses of white hair: selected white European natural hair (2 g) are treated with 0.5 g of the color styling mousse Example 23/variant 2 and the color styling mousse is left on the hair for 15 min. The hair tresses are then washed with water and cleansed with shampoo Example 21 and dried. The hair is completely red in color. White hair can no longer be detected. One tress is retained as comparison.

The second tress is then washed five times with shampoo Example 21 and dried. After the fifth wash, the tress is compared with the retained sample. The tress washed five times is still completely red in color. The two tresses have the same red shade. Visually, no difference in the color depth can be seen.

### Example 29

Two tresses of blond unbleached hair: European natural hair, color 9/0, natural blond (2 g) are treated with 0.5 g of the color styling mousse Example 24/variant 1 and the color styling mousse is left on the hair for 15 min. The hair tresses are then washed with water and cleansed with shampoo Example 21 and dried. The hair is completely red in color. Blond hair can no longer be detected. One tress is retained as comparison.

The second tress is then washed five times with shampoo Example 21 and dried. After the fifth wash, the tress is compared with the retained sample. The tress washed five times is still completely red in color. The two tresses have the same red shade. Visually, no difference in the color depth can be seen.

### Example 30

Two tresses of white hair: selected white European natural hair (2 g) are treated with 0.5 g of the color styling mousse Example 23/variant 1 and the color styling mousse is left on the hair for 15 min. The hair tresses are then washed with water and cleansed with shampoo Example 21 and dried. The hair is completely red in color. White hair can no longer be detected. One tress is retained as comparison.

The second tress is then washed five times with shampoo Example 21 and dried. After the fifth wash, the tress is compared with the retained sample. The tress washed five times is still completely red in color. The two tresses have the same red shade. Visually, no difference in the color depth can be seen.

### Example 31

### Use of the KBD in an Emulsion for Daycare—O/W Type

| | |
|---|---|
| % | Ingredient (INCI) |

Al 1%:

| | | |
|---|---|---|
| A | 1.7 | Ceteareth-6, Stearyl Alcohol |
| | 0.7 | Ceteareth-25 |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 69.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, Tocopherol, |
| | | Retinol |
| | 1.0 | Aqueous solution with about 5% keratin-binding domain- |
| | | reactive dye 14_264 |
| E | q.s. | Sodium Hydroxide |

Al 5%:

| | | |
|---|---|---|
| A | 1.7 | Ceteareth-6, Stearyl Alcohol |
| | 0.7 | Ceteareth-25 |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 65.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, Tocopherol, |
| | | Retinol |
| | 5.0 | Aqueous solution with about 5% keratin-binding domain- |
| | | reactive dye 14_264 |
| E | q.s. | Sodium Hydroxide |

Preparation: Heat phases A and B separately from one another to about 80° C. Stir Phase B into phase A and homogenize. Stir phase C into the combined phases A and B and homogenize again. Cool with stirring to about 40° C., add phase D, adjust the pH to about 6.5 using phase E, homogenize and cool to room temperature with stirring.

Note: The formulation is prepared without protective gas. Bottling must take place into oxygen-impermeable packagings, e.g. aluminum tubes.

### Example 32

### Use of the KBD in a Protective Day Cream—O/W Type

| | |
|---|---|
| % | Ingredient (INCI) |

Al 1%:

| | | |
|---|---|---|
| A | 1.7 | Ceteareth-6, Stearyl Alcohol |
| | 0.7 | Ceteareth-25 |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 70.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 1.0 | Aqueous solution with about 5% keratin-binding domain- |
| | | reactive dye 14_264 |
| E | q.s. | Sodium Hydroxide |

Al 5%:

| | | |
|---|---|---|
| A | 1.7 | Ceteareth-6, Stearyl Alcohol |
| | 0.7 | Ceteareth-25 |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 66.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 5.0 | Aqueous solution with about 5% keratin-binding domain- |
| | | reactive dye 14_264 |
| E | q.s. | Sodium Hydroxide |

Preparation: Heat phases A and B separately from one another to about 80° C. Stir phase B into phase A and homogenize. Incorporate phase C into the combined phases A and B and homogenize. Cool with stirring to about 40° C. Add phase D, adjust the pH to about 6.5 using phase E and homogenize. Cool to room temperature with stirring.

### Example 33

### Use of the KBD in a Face-Cleansing Lotion—O/W Type

| | |
|---|---|
| % | Ingredient (INCI) |

Al 1%:

| | | |
|---|---|---|
| A | 10.0 | Cetearyl Ethylhexanoate |
| | 10.0 | Caprylic/Capric Triglyceride |
| | 1.5 | Cyclopentasiloxane, Cyclohexasiloxane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 1.0 | Aqueous solution with about 5% keratin-binding domain- |
| | | reactive dye 14_264 |
| | 60.7 | Aqua dem. |

Al 5%:

| | | |
|---|---|---|
| A | 10.0 | Cetearyl Ethylhexanoate |
| | 10.0 | Caprylic/Capric Triglyceride |
| | 1.5 | Cyclopentasiloxane, Cyclohexasiloxane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 5.0 | Aqueous solution with about 5% keratin-binding domain- |
| | | reactive dye 14_264 |
| | 56.7 | Aqua dem. |

Preparation: Dissolve phase A. Stir phase B into phase A. Incorporate phase C into the combined phases A and B. Dissolve phase D, stir into the combined phases A, B and C and homogenize. After-stir for 15 min.

### Example 34

### Use of the KBD in a Daily Care Body Spray

| | |
|---|---|
| % | Ingredient (INCI) |

Al 1%:

| | | |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 1.0 | Aqueous solution with about 5% keratin-binding domain- |
| | | reactive dye 14_264 |
| | 59.2 | Alcohol |

Al 5%:

| | | |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 5.0 | Aqueous solution with about 5% keratin-binding domain- |
| | | reactive dye 14_264 |
| | 55.2 | Alcohol |

Preparation: Weigh in the components of phase A and dissolve until clear.

### Example 35

### Use of the KBD in a Skin Care Gel

| | |
|---|---|
| % | Ingredient (INCI) |

Al 1%:

| | | |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 1.0 | Aqueous solution with about 5% keratin-binding domain- |
| | | reactive dye 14_264 |
| | 75.4 | Aqua dem. |
| C | 0.8 | Triethanolamine |

Al 5%:

| | | |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 5.0 | Aqueous solution with about 5% keratin-binding domain- |
| | | reactive dye 14_264 |
| | 71.4 | Aqua dem. |
| C | 0.8 | Triethanolamine |

Preparation: Dissolve phase A until clear. Allow phase B to swell and neutralize with phase C. Stir phase A into the homogenized phase B and homogenize.

### Example 36

### Use of the KBD in an After Shave Lotion

| | |
|---|---|
| % | Ingredient (INCI) |

Al 1%:

| | | |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 1.0 | Aqueous solution with about 5% keratin-binding domain- |
| | | reactive dye 14_264 |
| | 0.1 | Triethanolamine |
| | 63.5 | Aqua dem. |

Al 5%:

| | | |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 5.0 | Aqueous solution with about 5% keratin-binding domain- |
| | | reactive dye 14_264 |
| | 0.1 | Triethanolamine |
| | 59.5 | Aqua dem. |

Preparation: Mix the components of phase A. Dissolve phase B, incorporate into phase A and homogenize.

### Example 37

### Use of the KBD in an After Sun Lotion

| | |
|---|---|
| % | Ingredient (INCI) |

Al 1%:

| | | |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 1.0 | Aqueous solution with about 5% keratin-binding domain- |
| | | reactive dye 14_264 |
| | 63.2 | Aqua dem. |
| C | 0.2 | Triethanolamine |

Al 5%:

| | | |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 5.0 | Aqueous solution with about 5% keratin-binding domain- |
| | | reactive dye 14_264 |
| | 59.2 | Aqua dem. |
| C | 0.2 | Triethanolamine |

Preparation: Mix the components of phase A. Stir phase B into phase A with homogenization. Neutralize with phase C and homogenize again.

### Example 38

### Use of the KBD in a Sunscreen Lotion

| | |
|---|---|
| % | Ingredient (INCI) |

Al 1%:

| | | |
|---|---|---|
| A | 4.5 | Ethylhexyl Methoxycinnamate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 61.7 | Aqua dem. |
| D | 1.0 | Aqueous solution with about 5% keratin-binding domain- |
| | | reactive dye 14_264 |
| | 1.0 | Phenoxyethanol, Methylparaben, Ethylparaben, Butylparaben, |
| | | Propyl-paraben, Isobutylparaben |
| | 0.3 | Bisabolol |

Al 5%:

| | | |
|---|---|---|
| A | 4.5 | Ethylhexyl Methoxycinnamate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 57.7 | Aqua dem. |
| D | 5.0 | Aqueous solution with about 5% keratin-binding domain- |
| | | reactive dye 14_264 |
| | 1.0 | Phenoxyethanol, Methylparaben, Ethylparaben, Butylparaben, |
| | | Propyl-paraben, Isobutylparaben |
| | 0.3 | Bisabolol |

Preparation: Heat the components of phases A and B separately from one another to about 80° C. Stir phase B into phase A and homogenize. Heat phase C to about 80° C. and stir into the combined phases A and B with homogenization. Cool to about 40° C. with stirring, add phase D and homogenize again.

### Example 39

### Use of the KBD in a Sunscreen Lotion—O/W Type

| | |
|---|---|
| % | Ingredient (INCI) |

Al 1%:

| | | |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, Squalane, Polysorbate 60 |
| | 0.2 | Disodium EDTA |
| | 5.0 | Propylene Glycol |
| | 0.5 | Panthenol |
| | 60.9 | Aqua dem. |
| D | 1.0 | Aqueous solution with about 5% keratin-binding domain- |
| | | reactive dye 14_264 |
| | 0.5 | Phenoxyethanol, Methylparaben, Butylparaben, Ethylparaben, |
| | | Propyl-paraben, Isopropylparaben |
| | 1.0 | Tocopheryl Acetate |
| | 0.2 | Bisabolol |

Al 5%:

| | | |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, Squalane, Polysorbate 60 |
| | 0.2 | Disodium EDTA |
| | 5.0 | Propylene Glycol |
| | 0.5 | Panthenol |
| | 56.9 | Aqua dem. |
| D | 5.0 | Aqueous solution with about 5% keratin-binding domain- |
| | | reactive dye 14_264 |
| | 0.5 | Phenoxyethanol, Methylparaben, Butylparaben, Ethylparaben, |
| | | Propyl-paraben, Isopropylparaben |
| | 1.0 | Tocopheryl Acetate |
| | 0.2 | Bisabolol |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### Example 40

### Use of the KBD in a Sunscreen Lotion—O/W Type

| | |
|---|---|
| % | Ingredient (INCI) |

Al 1%:

| | | |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 56.3 | Aqua dem. |
| D | 1.0 | Aqueous solution with about 5% keratin-binding domain- |
| | | reactive dye 14_264 |
| | 1.0 | Tocopheryl Acetate |
| | 0.2 | Bisabolol |
| | q.s. | Perfume oil |
| | q.s. | Preservative |

Al 5%:

| | | |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 52.3 | Aqua dem. |
| D | 5.0 | Aqueous solution with about 5% keratin-binding domain- |
| | | reactive dye 14_264 |
| | 1.0 | Tocopheryl Acetate |
| | 0.2 | Bisabolol |
| | q.s. | Perfume oil |
| | q.s. | Preservative |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### Example 41

### Use of the KBD in a Foot Balsam

| | |
|---|---|
| % | Ingredient (INCI) |

Al 1%:

| | | |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 69.3 | Aqua dem. |
| | q.s. | Preservative |
| C | 1.0 | Bisabolol |
| | 1.0 | Tocopheryl Acetate |
| D | 1.0 | Aqueous solution with about 5% keratin-binding domain- |
| | | reactive dye 14_264 |
| | 5.0 | Witch Hazel Extract |

Al 5%:

| | | |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 65.3 | Aqua dem. |
| | q.s. | Preservative |
| C | 1.0 | Bisabolol |
| | 1.0 | Tocopheryl Acetate |
| D | 5.0 | Aqueous solution with about 5% keratin-binding domain- |
| | | reactive dye 14_264 |
| | 5.0 | Witch Hazel Extract |

Preparation: Heat the components of phases A and B separately from one another to about 80° C. Stir phase B into phase A with homogenization. Cool to about 40° C. with stirring, add phases C and D and briefly after-homogenize. Cool to room temperature with stirring.

### Example 42

### Use of the KBD in a W/O Emulsion with Bisabolol

| | |
|---|---|
| % | Ingredient (INCI) |

Al 1%:

| | | |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 55.6 | Aqua dem. |
| C | 1.0 | Aqueous solution with about 5% keratin-binding domain- |
| | | reactive dye 14_264 |
| | 0.5 | Tocopheryl Acetate |
| | 0.6 | Bisabolol |

Al 5%:

| | | |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 51.6 | Aqua dem. |
| C | 5.0 | Aqueous solution with about 5% keratin-binding domain- |
| | | reactive dye 14_264 |
| | 0.5 | Tocopheryl Acetate |
| | 0.6 | Bisabolol |

Preparation: Heat phases A and B separately from one another to about 85° C. Stir phase B into phase A and homogenize. Cool to about 40° C. with stirring, add phase C and briefly homogenize again. Cool to room temperature with stirring. List of formulations for patent keratin-binding domain - haircare

### Example 43

### Foam Conditioner with Setting Agent

| | |
|---|---|
| % | Ingredient (INCI) |

Al 1%

| | | |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume oil |
| | 10.0 | Alcohol |
| | 1.0 | Aqueous solution with about 5% keratin-binding |
| | | domain-reactive dye 14_264 |
| | 68.1 | Aqua dem. |
| | 10.0 | Propane/Butane |

Al 5%

| | | |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume oil |
| | 10.0 | Alcohol |
| | 5.0 | Aqueous solution with about 5% keratin-binding |
| | | domain-reactive dye 14_264 |
| | 64.1 | Aqua dem. |
| B | 10.0 | Propane/Butane |

Preparation: Weigh the components of phase A together, stir until everything has dissolved and bottle with phase B.

### Example 44

### Hair-Coloring Foam Conditioner

| | |
|---|---|
| % | Ingredient (INCI) |

Al 1%

| | | |
|---|---|---|
| A | 1.0 | Polyquaternium-4 |
| | 0.5 | Hydroxyethyl Cetyldimonium Phosphate |
| | 1.0 | Aqueous solution with about 5% keratin-binding |
| | | domain-reactive dye 14_264 |
| | q.s. | Perfume oil |
| | q.s. | Preservative |
| | 91.5 | Aqua dem. |
| B | 6.0 | Propane/Butane |

Al 5%

| | | |
|---|---|---|
| A | 1.0 | Polyquaternium-4 |
| | 0.5 | Hydroxyethyl Cetyldimonium Phosphate |
| | 5.0 | Aqueous solution with about 5% keratin-binding |
| | | domain-reactive dye 14_264 |
| | q.s. | Perfume oil |
| | q.s. | Preservative |
| | 87.5 | Aqua dem. |
| B | 6.0 | Propane/Butane |

Preparation: Weigh the components of phase A together, stir until everything has dissolved to give a clear solution and bottle with phase B.

### Example 45

### Hair-Coloring Foam Conditioner

| | |
|---|---|
| % | Ingredient (INCI) |

Al 1%

| | | |
|---|---|---|
| A | 1.0 | Polyquaternium-11 |
| | 0.5 | Hydroxyethyl Cetyldimonium Phosphate |
| | 1.0 | Aqueous solution with about 5% keratin-binding |
| | | domain-reactive dye 14_264 |
| | q.s. | Perfume oil |
| | q.s. | Preservative |
| | 91.5 | Aqua dem. |
| B | 6.0 | Propane/Butane |

Al 5%

| | | |
|---|---|---|
| A | 1.0 | Polyquaternium-11 |
| | 0.5 | Hydroxyethyl Cetyldimonium Phosphate |
| | 5.0 | Aqueous solution with about 5% keratin-binding |
| | | domain-reactive dye 14_264 |
| | q.s. | Perfume oil |
| | q.s. | Preservative |
| | 87.5 | Aqua dem. |
| B | 6.0 | Propane/Butane |

Preparation: Weigh the components of phase A together, stir until everything has dissolved to give a clear solution and bottle with phase B.

### Example 46

### Hair-Coloring Styling Foam

| | |
|---|---|
| % | Ingredient (INCI) |

Al 1%

| | | |
|---|---|---|
| A | 0.5 | Laureth-4 |
| | q.s. | Perfume oil |
| B | 77.3 | Aqua dem. |
| | 10.0 | Polyquaternium-28 |
| | 1.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Al 5%

| | | |
|---|---|---|
| A | 0.5 | Laureth-4 |
| | q.s. | Perfume oil |
| B | 73.3 | Aqua dem. |
| | 10.0 | Polyquaternium-28 |
| | 5.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Bottle with phase C.

### Example 47

### Hair-Coloring Styling Foam

| | |
|---|---|
| % | Ingredient (INCI) |

Al 1%

| | | |
|---|---|---|
| A | 2.0 | Cocotrimonium Methosulfate |
| | q.s. | Perfume oil |
| B | 78.5 | Aqua dem. |
| | 6.7 | Acrylates Copolymer |
| | 0.6 | AMP |
| | 1.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Al 5%

| | | |
|---|---|---|
| A | 2.0 | Cocotrimonium Methosulfate |
| | q.s. | Perfume oil |
| B | 74.5 | Aqua dem. |
| | 6.7 | Acrylates Copolymer |
| | 0.6 | AMP |
| | 5.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Bottle with phase C.

### Example 48

### Hair-Coloring Styling Foam

| | |
|---|---|
| % | Ingredient (INCI) |

Al 1%

| | | |
|---|---|---|
| A | 2.0 | Cocotrimonium Methosulfate |
| | q.s. | Perfume oil |
| B | 7.70 | Polyquaternium-44 |
| | 1.0 | Aqueous solution with about 5% keratin-binding |
| | | domain-reactive dye 14_264 |
| | q.s. | Preservative |
| | 79.3 | Aqua dem. |
| C | 10.0 | Propane/Butane |

Al 5%

| | | |
|---|---|---|
| A | 2.0 | Cocotrimonium Methosulfate |
| | q.s. | Perfume oil |
| B | 7.70 | Polyquaternium-44 |
| | 5.0 | Aqueous solution with about 5% keratin-binding |
| | | domain-reactive dye 14_264 |
| | q.s. | Preservative |
| | 75.3 | Aqua dem. |
| C | 10.0 | Propane/Butane |

Preparation: Mix the components of phase A. Dissolve the components of phase B until clear, then stir phase B into phase A. Adjust the pH to 6-7, bottle with phase C.

### Example 49

### Hair-Coloring Styling Foam

| | |
|---|---|
| % | Ingredient (INCI) |

Al 1%

| | | |
|---|---|---|
| A | 2.00 | Cocotrimonium Methosulfate |
| | q.s. | Perfume oil |
| B | 72.32 | Aqua dem. |
| | 2.00 | VP/Acrylates/Lauryl Methacrylate Copolymer |
| | 0.53 | AMP |
| | 1.00 | Aqueous solution with about 5% keratin-binding |
| | | domain-reactive dye 14_264 |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Al 5%

| | | |
|---|---|---|
| A | 2.00 | Cocotrimonium Methosulfate |
| | q.s. | Perfume oil |
| B | 68.32 | Aqua dem. |
| | 2.00 | VP/Acrylates/Lauryl Methacrylate Copolymer |
| | 0.53 | AMP |
| | 5.00 | Aqueous solution with about 5% keratin-binding |
| | | domain-reactive dye 14_264 |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Allow phase C to swell in the mixture of A and B, then adjust the pH to 6-7. Bottle with phase D.

### Example 50

### Hair-Coloring Styling Foam

| | |
|---|---|
| % | Ingredient (INCI) |

Al 1%

| | | |
|---|---|---|
| A | 2.00 | Cetrimonium Chloride |
| | q.s. | Perfume oil |
| B | 67.85 | Aqua dem. |
| | 7.00 | Polyquaternium-46 |
| | 1.00 | Aqueous solution with about 5% keratin-binding |
| | | domain-reactive dye 14_264 |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Al 5%

| | | |
|---|---|---|
| A | 2.00 | Cetrimonium Chloride |
| | q.s. | Perfume oil |
| B | 63.85 | Aqua dem. |
| | 7.00 | Polyquaternium-46 |
| | 5.00 | Aqueous solution with about 5% keratin-binding |
| | | domain-reactive dye 14_264 |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Allow phase C to swell in the mixture of A and B, then adjust the pH to 6-7. Bottle with phase D.

### Example 51

### Hair-Coloring Styling Foam

| | |
|---|---|
| % | Ingredient (INCI) |

Al 1%

| | | |
|---|---|---|
| A | q.s. | PEG-40 Hydrogenated Castor Oil |
| | q.s. | Perfume oil |
| | 85.5 | Aqua dem. |
| B | 7.0 | Sodium Polystyrene Sulfonate |
| | 1.0 | Aqueous solution with about 5% keratin-binding |
| | | domain-reactive dye 14_264 |
| | 0.5 | Cetrimonium Bromide |
| | q.s. | Preservative |
| C | 6.0 | Propane/Butane |

Al 5%

| | | |
|---|---|---|
| A | q.s. | PEG-40 Hydrogenated Castor Oil |
| | q.s. | Perfume oil |
| | 81.5 | Aqua dem. |
| B | 7.0 | Sodium Polystyrene Sulfonate |
| | 5.0 | Aqueous solution with about 5% keratin-binding |
| | | domain-reactive dye 14_264 |
| | 0.5 | Cetrimonium Bromide |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### Example 52

### Hair-Coloring Styling Foam

| | |
|---|---|
| % | Ingredient (INCI) |

Al 1%

| | | |
|---|---|---|
| A | q.s. | PEG-40 Hydrogenated Castor Oil |
| | q.s. | Perfume oil |
| | 92.0 | Aqua dem. |
| B | 0.5 | Polyquaternium-10 |
| | 1.0 | Aqueous solution with about 5% keratin-binding |
| | | domain-reactive dye 14_264 |
| | 0.5 | Cetrimonium Bromide |
| | q.s. | Preservative |
| C | 6.0 | Propane/Butane |

Al 5%

| | | |
|---|---|---|
| A | q.s. | PEG-40 Hydrogenated Castor Oil |
| | q.s. | Perfume oil |
| | 88.0 | Aqua dem. |
| B | 0.5 | Polyquaternium-10 |
| | 5.0 | Aqueous solution with about 5% keratin-binding |
| | | domain-reactive dye 14_264 |
| | 0.5 | Cetrimonium Bromide |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### Example 53

### Hair-Coloring Styling Foam

| | |
|---|---|
| % | Ingredient (INCI) |

Al 1%

| | | |
|---|---|---|
| A | q.s. | PEG-40 Hydrogenated Castor Oil |
| | q.s. | Perfume oil |
| | 82.5 | Aqua dem. |
| B | 10.0 | Polyquaternium-16 |
| | 1.0 | Aqueous solution with about 5% keratin-binding |
| | | domain-reactive dye 14_264 |
| | 0.5 | Hydroxyethyl Cetyldimonium Phosphate |
| | q.s. | Preservative |
| C | 6.0 | Propane/Butane |

Al 5%

| | | |
|---|---|---|
| A | q.s. | PEG-40 Hydrogenated Castor Oil |
| | q.s. | Perfume oil |
| | 78.5 | Aqua dem. |
| B | 10.0 | Polyquaternium-16 |
| | 5.0 | Aqueous solution with about 5% keratin-binding |
| | | domain-reactive dye 14_264 |
| | 0.5 | Hydroxyethyl Cetyldimonium Phosphate |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### Example 54

### Hair-Tinting Styling Foam

| | |
|---|---|
| % | Ingredient (INCI) |

Al 1%

| | | |
|---|---|---|
| A | 2.0 | Cocotrimonium Methosulfate |
| | q.s. | Perfume oil |
| B | 84.0 | Aqua dem. |
| | 2.0 | Chitosan |
| | 1.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| C | 10.0 | HFC 152 A |

Al 5%

| | | |
|---|---|---|
| A | 2.0 | Cocotrimonium Methosulfate |
| | q.s. | Perfume oil |
| B | 80.0 | Aqua dem. |
| | 2.0 | Chitosan |
| | 5.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| C | 10.0 | HFC 152 A |

Preparation: Mix the components of phase A. Add the components of phase B one after the other and dissolve. Bottle with phase C.

### Example 55

### Care Shampoo

| | |
|---|---|
| % | Ingredient (INCI) |

Al 1%

| | | |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, |
| | | Cocamide MEA, Laureth-10 |
| | 1.0 | Aqueous solution with about 5% keratin-binding |
| | | domain-reactive dye 14_264 |
| | 7.7 | Polyquaternium-44 |
| | 2.0 | Amodimethicone |
| | q.s. | Perfume oil |
| | q.s. | Preservative |
| | 1.0 | Sodium Chloride |
| | 43.3 | Aqua dem. |
| B | q.s. | Citric Acid |

Al 5%

| | | |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, |
| | | Cocamide MEA, Laureth-10 |
| | 5.0 | Aqueous solution with about 5% keratin-binding |
| | | domain-reactive dye 14_264 |
| | 7.7 | Polyquaternium-44 |
| | 2.0 | Amodimethicone |
| | q.s. | Perfume oil |
| | q.s. | Preservative |
| | 1.0 | Sodium Chloride |
| | 39.3 | Aqua dem. |
| B | q.s. | Citric Acid |

Preparation: Mix the components of phase A and dissolve. Adjust the pH to 6-7 with citric acid.

### Example 56

### Shower Gel

| | |
|---|---|
| % | Ingredient (INCI) |

Al 1%

| | | |
|---|---|---|
| A | 40.0 | Sodium Laureth Sulfate |
| | 5.0 | Decyl Glucoside |
| | 5.0 | Cocamidopropyl Betaine |
| | 1.0 | Aqueous solution with about 5% keratin-binding |
| | | domain-reactive dye 14_264 |
| | 1.0 | Panthenol |
| | q.s. | Perfume oil |
| | q.s. | Preservative |
| | 2.0 | Sodium Chloride |
| | 46.0 | Aqua dem. |
| B | q.s. | Citric Acid |

Al 5%

| | | |
|---|---|---|
| A | 40.0 | Sodium Laureth Sulfate |
| | 5.0 | Decyl Glucoside |
| | 5.0 | Cocamidopropyl Betaine |
| | 5.0 | Aqueous solution with about 5% keratin-binding |
| | | domain-reactive dye 14_264 |
| | 1.0 | Panthenol |
| | q.s. | Perfume oil |
| | q.s. | Preservative |
| | 2.0 | Sodium Chloride |
| | 42.0 | Aqua dem. |
| B | q.s. | Citric Acid |

Preparation: Mix the components of phase A and dissolve. Adjust the pH to 6-7 with citric acid.

### Example 57

### Shampoo

| | |
|---|---|
| % | Ingredient (INCI) |

Al 1%

| | | |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume oil |
| | 0.1 | Phytantriol |
| | 44.6 | Aqua dem. |
| | 1.0 | Aqueous solution with about 5% keratin-binding |
| | | domain-reactive dye 14_264 |
| | 0.3 | Polyquaternium-10 |
| | 1.0 | Panthenol |
| | q.s. | Preservative |
| | 1.0 | Laureth-3 |
| | 2.0 | Sodium Chloride |

Al 5%

| | | |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume oil |
| | 0.1 | Phytantriol |
| | 40.6 | Aqua dem. |
| | 5.0 | Aqueous solution with about 5% keratin-binding |
| | | domain-reactive dye 14_264 |
| | 0.3 | Polyquaternium-10 |
| | 1.0 | Panthenol |
| | q.s. | Preservative |
| | 1.0 | Laureth-3 |
| | 2.0 | Sodium Chloride |

Preparation: Mix the components of phase A and dissolve. Adjust the pH to 6-7 with citric acid.

### Example 58

### Hair-Coloring Shampoo

| | |
|---|---|
| % | Ingredient (INCI) |

Al 1%

| | | |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 1.00 | Aqueous solution with about 5% keratin-binding |
| | | domain-reactive dye 14_264 |
| | 0.15 | Guar Hydroxypropyltrimonium Chloride |
| | 2.00 | Laureth-3 |
| | 58.00 | Aqua dem. |
| | q.s. | Citric Acid |
| B | 3.00 | PEG-150 Distearate |

Al 5%

| | | |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 5.00 | Aqueous solution with about 5% keratin-binding |
| | | domain-reactive dye 14_264 |
| | 0.15 | Guar Hydroxypropyltrimonium Chloride |
| | 2.00 | Laureth-3 |
| | 54.00 | Aqua dem. |
| | q.s. | Citric Acid |
| B | 3.00 | PEG-150 Distearate |

Preparation: Weigh in the components of phase A and dissolve. Adjust the pH to 6-7. Add phase B and heat to about 40° C. Cool to room temperature with stirring.

### Example 59

### Moisturizing Bodycare Cream

| | |
|---|---|
| % | Ingredient (INCI) |

Al 1%

| | | |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 1.0 | Panthenol |
| | 0.5 | Magnesium Aluminum Silicate |
| | q.s | Preservative |
| | 65.5 | Aqua dem. |
| C | q.s. | Perfume oil |
| | 1.0 | Aqueous solution with about 5% keratin-binding |
| | | domain-reactive dye 14_264 |
| D | q.s. | Citric Acid |

Al 5%

| | | |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| | 1.0 | Panthenol |
| B | 5.0 | Propylene Glycol |
| | 0.5 | Magnesium Aluminum Silicate |
| | q.s | Preservative |
| | 61.5 | Aqua dem. |
| C | q.s. | Perfume oil |
| | 5.0 | Aqueous solution with about 5% keratin-binding |
| | | domain-reactive dye 14_264 |
| D | q.s. | Citric Acid |

Preparation: Heat phases A and B separately to about 80° C. Briefly prehomogenize phase B, then stir phase B into phase A and homogenize again. Cool to about 40° C., add phase C and homogenize thoroughly again. Adjust the pH to 6-7 with citric acid.

### Example 60

### Moisturizing Bodycare Cream

| | |
|---|---|
| % | Ingredient (INCI) |

Al 1%

| | | |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 62.9 | Aqua dem. |
| C | q.s. | Perfume oil |
| | 1.0 | Aqueous solution with about 5% keratin-binding |
| | | domain-reactive dye 14_264 |

Al 5%

| | | |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 58.9 | Aqua dem. |
| C | q.s. | Perfume oil |
| | 5.0 | Aqueous solution with about 5% keratin-binding |
| | | domain-reactive dye 14_264 |

Preparation: Heat phases A and B separately to about 80° C. Stir phase B into phase A and homogenize. Cool to about 40° C. with stirring, add phase C and homogenize again. Allow to cool to room temperature with stirring.

### Example 61

### Liquid Make-Up—O/W Type

| | |
|---|---|
| % | Ingredient (INCI) |

Al 1%

| | | |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 61.9 | Aqua dem. |
| C | 0.1 | Bisabolol |
| | 1.0 | Aqueous solution with about 5% keratin-binding |
| | | domain-reactive dye 14_264 |
| | q.s. | Perfume oil |
| D | 5.7 | C.I. 77 891, Titanium Dioxide |
| | 1.1 | Iron Oxides |

Al 5%

| | | |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 57.9 | Aqua dem. |
| C | 0.1 | Bisabolol |
| | 5.0 | Aqueous solution with about 5% keratin-binding |
| | | domain-reactive dye 14_264 |
| | q.s. | Perfume oil |
| D | 5.7 | C.I. 77 891, Titanium Dioxide |
| | 1.1 | Iron Oxides |

Preparation: Heat phases A and B separately to about 80° C. Stir phase B into phase A and homogenize. Cool to about 40° C. with stirring, add phases C and D and thoroughly homogenize again. Allow to cool to room temperature with stirring.

### Example 62

The specified keratin-binding domain-reactive dye 14_{—}264 is used as an approximately 5% strength by weight aqueous solution. The following data are parts by weight.

Clear Hair Coloring Shampoo

| Ingredients (INCI) | 1 | 2 | 3 | 4 | 5 |
|---|---|---|---|---|---|
| Sodium Laureth Sulfate | — | 15.00 | 10.50 | 12.50 | — |
| Codamidopropyl Betaine | 7.50 | 7.00 | 5.00 | 5.50 | 10.00 |
| PEG-7 Glyceryl Cocoate | 2.00 | 2.50 | 3.50 | 5.00 | 2.30 |
| Perfume oil | 0.10 | 0.10 | 0.10 | 0.10 | 0.10 |
| Keratin-binding domain- | 1.0 | 5.0 | 0.1 | 0.5 | 10.0 |
| reactive dye 14_264 | | | | | |
| D-Panthenol USP | 1.00 | 1.50 | 1.80 | 1.70 | 1.40 |
| Preservative | 0.10 | 0.10 | 0.10 | 0.10 | 0.10 |
| Citric Acid | 0.10 | 0.10 | 0.10 | 0.10 | 0.10 |
| Luviquat ® Ultra Care | 1.50 | 1.00 | 1.50 | 1.20 | 1.10 |
| Sodium Chloride | 1.50 | 1.40 | 1.40 | 1.30 | 1.50 |
| Aqua dem. | ad 100 | ad 100 | ad 100 | ad 100 | ad 100 |

Hair-Coloring Shampoo

| Ingredients (INCI) | 1 | 2 | 3 | 4 | 5 |
|---|---|---|---|---|---|
| Sodium Laureth Sulfate | 35.00 | −0 | 5.00 | 1.00 | — |
| Decyl Glucoside | 5.00 | 5.50 | 4.90 | 3.50 | 7.00 |
| Cocamidopropyl Betaine | 10.00 | 5.00 | 12.50 | 7.50 | 15.00 |
| Perfume oil | 0.10 | 0.10 | 0.10 | 0.10 | 0.10 |
| Keratin-binding domain- | 1.0 | 5.0 | 0.1 | 20.0 | 5.0 |
| reactive dye 14_264 | | | | | |
| D-Panthenol USP | 0.50 | 1.00 | 0.80 | 1.50 | 0.50 |
| Preservative | 0.10 | 0.10 | 0.10 | 0.10 | 0.10 |
| Citric Acid | 0.10 | 0.10 | 0.10 | 0.10 | 0.10 |
| Laureth-3 | 0.50 | 2.00 | 0.50 | 0.50 | 2.00 |
| Sodium Chloride | 1.50 | 1.50 | 1.50 | 1.50 | 1.50 |
| Aqua dem. | ad 100 | ad 100 | ad 100 | ad 100 | ad 100 |

Clear Conditioner Shampoo for Hair Coloring

| Ingredients (INCI) | 1 | 2 | 3 | 4 | 5 |
|---|---|---|---|---|---|
| ® Disodium | 10.00 | 15.00 | 20.00 | 12.00 | 17.00 |
| Cocoamphodiacetate | | | | | |
| ® Decyl Glucoside | 5.00 | 6.00 | 7.00 | 8.00 | 4.00 |
| ® Cocamidopropyl | 15.00 | 12.00 | 10.00 | 18.00 | 20.00 |
| Betaine | | | | | |
| Luviquat ® FC 550 | 0.30 | 0.20 | 0.20 | 0.20 | 0.30 |
| Perfume oil | 0.10 | 0.10 | 0.10 | 0.10 | 0.10 |
| Keratin-binding domain- | 20.0 | 5.0 | 1.0 | 0.5 | 10.0 |
| reactive dye 14_264 | | | | | |
| Cremophor ® PS 20 | 5.00 | 1.00 | 1.00 | 7.00 | 5.00 |
| Preservative | 0.10 | 0.10 | 0.10 | 0.10 | 0.10 |
| Laureth-3 | — | 1.00 | 0.50 | 2.00 | — |
| Citric Acid | 0.20 | 0.20 | 0.20 | 0.20 | 0.20 |
| PEG-12 Distearate | 3.00 | 2.00 | 2.00 | 3.00 | 2.50 |
| Aqua dem. | ad 100 | ad 100 | ad 100 | ad 100 | ad 100 |

Hair-Tinting Foam O/W Emulsions

| | Emulsion 1 | % by | Emulsion 2 | % by |
|---|---|---|---|---|
| | % by wt. | vol. | % by wt. | vol. |
| Stearic Acid | 5.00 | | 1.00 | |
| Cetyl Alcohol | 5.50 | | | |
| Cetearyl Alcohol | | | 2.00 | |
| PEG-40 Stearate | 8.50 | | | |
| PEG-20 Stearate | | | 1.00 | |
| Caprylic/Capric Triglyceride | 4.00 | | 2.00 | |
| C12-15 Alkyl Benzoate | 10.00 | | 15.00 | |
| Cyclomethicone | 4.00 | | | |
| Dimethicone | | | 0.50 | |
| Keratin-binding domain- | 5.0 | | 10.0 | |
| reactive dye 14_264 | | | | |
| Ethylhexyl Isostearate | | | 5.00 | |
| Myristyl Myristate | | | 2.00 | |
| Ceresin | 1.50 | | | |
| Glycerin | | | 3.00 | |
| Hydroxypropyl Starch | 1.00 | | 3.50 | |
| Phosphate | | | | |
| BHT | | | 0.02 | |
| Disodium EDTA | 0.50 | | 0.10 | |
| Perfume oil, Preservative | q.s. | | q.s. | |
| Potassium Hydroxide | q.s. | | q.s. | |
| Aqua dem. | ad 100 | | ad 100 | |
| | adjust pH | | adjust pH | |
| | to 6.5-7.5 | | to 5.0-6.0 | |
| Emulsion 1 | | 70 | | |
| Emulsion 2 | | | | 35 |
| Nitrogen | | 30 | | |
| Propane/Butane | | | | 65 |

Hair-Coloring Conditioner Shampoo with Pearlescence

| | 1 | 2 | 3 |
|---|---|---|---|
| Polyquaternium-10 | 0.50 | 0.50 | 0.40 |
| Sodium Laureth Sulfate | 9.00 | 8.50 | 8.90 |
| Cocamidopropyl Betaine | 2.50 | 2.60 | 3.00 |
| Uvinul ® MS 40 | 1.50 | 0.50 | 1.00 |
| Keratin-binding domain-reactive dye | 1.0 | 5.0 | 0.5 |
| 14_264 | | | |
| Pearlescent solution | 2.00 | 2.50 | |
| Disodium EDTA | 0.10 | 0.15 | 0.05 |
| Preservative, Perfume oil, Thickener | q.s. | q.s. | q.s. |
| Aqua dem. | ad 100 | ad 100 | ad 100 |

adjust pH to 6.0

Clear Hair-Coloring Conditioner Shampoo

| | 1 | 2 | 3 |
|---|---|---|---|
| Polyquaternium-10 | 0.50 | 0.50 | 0.50 |
| Sodium Laureth Sulfate | 0.2 | — | 0.1 |
| Keratin-binding domain-reactive dye | 5.0 | 10.0 | 20.0 |
| 14_264 | | | |
| Uvinul ® M 40 | 1.00 | 1.50 | 0.50 |
| Preservative, Perfume oil, Thickener | q.s. | q.s. | q.s. |
| Aqua dem. | ad 100 | ad 100 | ad 100 |

adjust pH to 6.0

Clear Conditioner Shampoo with Volume Effect for Hair Coloring

| | 1 | 2 | 3 |
|---|---|---|---|
| Sodium Laureth Sulfate | — | 0.5 | 1.00 |
| Uvinul ® MC 80 | 2.00 | 1.50 | 2.30 |
| Keratin-binding domain-reactive dye | 10.0 | 15.0 | 5.0 |
| 14_264 | | | |
| Cocamidopropyl Betaine | 2.50 | 2.60 | 2.20 |
| Disodium EDTA | 0.01 | 0.10 | 0.01 |
| Preservative, Perfume oil, Thickener | q.s. | q.s. | q.s. |
| Aqua dem. | ad 100 | ad 100 | ad 100 |

adjust pH to 6.0

Hair-Tinting Gel Cream

| | 1 | 2 | 3 | 4 |
|---|---|---|---|---|
| Acrylates/C10-30 Alkylacrylate | 0.40 | 0.35 | 0.40 | 0.35 |
| Crosspolymer | | | | |
| Carbomer | 0.20 | 0.22 | 0.20 | 0.22 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Uvinul ® A Plus ™ | 2.00 | 1.50 | 0.75 | 1.00 |
| UvaSorb ® k2A | | 3.00 | | |
| Ethylhexyl Bis- | | | | |
| Isopentylbenzoxazolylphenyl | | | | |
| Melamine | | | | |
| Uinul ® MC 80 | 3.00 | | 1.00 | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl | 2.50 | | 0.50 | 2.00 |
| Dibenzimidazole Tetrasulfonate | | | | |
| Uvinul ® T 150 | 4.00 | | 3.00 | 4.00 |
| Uvinul ® N 539 T | | 4.00 | | |
| Diethylhexyl Butamido Triazone | 1.00 | | | 2.00 |
| Phenylbenzimidazole | 0.50 | | 3.00 | |
| Sulfonic Acid | | | | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | 3.50 | 4.00 | 7.00 | 9.00 |
| Titanium Dioxide-microfine | 1.00 | | 3.00 | |
| Zinc Oxide-microfine | | | | 0.25 |
| Keratin-binding domain-reactive | 5.0 | 20.0 | 1.0 | 10.0 |
| dye 14_264 | | | | |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume oil | 0.20 | | 0.20 | |
| Aqua dem. | ad 100 | ad 100 | ad 100 | ad 100 |
| adjust pH to 6.0 | | | | |

OW Sunscreen Formulation

| | 1 | 2 | 3 | 4 | 5 | 6 | 7 |
|---|---|---|---|---|---|---|---|
| Glyceryl Stearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Sodium Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Keratin-binding domain-reactive | 10.0 | 0.5 | 3.0 | 5.0 | 0.1 | 15.0 | 7.5 |
| dye 14_264 | | | | | | | |
| UvaSorb ® k2A | | | | | | | |
| Ethylhexyl Bis- | | | | | | | |
| Isopentylbenzoxazolylphenyl | | | | | | | |
| Melamine | | | | | | | |
| Ethylhexyl Methoxycinnamate | | | | | 5.00 | 6.00 | 8.00 |
| Uvinul ® MC 80 | | | | | | | |
| Bis-Ethylhexyloxyphenol | | 1.50 | | 2.00 | 2.50 | | 2.50 |
| Methoxyphenyl Triazine | | | | | | | |
| Butyl Methoxydlbenzoylmethane | | | 2.00 | | 2.00 | 1.50 | |
| Disodium Phenyl Dibenzimidazole | 2.50 | | 0.50 | 2.00 | | 0.30 | |
| Tetrasulfonate | | | | | | | |
| Ethylhexyl Triazone Uvinul ® T 150 | 4.00 | | 3.00 | 4.00 | | 2.00 | |
| Octocrylene | | 4.00 | | | | | 7.50 |
| Uvinul ® N 539 T | | | | | | | |
| Diethylhexyl Butamido Triazone | 1.00 | | | 2.00 | 1.00 | | 1.00 |
| Phenylbenzimidazole Sulfonic Acid | 0.50 | | 3.00 | | | | |
| Methylene Bis-Benzotriazolyl | 2.00 | | 0.50 | 1.50 | 2.50 | | |
| Tetramethylbutylphenol | | | | | | | |
| Ethylhexyl Salicylate | | | 3.00 | | | | 5.00 |
| Drometrizole Trisiloxane | | | 0.50 | | | 1.00 | |
| Terephthalidene Dicamphor | | 1.50 | | 1.00 | 1.00 | | 0.50 |
| Sulfonic Acid | | | | | | | |
| Diethylhexyl 2,6-Naphthalate | 3.50 | | 7.00 | | 6.00 | 9.00 | |
| Titanium Dioxide-microfine | 1.00 | | 3.00 | | 3.50 | | 1.50 |
| Zinc Oxide-microfine | | | | 0.25 | | 2.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglyceride | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccharide Gum-1 | | 3.00 | 10.00 | | | | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium Iminodisuccinate | | | | 0.25 | 1.00 | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad100 | ad 100 |

Hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Sodium Carbomer | | 0.20 | | 0.30 | |
| Acrylates/C10-30 Alkyl Acrylate | 0.50 | | 0.40 | 0.10 | 0.50 |
| Crosspolymer | | | | | |
| Xanthan Gum | | 0.30 | 0.15 | | |
| Keratin-binding domain-reactive dye | 5.0 | 0.5 | 3.0 | 0.1 | 10.0 |
| 14_264 | | | | | |
| UvaSorb ® k2A Ethylhexyl Bis- | | 3.50 | | | |
| Isopentylbenzoxazolylphenyl Melamine | | | | | |
| Ethylhexyl Methoxycinnamate Uvinul ® MC 80 | | | | | 5.00 |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethylhexyl Triazone Uvinul ® T 150 | 4.00 | | 3.00 | 4.00 | |
| Octocrylene | | 4.00 | | | |
| Uvinul ® N 539 T | | | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic Acid | | 1.50 | | 1.00 | 1.00 |
| Diethylhexyl 2,6-Naphthalate | | | 7.00 | | 9.00 |
| Titanium Dioxide-microfine | 1.00 | | 3.00 | | 3.50 |
| Zinc Oxide-microfine | | | | 0.25 | |
| C12-15 Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| Biosaccharide Gum-1 | | 2.50 | 0.50 | | 2.00 |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume oil | 0.20 | | 0.05 | 0.40 | |
| Aqua dem. | ad 100 | ad 100 | ad 100 | ad 100 | ad 100 |

WO Sunscreen Emulsion

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Keratin-binding domain-reactive dye 14_264 | 5.0 | 1.0 | 10.0 | 0.5 | 15.0 |
| UvaSorb ® k2A | | 2.00 | | | |
| Ethylhexyl Bis-Isopentylbenzoxazolylphenyl | | | | | |
| Melamine | | | | | |
| Ethylhexyl Methoxycinnamate Uvinul ® MC | | | | | 5.00 |
| 80 | | | | | |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethyhexyl Triazone Uvinul ® T 150 | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic Acid | | 1.50 | | 1.00 | 1.00 |
| Diethylhexyl 2,6-Naphthalate | | | 7.00 | | 4.00 |
| Titanium Dioxide-microfine | 1.00 | | 3.00 | | 3.50 |
| Zinc Oxide-microfine | | | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soja (Soybean) Oil | | 1.00 | 1.50 | | 1.00 |
| Magnesium Sulfate MgSO₄ | 1.00 | 0.50 | | 0.50 | |
| Magnesium Chloride MgCl₂ | | | 1.00 | | 0.70 |
| Vitamin E Acetate | 0.50 | | 0.25 | | 1.00 |
| Ascorbyl Palmitate | 0.50 | | | 2.00 | |
| Biosaccharide Gum-1 | | | | 3.50 | 1.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume oil | 0.20 | | 0.40 | 0.35 | |
| Aqua dem. | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/Dicaprate | | | | 12.00 |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) Wax | 1.50 | 2.00 | 2.00 | 1.50 |
| Cera Alba | 0.50 | 0.50 | 0.50 | 0.40 |
| C16-40 Alkyl Stearate | | 1.50 | 1.50 | 1.50 |
| Keratin-binding domain-reactive dye 14_264 | 0.5 | 3.0 | 1.0 | 5.0 |
| UvaSorb ® k2A | | 2.00 | | 4.00 |
| Ethylhexyl Bis-Isopentylbenzoxazolylphenyl | | | | |
| Melamine | | | | |
| Ethylhexyl Methoxycinnamate Uvinul ® MC | | 3.00 | | |
| 80 | | | | |
| Bis-Ethylhexyloxyphenol Methoxyphenyl | | 1.50 | | 2.00 |
| Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Ethylhexyl Triazone Uvinul ® T 150 | 4.00 | | 3.00 | 4.00 |
| Octocrylene | | 4.00 | | |
| Diethylhexyl Butamido Triazone | 1.00 | | | 2.00 |
| Phenylbenzimidazole Sulfonic Acid | 0.50 | | 3.00 | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor Sulfonic Acid | | 1.50 | | 1.00 |
| Diethylhexyl 2,6-Naphthalate | | | 7.00 | |
| Titanium Dioxide-microfine | 1.00 | | 3.00 | |
| Zinc Oxide-microfine | | | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

PIT Emulsion

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone | | | | 0.50 | | 1.00 | | |
| Copolyol | | | | | | | | |
| Polyglyceryl-2 Dioleate | | | | 0.75 | 0.30 | | | |
| Keratin-binding domain- | 0.1 | 5.0 | 0.01 | 0.5 | 20.0 | 0.25 | 10.0 | 3.0 |
| reactive dye 14_264 | | | | | | | | |
| UvaSorb ® k2A | | | 4.00 | | | | 1.50 | |
| Ethylhexyl Bis- | | | | | | | | |
| Isopentylbenzoxazolyl- | | | | | | | | |
| phenyl Melamine | | | | | | | | |
| Ethylhexyl | | | | | 5.00 | 6.00 | 8.00 | 5.00 |
| Methoxycinnamate | | | | | | | | |
| Uvinul ® MC 80 | | | | | | | | |
| Bis-Ethylhexyloxyphenol | | 1.50 | | 2.00 | 2.50 | | 2.50 | 2.50 |
| Methoxyphenyl Triazine | | | | | | | | |
| Butyl Methoxydibenzoyl- | | | 2.00 | | 2.00 | 1.50 | | 2.00 |
| methane | | | | | | | | |
| Disodium Phenyl | 2.50 | | 0.50 | 2.00 | | 0.30 | | |
| Dibenzimidazole | | | | | | | | |
| Tetrasulfonate | | | | | | | | |
| Ethylhexyl Triazone | 4.00 | | 3.00 | 4.00 | | 2.00 | | |
| Uvinul ® T 150 | | | | | | | | |
| Octocrylene | | 4.00 | | | | | 7.50 | |
| Diethylhexyl Butamido | 1.00 | | | 2.00 | 1.00 | | 1.00 | 1.00 |
| Triazone | | | | | | | | |
| Phenylbenzimidazole | 0.50 | | 3.00 | | | | | |
| Sulfonic Acid | | | | | | | | |
| Methylene Bis- | 2.00 | | 0.50 | 1.50 | 2.50 | | | 2.50 |
| Benzotriazolyl | | | | | | | | |
| Tetramethylbutylphenol | | | | | | | | |
| Ethylhexyl Salicylate | | | 3.00 | | | | 5.00 | |
| Drometrizole Trisiloxane | | | 0.50 | | | 1.00 | | |
| Terephthalylidene | | 1.50 | | 1.00 | 1.00 | | 0.50 | 1.00 |
| Dicamphor Sulfonic Acid | | | | | | | | |
| Diethylhexyl 2,6- | | | 7.00 | | 10.00 | 7.50 | | 8.00 |
| Naphthalate | | | | | | | | |
| Titanium | 1.00 | | 3.00 | | 3.50 | | 1.50 | 3.50 |
| Dioxide-microfine | | | | | | | | |
| Zinc Oxide-microfine | | | | 0.25 | | 2.00 | | |
| C12-15 Alkyl Benzoate | 3.50 | | | 6.35 | | | | 0.10 |
| Cocoglyceride | | 3.00 | | 3.00 | | | | 1.00 |
| Dicaprylyl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C1-5 Alkyl | | 0.50 | | | 2.00 | | | |
| Galactomannan | | | | | | | | |
| Hydrogenated Coco- | | | | | 3.00 | 4.00 | | |
| Glycerides | | | | | | | | |
| Behenoxy Dimethicone | | | | | | 1.50 | 2.00 | |
| VP/Hexadecene | | | | 1.00 | 1.20 | | | |
| Copolymer | | | | | | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii | | 2.00 | | 3.60 | | 2.00 | | |
| (Shea Butter) | | | | | | | | |
| Iodopropyl | 0.12 | | | | 0.20 | | | |
| Butylcarbamate | | | | | | | | |
| Biosaccharide Gum-1 | | | | 0.10 | | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume oil | 0.20 | | 0.20 | | 0.24 | 0.16 | 0.10 | 0.10 |
| Aqua dem. | ad 100 | ad 100 | ad 100 | ad 100 | ad 100 | ad 100 | ad 100 | ad 100 |

Skin, or Hair-Tinting Gel Cream

| | 1 | 2 | 3 | 4 |
|---|---|---|---|---|
| Acrylates/C10-30 Alkylacrylate | 0.40 | 0.35 | 0.40 | 0.35 |
| Crosspolymer | | | | |
| Carbomer | 0.20 | 0.22 | 0.20 | 0.22 |
| Luvigel ® EM | 1.50 | 2.50 | 2.80 | 3.50 |
| Xanthan Gum | 0.10 | 0.13 | 0.10 | 0.13 |
| Cetearyl Alcohol | 3.00 | 2.50 | 3.00 | 2.50 |
| C12-15 Alkyl benzoate | 4.00 | 4.50 | 4.00 | 4.50 |
| Caprylic/Capric Triglyceride | 3.00 | 3.50 | 3.00 | 3.50 |
| Titanium Dioxide-microfine | 1.00 | | 1.50 | |
| Zinc Oxide-microfine | | 2.00 | | 0.25 |
| Keratin-binding domain-reactive | 0.5 | 10.0 | 3.0 | 5.0 |
| dye 14_264 | | | | |
| Dihydroxyacetone | | | 3.00 | 5.00 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume oil | 0.20 | | 0.20 | |
| Aqua dem. | ad 100 | ad 100 | ad 100 | ad 100 |
| adjust pH to 6.0 | | | | |

OW Self-Tanning Formulation

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Keratin-binding domain- | 0.1 | 0.5 | 0.025 | 5.0 | 3.0 | 10.0 | 1.0 |
| reactive dye 14_264 | | | | | | | |
| Dihydroxyacetone | | | 3.00 | 5.00 | | 4 | |
| Titanium Dioxide-microfine | 1.00 | | | | 1.50 | | 1.50 |
| Zinc Oxide-microfine | | | | 0.25 | | 2.00 | |
| C12-15 Alkyl Benzoate | | 0.25 | | | 4.00 | 7.00 | |
| Dicaprylyl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccharide Gum-1 | | 3.00 | 10.00 | | | | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad 100 | ad 100 |

OW Make Up

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Keratin-binding domain- | 3.0 | 5.0 | 2.0 | 0.5 | 1.0 | 5.0 | 10.0 |
| reactive dye 14_264 | | | | | | | |
| Titanium Dioxide | 10.00 | 12.00 | 9.00 | 8.50 | 11.00 | 9.50 | 10.00 |
| Iron Oxide | 2.00 | — | 3.00 | 5.00 | 0.2 | 6.00 | 4.40 |
| Zinc Oxide | | — | | 2.00 | | 3.00 | |
| C12-15 Alkyl Benzoate | | 0.25 | | | 4.00 | 7.00 | |
| Dicaprylyl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad 100 | ad 100 |

Self-Tanning Hydrodispersion with Tinting Effect

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl Acrylate | 0.50 | | 0.40 | 0.10 | 0.50 |
| Crosspolymer | | | | | |
| Xanthan Gum | | 0.30 | 0.15 | | |
| Keratin-binding domain-reactive dye | 3.0 | 1.0 | 0.5 | 0.1 | 5.0 |
| 14_264 | | | | | |
| Dihydroxyacetone | | | 3.00 | 5.00 | |
| Titanium Dioxide - microfine | 1.00 | | 1.00 | | 1.00 |
| Zinc Oxide - microfine | | 1.90 | | 0.25 | |
| C12-15 Alkyl Benzoate | 2.00 | 2.50 | | | |
| Dicaprylyl Ether | | 4.00 | | | |
| Butylene Glycol Dicaprylate/Dicaprate | 4.00 | | 2.00 | 6.00 | |
| Dicaprylyl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume oil | 0.20 | | 0.05 | 0.40 | |
| Aqua dem. | ad 100 | ad 100 | ad 100 | ad 100 | ad 100 |

After-Sun Hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl Acrylate | 0.50 | 0.30 | 0.40 | 0.10 | 0.50 |
| Crosspolymer | | | | | |
| Xanthan Gum | | 0.30 | 0.15 | | |
| Keratin-binding domain-reactive dye | 0.1 | 5.0 | 0.5 | 3.0 | 10.0 |
| 14_264 | | | | | |
| C12-15 Alkyl Benzoate | 2.00 | 2.50 | | | |
| Dicaprylyl Ether | | 4.00 | | | |
| Butylene Glycol Dicaprylate/Dicaprate | 4.00 | | 2.00 | 6.00 | |
| Dicaprylyl Carbonate | | 2.00 | 6.00 | | |
| Dimethicone | | 0.50 | 1.00 | | |
| Phenyl Trimethicone | 2.00 | | 0.50 | | |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| Trisodium EDTA | | 0.01 | 0.05 | | 0.10 |
| Ethanol | 15.00 | 10.00 | 8.00 | 12.00 | 9.00 |
| Perfume oil | 0.20 | | 0.05 | 0.40 | |
| Aqua dem. | ad 100 | ad 100 | ad 100 | ad 100 | ad 100 |

WO Emulsion

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Keratin-binding domain-reactive dye 14_264 | 7.0 | 12.0 | 0.1 | 0.5 | 1.0 |
| Titanium Dioxide-microfine | 1.00 | | 3.00 | | 3.50 |
| Zinc Oxide-microfine | | 0.90 | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soja (Soybean) Oil | | 1.00 | 1.50 | | 1.00 |
| Magnesium Sulfate | 1.00 | 0.50 | | 0.50 | |
| Magnesium Chloride | | | 1.00 | | 0.70 |
| Vitamin E Acetate | 0.50 | | 0.25 | | 1.00 |
| Ascorbyl Palmitate | 0.50 | | | 2.00 | |
| Biosaccharide Gum-1 | | | | 3.50 | 7.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume oil | 0.20 | | 0.40 | 0.35 | |
| Aqua dem. | ad 100 | ad 100 | ad 100 | ad 100 | ad 100 |

Solids-Stabilized Emulsion (Pickering Emulsions)

| | 1 | 2 | 3 | 4 | 5 |
|---|---|---|---|---|---|
| Mineral Oil | | | 16.00 | 16.00 | |
| Octyldodecanol | 9.00 | 9.00 | 5.00 | | |
| Caprylic/Capric Triglyceride | 9.00 | 9.00 | 6.00 | | |
| C12-15 Alkyl Benzoate | | | | 5.00 | 8.00 |
| Butylene Glycol Dicaprylate/Dicaprate | | | | | 8.00 |
| Dicaprylyl Ether | 9.00 | | | 4.00 | |
| Dicaprylyl Carbonate | | 9.00 | | | |
| Hydroxyoctacosanyl Hydroxystearate | 2.00 | 2.00 | 2.20 | 2.50 | 1.50 |
| Disteardimonium Hectorite | 1.00 | 0.75 | | 0.50 | 0.25 |
| Cera Microcristalline + Paraffinum Liquidum | | 0.35 | | | 5.00 |
| Hydroxypropyl Methylcellulose | | | 0.10 | | 0.05 |
| Dimethicone | | | | | 3.00 |
| Keratin-binding domain-reactive dye 14_264 | 1.0 | 15.0 | 0.1 | 3.0 | 5.0 |
| Titanium Dioxide + Alumina + Simethicone + | | 3.00 | | | |
| Aqua | | | | | |
| Titanium Dioxide + Trimethoxycaprylylsilane | | 2.00 | 4.00 | 2.00 | 4.00 |
| Silica Dimethyl Silylate | 2.50 | | | 6.00 | 2.50 |
| Boron Nitride | | | 1.00 | | |
| Starch/Sodium Metaphosphate Polymer | 2.00 | | | | |
| Tapioca Starch | | 0.50 | | | |
| Sodium Chloride | 5.00 | 7.00 | 8.50 | 3.00 | 4.50 |
| Glycerin | | | | 1.00 | |
| Trisodium EDTA | 1.00 | 1.00 | 1.00 | 1.00 | 1.00 |
| Vitamin E Acetate | 5.00 | 10.00 | 3.00 | 6.00 | 10.00 |
| Ascorbyl Palmitate | 1.00 | 1.00 | | 1.00 | |
| Methylparaben | | 0.60 | | | 0.20 |
| Propylparaben | | | | | 0.20 |
| Phenoxyethanol | | | 0.20 | | |
| Hexamidine Diisethionate | | | 0.40 | 0.50 | 0.40 |
| Diazolidinyl Urea | | | | | 0.08 |
| Ethanol | | | 0.23 | 0.20 | |
| Perfume oil | 5.00 | | 3.00 | 4.00 | |
| Aqua dem. | 0.20 | | 0.30 | 0.10 | |
| | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/ | | | | 12.00 |
| Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) | 1.50 | 2.00 | 2.00 | 1.50 |
| Wax | | | | |
| Cera Alba | 0.50 | 0.50 | 0.50 | 0.40 |
| C16-40 Alkyl Stearate | | 1.50 | 1.50 | 1.50 |
| Keratin-binding domain-reactive | 10.0 | 1.0 | 3.0 | 0.1 |
| dye 14_264 | | | | |
| Uvinul ® A Plus ™ | 2.00 | 1.50 | 0.75 | 9.00 |
| Titanium Dioxide-microfine | 1.00 | | 3.00 | |
| Zinc Oxide-microfine | | 1.00 | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

Self-Tanning PIT Emulsions

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone Copolyol | | | | 0.50 | | 1.00 | | |
| Polyglyceryl-2 | | | | 0.75 | 0.30 | | | |
| Keratin-binding domain-reactive | 0.1 | 0.5 | 0.01 | 5.0 | 0.5 | 3.0 | 0.025 | 10.0 |
| dye 14_264 | | | | | | | | |
| Dihydroxyacetone | | | 3.00 | 5.00 | | | 4.00 | |
| Titanium Dioxide - microfine | 1.00 | | 1.50 | | 3.50 | | 1.50 | 1.00 |
| Zinc Oxide - microfine | | 1.00 | | 0.25 | | 2.00 | | 1.50 |
| C12-15 Alkyl Benzoate | 3.50 | | | 6.35 | | | | 0.10 |
| Cocoglycerides | | 3.00 | | 3.00 | | | | 1.00 |
| Dicaprylyl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C1-5 Alkyl Galactomannan | | 0.50 | | | 2.00 | | | |
| Hydrogenated Cocoglycerides | | | | | 3.00 | 4.00 | | |
| Behenoxy Dimethicone | | | | | | 1.50 | 2.00 | |
| VP/Hexadecene Copolymer | | | | 1.00 | 1.20 | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii (Shea | | 2.00 | | 3.60 | | 2.00 | | |
| Butter) | | | | | | | | |
| Iodopropyl Butylcarbamate | 0.12 | | | | 0.20 | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume oil | 0.20 | | 0.20 | | 0.24 | 0.16 | 0.10 | 0.10 |
| Aqua dem. | ad 100 | ad 100 | ad 100 | ad 100 | ad 100 | ad 100 | ad 100 | ad 100 |

Oil Gel

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol | | | | 12.00 |
| Dicaprylate/Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Myristyl Myristate | 3.50 | 3.00 | 4.00 | 3.00 |
| Quaternium-18 Bentonite | 5.00 | 5.00 | 6.00 | 6.00 |
| Propylene Carbonate | 15.00 | 20.00 | 18.00 | 19.50 |
| Keratin-binding domain-reactive | 1.0 | 0.5 | 3.0 | 5.0 |
| dye 14_264 | | | | |
| Vitamin E Acetate | 0.50 | 1.00 | | |
| Ascorbyl Palmitate | 0.05 | | 0.05 | |
| Buxus Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

### Example 63

In the following formulations cosmetic sunscreen preparations comprising a combination of at least one inorganic pigment, preferably zinc oxide and/or titanium oxide, Uvinul A Plus and further organic UV-A and UV-B filters are described.

The inorganic pigments here may be present in coated form, i.e. that they are treated superficially. This surface treatment can, for example, consist in providing the pigments with a thin hydrophobic layer by a method known per se, as described in DE-A-33 14 742.

The formulations given below are prepared in a customary way known to the person skilled in the art.

Dermocosmetic preparations according to the invention are described below comprising the keratin-binding effector molecule prepared according to Example 19 (keratin-binding domain according to SEQ ID No.: ID 166 coupled with the dye 14_{—}264). Said keratin-binding effector molecule is referred to in the examples below as keratin-binding domain-reactive dye 14_{—}264. It goes without saying for the person skilled in the art that all of the other dyes described in Tables 6 and 7 can also be coupled with the KBD according to Example 19, 19 a or 19 b and can be used in the preparations given below.

The content of keratin-binding domain-reactive dye 14_{—}264 refers to 100% of active ingredient. The active ingredient according to the invention can be used either in pure form or as an aqueous solution. In the case of the aqueous solution, the content of water dem. in the particular formulation must be adjusted.

| | | | |
|---|---|---|---|
| A | 7.50 | Uvinul ® MC 80 | Ethylhexyl Methoxycinnamate |
| | 2.00 | Uvinul ® M 40 | Benzophenone-3 |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | T-Lite ® SF | Titanium Dioxide, Alumina Hydrate, |
| | | | Dimethicone/Methicone Copolymer |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 1.00 | Sepigel 305 | Polyacrylamide. C13-14 Isoparaffin, Laureth-7 |
| | 1.00 | Keratin-binding domain- | |
| | | reactive dye 14_264 | |
| | q.s. | Preservative | |
| A | 7.50 | Uvinul ® MC 80 | Ethylhexyl Methoxycinnamate |
| | 2.00 | Uvinul ® M 40 | Benzophenone-3 |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 7.50 | Uvinul ® MC 80 | Ethylhexyl Methoxycinnamate |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 4.00 | Uvinul ® MC 80 | Ethylhexyl Methoxycinnamate |
| | 2.00 | Uvinul ® T 150 | Ethylhexyl Triazone |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 4.00 | Uvinul ® MC 80 | Ethylhexyl Methoxycinnamate |
| | 2.00 | Uvinul ® T 150 | Ethylhexyl Triazone |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | T-Lite ® SF | Titanium Dioxide, Alumina Hydrate, |
| | | | Dimethicone/Methicone Copolymer |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 4.00 | Uvinul ® MC 80 | Ethylhexyl Methoxycinnamate |
| | 2.00 | Uvinul T ® 150 | Ethylhexyl Triazone |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 4.00 | Uvinul ® MC 80 | Ethylhexyl Methoxycinnamate |
| | 1.00 | Uvinul ® T 150 | Ethylhexyl Triazone |
| | 2.00 | Parsol ® 1789 | Butyl Methoxydibenzoylmethane |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | T-Lite ® SF-S | Titanium Dioxide, Silica Hydrate, Alumina |
| | | | Hydrate, Methicone/Dimethicone Copolymer |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.70 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 0.50 | Keratin-binding domain- | |
| | | reactive dye 14_264 | |
| | q.s. | Preservative | |
| A | 4.00 | Uvinul ® MC 80 | Ethylhexyl Methoxycinnamate |
| | 1.00 | Uvinul ® T 150 | Ethylhexyl Triazone |
| | 2.00 | Parsol ® 1789 | Butyl Methoxydibenzoylmethane |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 67.10 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 0.10 | Keratin-binding domain- | |
| | | reactive dye 14_264 | |
| | q.s. | Preservative | |
| A | 5.00 | Uvinul ® N 539 T | Octocrylene |
| | 2.00 | Parsol ® 1789 | Butyl Methoxydibenzoylmethane |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | T-Lite ® SF-S | Titanium Dioxide, Silica Hydrate, Alumina |
| | | | Hydrate, Methicone/Dimethicone Copolymer |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 5.00 | Uvinul N ® 539 T | Octocrylene |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | T-Lite ® SF | Titanium Dioxide, Alumina Hydrate, |
| | | | Dimethicone/Methicone Copolymer |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 65.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 2.00 | Keratin-binding domain- | |
| | | reactive dye 14_264 | |
| | q.s. | Preservative | |
| A | 5.00 | Uvinul ® N 539 T | Octocrylene |
| | 2.00 | Uvinul ® A Plus | Diethylamino Hydroxybenzoyl Hexyl |
| | | | Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.70 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 0.50 | Keratin-binding domain- | |
| | | reactive dye 14_264 | |
| | q.s. | Preservative | |
| A | 5.00 | Uvinul ® N 539 T | Octocrylene |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 2.0 | Mexoryl ® SX | Terephthalidene Dicamphor Sulfonic Acid |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 5.00 | Uvinul ® N 539 T | Octocrylene |
| | 2.00 | Uvinul ® A Plus | Diethylamino Hydrobenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 2.0 | Mexoryl ® SX | Terephthalidene Dicamphor Sulfonic Acid |
| | 65.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 2.00 | Keratin-binding domain- | |
| | | reactive dye 14_264 | |
| | q.s. | Preservative | |
| A | 5.00 | Uvinul ® N 539 T | Octocrylene |
| | 2.00 | Uvinul ® A Plus | Diethylamino Hydroxybenzoyl Hexyl |
| | | | Benzoate |
| | 2.00 | Mexoryl ® XL | Drometrizole Trisiloxane |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 67.10 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 0.10 | Keratin-binding domain- | |
| | | reactive dye 14_264 | |
| | q.s. | Preservative | |
| A | 5.00 | Uvinul ® N 539 T | Octocrylene |
| | 2.00 | Tinosorb ® S | Bis-Ethylhexyloxyphenol Methoxyphenyl |
| | | | Triazine |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 7.50 | Uvinul ® MC 80 | Ethylhexyl Methoxycinnamate |
| | 5.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | Z-COTE ® MAX | Zinc Oxide, Diphenyl Capryl |
| | | | Methicone |
| C | 4.00 | 1.2-Propylene Glykol Care | Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 65.30 | Water demin. | Aqua dem. |
| | q.s. | Preservative | |
| | 0.50 | Vitamin E Acetate | Tocopheryl Acetate |
| | 2.00 | Keratin-binding domain-reactive | |
| | | dye 14_264 | |
| | 1.00 | RetiSTAR ™ | Caprylic/Capric Triglyceride, Sodium |
| | | | Ascorbate, Tocopherol, Retinol |
| A | 7.50 | Uvinul ® MC 80 | Ethylhexyl Methoxycinnamate |
| | 2.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | T-Lite ® SF-S | Titanium Dioxide, Silica Hydrate, |
| | | | Alumina Hydrate, |
| | | | Methicone/Dimethicone Copolymer |
| C | 4.00 | 1,2-Propylene Glycol Care | Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 68.3 | Water demin. | Aqua dem. |
| | q.s. | Preservative | |
| | 0.50 | Vitamin E Acetate | Tocopheryl Acetate |
| | 1.00 | Keratin-binding domain-reactive | |
| | | dye 14_264 | |
| | 2.00 | RetiSTAR ™ | Caprylic/Capric Triglyceride, Sodium |
| | | | Ascorbate, Tocopherol, Retinol |
| A | 7.50 | Uvinul ® MC 80 | Ethylhexyl Methoxycinnamate |
| | 5.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | T-Lite ® SF | Titanium Dioxide, Alumina Hydrate, |
| | | | Dimethicone/Methicone Copolymer |
| C | 4.00 | 1,2-Propylene Glycol Care | Propylene Glycol |
| | 1.00 | D-Panthenol 50 P | Panthenol, Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 65.30 | Water demin. | Aqua dem. |
| | q.s. | Preservative | |
| | 0.50 | Vitamin E Acetate | Tocopheryl Acetate |
| | 1.00 | Keratin-binding domain-reactive | |
| | | dye 14_264 | |
| | 1.00 | RetiSTAR ™ | Caprylic/Capric Triglyceride, Sodium |
| | | | Ascorbate, Tocopherol, Retinol |
| A | 5.50 | Uvinul ® MC 80 | Ethylhexyl Methoxycinnamate |
| | 2.00 | Uvinul ® A Plus | Diethylamino Hydroxybenzoyl Hexyl |
| | | | Benzoate |
| | 3.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | T-Lite ® SF | Titanium Dioxide, Alumina Hydrate, |
| | | | Dimethicone/Methicone Copolymer |
| C | 4.00 | 1,2-Propylene Glycol Care | Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 67.30 | Water demin. | Aqua dem. |
| | q.s. | Preservative | |
| | 0.50 | Vitamin E Acetate | Tocopheryl Acetate |
| | 2.00 | Keratin-binding domain-reactive | |
| | | dye 14_264 | |
| | 1.00 | RetiSTAR ™ | Caprylic/Capric Triglyceride, Sodium |
| | | | Ascorbate, Tocopherol, Retinol |
| A | 1.00 | Abil ® Care 85 | Bis-PEG/PPG-16/16 PEG/PPG-16/16 |
| | | | Dimethicone, Caprylic/Capric |
| | | | Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 2.00 | Parsol 1789 | Butyl Methoxydibenzoylmethane |
| | 2.00 | Mexoryl ® XL | Drometrizole Trisiloxane |
| | 10.00 | Witconol APM | PPG-3 Myristyl Ether |
| | 1.00 | Uvinul ® T 150 | Ethylhexyl Triazone |
| | 1.00 | Dow Corning 345 Fluid | Cyclopentasiloxane, Cyclohexasiloxane |
| | 5.00 | Uvinul ® N 539 T | Octocrylene |
| B | 3.00 | Z-COTE ® MAX | Zinc Oxide, Diphenyl Capryl Methicone |
| C | 5.00 | 1,2-Propylene Glykol | Propylene Glycol |
| | 1.00 | Mexoryl ® SX | Terephthalidene Dicamphor Sulfonic |
| | | | Acid |
| | 0.20 | Keltrol ® | Xanthan Gum |
| | 0.06 | Edeta BD | Disodium EDTA |
| | 0.04 | TEA | Triethanolamine |
| | 0.30 | Carbopol ® Ultrez 10P | Carbomer |
| | 63.50 | Water demin. | Aqua dem. |
| | 1.00 | Keratin-binding domain-reactive | |
| | | dye 14_264 | |
| D | q.s. | Perfume oil | |
| | 0.50 | Glydant | DMDM Hydantoin |
| A | 5.50 | Uvinul ® MC 80 | Ethylhexyl Methoxycinnamate |
| | 2.00 | Uvinul ® A Plus | Diethylamino Hydroxybenzoyl Hexyl |
| | | | Benzoate |
| | 3.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | T-Lite ® SF-S | Titanium Dioxide, Silica Hydrate, |
| | | | Alumina Hydrate, |
| | | | Methicone/Dimethicone Copolymer |
| C | 4.00 | 1,2-Propylene Glycol Care | Propylene Glycol |
| | 0.20 | Keltrol ® | Xanthan Gum |
| | 2.00 | Simulgel ® NS | Hydroxyethyl Acrylate/Sodium |
| | | | Acryloyldimethyl Taurate Copolymer, |
| | | | Squalane, Polysorbate 60 |
| | 66.30 | Water demin. | Aqua dem. |
| | q.s. | Preservative | |
| | 0.50 | Vitamin E Acetate | Tocopheryl Acetate |
| | 0.50 | Keratin-binding domain-reactive | |
| | | dye 14_264 | |
| | 1.00 | RetiSTAR ™ | Caprylic/Capric Triglyceride, Sodium |
| | | | Ascorbate, Tocopherol, Retinol |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 5.00 | Uvinul ® MC 80 | Ethylhexyl Methoxycinnamate |
| | 1.00 | Ganex ® V-220 | VP/Eicosene Copolymer |
| | 7.00 | Isopropyl Palmitate | Isopropyl Palmitate |
| B | 3.00 | Z-COTE ® MAX | Zinc Oxide, Diphenyl Capryl |
| | | | Methicone |
| C | 0.20 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 65.10 | Water demin. | Aqua dem. |
| | 0.20 | Edeta BD | Disodium EDTA |
| | 5.00 | 1,2-Propylene Glycol Care | Propylene Glycol |
| | 0.50 | Keratin-binding domain-reactive dye | |
| | | 14_264 | |
| D | 1.00 | Vitamin E Acetate | Tocopheryl Acetate |
| | q.s. | Preservative | |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 5.00 | Uvinul ® MC 80 | Ethylhexyl Methoxycinnamate |
| | 1.00 | Ganex ® V-220 | VP/Eicosene Copolymer |
| | 7.00 | Isopropyl Palmitate | Isopropyl Palmitate |
| B | 3.00 | T-Lite ® SF | Titanium Dioxide, Alumina Hydrate, |
| | | | Dimethicone/Methicone Copolymer |
| C | 0.50 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 65.10 | Water demin. | Aqua dem. |
| | 0.20 | Edeta BD | Disodium EDTA |
| | 5.00 | 1,2-Propylene Glycol Care | Propylene Glycol |
| | 0.50 | Keratin-binding domain-reactive dye | |
| | | 14_264 | |
| D | 1.00 | Vitamin E Acetate | Tocopheryl Acetate |
| | q.s. | Preservative | |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 5.00 | Uvinul ® MC 80 | Ethylhexyl Methoxycinnamate |
| | 1.00 | Ganex ® V-220 | VP/Eicosene Copolymer |
| | 7.00 | Isopropyl Palmitate | Isopropyl Palmitate |
| B | 3.00 | T-Lite ® SF-S | Titanium Dioxide, Silica Hydrate, |
| | | | Alumina Hydrate, |
| | | | Methicone/Dimethicone Copolymer |
| C | 0.50 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 64.60 | Water demin. | Aqua dem. |
| | 0.20 | Edeta BD | Disodium EDTA |
| | 5.00 | 1,2-Propylene Glycol Care | Propylene Glycol |
| | 1.00 | Keratin-binding domain-reactive dye | |
| | | 14_264 | |
| D | 1.00 | Vitamin E Acetate | Tocopheryl Acetate |
| | q.s. | Preservative | |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 5.00 | Uvinul ® MC 80 | Ethylhexyl Methoxycinnamate |
| | 1.00 | Ganex ® V-220 | VP/Eicosene Copolymer |
| | 7.00 | Isopropyl Palmitate | Isopropyl Palmitate |
| B | 3.00 | T-Lite ® SF-S | Titanium Dioxide, Silica Hydrate, |
| | | | Alumina Hydrate, |
| | | | Methicone/Dimethicone Copolymer |
| C | 0.50 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 64.10 | Water demin. | Aqua dem. |
| | 2.00 | Neo Heliopan ® AP | Disodium Phenyl Dibenzimidazole |
| | | | Tetrasulfonate |
| | 0.20 | Edeta BD | Disodium EDTA |
| | 5.00 | 1,2-Propylene Glycol Care | Propylene Glycol |
| D | 1.00 | Vitamin E Acetate | Tocopheryl Acetate |
| | 1.00 | Keratin-binding domain-reactive dye | |
| | | 14_264 | |
| | q.s. | Preservative | |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 7.00 | Uvinul ® N 539 T | Octocrylene |
| | 1.00 | Ganex ® V-220 | VP/Eicosene Copolymer |
| | 7.00 | Isopropyl Palmitate | Isopropyl Palmitate |
| B | 3.00 | T-Lite ® SF-S | Titanium Dioxide, Silica Hydrate, |
| | | | Alumina Hydrate, |
| | | | Methicone/Dimethicone Copolymer |
| C | 0.50 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 64.60 | Water demin. | Aqua dem. |
| | 1.00 | Mexoryl ® SX | Terephthalidene Dicamphor Sulfonic |
| | | | Acid |
| | 0.20 | Edeta BD | Disodium EDTA |
| | 5.00 | 1,2-Propylene Glycol Care | Propylene Glycol |
| D | 1.00 | Vitamin E Acetate | Tocopheryl Acetate |
| | 1.00 | Keratin-binding domain-reactive dye | |
| | | 14_264 | |
| | q.s. | Preservative | |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 2.00 | Mexoryl ® XL | Drometrizole Trisiloxane |
| | 5.00 | Uvinul ® N 539 T | Octocrylene |
| | 1.00 | Ganex ® V-220 | VP/Eicosene Copolymer |
| | 7.00 | Isopropyl Palmitate | Isopropyl Palmitate |
| B | 3.00 | T-Lite ® SF | Titanium Dioxide, Alumina Hydrate, |
| | | | Dimethicone/Methicone Copolymer |
| C | 0.50 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 65.10 | Water demin. | Aqua dem. |
| | 0.20 | Edeta BD | Disodium EDTA |
| | 5.00 | 1,2-Propylene Glycol Care | Propylene Glycol |
| D | 1.00 | Vitamin E Acetate | Tocopheryl Acetate |
| | 0.50 | Keratin-binding domain-reactive dye | |
| | | 14_264 | |
| | q.s. | Preservative | |
| | 1.00 | Urea | Urea |
| | 2.00 | Neo Heliopan ® AP | Disodium Phenyl Dibenzimidazole |
| | | | Tetrasulfonate |
| | 2.00 | Glycerin 87% | Glycerin |
| | 1.20 | Aristoflex ™ AVC | Ammonium Acryloyldimethyl |
| | | | Taurate/VP Copolymer |
| | 71.30 | Water demin. | Aqua dem. |
| B | 3.00 | Cremophor ® CO 40 | PEG-40 Hydrogenated Castor Oil |
| | q.s. | Perfume oil | |
| | 5.00 | Miglyol 812 | Caprylic/Capric Triglyceride |
| | 2.00 | Uvinul ® N 539 T | Octocrylene |
| | 8.00 | Z-COTE ® MAX | Zinc Oxide, Diphenyl Capryl |
| | | | Methicone |
| | 5.00 | Keratin-binding domain-reactive dye | |
| | | 14 264 | |
| | q.s. | Preservative | |
| | 5.00 | Z-COTE ® MAX | Zinc Oxide, Diphenyl Capryl |
| | | | Methicone |
| | 2.00 | Parsol ® 1789 | Butyl Methoxydibenzoylmethane |
| | 1.00 | Uvinul ® T 150 | Ethylhexyl Triazone |
| | 5.00 | Uvinul ® MC 80 | Ethylhexyl Methoxycinnamate |
| | 2.00 | Uvinul ® T 539 N | Octocrylene |
| | 0.50 | Abil ® 350 | Dimethicone |
| | 2.75 | Carnico wax LT 20 | Copernica Cerifera (Carnauba) Wax, |
| | | | Paraffin |
| | 3.70 | Candelilla wax LT 281 LJ | Euphorbia Cerifera (Candelilla) Wax |
| | 1.80 | Beeswax 3050 PH | Beeswax |
| | 3.20 | TeCero wax 30445 | Microcrystalline Wax |
| | 3.20 | TeCero wax 1030 K | Microcrystalline Wax |
| | 1.34 | Cutina CP | Cetyl Palmitate |
| | 6.40 | Vaseline | Petrolatum |
| | 7.30 | Softisan 100 | Hydrogenated Cocoglycerides |
| | 10.00 | Luvitol ® EHO | Cetearyl Ethylhexanoate |
| | 0.17 | Bisabolol nat. | Bisabolol |
| | 1.84 | Vitamin E Acetate | Tocopheryl Acetate |
| | 0.42 | D,L-Alpha-Tocopherol | Tocopherol |
| | 1.00 | Keratin-binding domain-reactive dye | |
| | | 14_264 | |
| | 40.38 | Castor Oil | Ricinus Communis (Castor) Oil |
| | 5.00 | T-Lite ® SF | Titanium Dioxide, Alumina Hydrate, |
| | | | Dimethicone/Methicone Copolymer |
| | 2.00 | Parsol ® 1789 | Butyl Methoxydibenzoylmethane |
| | 1.00 | Uvinul ® T 150 | Ethylhexyl Triazone |
| | 3.00 | Uvinul ® MC 80 | Ethylhexyl Methoxycinnamate |
| | 2.00 | Uvinul ® N 539 T | Octocrylene |
| | 0.50 | Abil ® 350 | Dimethicone |
| | 2.75 | Carnico wax LT 20 | Copernica Cerifera (Carnauba) Wax, |
| | | | Paraffin |
| | 3.70 | Candelilla wax LT 281 LJ | Euphorbia Cerifera (Candelilla) Wax |
| | 1.80 | Beeswax 3050 PH | Beeswax |
| | 3.20 | TeCero wax 30445 | Microcrystalline Wax |
| | 3.20 | TeCero wax 1030 K | Microcrystalline Wax |
| | 1.34 | Cutina CP | Cetyl Palmitate |
| | 6.40 | Vaseline | Petrolatum |
| | 7.30 | Softisan 100 | Hydrogenated Cocoglycerides |
| | 10.00 | Luvitol ® EHO | Cetearyl Ethylhexanoate |
| | 0.17 | Bisabolol nat. | Bisabolol |
| | 1.84 | Vitamin E Acetate | Tocopheryl Acetate |
| | 0.42 | D,L-Alpha-Tocopherol | Tocopherol |
| | 1.00 | Keratin-binding domain-reactive dye | |
| | | 14_264 | |
| | 40.38 | Castor Oil | Ricinus Communis (Castor) Oil |
| | 5.00 | T-Lite ® SF-S | Titanium Dioxide, Silica Hydrate. |
| | | | Alumina Hydrate, |
| | | | Methicone/Dimethicone Copolymer |
| | 2.00 | Uvinul ® A Plus | Diethylamino Hydroxybenzoyl Hexyl |
| | | | Benzoate |
| | 2.00 | Mexoryl ® XL | Drometrizole Trisiloxane |
| | 3.00 | Uvinul ® MC 80 | Ethylhexyl Methoxycinnamate |
| | 0.50 | Abil ® 350 | Dimethicone |
| | 2.75 | Carnico wax LT 20 | Copernica Cerifera (Carnauba) |
| | | | Wax, Paraffin |
| | 3.70 | Candelilla wax LT 281 LJ | Euphorbia Cerifera (Candelilla) |
| | | | Wax |
| | 1.80 | Beeswax 3050 PH | Beeswax |
| | 3.20 | TeCero wax 30445 | Microcrystalline Wax |
| | 3.20 | TeCero wax 1030 K | Microcrystalline Wax |
| | 1.34 | Cutina CP | Cetyl Palmitate |
| | 6.40 | Vaseline | Petrolatum |
| | 7.30 | Softisan 100 | Hydrogenated Cocoglycerides |
| | 10.00 | Luvitol ® EHO | Cetearyl Ethylhexanoate |
| | 0.17 | Bisabolol nat. | Bisabolol |
| | 1.84 | Vitamin E Acetate | Tocopheryl Acetate |
| | 0.42 | D,L-Alpha-Tocopherol | Tocopherol |
| | 2.00 | Keratin-binding domain-reactive dye | |
| | | 14_264 | |
| | 39.38 | Castor Oil | Ricinus Communis (Castor) Oil |
| A | 6.00 | Cremophor ® WO 7 | PEG-7 Hydrogenated Castor Oil |
| | 2.00 | Elfacos ® ST 9 | PEG-45/Dodecyl Glycol Copolymer |
| | 3.00 | Isopropyl Myristate | Isopropyl Myristate |
| | 8.00 | Jojoba oil | Simmondsia Chinensis (Jojoba) Seed |
| | | | Oil |
| | 4.00 | Uvinul ® MC 80 | Ethylhexyl Methoxycinnamate |
| | 2.00 | Uvinul ® A Plus | Diethylamino Hydroxybenzoyl Hexyl |
| | | | Benzoate |
| | 1.00 | Abil ® 350 | Dimethicone |
| B | 5.00 | Z-COTE ® MAX | Zinc Oxide, Diphenyl Capryl Methicone |
| | 3.00 | T-Lite ® SF | Titanium Dioxide, Alumina Hydrate, |
| | | | Dimethicone/Methicone Copolymer |
| C | 0.20 | Edeta BD | Disodium EDTA |
| | 5.00 | Glycerin 87% | Glycerin |
| | 0.30 | Chemag 2000 | Imidazolidinyl Urea |
| | 59.00 | Water demin. | Aqua dem. |
| D | q.s. | Perfume oil | |
| | 1.00 | Keratin-binding domain-reactive | |
| | | dye 14_264 | |
| | q.s. | Preservative | |
| | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 5.00 | Uvinul ® MC 80 | Ethylhexyl Methoxycinnamate |
| | 1.00 | Uvinul ® T 150 | Ethylhexyl Triazone |
| | 1.00 | Ganex ® V-220 | VP/Eicosene Copolymer |
| | 7.00 | Isopropyl Myristate | Isopropyl Myristate |
| B | 5.00 | Z-COTE ® MAX | Zinc Oxide, Diphenyl Capryl |
| | | | Methicone |
| C | 0.20 | Keltrol ® | Xanthan Gum |
| | 0.50 | Simulgel ® NS | Hydroxyethyl Acrylate/Sodium |
| | | | Acryloyldimethyl Taurate |
| | | | Copolymer, Squalane, |
| | | | Polysorbate 60 |
| | 61.10 | Water demin. | Aqua dem. |
| | 0.20 | Edeta BD | Disodium EDTA |
| | 5.00 | 1,2-Propylene Glycol Care | Propylene Glycol |
| D | 1.00 | Vitamin E Acetate | Tocopheryl Acetate |
| | 1.50 | Keratin-binding domain-reactive dye | |
| | | 14_264 | |
| | q.s. | Preservative | |
| A | 2.00 | Luvitol ® EHO | Cetearyl Ethylhexanoate |
| | 5.00 | Paraffin oil | Mineral Oil |
| | 5.00 | Z-COTE ® MAX | Zinc Oxide, Diphenyl Capryl |
| | | | Methicone |
| B | 1.00 | Cremophor ® A 6 | Ceteareth-6, Stearyl Alcohol |
| | 2.00 | Cremophor ® A 25 | Ceteareth-25 |
| | 3.00 | Glycerinmonostearate | Glyceryl Stearate |
| | 4.00 | Cetylstearyl alcohol | Cetearyl Alcohol |
| C | 5.00 | 1,2-Propylene Glycol Care | Propylene Glycol |
| | 7.50 | Luviquat Care | Polyquaternium-44 |
| | q.s. | Preservative | |
| | q.s. | Perfume oil | |
| | 1.00 | Keratin-binding domain-reactive dye | |
| | | 14_264 | |
| | 64.50 | Water demin. | Aqua dem. |
| | 5.00 | T-Lite ® SF | Titanium Dioxide, Alumina |
| | | | Hydrate, Dimethicone/Methicone |
| | | | Copolymer |
| | 10.00 | Z-COTE ® MAX | Zinc Oxide, Diphenyl Capryl |
| | | | Methicone |
| | 12.00 | Beeswax 3044 PH | Beeswax |
| | 8.00 | Candelilla wax LT 281 LJ | Euphorbia Cerifera (Candelilla) |
| | | | Wax |
| | 5.00 | Tegin | Glyceryl Stearate SE |
| | 5.00 | Dow Corning ® 345 Fluid | Cyclopentasiloxane, |
| | | | Cyclohexasiloxane |
| | 5.00 | Witconol ® APM | PPG-3 Myristyl Ether |
| | 5.00 | Softisan 154 | Hydrogenated Palm Oil |
| | 8.00 | Paraffin oil, high viscosity | Mineral Oil |
| | 3.00 | Vaseline | Petrolatum |
| | 2.00 | Keratin-binding domain-reactive dye | |
| | | 14_264 | |
| | 32.00 | Castor Oil | Ricinus Communis (Castor) Oil |
| | 3.00 | Uvinul ® MC 80 | Ethylhexyl Methoxycinnamate |
| | 2.00 | Uvinul ® T 150 | Ethylhexyl Triazone |
| | 2.00 | Uvinul ® A Plus | Diethylamino Hydroxybenzoyl |
| | | | Hexyl Benzoate |
| | 10.00 | Z-COTE ® MAX | Zinc Oxide, Diphenyl Capryl |
| | | | Methicone |
| | 12.00 | Beeswax 3044 PH | Beeswax |
| | 3.00 | Vaseline | Petrolatum |
| | 8.00 | Candelilla wax LT 281 LJ | Euphorbia Cerifera (Candelilla) |
| | | | Wax |
| | 8.00 | Paraffin oil, high viscosity | Mineral Oil |
| | 5.00 | Tegin | Glyceryl Stearate SE |
| | 5.00 | Softisan 154 | Hydrogenated Palm Oil |
| | 5.00 | Witconol APM | PPG-3 Myristyl Ether |
| | 5.00 | Dow Corning 345 Fluid | Cyclopentasiloxane, |
| | | | Cyclohexasiloxane |
| | 0.10 | Keratin-binding domain-reactive dye | |
| | | 14_264 | |
| | 28.90 | Castor Oil | Ricinus Communis (Castor) Oil |
| | 5.00 | T-Lite ® SF | Titanium Dioxide, Alumina |
| | | | Hydrate, Dimethicone/Methicone |
| | | | Copolymer |
| | 6.00 | Finsolv ® TN | C12-15 Alkyl Benzoate |
| | 10.00 | Uvinul ® MC 80 | Ethylhexyl Methoxycinnamate |
| | 6.00 | Miglyol ® 812 | Caprylic/Capric Triglyceride |
| | 5.00 | Arlacel ® P 135 | PEG-30 Dipolyhydroxystearate |
| | 2.00 | Ganex ® V 216 | PVP/Hexadecene Copolymer |
| | 2.00 | Elfacos ® ST 9 | PEG-45/Dodecyl Glycol |
| | | | Copolymer |
| B | 3.00 | 1,2-Propylene Glycol Care | Propylene Glycol |
| | 0.10 | Edeta BD | Disodium EDTA |
| | 1.00 | Magnesium sulfate 7-hydrate | Magnesium Sulfate |
| | 59.40 | Water demin. | Aqua dem. |
| | 0.50 | Keratin-binding domain-reactive dye | |
| | | 14_264 | |
| | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 4.00 | Uvinul ® MC 80 | Ethylhexyl Methoxycinnamate |
| | 2.00 | Uvinul ® N 539 T | Octocrylene |
| B | 5.00 | Z-COTE ® MAX | Zinc Oxide, Diphenyl Capryl |
| | | | Methicone |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 66.20 | Water demin. | Aqua dem. |
| | 1.50 | Keratin-binding domain-reactive dye | |
| | | 14_264 | |
| D | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 4.00 | Uvinul ® MC 80 | Ethylhexyl Methoxycinnamate |
| | 2.00 | Uvinul ® N 539 T | Octocrylene |
| B | 5.00 | T-Lite ® SF-S | Titanium Dioxide, Silica Hydrate, |
| | | | Alumina Hydrate, |
| | | | Methicone/Dimethicone Copolymer |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 1.00 | Keratin-binding domain-reactive dye | |
| | | 14_264 | |
| | 66.70 | Water demin. | Aqua dem. |
| D | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 8.00 | Uvinul ® A Plus B | Ethylhexyl Methoxycinnamate and |
| | | | Diethylamino Hydroxybenzoyl |
| | | | hexyl Benzoate |
| | 2.00 | Uvinul ® N 539 T | Octocrylene |
| B | 5.00 | Z-COTE ® MAX | Zinc Oxide, Diphenyl Capryl |
| | | | Methicone |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 0.10 | Keratin-binding domain-reactive dye | |
| | | 14_264 | |
| | 63.60 | Water demin. | Aqua dem. |
| | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 8.00 | Uvinul ® A Plus B | Ethylhexyl Methoxycinnamate and |
| | | | Diethylamino Hydroxybenzoyl |
| | | | Hexyl Benzoate |
| | 2.00 | Uvinul ® N 539 T | Octocrylene |
| B | 5.00 | T-Lite ® SF | Titanium Dioxide, Alumina Hydrate, |
| | | | Dimethicone/Methicone Copolymer |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 63.50 | Water demin. | Aqua dem. |
| | 0.20 | Keratin-binding domain-reactive dye | |
| | | 14_264 | |
| | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 8.00 | Uvinul ® A Plus B | Ethylhexyl Methoxycinnamate and |
| | | | Diethylamino Hydroxybenzoyl |
| | | | hexyl Benzoate |
| | 2.00 | Uvinul ® N 539 T | Octocrylene |
| B | 5.00 | T-Lite ® SF-S | Titanium Dioxide, Silica Hydrate, |
| | | | Alumina Hydrate, |
| | | | Methicone/Dimethicone Copolymer |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 0.50 | Keratin-binding domain-reactive dye | |
| | | 14_264 | |
| | 63.20 | Water demin. | Aqua dem. |
| | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 7.00 | Uvinul ® MC 80 | Ethylhexyl Methoxycinnamate |
| | 2.00 | Mexoryl ® XL | Drometrizole Trisiloxane |
| B | 5.00 | Z-COTE ® MAX | Zinc Oxide, Diphenyl Capryl |
| | | | Methicone |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 64.69 | Water demin. | Aqua dem. |
| | 0.01 | Keratin-binding domain-reactive dye | |
| | | 14_264 | |
| | q.s. | Preservative | |
| A | 7.50 | Uvinul ® MC 80 | Ethylhexyl Methoxycinnamate |
| | 3.00 | Uvinul ® N 539 T | Octocrylene |
| | 2.00 | Uvinul ® A Plus | Diethylamino Hydroxybenzoyl |
| | | | Hexyl Benzoate |
| | 1.00 | Cremophor ® CO 40 | PEG-40 Hydrogenated Castor Oil |
| | 10.00 | Miglyol 812 | Caprylic/Capric Triglyceride |
| | 1.50 | Dow Corning ® 345 Fluid | Cyclopentasiloxane, |
| | | | Cyclohexasiloxane |
| B | 3.50 | Luvigel ® EM | Caprylic/Capric Triglyceride, |
| | | | Sodium Acrylates Copolymer |
| C | 46.00 | Water demin. | Aqua dem. |
| D | 5.00 | 1,2-Propylene Glycol Care | Propylene Glycol |
| | 0.50 | Cremophor ® A 25 | Ceteareth-25 |
| | 0.05 | Keratin-binding domain-reactive dye | |
| | | 14_264 | |
| | 20.00 | Ethanol 96% | Alcohol |
| A | 1.00 | Uvinul ® A Plus | Diethylamino Hydroxybenzoyl |
| | | | Hexyl Benzoate |
| | 1.00 | Tinosorb ® S | Bis-Ethylhexyloxyphenol |
| | | | Methoxyphenyl Triazine |
| | 3.00 | Uvinul ® MC 80 | Ethylhexyl Methoxycinnamate |
| | 8.00 | Miglyol ® 812 | Caprylic/Capric Triglyceride |
| | 1.50 | Dow Corning ® 350 Fluid | Dimethicone |
| | 3.00 | Z-COTE ® MAX | Zinc Oxide, Diphenyl Capryl |
| | | | Methicone |
| | 3.00 | Finsolv ® TN | C12-15 Alkyl Benzoate |
| | 1.00 | Cremophor ® CO 40 | PEG-40 Hydrogenated Castor Oil |
| B | 2.00 | Luvigel ® EM | Caprylic/Capric Triglyceride, |
| | | | Sodium Acrylates Copolymer |
| C | 54.80 | Water demin. | Aqua dem. |
| D | 15.00 | Ethanol 96% | Alcohol |
| | 5.00 | 1,2-Propylene Glycol Care | Propylene Glycol |
| | 0.50 | Cremophor ® A 25 | Ceteareth-25 |
| | 1.00 | Keratin-binding domain-reactive dye | |
| | | 14_264 | |
| | 1.00 | Vitamin E Acetate | Tocopheryl Acetate |
| | 0.20 | Bisabolol rac. | Bisabolol |
| A | 4.00 | Dehymuls ® SBL | Polyglyceryl-2 |
| | | | Dipolyhydroxystearate, |
| | | | Dicaprylyl Ether, |
| | | | Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 6.00 | Finsolv ® TN | C12-15 Alkyl Benzoate |
| | 6.00 | Miglyol ® 812 | Caprylic/Capric Triglyceride |
| | 5.00 | Uvinul ® MC 80 | Ethylhexyl Methoxycinnamate |
| | 3.00 | Neo Heliopan ® HMS | Homosalate |
| B | 5.00 | Z-COTE ® MAX | Zinc Oxide, Diphenyl Capryl |
| | | | Methicone |
| C | 3.00 | 1,2-Propylene Glycol Care | Propylene Glycol |
| | 0.30 | Chemag 2000 | Imidazolidinyl Urea |
| | 1.00 | Magnesium sulfate 7-hydrate | Magnesium Sulfate |
| | 65.10 | Water demin. | Aqua dem. |
| | 0.10 | Keratin-binding domain-reactive dye | |
| | | 14_264 | |
| | q.s. | Preservative | |
| A | 4.00 | Tego ® Care 450 | Polyglyceryl-3 Methyl Glucose |
| | | | Distearate |
| | 4.50 | Uvinul ® MC 80 | Ethylhexyl Methoxycinnamate |
| | 3.00 | Uvinul ® N 539 T | Octocrylene |
| | 2.00 | Uvinul ® T 150 | Ethylhexyl Triazone |
| | 0.50 | Vitamin E Acetate | Tocopheryl Acetate |
| B | 5.00 | Isohexadecane | Isohexadecane |
| | 5.00 | Cosmacol ® EMI | Di-C12-13 Alkyl Malate |
| | 3.50 | Cetiol ® SN | Cetearyl Isononanoate |
| | 1.00 | Ganex ® V-220 | VP/Eicosene Copolymer |
| | 5.00 | T-Lite ® SF | Titanium Dioxide, Alumina |
| | | | Hydrate, Dimethicone/Methicone |
| | | | Copolymer |
| C | 60.00 | Water demin. | Water |
| | 5.00 | Glycerin 87% | Glycerin |
| | 1.00 | Lanette E | Sodium Cetearyl Sulfate |
| | 0.30 | Keltrol ® | Xanthan Gum |
| | 0.20 | Keratin-binding domain-reactive dye | |
| | | 14 264 | |
| D | q.s. | Preservative | |
| A | 4.00 | Tego ® Care 450 | Polyglyceryl-3 Methyl Glucose |
| | | | Distearate |
| | 4.50 | Uvinul ® MC 80 | Ethylhexyl Methoxycinnamate |
| | 3.00 | Uvinul ® N 539 T | Octocrylene |
| | 2.00 | Neo Heliopan ® HMS | Homosalate |
| | 0.50 | Vitamin E Acetate | Tocopheryl Acetate |
| B | 5.00 | Isohexadecane | Isohexadecane |
| | 5.00 | Cosmacol ® EMI | Di-C12-13 Alkyl Malate |
| | 3.50 | Cetiol ® SN | Cetearyl Isononanoate |
| | 1.00 | Ganex ® V-220 | VP/Eicosene Copolymer |
| | 5.00 | T-Lite ® SF | Titanium Dioxide, Alumina |
| | | | Hydrate, |
| | | | Dimethicone/Methicone |
| | | | Copolymer |
| C | 59.20 | Water demin. | Aqua dem. |
| | 5.00 | Glycerin 87% | Glycerin |
| | 1.00 | Lanette E | Sodium Cetearyl Sulfate |
| | 0.30 | Keltrol ® | Xanthan Gum |
| | 1.00 | Keratin-binding domain-reactive dye | |
| | | 14_264 | |
| D | q.s. | Preservative | |
| A | 4.00 | Tego ® Care 450 | Polyglyceryl-3 Methyl Glucose |
| | | | Distearate |
| | 4.50 | Uvinul ® MC 80 | Ethylhexyl Methoxycinnamate |
| | 3.00 | Neo Heliopan ® OS | Octisalate |
| | 2.00 | Neo Heliopan ® HMS | Homosalate |
| | 0.50 | Vitamin E Acetate | Tocopheryl Acetate |
| B | 5.00 | Isohexadecane | Isohexadecane |
| | 5.00 | Cosmacol ® EMI | Di-C12-13 Alkyl Malate |
| | 3.50 | Cetiol ® SN | Cetearyl Isononanoate |
| | 1.00 | Ganex ® V-220 | VP/Eicosene Copolymer |
| | 5.00 | Z-COTE ® MAX | Zinc Oxide, Diphenyl Capryl |
| | | | Methicone |
| C | 60.15 | Water demin. | Aqua dem. |
| | 5.00 | Glycerin 87% | Glycerin |
| | 1.00 | Lanette E | Sodium Cetearyl Sulfate |
| | 0.30 | Keltrol ® | Xanthan Gum |
| | 0.05 | Keratin-binding domain-reactive dye | |
| | | 14_264 | |
| D | q.s. | Preservative | |
| A | 4.00 | Tego ® Care 450 | Polyglyceryl-3 Methyl Glucose |
| | | | Distearate |
| | 4.50 | Uvinul ® N 539 T | Octocrylene |
| | 3.00 | Neo Heliopan ® OS | Octisalate |
| | 2.00 | Neo Heliopan ® HMS | Homosalate |
| | 0.50 | Vitamin E Acetate | Tocopheryl Acetate |
| B | 5.00 | Isohexadecane | Isohexadecane |
| | 5.00 | Cosmacol ® EMI | Di-C12-13 Alkyl Malate |
| | 3.50 | Cetiol ® SN | Cetearyl Isononanoate |
| | 1.00 | Ganex ® V-220 | VP/Eicosene Copolymer |
| | 5.00 | Z-COTE ® MAX | Zinc Oxide, Diphenyl Capryl |
| | | | Methicone |
| C | 60.19 | Water demin. | Aqua dem. |
| | 5.00 | Glycerin 87% | Glycerin |
| | 1.00 | Lanette E | Sodium Cetearyl Sulfate |
| | 0.30 | Keltrol ® | Xanthan Gum |
| | 0.01 | Keratin-binding domain-reactive dye | |
| | | 14_264 | |
| D | q.s. | Preservative | |
| A | 4.00 | Tego ® Care 450 | Polyglyceryl-3 Methyl Glucose |
| | | | Distearate |
| | 4.50 | Uvinul ® N 539 T | Octocrylene |
| | 3.00 | Neo Heliopan ® OS | Octisalate |
| | 2.00 | Neo Heliopan ® HMS | Homosalate |
| | 0.50 | Vitamin E Acetate | Tocopheryl Acetate |
| B | 5.00 | Isohexadecane | Isohexadecane |
| | 5.00 | Cosmacol ® EMI | Di-C12-13 Alkyl Malate |
| | 3.50 | Cetiol ® SN | Cetearyl Isononanoate |
| | 1.00 | Ganex ® V-220 | VP/Eicosene Copolymer |
| | 5.00 | T-Lite ® SF-S | Titanium Dioxide, Silica |
| | | | Hydrate, Alumina Hydrate, |
| | | | Methicone/Dimethicone |
| | | | Copolymer |
| C | 59.20 | Water demin. | Aqua dem. |
| | 5.00 | Glycerin 87% | Glycerin |
| | 1.00 | Lanette E | Sodium Cetearyl Sulfate |
| | 0.30 | Keltrol ® | Xanthan Gum |
| | 1.00 | Keratin-binding domain-reactive dye | |
| | | 14_264 | |
| D | q.s. | Preservative | |
| A | 8.00 | Cetiol ® B | Dibutyl Adipate |
| | 8.00 | Finsolv ® TN | C12-15 Alkyl Benzoate |
| | 12.00 | Myritol ® 331 | Cocoglycerides |
| | 1.00 | Lanette E | Sodium Cetearyl Sulfate |
| | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 3.00 | Tinosorb ® S | Bis-Ethylhexyloxyphenol |
| | | | Methoxyphenyl Triazine |
| | 1.00 | Vitamin E Acetate | Tocopheryl Acetate |
| B | 4.00 | Z-COTE ® MAX | Zinc Oxide, Diphenyl Capryl |
| | | | Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 1.50 | Veegum Ultra | Magnesium Aluminum Silicate |
| | 49.45 | Water demin. | Aqua dem. |
| | 1.00 | Keratin-binding domain-reactive dye | |
| | | 14_264 | |
| D | q.s. | Preservative | |
| A | 8.00 | Cetiol ® B | Dibutyl Adipate |
| | 8.00 | Finsolv ® TN | C12-15 Alkyl Benzoate |
| | 12.00 | Myritol ® 331 | Cocoglycerides |
| | 1.00 | Lanette E | Sodium Cetearyl Sulfate |
| | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 3.00 | Tinosorb ® S | Bis-Ethylhexyloxyphenol |
| | | | Methoxyphenyl Triazine |
| | 1.00 | Vitamin E Acetate | Tocopheryl Acetate |
| B | 4.00 | T-Lite ® SF | Titanium Dioxide, Alumina |
| | | | Hydrate, Dimethicone/Methicone |
| | | | Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 1.50 | Veegum Ultra | Magnesium Aluminum Silicate |
| | 50.25 | Water demin. | Aqua dem. |
| D | 0.50 | Citric Acid | Citric Acid |
| | q.s. | Perfume oil | |
| | 0.20 | Keratin-binding domain-reactive dye | |
| | | 14_264 | |
| | q.s. | Preservative | |
| A | 5.00 | Cosmacol ® EMI | Di-C12-13 Alkyl Malate |
| | 4.50 | Uvinul ® MC 80 | Ethylhexyl Methoxycinnamate |
| | 3.00 | Uvinul ® N 539 T | Octocrylene |
| | 2.00 | Uvinul ® T 150 | Ethylhexyl Triazone |
| B | 4.00 | Tego ® Care 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 2.50 | T-Lite ® SF | Titanium Dioxide, Alumina |
| | | | Hydrate, |
| | | | Dimethicone/Methicone |
| | | | Copolymer |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 42.00 | Water demin. | Aqua dem. |
| D | 5.00 | Eusolex ® 232 | Phenylbenzimidazole Sulfonic |
| | | | Acid |
| | 10.00 | Water demin. | Aqua dem. |
| E | 0.70 | Sodium hydroxide | Sodium Hydroxide |
| | 1.00 | Keratin-binding domain-reactive dye | |
| | | 14_264 | |
| | q.s. | Preservative | |
| A | 5.00 | Cosmacol ® EMI | Di-C12-13 Alkyl Malate |
| | 4.50 | Uvinul ® MC 80 | Ethylhexyl Methoxycinnamate |
| | 3.00 | Uvinul ® N 539 T | Octocrylene |
| | 2.00 | Uvinul ® A Plus | Diethylamino Hydrobenzoyl |
| | | | Hexyl Benzoate |
| B | 4.00 | Tego ® Care 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 2.50 | T-Lite ® SF | Titanium Dioxide, Alumina |
| | | | Hydrate, |
| | | | Dimethicone/Methicone |
| | | | Copolymer |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 41.00 | Water demin. | Aqua dem. |
| D | 5.00 | Eusolex ® 232 | Phenylbenzimidazole Sulfonic |
| | | | Acid |
| | 2.00 | Keratin-binding domain-reactive dye | |
| | | 14_264 | |
| | 10.00 | Water demin. | Aqua dem. |
| E | 0.70 | Sodium hydroxide | Sodium Hydroxide |
| | q.s. | Preservative | |
| A | 5.00 | Cosmacol ® EMI | Di-C12-13 Alkyl Malate |
| | 4.50 | Uvinul ® MC 80 | Ethylhexyl Methoxycinnamate |
| | 3.00 | Uvinul ® N 539 T | Octocrylene |
| | 2.00 | Uvinul ® A Plus | Diethylamino Hydrobenzoyl |
| | | | Hexyl Benzoate |
| B | 4.00 | Tego Care 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 2.50 | Z-COTE ® MAX | Zinc Oxide, Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 42.50 | Water demin. | Aqua dem. |
| D | 5.00 | Eusolex ® 232 | Phenylbenzimidazole Sulfonic |
| | | | Acid |
| | 0.50 | Keratin-binding domain-reactive dye | |
| | | 14_264 | |
| | 10.00 | Water demin. | Aqua dem. |
| E | 0.70 | Sodium hydroxide | Sodium Hydroxide |
| | q.s. | Preservative | |
| A | 5.00 | Cosmacol ® EMI | Di-C12-13 Alkyl Malate |
| | 7.50 | Uvinul ® N 539 T | Octocrylene |
| | 2.00 | Mexoryl ® XL | Drometrizole Trisiloxane |
| B | 4.00 | Tego ® Care 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 5.00 | Z-COTE ® MAX | Zinc Oxide, Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 40.50 | Water demin. | Aqua dem. |
| D | 5.00 | Eusolex ® 232 | Phenylbenzimidazole Sulfonic |
| | | | Acid |
| | 1.00 | Keratin-binding domain-reactive dye | |
| | | 14_264 | |
| | 10.00 | Water demin. | Aqua dem. |
| E | 0.70 | Sodium hydroxide | Sodium Hydroxide |
| | q.s. | Preservative | |
| A | 5.00 | Cosmacol ® EMI | Di-C12-13 Alkyl Malate |
| | 5.50 | Uvinul ® N 539 T | Octocrylene |
| | 2.00 | Uvinul ® A Plus | Diethylamino Hydrobenzoyl |
| | | | Hexyl Benzoate |
| | 2.00 | Mexoryl ® XL | Drometrizole Trisiloxane |
| B | 4.00 | Tego ® Care 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 5.00 | Z-COTE ® MAX | Zinc Oxide, Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 41.50 | Water demin. | Aqua dem. |
| D | 5.00 | Eusolex ® 232 | Phenylbenzimidazole Sulfonic |
| | | | Acid |
| | 0.50 | Keratin-binding domain-reactive dye | |
| | | 14_264 | |
| | 9.50 | Water demin. | Aqua dem. |
| E | 0.70 | Sodium hydroxide | Sodium Hydroxide |
| | q.s. | Preservative | |
| A | 5.00 | Cosmacol ® EMI | Di-C12-13 Alkyl Malate |
| | 5.50 | Uvinul ® N 539 T | Octocrylene |
| | 2.00 | Uvinul ® A Plus | Diethylamino Hydrobenzoyl |
| | | | Hexyl Benzoate |
| | 2.00 | Mexoryl ® XL | Drometrizole Trisiloxane |
| B | 4.00 | Tego Care ® 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 5.00 | T-Lite ® SF-S | Titanium Dioxide, Silica |
| | | | Hydrate, Alumina Hydrate, |
| | | | Methicone/Dimethicone |
| | | | Copolymer |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 40.50 | Water demin. | Aqua dem. |
| D | 5.00 | Eusolex ® 232 | Phenylbenzimidazole Sulfonic |
| | | | Acid |
| | 1.00 | Keratin-binding domain-reactive dye | |
| | | 14_264 | |
| | 10.00 | Water demin. | Aqua dem. |
| E | 0.70 | Sodium hydroxide | Sodium Hydroxide |
| | q.s. | Preservative | |
| A | 8.00 | Cetiol ® B | Dibutyl Adipate |
| | 8.00 | Finsolv ® TN | C12-15 Alkyl Benzoate |
| | 10.00 | Myritol ® 331 | Cocoglycerides |
| | 1.00 | Lanette E | Sodium Cetearyl Sulfate |
| | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb ® S | Bis-Ethylhexyloxyphenol |
| | | | Methoxyphenyl Triazine |
| | 1.00 | Uvinul ® T 150 | Ethylhexyl Triazone |
| | 1.00 | Vitamin E Acetate | Tocopheryl Acetate |
| B | 5.00 | T-Lite ® SF | Titanium Dioxide, Alumina |
| | | | Hydrate, |
| | | | Dimethicone/Methicone |
| | | | Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 2.00 | Keratin-binding | domain-reactive dye |
| | | 14_264 | |
| | 1.50 | Veegum Ultra | Magnesium Aluminum Silicate |
| | 51.95 | Water demin. | Aqua dem. |
| D | q.s. | Perfume oil | |
| | q.s. | Preservative | |
| A | 8.00 | Cetiol ® B | Dibutyl Adipate |
| | 8.00 | Finsolv ® TN | C12-15 Alkyl Benzoate |
| | 10.00 | Myritol ® 331 | Cocoglycerides |
| | 1.00 | Lanette E | Sodium Cetearyl Sulfate |
| | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb ® S | Bis-Ethylhexyloxyphenol |
| | | | Methoxyphenyl triazine |
| | 1.00 | Uvinul ® T 150 | Ethylhexyl Triazone |
| | 1.00 | Vitamin E Acetate | Tocopheryl Acetate |
| B | 5.00 | Z-COTE ® MAX | Zinc Oxide, Diphenyl Capryl |
| | | | Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 1.00 | Keratin-binding domain-reactive dye | |
| | | 14_264 | |
| | 1.50 | Veegum Ultra | Magnesium Aluminum Silicate |
| | 52.95 | Water demin. | Aqua dem. |
| D | q.s. | Perfume oil | |
| | q.s. | Preservative | |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare ® 2000 | Decyl Glucoside |
| | 0.30 | Keltrol ® T | Xanthan Gum |
| | 57.00 | Water demin. | Aqua dem. |
| B | 5.00 | T-Lite ® SF-S | Titanium Dioxide, Silica |
| | | | Hydrate, Alumina Hydrate, |
| | | | Methicone/Dimethicone |
| | | | Copolymer |
| C | 7.50 | Uvinul ® MC 80 | Ethylhexyl Methoxycinnamate |
| | 2.00 | Uvinul ® A Plus | Diethylamino Hydroxybenzoyl |
| | | | Hexyl Benzoate |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol ® 812 | Caprylic/Capric Triglyceride |
| D | 1.00 | Vitamin E Acetate | Tocopheryl Acetate |
| | 2.00 | Keratin-binding domain-reactive dye | |
| | | 14_264 | |
| | 0.20 | Bisabolol rac. | Bisabolol |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol ® T | Xanthan Gum |
| | 58.00 | Water demin. | Aqua dem. |
| B | 5.00 | Z-COTE ® MAX | Zinc Oxide, Diphenyl Capryl |
| | | | Methicone |
| C | 7.50 | Uvinul ® MC 80 | Ethylhexyl Methoxycinnamate |
| | 2.00 | Uvinul ® A Plus | Diethylamino Hydroxybenzoyl |
| | | | Hexyl Benzoate |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol ® 812 | Caprylic/Capric Triglyceride |
| D | 1.00 | Vitamin E Acetate | Tocopheryl Acetate |
| | 1.00 | Keratin-binding domain-reactive dye | |
| | | 14_264 | |
| | 0.20 | Bisabolol rac. | Bisabolol |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare ® 2000 | Decyl Glucoside |
| | 0.30 | Keltrol ® T | Xanthan Gum |
| | 58.50 | Water demin. | Aqua dem. |
| B | 5.00 | T-Lite ® SF-S | Titanium Dioxide, Silica |
| | | | Hydrate, Alumina Hydrate, |
| | | | Methicone/Dimethicone |
| | | | Copolymer |
| C | 7.50 | Uvinul ® MC 80 | Ethylhexyl Methoxycinnamate |
| | 5.00 | Uvinul ® N 539 T | Octocrylene |
| | 2.00 | Parsol ® 1789 | Butyl |
| | | | Methoxydibenzoylmethane |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol ® 812 | Caprylic/Capric Triglyceride |
| D | 1.00 | Vitamin E Acetate | Tocopheryl Acetate |
| | 0.50 | Keratin-binding domain-reactive dye | |
| | | 14_264 | |
| | 0.20 | Bisabolol rac. | Bisabolol |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol ® T | Xanthan Gum |
| | 2.00 | Mexoryl ® SL | Terephthalidene Dicamphor |
| | | | Sulfonic Acid |
| | 58.40 | Water demin. | Aqua dem. |
| B | 6.00 | T-Lite ® SF-S | Titanium Dioxide, Silica |
| | | | Hydrate, Alumina Hydrate, |
| | | | Methicone/Dimethicone |
| | | | Copolymer |
| | 6.00 | Uvinul ® N 539 T | Octocrylene |
| | 2.00 | Parsol ® 1789 | Butyl |
| | | | Methoxydibenzoylmethane |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 0.10 | Keratin-binding domain-reactive dye | |
| | | 14_264 | |
| | 0.20 | Bisabolol rac. | Bisabolol |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare ® 2000 | Decyl Glucoside |
| | 0.30 | Keltrol ® T | Xanthan Gum |
| | 2.00 | Mexoryl ® SL | Terephthalidene Dicamphor |
| | | | Sulfonic Acid |
| | 58.45 | Water demin. | Aqua dem. |
| B | 6.00 | Z-COTE ® MAX | Zinc Oxide, Diphenyl Capryl |
| | | | Methicone |
| | 6.00 | Uvinul ® N 539 T | Octocrylene |
| | 2.00 | Parsol ® 1789 | Butyl |
| | | | Methoxydibenzoylmethane |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 0.05 | Keratin-binding domain-reactive dye | |
| | | 14_264 | |
| | 0.20 | Bisabolol rac. | Bisabolol |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare ® 2000 | Decyl Glucoside |
| | 0.30 | Keltrol ® T | Xanthan Gum |
| | 2.00 | Neo Heliopan ® AP | Disodium Phenyl |
| | | | Dibenzimidazole Tetrasulfonate |
| | 57.50 | Water demin. | Aqua dem. |
| B | 6.00 | Z-COTE ® MAX | Zinc Oxide, Diphenyl Capryl |
| | | | Methicone |
| | 6.00 | Uvinul ® N 539 T | Octocrylene |
| | 2.00 | Parsol ® 1789 | Butyl |
| | | | Methoxydibenzoylmethane |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 1.00 | Keratin-binding domain-reactive dye | |
| | | 14_264 | |
| | 0.20 | Bisabolol rac. | Bisabolol |
| A | 25.00 | Dow Corning ® 345 Fluid | Cyclopentasiloxane, |
| | | | Cyclohexasiloxane |
| | 20.00 | Dow Corning ® 245 Fluid | Cyclopentasiloxane |
| | 8.00 | Uvinul ® MC 80 | Ethylhexyl Methoxycinnamate |
| | 4.00 | Abil ® EM 90 | Cetyl PEG/PPG-10/1 |
| | | | Dimethicone |
| | 7.00 | T-Lite ® SF | Titanium Dioxide, Alumina |
| | | | Hydrate, |
| | | | Dimethicone/Methicone |
| | | | Copolymer |
| B | 17.00 | Ethanol 95% | Alcohol |
| | 10.00 | Z-COTE ® MAX | Zinc Oxide, Diphenyl Capryl |
| | | | Methicone |
| | 4.50 | Water demin. | Aqua dem. |
| | 3.00 | Glycerin 87% | Glycerin |
| | 0.50 | Keratin-binding domain-reactive dye | |
| | | 14_264 | |
| | 1.00 | Talc | Talc |
| A | 25.00 | Dow Corning ® 345 Fluid | Cyclopentasiloxane, |
| | | | Cyclohexasiloxane |
| | 20.00 | Dow Corning ® 245 Fluid | Cyclopentasiloxane |
| | 8.00 | Uvinul ® MC 80 | Ethylhexyl Methoxycinnamate |
| | 4.00 | Abil ® EM 90 | Cetyl PEG/PPG-10/1 |
| | | | Dimethicone |
| | 7.00 | T-Lite ® SF-S | Titanium Dioxide, Silica |
| | | | Hydrate, Alumina Hydrate, |
| | | | Methicone/Dimethicone |
| | | | Copolymer |
| B | 17.00 | Ethanol 95% | Alcohol |
| | 9.99 | Z-COTE ® MAX | Zinc Oxide, Diphenyl Capryl |
| | | | Methicone |
| | 5.00 | Water demin. | Aqua dem. |
| | 3.00 | Glycerin 87% | Glycerin |
| | 0.01 | Keratin-binding domain-reactive dye | |
| | | 14_264 | |
| | 1.00 | Talc | Talc |
| A | 20.00 | Dow Corning ® 345 Fluid | Cyclopentasiloxane, |
| | | | Cyclohexasiloxane |
| | 10.00 | Dow Corning ® 245 Fluid | Cyclopentasiloxane |
| | 7.50 | Uvinul ® MC 80 | Ethylhexyl Methoxycinnamate |
| | 2.00 | Uvinul ® T 150 | Ethylhexyl Triazone |
| | 12.00 | Cosmacol ® EMI | Di-C12-13 Alkyl Malate |
| | 4.00 | Abil ® EM 90 | Cetyl PEG/PPG-10/1 |
| | | | Dimethicone |
| B | 7.00 | T-Lite ® SF | Titanium Dioxide, Alumina |
| | | | Hydrate, |
| | | | Dimethicone/Methicone |
| | | | Copolymer |
| C | 15.00 | Ethanol 95% | Alcohol |
| | 10.00 | Z-COTE ® MAX | Zinc Oxide, Diphenyl Capryl |
| | | | Methicone |
| | 3.00 | Glycerin 87% | Glycerin |
| | 1.50 | Keratin-binding domain-reactive dye | |
| | | 14_264 | |
| | 8.00 | Water demin. | Aqua dem. |
| A | 20.00 | Dow Corning ® 345 Fluid | Cyclopentasiloxane, |
| | | | Cyclohexasiloxane |
| | 10.00 | Dow Corning ® 245 Fluid | Cyclopentasiloxane |
| | 7.50 | Uvinul ® MC 80 | Ethylhexyl Methoxycinnamate |
| | 2.00 | Tinosorb ® S | Bis-Ethylhexyloxyphenol |
| | | | Methoxyphenyl Triazine |
| | 12.00 | Cosmacol ® EMI | Di-C12-13 Alkyl Malate |
| | 4.00 | Abil ® EM 90 | Cetyl PEG/PPG-10/1 |
| | | | Dimethicone |
| B | 7.00 | T-Lite ® SF | Titanium Dioxide, Alumina |
| | | | Hydrate, |
| | | | Dimethicone/Methicone |
| | | | Copolymer |
| C | 15.00 | Ethanol 95% | Alcohol |
| | 10.00 | Z-COTE ® MAX | Zinc Oxide, Diphenyl Capryl |
| | | | Methicone |
| | 3.00 | Glycerin 87% | Glycerin |
| | 0.50 | Keratin-binding domain-reactive dye | |
| | | 14_264 | |
| | 9.00 | Water demin. | Aqua dem. |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 3.00 | T-Lite ® SF | Titanium Dioxide, Alumina |
| | | | Hydrate, |
| | | | Dimethicone/Methicone |
| | | | Copolymer |
| | 4.00 | Isopropyl Palmitate | Isopropyl Palmitate |
| | 1.00 | Abil ® 350 | Dimethicone |
| B | 7.00 | Uvinul ® MC 80 | Ethylhexyl Methoxycinnamate |
| | 1.00 | Uvinul ® T 150 | Ethylhexyl Triazone |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 61.30 | Water demin. | Aqua dem. |
| D | 1.00 | Vitamin E Acetate | Tocopheryl Acetate |
| | 1.00 | Keratin-binding domain-reactive dye | |
| | | 14_264 | |
| | q.s. | Preservative | |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 3.00 | Z-COTE ® MAX | Zinc Oxide, Diphenyl Capryl |
| | | | Methicone |
| | 4.00 | Isopropyl Palmitate | Isopropyl Palmitate |
| | 1.00 | Abil ® 350 | Dimethicone |
| B | 7.00 | Uvinul ® MC 80 | Ethylhexyl Methoxycinnamate |
| | 1.00 | Uvinul ® T 150 | Ethylhexyl Triazone |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 62.20 | Water demin. | Aqua dem. |
| D | 1.00 | Vitamin E Acetate | Tocopheryl Acetate |
| | 0.10 | Keratin-binding domain-reactive dye | |
| | | 14_264 | |
| | q.s. | Preservative | |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 3.00 | Z-COTE ® MAX | Zinc Oxide, Diphenyl Capryl |
| | | | Methicone |
| | 4.00 | Isopropyl Palmitate | Isopropyl Palmitate |
| | 1.00 | Abil ® 350 | Dimethicone |
| B | 7.00 | Uvinul ® MC 80 | Ethylhexyl Methoxycinnamate |
| | 1.00 | Tinosorb ® S | Bis-Ethylhexyloxyphenol |
| | | | Methoxyphenyl Triazine |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 60.30 | Water demin. | Aqua dem. |
| D | 1.00 | Vitamin E Acetate | Tocopheryl Acetate |
| | 2.00 | Keratin-binding domain-reactive dye | |
| | | 14_264 | |
| | q.s. | Preservative | |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 6.00 | Z-COTE ® MAX | Zinc Oxide, Diphenyl Capryl |
| | | | Methicone |
| | 4.00 | Isopropyl Palmitate | Isopropyl Palmitate |
| | 1.00 | Abil ® 350 | Dimethicone |
| B | 4.00 | Uvinul ® N 539 T | Octocrylene |
| | 1.00 | Tinosorb ® S | Bis-Ethylhexyloxyphenol |
| | | | Methoxyphenyl Triazine |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 62.10 | Water demin. | Aqua dem |
| D | 1.00 | Vitamin E Acetate | Tocopheryl Acetate |
| | 0.20 | Keratin-binding domain-reactive dye | |
| | | 14_264 | |
| | q.s. | Preservative | |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 6.00 | Z-COTE ® MAX | Zinc Oxide, Diphenyl Capryl |
| | | | Methicone |
| | 4.00 | Isopropyl Palmitate | Isopropyl Palmitate |
| | 1.00 | Abil ® 350 | Dimethicone |
| B | 3.00 | Uvinul ® N 539 T | Octocrylene |
| | 1.00 | Mexoryl ® XL | Drometrizole Trisiloxane |
| | 1.00 | Tinosorb ® S | Bis-Ethylhexyloxyphenol |
| | | | Methoxyphenyl Triazine |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 1.00 | Keratin-binding domain-reactive dye | |
| | | 14_264 | |
| | 61.30 | Water demin. | Aqua dem. |
| D | 1.00 | Vitamin E Acetate | Tocopheryl Acetate |
| | q.s. | Preservative | |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 6.00 | T-Lite ® SF-S | Titanium Dioxide, Silica |
| | | | Hydrate, Alumina Hydrate, |
| | | | Methicone/Dimethicone |
| | | | Copolymer |
| | 4.00 | Isopropyl Palmitate | Isopropyl Palmitate |
| | 1.00 | Abil ® 350 | Dimethicone |
| B | 3.00 | Uvinul ® N 539 T | Octocrylene |
| | 1.00 | Mexoryl ® XL | Drometrizole trisiloxane |
| | 1.00 | Tinosorb ® S | Bis-Ethylhexyloxyphenol |
| | | | Methoxyphenyl Triazine |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 61.80 | Water demin. | Aqua dem. |
| D | 1.00 | Vitamin E Acetate | Tocopheryl Acetate |
| | 0.5% | Keratin-binding domain-reactive | |
| | | dye 14_264 | |
| | q.s. | Preservative | |
| A | 4.50 | Uvinul ® MC 80 | Ethylhexyl Methoxycinnamate |
| | 3.00 | Uvinul ® N 539 T | Octocrylene |
| | 1.00 | Uvinul ® T 150 | Ethylhexyl Triazone |
| | 2.50 | Cosmacol ® EMI | Di-C12-13 Alkyl Malate |
| B | 0.50 | Vitamin E Acetate | Tocopheryl Acetate |
| | 4.00 | Tego Care ® 450 | Polyglyceryl-3 Methyl Glucose |
| | | | Distearate |
| C | 3.50 | Cetiol ® SN | Cetearyl Isononanoate |
| | 1.00 | Ganex ® V-220 | VP/Eicosene Copolymer |
| | 5.00 | Isohexadecane | Isohexadecane |
| | 2.50 | Cosmacol ® EMI | Di-C12-13 Alkyl Malate |
| | 5.00 | Z-COTE ® MAX | Zinc Oxide, Diphenyl Capryl |
| | | | Methicone |
| D | 44.00 | Water demin. | Aqua dem. |
| | 5.00 | Glycerin 87% | Glycerin |
| | 1.00 | Lanette E | Sodium Cetearyl Sulfate |
| | 15.00 | Luviquat ® Care | Polyquaternium-44 |
| | 1.00 | Keratin-binding domain-reactive dye | |
| | | 14_264 | |
| | 0.50 | Keltrol ® | Xanthan Gum |
| E | 1.00 | Phenonip | Phenoxyethanol, |
| | | | Methylparaben, Ethylparaben, |
| | | | Butylparaben, Propylparaben, |
| | | | Isobutylparaben |
| A | 4.50 | Uvinul ® MC 80 | Ethylhexyl Methoxycinnamate |
| | 3.00 | Uvinul ® N 539 T | Octocrylene |
| | 1.00 | Tinosorb ® S | Bis-Ethoxylhexyloxyphenol |
| | | | Methoxyphenyl Triazine |
| | 2.50 | Cosmacol ® EMI | Di-C12-13 Alkyl Malate |
| B | 0.50 | Vitamin E Acetate | Tocopheryl Acetate |
| | 4.00 | Tego Care ® 450 | Polyglyceryl-3 Methyl Glucose |
| | | | Distearate |
| C | 3.50 | Cetiol ® SN | Cetearyl Isononanoate |
| | 1.00 | Ganex ® V-220 | VP/Eicosene Copolymer |
| | 5.00 | Isohexadecane | Isohexadecane |
| | 2.50 | Cosmacol ® EMI | Di-C12-13 Alkyl Malate |
| | 5.00 | Z-COTE ® MAX | Zinc Oxide, Diphenyl Capryl |
| | | | Methicone |
| D | 44.90 | Water demin. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 0.10 | Keratin-binding domain-reactive dye | |
| | | 14_264 | |
| | 0.50 | Keltrol ® | Xanthan Gum |
| E | 1.00 | Phenonip | Phenoxyethanol, |
| | | | Methylparaben, Ethylparaben, |
| | | | Butylparaben, Propylparaben, |
| | | | Isobutylparaben |
| A | 1.50 | Mexoryl ® XL | Drometrizole Trisiloxane |
| | 5.00 | Uvinul ® N 539 T | Octocrylene |
| | 1.00 | Tinosorb ® S | Bis-Ethylhexyloxyphenol |
| | | | Methoxyphenyl Triazine |
| | 2.50 | Cosmacol ® EMI | Di-C12-13 Alkyl Malate |
| B | 0.50 | Vitamin E Acetate | Tocopheryl Acetate |
| | 4.00 | Tego Care ® 450 | Polyglyceryl-3 Methyl Glucose |
| | | | Distearate |
| C | 3.50 | Cetiol ® SN | Cetearyl Isononanoate |
| | 1.00 | Ganex ® V-220 | VP/Eicosene Copolymer |
| | 5.00 | Isohexadecane | Isohexadecane |
| | 2.50 | Cosmacol ® EMI | Di-C12-13 Alkyl Malate |
| | 5.00 | Z-COTE ® MAX | Zinc Oxide, Diphenyl Capryl |
| | | | Methicone |
| D | 44.50 | Water demin. | Aqua dem. |
| | 5.00 | Glycerin 87% | Glycerin |
| | 1.00 | Lanette E | Sodium Cetearyl Sulfate |
| | 15.00 | Luviquat ® Care | Polyquaternium-44 |
| | 1.50 | Keratin-binding domain-reactive dye | |
| | | 14_264 | |
| | 0.50 | Keltrol ® | Xanthan Gum |
| E | 1.00 | Phenonip | Phenoxyethanol, |
| | | | Methylparaben, Ethylparaben, |
| | | | Butylparaben, Propylparaben, |
| | | | Isobutylparaben |
| A | 1.50 | Mexoryl ® XL | Drometrizole Trisiloxane |
| | 5.00 | Uvinul ® N 539 T | Octocrylene |
| | 1.00 | Tinosorb ® S | Bis-Ethylhexyloxyphenol |
| | | | Methoxyphenyl Triazine |
| | 2.50 | Cosmacol ® EMI | Di-C12-13 Alkyl Malate |
| B | 0.50 | Vitamin E Acetate | Tocopheryl Acetate |
| | 4.00 | Tego Care ® 450 | Polyglyceryl-3 Methyl Glucose |
| | | | Distearate |
| C | 3.50 | Cetiol ® SN | Cetearyl Isononanoate |
| | 1.00 | Ganex ® V-220 | VP/Eicosene Copolymer |
| | 5.00 | Isohexadecane | Isohexadecane |
| | 2.50 | Cosmacol ® EMI | Di-C12-13 Alkyl Malate |
| | 5.00 | T-Lite ® SF | Titanium Dioxide, Alumina |
| | | | Hydrate, |
| | | | Dimethicone/Methicone |
| | | | Copolymer |
| D | 45.00 | Water demin. | Aqua dem. |
| | 5.00 | Glycerin 87% | Glycerin |
| | 1.00 | Lanette E | Sodium Cetearyl Sulfate |
| | 15.00 | Luviquat ® Care | Polyquaternium-44 |
| | 1.00 | Keratin-binding domain-reactive dye | |
| | | 14_264 | |
| | 0.50 | Keltrol ® | Xanthan Gum |
| E | 1.00 | Phenonip | Phenoxyethanol, |
| | | | Methylparaben, Ethylparaben, |
| | | | Butylparaben, Propylparaben, |
| | | | Isobutylparaben |
| A | 1.00 | Abil ® Care 85 | Bis-PEG/PPG-16/16 PEG/PPG-16/16 |
| | | | Dimethicone, Caprylic/Capric |
| | | | Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 5.00 | Uvinul ® MC 80 | Ethylhexyl Methoxycinnamate |
| | 10.00 | Witconol APM | PPG-3 Myristyl Ether |
| | 2.00 | Uvinul ® T 150 | Ethylhexyl Triazone |
| | 1.00 | Dow Corning ® 345 Fluid | Cyclopentasiloxane, Cyclohexasiloxane |
| | 5.00 | Uvinul ® N 539 T | Octocrylene |
| B | 5.00 | Z-COTE ® MAX | Zinc Oxide, Diphenyl Capryl Methicone |
| C | 5.00 | 1,2-Propylene glycol | Propylene Glycol |
| | 0.20 | Keltrol ® | Xanthan Gum |
| | 0.10 | Edeta BD | Disodium EDTA |
| | 2.0% | Keratin-binding domain-reactive dye | |
| | | 14_264 | |
| | 1.50 | Simulgel ® 600 | Acrylamide/Sodium |
| | | | Acryloyldimethyltaurate Copolymer, |
| | | | Isohexadecane, Polysorbate 80 |
| | 58.40 | Water demin. | Aqua dem. |
| D | q.s. | Perfume oil | |
| | 0.50 | Glydant | DMDM Hydantoin |
| A | 2.00 | Abil ® Care 85 | Bis-PEG/PPG-16/16 PEG/PPG-16/16 |
| | | | Dimethicone, Caprylic/Capric |
| | | | Triglyceride |
| | 4.00 | Finsolv ® TN | C12-15 Alkyl Benzoate |
| | 1.50 | Miglyol ® 812 | Caprylic/Capric Triglyceride |
| | 0.50 | Vitamin E Acetate | Tocopheryl Acetate |
| | 7.50 | Uvinul ® MC 80 | Ethylhexyl Methoxycinnamate |
| | 4.00 | Cetiol ® B | Dibutyl Adipate |
| | 3.00 | Luvitol ® EHO | Cetearyl Ethylhexanoate |
| | 1.00 | Cremophor ® CO 40 | PEG-40 Hydrogenated Castor Oil |
| | 1.00 | Paraffin oil, low viscosity | Mineral Oil |
| | 3.00 | Plantacare ® 2000 | Decyl Glucoside |
| | 0.50 | Phenonip | Phenoxyethanol, Methylparaben, |
| | | | Ethylparaben, Butylparaben, |
| | | | Propylparaben, Isobutylparaben |
| | q.s. | Perfume oil | |
| B | 4.00 | Z-COTE ® MAX | Zinc Oxide, Diphenyl Capryl |
| | | | Methicone |
| C | 2.00 | Simulgel ® NS | Hydroxyethyl Acrylate/Sodium |
| | | | Acryloyldimethyl Taurate Copolymer, |
| | | | Squalane, Polysorbate 60 |
| | 0.20 | Keltrol ® | Xanthan Gum |
| | 0.10 | Edeta BD | Disodium EDTA |
| | 1.00 | Keratin-binding domain-reactive | |
| | | dye 14_264 | |
| | 64.20 | Water demin. | Aqua dem. |
| A | 6.00 | Gilugel ® SIL 5 | Cyclomethicone, Aluminium/Magnesium |
| | | | Hydroxide Stearate |
| | 5.00 | Uvinul ® MC 80 | Ethylhexyl Methoxycinnamate |
| | 2.00 | Uvinul ® A Plus | Diethylamino Hydroxybenzoyl Hexyl |
| | | | Benzoate |
| | 1.00 | Uvinul ® T 150 | Ethylhexyl Triazone |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, Hexyl |
| | | | Laurate |
| | 2.00 | Cosmacol ® EMI | Di-C12-13 Alkyl Malate |
| | 3.00 | Isopropyl palmitate | Isopropyl Palmitate |
| | 5.00 | Abil ® B 8839 | Cyclopentasiloxane, Cyclohexasiloxane |
| | 0.50 | Abil ® 350 | Dimethicone |
| B | 0.50 | Sodium chloride | Sodium Chloride |
| | 0.20 | Edeta BD | Disodium EDTA |
| | 0.20 | Keratin-binding domain-reactive | |
| | | dye 14_264 | |
| | 62.10 | Water demin. | Aqua dem. |
| C | 1.00 | Vitamin E Acetate | Tocopheryl Acetate |
| | 0.50 | Phenonip | Phenoxyethanol, Methylparaben, |
| | | | Ethylparaben, Butylparaben, |
| | | | Propylparaben, Isobutylparaben |
| A | 4.50 | Uvinul ® MC 80 | Ethylhexyl Methoxycinnamate |
| | 2.00 | Tinosorb ® S | Bis-Ethylhexyloxyphenol Methoxyphenyl |
| | | | Triazine |
| | 3.00 | Uvinul ® N 539 T | Octocrylene |
| | 2.50 | Cosmacol ® EMI | Di-C12-13 Alkyl Malate |
| | 0.50 | Vitamin E Acetate | Tocopheryl Acetate |
| | 4.00 | Tego Care ® 450 | Polyglyceryl-3 Methyl Glucose Distearate |
| B | 3.50 | Cetiol ® SN | Cetearyl Isononanoate |
| | 1.00 | Ganex ® V-220 | VP/Eicosene Copolymer |
| | 5.00 | Isohexadecane | Isohexadecane |
| | 2.50 | Cosmacol ® EMI | Di-C12-13 Alkyl Malate |
| | 3.00 | T-Lite ® SF | Titanium Dioxide, Alumina Hydrate, |
| | | | Dimethicone/Methicone Copolymer |
| C | 5.00 | Glycerin 87% | Glycerin |
| | 1.00 | Lanette E | Sodium Cetearyl Sulfate |
| | 0.50 | Keltrol ® | Xanthan Gum |
| | 1.00 | Keratin-binding domain- | |
| | | reactive dye 14_264 | |
| | 59.70 | Water demin. | Aqua dem. |
| D | 1.00 | Phenonip | Phenoxyethanol, Methylparaben, |
| | | | Ethylparaben, Butylparaben, |
| | | | Propylparaben, Isobutylparaben |
| | 0.30 | Bisabolol rac. | Bisabolol |
| A | 2.00 | Abil ® Care 85 | Bis-PEG/PPG-16/16 PEG/PPG-16/16 |
| | | | Dimethicone, Caprylic/Capric |
| | | | Triglyceride |
| | 4.00 | Finsolv ® TN | C12-15 Alkyl Benzoate |
| | 1.50 | Miglyol ® 812 | Caprylic/Capric Triglyceride |
| | 0.50 | Vitamin E Acetate | Tocopheryl Acetate |
| | 7.50 | Uvinul ® MC 80 | Ethylhexyl Methoxycinnamate |
| | 4.00 | Cetiol ® B | Dibutyl Adipate |
| | 1.00 | Luvitol ® EHO | Cetearyl Ethylhexanoate |
| | 1.00 | Cremophor ® CO 40 | PEG-40 Hydrogenated Castor Oil |
| | 1.00 | Paraffin oil, low viscosity | Mineral Oil |
| | 3.00 | Plantacare ® 2000 | Decyl Glucoside |
| | 1.00 | Uvinul ® A Plus | Diethylamino Hydroxybenzoyl Hexyl |
| | | | Benzoate |
| | 0.50 | Phenonip | Phenoxyethanol, Methylparaben, |
| | | | Ethylparaben, Butylparaben, |
| | | | Propylparaben, Isobutylparaben |
| | 2.50 | Uvinul ® T 150 | Ethylhexyl Triazone |
| | q.s. | Perfume oil | |
| B | 4.00 | Z-COTE ® MAX | Zinc Oxide, Diphenyl Capryl |
| | | | Methicone |
| C | 2.00 | Simulgel ® NS | Hydroxyethyl Acrylate/Sodium |
| | | | Acryloyldimethyl Taurate Copolymer, |
| | | | Squalane, Polysorbate 60 |
| | 0.10 | Keltrol ® | Xanthan Gum |
| | 0.10 | Edeta BD | Disodium EDTA |
| | 0.50 | Keratin-binding domain-reactive | |
| | | dye 14_264 | |
| | 59.90 | Water demin. | Aqua dem. |
| A | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol ® 331 | Cocoglycerides |
| | 8.00 | Finsolv ® TN | C12-15 Alkyl Benzoate |
| | 8.00 | Cetiol ® B | Dibutyl Adipate |
| B | 6.00 | T-Lite ® SF | Titanium Dioxide, Alumina Hydrate, |
| | | | Dimethicone/Methicone Copolymer |
| | 5.00 | Z-COTE ® MAX | Zinc Oxide, Diphenyl Capryl Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 2.00 | Keratin-binding domain- | |
| | | reactive dye 14_264 | |
| | 0.30 | Keltrol ® | Xanthan Gum |
| | 45.10 | Water demin. | Aqua dem. |
| D | 1.00 | Phenonip | Phenoxyethanol, Methylparaben, |
| | | | Ethylparaben, Butylparaben, |
| | | | Propylparaben, Isobutylparaben |
| A | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol ® 331 | Cocoglycerides |
| | 8.00 | Finsolv ® TN | C12-15 Alkyl Benzoate |
| | 8.00 | Cetiol ® B | Dibutyl Adipate |
| B | 6.00 | Uvinul ® N | Octocrylene |
| | 5.00 | Z-COTE ® MAX | Zinc Oxide, Diphenyl Capryl Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 0.30 | Carbopol ® Ultrez 10 P | Carbomer |
| | 1.00 | Keratin-binding domain- | |
| | | reactive dye 14_264 | |
| | 46.10 | Water demin. | Aqua dem. |
| D | 1.00 | Phenonip | Phenoxyethanol, Methylparaben, |
| | | | Ethylparaben, Butylparaben, |
| | | | Propylparaben, Isobutylparaben |
| A | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 10.00 | Myritol ® 331 | Cocoglycerides |
| | 8.00 | Finsolv ® TN | C12-15 Alkyl Benzoate |
| | 8.00 | Cetiol ® B | Dibutyl Adipate |
| B | 2.00 | Uvinul ® A PlusUvinul ® A Plus | Diethylamino Hydroxybenzoyl Hexyl |
| | | | Benzoate |
| | 5.00 | Z-COTE ® MAX | Zinc Oxide, Diphenyl Capryl Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 5.00 | Keratin-binding domain- | |
| | | reactive dye 14_264 | |
| | 0.30 | Carbopol ® Ultrez 10 P | Carbomer |
| | 48.60 | Water demin. | Aqua dem. |
| D | 1.00 | Phenonip | Phenoxyethanol, Methylparaben, |
| | | | Ethylparaben, Butylparaben, |
| | | | Propylparaben, Isobutylparaben |
| A | 3.50 | Cremophor ® A 6 | Ceteareth-6, Stearyl Alcohol |
| | 1.50 | Cremophor ® A 25 | Ceteareth-25 |
| | 7.50 | Uvinul ® MC 80 | Ethylhexyl Methoxycinnamate |
| | 2.00 | Uvinul ® A Plus | Diethylamino Hydroxybenzoyl Hexyl |
| | | | Benzoate |
| | 2.00 | Dow Corning ® 345 | Cyclopentasiloxane, Cyclohexasiloxane |
| | | | Fluid |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| B | 5.00 | T-Lite ® SF-S | Titanium Dioxide, Silica Hydrate, Alumina |
| | | | Hydrate, Methicone/Dimethicone |
| | | | Copolymer |
| C | 3.00 | Glycerin 87% | Glycerin |
| | 0.20 | Edeta BD | Disodium EDTA |
| | 0.30 | Keltrol ® T | Xanthan Gum |
| | 1.00 | Plantacare ® 2000 | Decyl Glucoside |
| | 2.00 | Keratin-binding domain- | |
| | | reactive dye 14_264 | |
| | 57.30 | Water demin. | Aqua dem. |
| D | 1.00 | Vitamin E Acetate | Tocopheryl Acetate |
| | 0.20 | Bisabolol rac. | Bisabolol |
| A | 10.00 | Uvinul ® A Plus B | Ethylhexyl Methoxycinnamate, |
| | | | Diethylamino Hydroxybenzoyl Hexyl |
| | | | Benzoate |
| | 10.00 | Uvinul ® N 539 T | Octocrylene |
| | 4.00 | Eumulgin ® VL 75 | Lauryl Glucoside, Polyglyceryl-2 |
| | | | Dipolyhydroxystearate, Glycerin |
| | 8.00 | Cetiol ® B | Dibutyl Adipate |
| | 8.00 | Finsolv ® TN | C12-15 Alkyl Benzoate |
| | 12.00 | Myritol ® 331 | Cocoglycerides |
| | 1.00 | Lanette E | Sodium Cetearyl Sulfate |
| | 2.00 | Lanette O | Cetearyl Alcohol |
| B | 3.00 | Z-COTE ® MAX | Zinc Oxide, Diphenyl Capryl Methicone |
| C | 35.08 | Water demin. | Aqua dem. |
| | 0.38 | Citric acid | Citric Acid |
| | 2.90 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 0.10 | Keratin-binding domain- | |
| | | reactive dye 14_264 | |
| | 1.50 | Veegum Ultra | Magnesium Aluminum Silicate |
| D | 0.50 | Phenonip | Phenoxyethanol, Methylparaben, |
| | | | Ethylparaben, Butylparaben, |
| | | | Propylparaben, Isobutylparaben |
| | 1.00 | Vitamin E Acetate | Tocopheryl Acetate |
| A | 0.70 | Cremophor ® A 25 | Ceteareth-25 |
| | 1.70 | Cremophor ® A 6 | Ceteareth-6, Stearyl Alcohol |
| | 2.00 | Uvinul ® A Plus | Diethylamino Hydroxybenzoyl Hexyl |
| | | | Benzoate |
| | 3.00 | Uvinul ® N 539 T | Octocrylene |
| | 3.00 | Z-COTE ® MAX | Zinc Oxide, Diphenyl Capryl Methicone |
| | 2.00 | Abil ® B 8843 | PEG-14 Dimethicone |
| | 3.60 | Lanette O | Cetearyl Alcohol |
| | 4.00 | Uvinul ® MC 80 | Ethylhexyl Methoxycinnamate |
| | 2.00 | Cetiol ® B | Dibutyl Adipate |
| B | 4.00 | Glycerin 87% | Glycerin |
| | 0.20 | Edeta BD | Disodium EDTA |
| | 71.00 | Water demin. | Panthenol |
| C | 4.00 | Luvigel EM | Caprylic/Capric Triglyceride, Sodium |
| | | | Acrylates Copolymer |
| D | 1.00 | Vitamin E Acetate | Tocopheryl Acetate |
| | 1.00 | Keratin-binding domain- | |
| | | reactive dye 14_264 | |
| | 0.20 | Bisabolol rac. | Bisabolol |
| | q.s. | Preservative | |
| A | 7.50 | Uvinul ® MC 80 | Ethylhexyl Methoxycinnamate |
| | 5.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | Z-COTE ® MAX | Zinc Oxide, Diphenyl Capryl |
| | | | Methicone |
| | 0.03 | Sicomet ® blue P 77 007 | C.I. 77 007. Ultramarines |
| C | 4.00 | 1,2-Propylene Glycol Care | Propylene Glycol |
| | 2.00 | D-Panthenol 50 P | Panthenol, Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 0.50 | Keratin-binding domain-reactive | |
| | | dye 14_264 | |
| | 65.27 | Water demin. | Aqua dem. |
| | q.s. | Preservative | |
| | 0.50 | Vitamin E Acetate | Tocopheryl Acetate |
| A | 6.00 | Giluge ® SIL 5 | Cyclomethicone (and) |
| | | | Aluminium/Magnesium Hydroxide |
| | | | Stearate |
| | 5.00 | Uvinul ® N 539 T | Octocrylene |
| | 2.00 | Mexoryl ® XL | Drometrizole Trisiloxane |
| | 1.00 | Uvinul ® T 150 | Ethylhexyl Triazone |
| | 3.00 | T-Lite ® SF-S | Titanium Dioxide, Silica Hydrate, Alumina |
| | | | Hydrate, Methicone/Dimethicone |
| | | | Copolymer |
| | 5.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, Hexyl |
| | | | Laurate |
| | 2.00 | Cosmacol ® EMI | Di-C12-13 Alkyl Malate |
| | 3.00 | Isopropyl palmitate | Isopropyl Palmitate |
| | 5.00 | Abil ® B 8839 | Cyclopentasiloxane, Cyclohexasiloxane |
| | 0.50 | Abil ® 350 | Dimethicone |
| B | 0.50 | Sodium chloride | Sodium Chloride |
| | 0.20 | Edeta BD | Disodium EDTA |
| | 1.00 | Keratin-binding domain-reactive | |
| | | dye 14_264 | |
| | 61.30 | Water demin. | Aqua dem. |
| C | 1.00 | Vitamin E Acetate | Tocopheryl Acetate |
| | 0.50 | Phenonip | Phenoxyethanol, Methylparaben, |
| | | | Ethylparaben, Butylparaben, |
| | | | Propylparaben, Isobutylparaben |
| A | 1.00 | Abil ® Care 85 | Bis-PEG/PPG-16/16 PEG/PPG-16/16 |
| | | | Dimethicone, Caprylic/Capric Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 5.00 | Uvinul ® N 539 T | Octocrylene |
| | 10.00 | Witconol APM | PPG-3 Myristyl Ether |
| | 2.00 | Uvinul ® T 150 | Ethylhexyl Triazone |
| | 1.00 | Dow Corning ® 345 Fluid | Cyclopentasiloxane, Cyclohexasiloxane |
| | 2.00 | Uvinul ® A Plus | Diethylamino Hydroxybenzoyl Hexyl |
| | | | Benzoate |
| B | 5.00 | Z-COTE ® MAX | Zinc Oxide, Diphenyl Capryl Methicone |
| C | 5.00 | 1,2-Propylene glycol | Propylene Glycol |
| | 0.20 | Keltrol ® | Xanthan Gum |
| | 0.10 | Edeta BD | Disodium EDTA |
| | 1.00 | Keratin-binding domain-reactive | |
| | | dye 14_264 | |
| | 1.50 | Simulgel ® 600 | Acrylamide/Sodium |
| | | | Acryloyldimethyltaurate Copolymer, |
| | | | Isohexadecane, Polysorbate 80 |
| | 59.40 | Water demin. | Aqua dem. |
| D | q.s. | Perfume oil | |
| | 0.50 | Glydant | DMDM Hydantoin |
| A | 7.00 | Uvinul ® A Plus B | Ethylhexyl Methoxycinnamate, |
| | | | Diethylamino Hydroxybenzoyl Hexyl |
| | | | Benzoate |
| | 1.00 | Tinosorb ® S | Bis-Ethylhexyloxyphenol Methoxyphenyl |
| | | | Triazine |
| | 1.00 | Uvinul ® T 150 | Ethylhexyl Triazone |
| | 7.00 | Uvinul ® N 539 T | Octocrylene |
| | 4.00 | Eumulgin ® VL 75 | Lauryl Glucoside, Polyglyceryl-2 |
| | | | Dipolyhydroxystearate, Glycerin |
| | 8.00 | Cetiol ® B | Dibutyl Adipate |
| | 8.00 | Finsolv ® TN | C12-15 Alkyl Benzoate |
| | 12.00 | Myritol ® 331 | Cocoglycerides |
| | 1.00 | Lanette E | Sodium Cetearyl Sulfate |
| | 2.00 | Lanette O | Cetearyl Alcohol |
| B | 5.00 | Z-COTE ® MAX | Zinc Oxide, Diphenyl Capryl Methicone |
| C | 35.35 | Water demin. | Aqua dem. |
| | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 0.1% | Keratin-binding domain-reactive | |
| | | dye 14_264 | |
| | 1.50 | Veegum Ultra | Magnesium Aluminum Silicate |
| D | 0.50 | Phenonip | Phenoxyethanol, Methylparaben, |
| | | | Ethylparaben, Butylparaben, |
| | | | Propylparaben, Isobutylparaben |
| | 1.00 | Vitamin E Acetate | Tocopheryl Acetate |
| A | 7.00 | Uvinul ® A Plus B | Ethylhexyl Methoxycinnamate, |
| | | | Diethylamino Hydroxybenzoyl Hexyl |
| | | | Benzoate |
| | 1.00 | Tinosorb ® S | Bis-Ethylhexyloxyphenol Methoxyphenyl |
| | | | Triazine |
| | 1.00 | Uvinul ® T 150 | Ethylhexyl Triazone |
| | 7.00 | Uvinul ® N 539 T | Octocrylene |
| | 4.00 | Eumulgin ® VL 75 | Lauryl Glucoside, Polyglyceryl-2 |
| | | | Dipolyhydroxystearate, Glycerin |
| | 8.00 | Cetiol ® B | Dibutyl Adipate |
| | 8.00 | Finsolv ® TN | C12-15 Alkyl Benzoate |
| | 12.00 | Myritol ® 331 | Cocoglycerides |
| | 1.00 | Lanette E | Sodium Cetearyl Sulfate |
| | 2.00 | Lanette O | Cetearyl Alcohol |
| B | 5.00 | T-Lite ® SF | Titanium Dioxide, Alumina Hydrate, |
| | | | Dimethicone/Methicone Copolymer |
| C | 30.45 | Water demin. | Aqua dem. |
| | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 5.00 | Keratin-binding domain- | |
| | | reactive dye 14_264 | |
| | 1.50 | Veegum Ultra | Magnesium Aluminum Silicate |
| D | 0.50 | Phenonip | Phenoxyethanol, Methylparaben, |
| | | | Ethylparaben, Butylparaben, |
| | | | Propylparaben, Isobutylparaben |
| | 1.00 | Vitamin E Acetate | Tocopheryl Acetate |
| | 5.00 | Z-COTE ® MAX | Zinc Oxide, Diphenyl Capryl |
| | | | Methicone |
| | 2.00 | Uvinul ® A Plus | Diethylamino Hydroxybenzoyl |
| | | | Hexyl Benzoate |
| | 2.00 | Mexoryl ® XL | Drometrizole Trisiloxane |
| | 3.00 | Uvinul ® MC 80 | Ethylhexyl Methoxycinnamate |
| | 0.50 | Abil ® 350 | Dimethicone |
| | 2.75 | Carnico wax LT 20 | Copernica Cerifera (Carnauba) |
| | | | Wax. Paraffin |
| | 3.70 | Candelilla wax LT 281 LJ | Euphorbia Cerifera (Candelilla) |
| | | | Wax |
| | 1.80 | Beeswax 3050 PH | Beeswax |
| | 3.20 | TeCero wax 30445 | Microcrystalline Wax |
| | 3.20 | TeCero wax 1030 K | Microcrystalline Wax |
| | 1.34 | Cutina CP | Cetyl Palmitate |
| | 6.40 | Vaseline | Petrolatum |
| | 7.30 | Softisan 100 | Hydrogenated Cocoglycerides |
| | 10.00 | Luvitol ® EHO | Cetearyl Ethylhexanoate |
| | 0.17 | Bisabolol nat. | Bisabolol |
| | 1.84 | Vitamin E Acetate | Tocopheryl Acetate |
| | 1.00 | Keratin-binding domain-reactive dye | |
| | | 14_264 | |
| | 0.42 | D,L-Alpha-Tocopherol | Tocopherol |
| | 40.38 | Castor Oil | Ricinus Communis (Castor) Oil |
| A | 1.00 | Abil ® Care 85 | Bis-PEG/PPG-16/16 PEG/PPG-16/16 |
| | | | Dimethicone, Caprylic/Capric Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 2.00 | Mexoryl ® XL | Drometrizole Trisiloxane |
| | 10.00 | Witconol ® APM | PPG-3 Myristyl Ether |
| | 1.00 | Uvinul ® T 150 | Ethylhexyl Triazone |
| | 1.00 | Dow Corning ® 345 Fluid | Cyclopentasiloxane, Cyclohexasiloxane |
| | 5.00 | Uvinul ® N 539 T | Octocrylene |
| B | 3.00 | T-Lite ® SF-S | Titanium Dioxide, Silica Hydrate, Alumina |
| | | | Hydrate, Methicone/Dimethicone |
| | | | Copolymer |
| C | 5.00 | 1,2-Propylen glykol | Propylene Glycol |
| | 1.00 | Mexoryl ® SX | Terephthalidene Dicamphor Sulfonic Acid |
| | 0.20 | Keltrol ® | Xanthan Gum |
| | 0.10 | Edeta BD | Disodium EDTA |
| | 1.00 | Keratin-binding domain-reactive | |
| | | dye 14_264 | |
| | 1.50 | Simulgel ® 600 | Acrylamide/Sodium |
| | | | Acryloyldimethyltaurate Copolymer, |
| | | | Isohexadecane, Polysorbate 80 |
| | 69.50 | Water demin. | Aqua dem. |
| D | q.s. | Perfume oil | |
| | 0.50 | Glydant | DMDM Hydantoin |
| A | 1.00 | Abil ® Care 85 | Bis-PEG/PPG-16/16 PEG/PPG-16/16 |
| | | | Dimethicone, Caprylic/Capric Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 2.00 | Parsol ® 1789 | Butyl Methoxydibenzoylmethane |
| | 2.00 | Mexoryl ® XL | Drometrizole Trisiloxane |
| | 10.00 | Witconol ® APM | PPG-3 Myristyl Ether |
| | 1.00 | Uvinul ® T 150 | Ethylhexyl Triazone |
| | 1.00 | Dow Corning ® 345 Fluid | Cyclopentasiloxane, Cyclohexasiloxane |
| | 5.00 | Uvinul ® N 539 T | Octocrylene |
| B | 3.00 | T-Lite ® SF-S | Titanium Dioxide, Silica Hydrate, Alumina |
| | | | Hydrate, Methicone/Dimethicone |
| | | | Copolymer |
| C | 5.00 | 1,2-Propylen glycol | Propylene Glycol |
| | 1.00 | Mexoryl ® SX | Terephthalidene Dicamphor Sulfonic Acid |
| | 0.20 | Keltrol ® | Xanthan Gum |
| | 0.10 | Edeta BD | Disodium EDTA |
| | 0.20 | Keratin-binding domain-reactive | |
| | | dye 14_264 | |
| | 1.50 | Simulgel ® 600 | Acrylamide/Sodium |
| | | | Acryloyldimethyltaurate Copolymer, |
| | | | Isohexadecane, Polysorbate 80 |
| | 68.30 | Water demin. | Aqua dem. |
| D | q.s. | Perfume oil | |
| | 0.50 | Glydant | DMDM Hydantoin |
| A | 0.70 | Cremophor ® A 25 | Ceteareth-25 |
| | 1.70 | Cremophor ® A 6 | Ceteareth-6, Stearyl Alcohol |
| | 2.00 | Parsol ® 1789 | Butyl Methoxydibenzoylmethane |
| | 5.00 | Uvinul ® N 539 T | Octocrylene |
| | 4.00 | Z-COTE ® MAX | Zinc Oxide, Diphenyl Capryl Methicone |
| | 2.00 | Abil ® B 8843 | PEG-14 Dimethicone |
| | 3.60 | Lanette O | Cetearyl Alcohol |
| | 1.00 | Uvinul ® T150 | Ethylhexyl Triazone |
| | 2.00 | Cetiol ® B | Dibutyl Adipate |
| B | 4.00 | Glycerin 87% | Glycerin |
| | 0.20 | Edeta BD | Disodium EDTA |
| | 71.00 | Water demin. | Panthenol |
| C | 4.00 | Luvigel ® EM | Caprylic/Capric Triglyceride, Sodium |
| | | | Acrylates Copolymer |
| | 1.00 | Keratin-binding domain- | |
| | | reactive dye 14_264 | |
| D | 1.00 | Vitamin E Acetate | Tocopheryl Acetate |
| | 0.20 | Bisabolol rac. | Bisabolol |
| | q.s. | Preservative | |
| A | 1.00 | Abil ® Care 85 | Bis-PEG/PPG-16/16 PEG/PPG-16/16 |
| | | | Dimethicone, Caprylic/Capric Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 2.00 | Parsol ® 1789 | Butyl Methoxydibenzoylmethane |
| | 2.00 | Mexoryl ® XL | Drometrizole Trisiloxane |
| | 10.00 | Witconol APM | PPG-3 Myristyl Ether |
| | 1.00 | Uvinul ® T 150 | Ethylhexyl Triazone |
| | 1.00 | Dow Corning ® 345 Fluid | Cyclopentasiloxane, Cyclohexasiloxane |
| | 5.00 | Uvinul ® N 539 T | Octocrylene |
| B | 3.00 | Z-COTE ® MAX | Zinc Oxide, Diphenyl Capryl Methicone |
| C | 5.00 | 1,2-Propylene glycol | Propylene Glycol |
| | 1.00 | Mexoryl ® SX | Terephthalidene Dicamphor Sulfonic Acid |
| | 0.20 | Keltrol ® | Xanthan Gum |
| | 0.10 | Edeta BD | Disodium EDTA |
| | 1.50 | Simulgel ® 600 | Acrylamide/Sodium |
| | | | Acryloyldimethyltaurate Copolymer, |
| | | | Isohexadecane, Polysorbate 80 |
| | 0.50 | Keratin-binding domain-reactive | |
| | | dye 14_264 | |
| | 68.00 | Water demin. | Aqua dem. |
| D | q.s. | Perfume oil | |
| | 0.50 | Glydant | DMDM Hydantoin |
| A | 2.00 | Uvinul ® A Plus | Diethylamino Hydroxybenzoyl Hexyl |
| | | | Benzoate |
| | 3.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.5 | Cremophor ® WO 7 | PEG-7 Hydrogenated Castor Oil |
| | 0.5 | Cremophor ® PS 20 | Polysorbate 20 |
| | 2.00 | Dry Flo Pure | Aluminum Starch Octenylsuccinate |
| B | 5.00 | Z-COTE ® MAX | Zinc Oxide, Diphenyl Capryl Methicone |
| C | 4.00 | 1,2-Propylene Glycol Care | Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 66.30 | Water demin. | Aqua dem. |
| D | q.s. | Preservative | |
| | 0.50 | Vitamin E Acetate | Tocopheryl Acetate |
| | 1.00 | Keratin-binding domain- | |
| | | reactive dye 14_264 | |
| | 1.00 | RetiSTAR ™ | Caprylic/Capric Triglyceride, Sodium |
| | | | Ascorbate, Tocopherol, Retinol |

### Example 65

### Hair Coloring with KBD-B-Dye Couplings

Firstly, a red reactive dye 14_{—}204 was coupled to KDD-D (SEQ ID No.: 168) according to the method described above (Ex. 19). In order to show that the binding of the KBD-D-dye coupling to hair is mediated by the protein and not by the free dye itself, the same dye was treated in each case only with cysteine but without KBD-D. Then, KBD-D-14_{—}264 and 14_{—}264 were each placed onto 5 mg of hair, which was briefly incubated, and nonbound KBD-D-dye or pure dye was washed off using a 15% strength Tween 20 solution. The result clearly shows that the binding to the hair is mediated by the KBD-D and coloring of the hair has taken place.

If, on the other hand, it is desired to decolor the hair again, a wash with a SDS fraction of 15%, or a treatment with a keratin-containing solution is suitable.

Dermocosmetic preparations according to the invention are described below, comprising the keratin-binding effector molecule prepared according to Example 20 (keratin-binding domain according to SEQ ID No.: ID 168 coupled with the dye 14 264). Said keratin-binding effector molecule is referred to in the examples below as keratin-binding domain-reactive dye 14_{—}264. It will be appreciated by the person skilled in the art that all other dyes described in Tables 6 and 7 can be coupled with the KBD according to Example 21 and be used in the preparations given below.

### Example 66

### Shampoo (Base Formulation without Keratin-Binding Domain-Reactive Dye)

| | % | Ingredient (INCI) |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 1.25 | Polyquaternium-16 |
| | 2.00 | Laureth-3 |
| | q.s. | Citric Acid |
| | 57.75 | Aqua dem. |
| B | 3.00 | PEG-150 Distearate |

Preparation: Weigh in the components of phase A and dissolve. Adjust the pH to 6-7. Add phase B and heat to about 50° C. Cool to room temperature with stirring.

The content of keratin-binding domain-reactive dye 14_{—}264 KBD-D (SEQ ID No.: 168) in the examples below refers to 100% keratin-binding domain-reactive dye 14_{—}264. The active ingredient according to the invention can be used either in pure form or as an aqueous solution. In the case of the aqueous solution, the content of water dem. in the respective formulation must be adapted.

### Example 67

### Hair-Coloring Shampoo

### Variant 1

% Ingredient (INCI)

### A 15.00 Cocamidopropyl Betaine

10.00 Disodium Cocoamphodiacetate
5.00 Polysorbate 20
5.00 Decyl Glucoside
q.s. Perfume oil
q.s. Preservative
1.25 Polyquaternium-16
2.00 Laureth-3
q.s. Citric Acid
1.00 Keratin-binding domain-reactive dye 14_{—}264
57.75 Aqua dem.

### B 3.00 PEG-150 Distearate

### Variant 2

% Ingredient (INCI)

### A 15.00 Cocamidopropyl Betaine

10.00 Disodium Cocoamphodiacetate
5.00 Polysorbate 20
5.00 Decyl Glucoside
q.s. Perfume oil
q.s. Preservative
1.25 Polyquaternium-16
2.00 Laureth-3
q.s. Citric Acid
5.00 Keratin-binding domain-reactive dye 14_{—}264
53.75 Aqua dem.

### B 3.00 PEG-150 Distearate

Preparation: Weigh in the components of phase A and dissolve. Adjust the pH to 6-7. Add phase B and heat to about 40° C. Cool rapidly to room temperature with stirring.

### Example 68

### Color Styling Mousse

| | |
|---|---|
| % | Ingredient (INCI) |

Variant 1

| | | |
|---|---|---|
| A | 2.00 | Cocotrimonium Methosulfate |
| | q.s. | Perfume oil |
| B | 6.70 | Acrylates Copolymer |
| | 0.60 | Aminomethyl Propanol |
| | 0.20 | Ceteareth-25 |
| | 0.20 | Panthenol |
| | 0.20 | Hydroxyethylcellulose |
| | 10.00 | Alcohol |
| | 69.97 | Aqua dem. |
| | 1.0 | Keratin-binding domain-reactive dye 14_264 |
| C | 10.00 | Propane/Butane |

Variant 2

| | | |
|---|---|---|
| A | 2.00 | Cocotrimonium Methosulfate |
| | q.s. | Perfume oil |
| B | 6.70 | Acrylates Copolymer |
| | 0.60 | Aminomethyl Propanol |
| | 0.20 | Ceteareth-25 |
| | 0.20 | Panthenol |
| | 0.20 | Hydroxyethylcellulose |
| | 10.00 | Alcohol |
| | 69.97 | Aqua dem. |
| | 0.5 | Keratin-binding domain-reactive dye 14_264 |
| C | 10.00 | Propane/Butane |

Preparation: Mix the components of phase A. Weigh in phase B and dissolve to give a clear solution, then stir into phase A. Bottle with phase C.

### Example 69

### Color Styling Mousse

| | |
|---|---|
| % | Ingredient (INCI) |

Variant 1

| | | |
|---|---|---|
| A | 2.00 | Cocotrimonium Methosulfate |
| | q.s. | Perfume oil |
| B | 7.00 | Polyquaternium-46 |
| | 2.00 | Polyquaternium-11 |
| | 78.87 | Aqua dem. |
| | 2.0 | Keratin-binding domain-reactive dye 14_264 |
| C | 10.00 | Propane/Butane |

Variant 2

| | | |
|---|---|---|
| A | 2.00 | Cocotrimonium Methosulfate |
| | q.s. | Perfume oil |
| B | 7.00 | Polyquaternium-46 |
| | 2.00 | Polyquaternium-11 |
| | 78.87 | Aqua dem. |
| | 1.0 | Keratin-binding domain-reactive dye 14_264 |
| C | 10.00 | Propane/Butane |

Preparation: Mix the components of phase A. Weigh in phase B and dissolve to give a clear solution, then stir into phase A. Bottle with phase C.

### Example 70

Two tresses of blond unbleached hair: European natural hair, color 9/0, natural blond (2 g) are treated with 0.5 g of the hair-coloring shampoo Example 67/variant 1 and the hair-coloring shampoo is left on the hair for 15 min. The hair tresses are then washed with water and cleansed using shampoo Example 66 and dried. The hair is completely red in color. Blond hair can no longer be detected. One tress is retained as comparison.

The second tress is then washed five times with shampoo Example 66 and dried. After the fifth wash, the tress is compared with the retained sample. The tress washed five times is still completely red in color. The two tresses have the same red shade. Visually, no difference in the color depth can be seen.

### Example 71

Two tresses of white hair: selected, white European natural hair (2 g) are treated with 0.5 g of the hair-coloring shampoo Example 67/variant 1 and the hair-coloring shampoo is left on the hair for 15 min. The hair tresses are then washed with water and cleansed using shampoo Example 66 and dried. The hair is completely red in color. White hair can no longer be detected. One tress is retained as comparison.

The second tress is then washed five times with shampoo Example 66 and dried. After the fifth wash, the tress is compared with the retained sample. The tress washed five times is still completely red in color. The two tresses have the same red shade. Visually, no difference in the color depth can be seen.

### Example 72

Two tresses of blond unbleached hair: European natural hair, color 9/0, natural blond (2 g) are treated with 0.5 g of the color styling mousse Example 68/variant 2 and the color styling mousse is left on the hair for 15 min. The hair tresses are then washed with water and cleansed using shampoo Example 66 and dried. The hair is completely red in color. Blond hair can no longer be detected. One tress is retained as comparison.

The second tress is then washed five times with shampoo Example 66 and dried. After the fifth wash, the tress is compared with the retained sample. The tress washed five times is still completely red in color. The two tresses have the same red shade. Visually, no difference in the color depth can be seen.

### Example 73

Two tresses of white hair: selected white European natural hair (2 g) are treated with 0.5 g of the color styling mousse Example 68/variant 2 and the color styling mousse is left on the hair for 15 min. The hair tresses are then washed with water and cleansed with shampoo Example 66 and dried. The hair is completely red in color. White hair can no longer be detected. One tress is retained as comparison.

The second tress is then washed five times with shampoo Example 66 and dried. After the fifth wash, the tress is compared with the retained sample. The tress washed five times is still completely red in color. The two tresses have the same red shade. Visually, no difference in the color depth can be seen.

### Example 74

Two tresses of blond unbleached hair: European natural hair, color 9/0, natural blond (2 g) are treated with 0.5 g of the color styling mousse Example 69/variant 1 and the color styling mousse is left on the hair for 15 min. The hair tresses are then washed with water and cleansed with shampoo Example 66 and dried. The hair is completely red in color. Blond hair can no longer be detected. One tress is retained as comparison.

The second tress is then washed five times with shampoo Example 66 and dried. After the fifth wash, the tress is compared with the retained sample. The tress washed five times is still completely red in color. The two tresses have the same red shade. Visually, no difference in the color depth can be seen.

### Example 75

Two tresses of white hair: selected white European natural hair (2 g) are treated with 0.5 g of the color styling mousse Example 69/variant 1 and the color styling mousse is left on the hair for 15 min. The hair tresses are then washed with water and cleansed with shampoo Example 66 and dried. The hair is completely red in color. White hair can no longer be detected. One tress is retained as comparison.

The second tress is then washed five times with shampoo Example 66 and dried. After the fifth wash, the tress is compared with the retained sample. The tress washed five times is still completely red in color. The two tresses have the same red shade. Visually, no difference in the color depth can be seen.

### Example 76

### Use of the KBD in an Emulsion for Day Care—O/W Type

| | |
|---|---|
| % | Ingredient (INCI) |

Al 1%:

| | | |
|---|---|---|
| A | 1.7 | Ceteareth-6, Stearyl Alcohol |
| | 0.7 | Ceteareth-25 |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 69.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, |
| | | Tocopherol. Retinol |
| | 1.0 | Aqueous solution with about 5% keratin-binding |
| | | domain-reactive dye 14_264 |
| E | q.s. | Sodium Hydroxide |

Al 5%:

| | | |
|---|---|---|
| A | 1.7 | Ceteareth-6, Stearyl Alcohol |
| | 0.7 | Ceteareth-25 |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 65.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, |
| | | Tocopherol, Retinol |
| | 5.0 | Aqueous solution with about 5% keratin-binding |
| | | domain-reactive dye 14_264 |
| E | q.s. | Sodium Hydroxide |

Preparation: Heat phases A and B separately from one another to about 80° C. Stir Phase B into phase A and homogenize. Stir phase C into the combined phases A and B and homogenize again. Cool with stirring to about 40° C., add phase D, adjust the pH to about 6.5 using phase E, homogenize and cool to room temperature with stirring.

Note: The formulation is prepared without protective gas. Bottling must take place into oxygen-impermeable packagings. e.g. aluminum tubes.

### Example 77

### Use of the KBD in a Protective Day Cream—O/W Type

| | |
|---|---|
| % | Ingredient (INCI) |

Al 1%:

| | | |
|---|---|---|
| A | 1.7 | Ceteareth-6, Stearyl Alcohol |
| | 0.7 | Ceteareth-25 |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 70.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 1.0 | Aqueous solution with about 5% keratin-binding |
| | | domain-reactive dye 14_264 |
| E | q.s. | Sodium Hydroxide |

Al 5%:

| | | |
|---|---|---|
| A | 1.7 | Ceteareth-6, Stearyl Alcohol |
| | 0.7 | Ceteareth-25 |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 66.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 5.0 | Aqueous solution with about 5% keratin-binding |
| | | domain-reactive dye 14_264 |
| E | q.s. | Sodium Hydroxide |

Preparation: Heat phases A and B separately from one another to about 80° C. Stir phase B into phase A and homogenize. Incorporate phase C into the combined phases A and B and homogenize. Cool with stirring to about 40° C. Add phase D, adjust the pH to about 6.5 using phase E and homogenize. Cool to room temperature with stirring.

### Example 78

### Use of the KBD in a Face-Cleansing Lotion—O/W Type

| | |
|---|---|
| % | Ingredient (INCI) |

Al 1%:

| | | |
|---|---|---|
| A | 10.0 | Cetearyl Ethylhexanoate |
| | 10.0 | Caprylic/Capric Triglyceride |
| | 1.5 | Cyclopentasiloxane, Cyclohexasiloxane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 1.0 | Aqueous solution with about 5% keratin-binding |
| | | domain-reactive dye 14_264 |
| | 60.7 | Aqua dem. |

Al 5%:

| | | |
|---|---|---|
| A | 10.0 | Cetearyl Ethylhexanoate |
| | 10.0 | Caprylic/Capric Triglyceride |
| | 1.5 | Cyclopentasiloxane, Cyclohexasiloxane |
| | 2.0 | PEG-40 Hydrogenated Castor Oil |
| B | 3.5 | Caprylic/Capric Triglyceride. Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 5.0 | Aqueous solution with about 5% keratin-binding |
| | | domain-reactive dye 14_264 |
| | 56.7 | Aqua dem. |

Preparation: Dissolve phase A. Stir phase B into phase A. Incorporate phase C into the combined phases A and B. Dissolve phase D, stir into the combined phases A, B and C and homogenize. After-stir for 15 min.

### Example 79

### Use of the KBD in a Daily Care Body Spray

| | |
|---|---|
| % | Ingredient (INCI) |

Al 1%:

| | | |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 1.0 | Aqueous solution with about 5% keratin-binding domain- |
| | | reactive dye 14_264 |
| | 59.2 | Alcohol |

Al 5%:

| | | |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 5.0 | Aqueous solution with about 5% keratin-binding domain- |
| | | reactive dye 14_264 |
| | 55.2 | Alcohol |

Preparation: Weigh in the components of phase A and dissolve until clear.

### Example 80

### Use of the KBD in a Skin Care Gel

| | |
|---|---|
| % | Ingredient (INCI) |

Al 1%:

| | | |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 1.0 | Aqueous solution with about 5% keratin-binding domain- |
| | | reactive dye 14_264 |
| | 75.4 | Aqua dem. |
| C | 0.8 | Triethanolamine |

Al 5%:

| | | |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 5.0 | Aqueous solution with about 5% keratin-binding domain- |
| | | reactive dye 14_264 |
| | 71.4 | Aqua dem. |
| C | 0.8 | Triethanolamine |

Preparation: Dissolve phase A until clear. Allow phase B to swell and neutralize with phase C. Stir phase A into the homogenized phase B and homogenize.

### Example 81

### Use of the KBD in an After Shave Lotion

| | |
|---|---|
| % | Ingredient (INCI) |

Al 1%:

| | | |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 1.0 | Aqueous solution with about 5% keratin-binding domain- |
| | | reactive dye 14_264 |
| | 0.1 | Triethanolamine |
| | 63.5 | Aqua dem. |

Al 5%:

| | | |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 5.0 | Aqueous solution with about 5% keratin-binding domain- |
| | | reactive dye 14_264 |
| | 0.1 | Triethanolamine |
| | 59.5 | Aqua dem. |

Preparation: Mix the components of phase A. Dissolve phase B, incorporate into phase A and homogenize.

### Example 82

### Use of the KBD in an Aftersun Lotion

| | |
|---|---|
| % | Ingredient (INCI) |

Al 1%:

| | | |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 1.0 | Aqueous solution with about 5% keratin-binding domain- |
| | | reactive dye 14_264 |
| | 63.2 | Aqua dem. |
| C | 0.2 | Triethanolamine |

Al 5%:

| | | |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 5.0 | Aqueous solution with about 5% keratin-binding domain- |
| | | reactive dye 14_264 |
| | 59.2 | Aqua dem. |
| C | 0.2 | Triethanolamine |

Preparation: Mix the components of phase A. Stir phase B into phase A with homogenization. Neutralize with phase C and homogenize again.

### Example 83

### Use of the KBD in a Sunscreen Lotion

| | |
|---|---|
| % | Ingredient (INCI) |

Al 1%:

| | | |
|---|---|---|
| A | 4.5 | Ethylhexyl Methoxycinnamate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 61.7 | Aqua dem. |
| D | 1.0 | Aqueous solution with about 5% keratin-binding domain- |
| | | reactive dye 14_264 |
| | 1.0 | Phenoxyethanol, Methylparaben, Ethylparaben, |
| | | Butylparaben, Propyl-paraben Isobutylparaben |
| | 0.3 | Bisabolol |

Al 5%:

| | | |
|---|---|---|
| A | 4.5 | Ethylhexyl Methoxycinnamate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 57.7 | Aqua dem. |
| D | 5.0 | Aqueous solution with about 5% keratin-binding domain- |
| | | reactive dye 14_264 |
| | 1.0 | Phenoxyethanol, Methylparaben, Ethylparaben, |
| | | Butylparaben, Propyl-paraben, Isobutylparaben |
| | 0.3 | Bisabolol |

Preparation: Heat the components of phases A and B separately from one another to about 80° C.. Stir phase B into phase A and homogenize. Heat phase C to about 80° C. and stir into the combined phases A and B with homogenization. Cool to about 40° C. with stirring, add phase D and homogenize again.

### Example 84

### Use of the KBD in a Sunscreen Lotion—O/W Type

| | |
|---|---|
| % | Ingredient (INCI) |

Al 1%:

| | | |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, Squalane, Polysorbate 60 |
| | 0.2 | Disodium EDTA |
| | 5.0 | Propylene Glycol |
| | 0.5 | Panthenol |
| | 60.9 | Aqua dem. |
| D | 1.0 | Aqueous solution with about 5% keratin-binding domain- |
| | | reactive dye 14_264 |
| | 0.5 | Phenoxyethanol, Methylparaben, Butylparaben, Ethylparaben, |
| | | Propyl-paraben, Isopropylparaben |
| | 1.0 | Tocopheryl Acetate |
| | 0.2 | Bisabolol |

Al 5%:

| | | |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, Squalane, Polysorbate 60 |
| | 0.2 | Disodium EDTA |
| | 5.0 | Propylene Glycol |
| | 0.5 | Panthenol |
| | 56.9 | Aqua dem. |
| D | 5.0 | Aqueous solution with about 5% keratin-binding domain- |
| | | reactive dye 14_264 |
| | 0.5 | Phenoxyethanol, Methylparaben, Butylparaben, Ethylparaben, |
| | | Propyl-paraben, Isopropylparaben |
| | 1.0 | Tocopheryl Acetate |
| | 0.2 | Bisabolol |

Preparation: Heat phase A to about 80° C., stir in phase B and homogenize for 3 min. Likewise heat phase C to 80° C. and stir into the combined phases A and B with homogenization. Cool to about 40° C.. stir in phase D and homogenize again.

### Example 85

### Use of the KBD in a Sunscreen Lotion—O/W Type

| | |
|---|---|
| % | Ingredient (INCI) |

Al 1%:

| | | |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 56.3 | Aqua dem. |
| D | 1.0 | Aqueous solution with about 5% keratin-binding domain- |
| | | reactive dye 14_264 |
| | 1.0 | Tocopheryl Acetate |
| | 0.2 | Bisabolol |
| | q.s. | Perfume oil |
| | q.s. | Preservative |

Al 5%:

| | | |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 52.3 | Aqua dem. |
| D | 5.0 | Aqueous solution with about 5% keratin-binding domain- |
| | | reactive dye 14_264 |
| | 1.0 | Tocopheryl Acetate |
| | 0.2 | Bisabolol |
| | q.s. | Perfume oil |
| | q.s. | Preservative |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### Example 86

### Use of the KBD in a Foot Balsam

| | |
|---|---|
| % | Ingredient (INCI) |

Al 1%:

| | | |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 69.3 | Aqua dem. |
| | q.s. | Preservative |
| C | 1.0 | Bisabolol |
| | 1.0 | Tocopheryl Acetate |
| D | 1.0 | Aqueous solution with about 5% keratin-binding domain- |
| | | reactive dye 14_264 |
| | 5.0 | Witch Hazel Extract |

Al 5%:

| | | |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 65.3 | Aqua dem. |
| | q.s. | Preservative |
| C | 1.0 | Bisabolol |
| | 1.0 | Tocopheryl Acetate |
| D | 5.0 | Aqueous solution with about 5% keratin-binding domain- |
| | | reactive dye 14_264 |
| | 5.0 | Witch Hazel Extract |

Preparation: Heat the components of phases A and B separately from one another to about 80° C.. Stir phase B into phase A with homogenization. Cool to about 40° C. with stirring, add phases C and D and briefly after-homogenize. Cool to room temperature with stirring.

### Example 87

### Use of the KBD in a W/O Emulsion with Bisabolol

| | |
|---|---|
| % | Ingredient (INCI) |

Al 1%:

| | | |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 55.6 | Aqua dem. |
| C | 1.0 | Aqueous solution with about 5% keratin-binding domain- |
| | | reactive dye 14_264 |
| | 0.5 | Tocopheryl Acetate |
| | 0.6 | Bisabolol |

Al 5%:

| | | |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 51.6 | Aqua dem. |
| C | 5.0 | Aqueous solution with about 5% keratin-binding domain- |
| | | reactive dye 14_264 |
| | 0.5 | Tocopheryl Acetate |
| | 0.6 | Bisabolol |

Preparation: Heat phases A and B separately from one another to about 85° C.. Stir phase B into phase A and homogenize. Cool to about 40° C. with stirring, add phase C and briefly homogenize again. Cool to room temperature with stirring. List of formulations for patent keratin-binding domain - haircare

### Example 88

### Foam Conditioner with Setting Agent

| | |
|---|---|
| % | Ingredient (INCI) |

Al 1%

| | | |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume oil |
| | 10.0 | Alcohol |
| | 1.0 | Aqueous solution with about 5% keratin-binding |
| | | domain-reactive dye 14_264 |
| | 68.1 | Aqua dem. |
| | 10.0 | Propane/Butane |

Al 5%

| | | |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume oil |
| | 10.0 | Alcohol |
| | 5.0 | Aqueous solution with about 5% keratin-binding |
| | | domain-reactive dye 14_264 |
| | 64.1 | Aqua dem. |
| B | 10.0 | Propane/Butane |

Preparation: Weigh the components of phase A together, stir until everything has dissolved and bottle with phase B.

### Example 89

### Hair-Coloring Foam Conditioner

| | |
|---|---|
| % | Ingredient (INCI) |

Al 1%

| | | |
|---|---|---|
| A | 1.0 | Polyquaternium-4 |
| | 0.5 | Hydroxyethyl Cetyldimonium Phosphate |
| | 1.0 | Aqueous solution with about 5% keratin-binding |
| | | domain-reactive dye 14_264 |
| | q.s. | Perfume oil |
| | q.s. | Preservative |
| | 91.5 | Aqua dem. |
| B | 6.0 | Propane/Butane |

Al 5%

| | | |
|---|---|---|
| A | 1.0 | Polyquaternium-4 |
| | 0.5 | Hydroxyethyl Cetyldimonium Phosphate |
| | 5.0 | Aqueous solution with about 5% keratin-binding |
| | | domain-reactive dye 14 264 |
| | q.s. | Perfume oil |
| | q.s. | Preservative |
| | 87.5 | Aqua dem. |
| B | 6.0 | Propane/Butane |

Preparation: Weigh the components of phase A together, stir until everything has dissolved to give a clear solution and bottle with phase B.

### Example 90

### Hair-Coloring Foam Conditioner

| | |
|---|---|
| % | Ingredient (INCI) |

Al 1%

| | | |
|---|---|---|
| A | 1.0 | Polyquaternium-11 |
| | 0.5 | Hydroxyethyl Cetyldimonium Phosphate |
| | 1.0 | Aqueous solution with about 5% keratin-binding |
| | | domain-reactive dye 14_264 |
| | q.s. | Perfume oil |
| | q.s. | Preservative |
| | 91.5 | Aqua dem. |
| B | 6.0 | Propane/Butane |

Al 5%

| | | |
|---|---|---|
| A | 1.0 | Polyquaternium-11 |
| | 0.5 | Hydroxyethyl Cetyldimonium Phosphate |
| | 5.0 | Aqueous solution with about 5% keratin-binding |
| | | domain-reactive dye 14_264 |
| | q.s. | Perfume oil |
| | q.s. | Preservative |
| | 87.5 | Aqua dem. |
| B | 6.0 | Propane/Butane |

Preparation: Weigh the components of phase A together, stir until everything has dissolved to give a clear solution and bottle with phase B.

### Example 91

### Hair-Coloring Styling Foam

| | |
|---|---|
| % | Ingredient (INCI) |

Al 1%

| | | |
|---|---|---|
| A | 0.5 | Laureth-4 |
| | q.s. | Perfume oil |
| B | 77.3 | Aqua dem. |
| | 10.0 | Polyquaternium-28 |
| | 1.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Al 5%

| | | |
|---|---|---|
| A | 0.5 | Laureth-4 |
| | q.s. | Perfume oil |
| B | 73.3 | Aqua dem. |
| | 10.0 | Polyquaternium-28 |
| | 5.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Bottle with phase C.

### Example 92

### Hair-Coloring Styling Foam

| | |
|---|---|
| % | Ingredient (INCI) |

Al 1%

| | | |
|---|---|---|
| A | 2.0 | Cocotrimonium Methosulfate |
| | q.s. | Perfume oil |
| B | 78.5 | Aqua dem. |
| | 6.7 | Acrylates Copolymer |
| | 0.6 | AMP |
| | 1.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Al 5%

| | | |
|---|---|---|
| A | 2.0 | Cocotrimonium Methosulfate |
| | q.s. | Perfume oil |
| B | 74.5 | Aqua dem. |
| | 6.7 | Acrylates Copolymer |
| | 0.6 | AMP |
| | 5.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Bottle with phase C.

### Example 93

### Hair-Coloring Styling Foam

| | |
|---|---|
| % | Ingredient (INCI) |

Al 1%

| | | |
|---|---|---|
| A | 2.0 | Cocotrimonium Methosulfate |
| | q.s. | Perfume oil |
| B | 7.70 | Polyquaternium-44 |
| | 1.0 | Aqueous solution with about 5% keratin-binding |
| | | domain-reactive dye 14_264 |
| | q.s. | Preservative |
| | 79.3 | Aqua dem. |
| C | 10.0 | Propane/Butane |

Al 5%

| | | |
|---|---|---|
| A | 2.0 | Cocotrimonium Methosulfate |
| | q.s. | Perfume oil |
| B | 7.70 | Polyquaternium-44 |
| | 5.0 | Aqueous solution with about 5% keratin-binding |
| | | domain-reactive dye 14_264 |
| | q.s. | Preservative |
| | 75.3 | Aqua dem. |
| C | 10.0 | Propane/Butane |

Preparation: Mix the components of phase A. Dissolve the components of phase B until clear, then stir phase B into phase A. Adjust the pH to 6-7, bottle with phase C.

### Example 94

### Hair-Coloring Styling Foam

| | |
|---|---|
| % | Ingredient (INCI) |

Al 1%

| | | |
|---|---|---|
| A | 2.00 | Cocotrimonium Methosulfate |
| | q.s. | Perfume oil |
| B | 72.32 | Aqua dem. |
| | 2.00 | VP/Acrylates/Lauryl Methacrylate Copolymer |
| | 0.53 | AMP |
| | 1.00 | Aqueous solution with about 5% keratin-binding |
| | | domain-reactive dye 14_264 |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Al 5%

| | | |
|---|---|---|
| A | 2.00 | Cocotrimonium Methosulfate |
| | q.s. | Perfume oil |
| B | 68.32 | Aqua dem. |
| | 2.00 | VP/Acrylates/Lauryl Methacrylate Copolymer |
| | 0.53 | AMP |
| | 5.00 | Aqueous solution with about 5% keratin-binding |
| | | domain-reactive dye 14_264 |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Allow phase C to swell in the mixture of A and B, then adjust the pH to 6-7. Bottle with phase D.

### Example 95

### Hair-Coloring Styling Foam

| | |
|---|---|
| % | Ingredient (INCI) |

Al 1%

| | | |
|---|---|---|
| A | 2.00 | Cetrimonium Chloride |
| | q.s. | Perfume oil |
| B | 67.85 | Aqua dem. |
| | 7.00 | Polyquaternium-46 |
| | 1.00 | Aqueous solution with about 5% keratin-binding |
| | | domain-reactive dye 14_264 |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Al 5%

| | | |
|---|---|---|
| A | 2.00 | Cetrimonium Chloride |
| | q.s. | Perfume oil |
| B | 63.85 | Aqua dem. |
| | 7.00 | Polyquaternium-46 |
| | 5.00 | Aqueous solution with about 5% keratin-binding |
| | | domain-reactive dye 14_264 |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Allow phase C to swell in the mixture of A and B, then adjust the pH to 6-7. Bottle with phase D.

### Example 96

### Hair-Coloring Styling Foam

| | |
|---|---|
| % | Ingredient (INCI) |

Al 1%

| | | |
|---|---|---|
| A | q.s. | PEG-40 Hydrogenated Castor Oil |
| | q.s. | Perfume oil |
| | 85.5 | Aqua dem. |
| B | 7.0 | Sodium Polystyrene Sulfonate |
| | 1.0 | Aqueous solution with about 5% keratin-binding |
| | | domain-reactive dye 14_264 |
| | 0.5 | Cetrimonium Bromide |
| | q.s. | Preservative |
| C | 6.0 | Propane/Butane |

Al 5%

| | | |
|---|---|---|
| A | q.s. | PEG-40 Hydrogenated Castor Oil |
| | q.s. | Perfume oil |
| | 81.5 | Aqua dem. |
| B | 7.0 | Sodium Polystyrene Sulfonate |
| | 5.0 | Aqueous solution with about 5% keratin-binding |
| | | domain-reactive dye 14_264 |
| | 0.5 | Cetrimonium Bromide |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### Example 97

### Hair-Coloring Styling Foam

| | |
|---|---|
| % | Ingredient (INCI) |

Al 1%

| | | |
|---|---|---|
| A | q.s. | PEG-40 Hydrogenated Castor Oil |
| | q.s. | Perfume oil |
| | 92.0 | Aqua dem. |
| B | 0.5 | Polyquaternium-10 |
| | 1.0 | Aqueous solution with about 5% keratin-binding |
| | | domain-reactive dye 14_264 |
| | 0.5 | Cetrimonium Bromide |
| | q.s. | Preservative |
| C | 6.0 | Propane/Butane |

Al 5%

| | | |
|---|---|---|
| A | q.s. | PEG-40 Hydrogenated Castor Oil |
| | q.s. | Perfume oil |
| | 88.0 | Aqua dem. |
| B | 0.5 | Polyquaternium-10 |
| | 5.0 | Aqueous solution with about 5% keratin-binding |
| | | domain-reactive dye 14_264 |
| | 0.5 | Cetrimonium Bromide |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### Example 98

### Hair-Coloring Styling Foam

| | |
|---|---|
| % | Ingredient (INCI) |

Al 1%

| | | |
|---|---|---|
| A | q.s. | PEG-40 Hydrogenated Castor Oil |
| | q.s. | Perfume oil |
| | 82.5 | Aqua dem. |
| B | 10.0 | Polyquaternium-16 |
| | 1.0 | Aqueous solution with about 5% keratin-binding |
| | | domain-reactive dye 14_264 |
| | 0.5 | Hydroxyethyl Cetyldimonium Phosphate |
| | q.s. | Preservative |
| C | 6.0 | Propane/Butane |

Al 5%

| | | |
|---|---|---|
| A | q.s. | PEG-40 Hydrogenated Castor Oil |
| | q.s. | Perfume oil |
| | 78.5 | Aqua dem. |
| B | 10.0 | Polyquaternium-16 |
| | 5.0 | Aqueous solution with about 5% keratin-binding |
| | | domain-reactive dye 14_264 |
| | 0.5 | Hydroxyethyl Cetyldimonium Phosphate |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### Example 99

### Hair-Tinting Styling Foam

| | |
|---|---|
| % | Ingredient (INCI) |

Al 1%

| | | |
|---|---|---|
| A | 2.0 | Cocotrimonium Methosulfate |
| | q.s. | Perfume oil |
| B | 84.0 | Aqua dem. |
| | 2.0 | Chitosan |
| | 1.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| C | 10.0 | HFC 152 A |

Al 5%

| | | |
|---|---|---|
| A | 2.0 | Cocotrimonium Methosulfate |
| | q.s. | Perfume oil |
| B | 80.0 | Aqua dem. |
| | 2.0 | Chitosan |
| | 5.0 | Aqueous solution with about 5% keratin-binding |
| | | domain-reactive dye 14_264 |
| | 0.5 | Dimethicone Copolyol |
| | 0.2 | Ceteareth-25 |
| | 0.2 | Panthenol |
| | 0.1 | PEG-25 PABA |
| C | 10.0 | HFC 152 A |

Preparation: Mix the components of phase A. Add the components of phase B one after the other and dissolve. Bottle with phase C.

### Example 100

### Care Shampoo

| | |
|---|---|
| % | Ingredient (INCI) |

Al 1%

| | | |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, |
| | | Cocamide MEA, Laureth-10 |
| | 1.0 | Aqueous solution with about 5% keratin-binding |
| | | domain-reactive dye 14_264 |
| | 7.7 | Polyquaternium-44 |
| | 2.0 | Amodimethicone |
| | q.s. | Perfume oil |
| | q.s. | Preservative |
| | 1.0 | Sodium Chloride |
| | 43.3 | Aqua dem. |
| B | q.s. | Citric Acid |

Al 5%

| | | |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, |
| | | Cocamide MEA, Laureth-10 |
| | 5.0 | Aqueous solution with about 5% keratin-binding |
| | | domain-reactive dye 14_264 |
| | 7.7 | Polyquaternium-44 |
| | 2.0 | Amodimethicone |
| | q.s. | Perfume oil |
| | q.s. | Preservative |
| | 1.0 | Sodium Chloride |
| | 39.3 | Aqua dem. |
| B | q.s. | Citric Acid |

Preparation: Mix the components of phase A and dissolve. Adjust the pH to 6-7 with citric acid.

### Example 101

### Shower Gel

| | |
|---|---|
| % | Ingredient (INCI) |

Al 1%

| | | |
|---|---|---|
| A | 40.0 | Sodium Laureth Sulfate |
| | 5.0 | Decyl Glucoside |
| | 5.0 | Cocamidopropyl Betaine |
| | 1.0 | Aqueous solution with about 5% keratin-binding |
| | | domain-reactive dye 14_264 |
| | 1.0 | Panthenol |
| | q.s. | Perfume oil |
| | q.s. | Preservative |
| | 2.0 | Sodium Chloride |
| | 46.0 | Aqua dem. |
| B | q.s. | Citric Acid |

Al 5%

| | | |
|---|---|---|
| A | 40.0 | Sodium Laureth Sulfate |
| | 5.0 | Decyl Glucoside |
| | 5.0 | Cocamidopropyl Betaine |
| | 5.0 | Aqueous solution with about 5% keratin-binding |
| | | domain-reactive dye 14_264 |
| | 1.0 | Panthenol |
| | q.s. | Perfume oil |
| | q.s. | Preservative |
| | 2.0 | Sodium Chloride |
| | 42.0 | Aqua dem. |
| B | q.s. | Citric Acid |

Preparation: Mix the components of phase A and dissolve. Adjust the pH to 6-7 with citric acid.

### Example 102

### Shampoo

| | |
|---|---|
| % | Ingredient (INCI) |

Al 1%

| | | |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume oil |
| | 0.1 | Phytantriol |
| | 44.6 | Aqua dem. |
| | 1.0 | Aqueous solution with about 5% keratin-binding |
| | | domain-reactive dye 14_264 |
| | 0.3 | Polyquaternium-10 |
| | 1.0 | Panthenol |
| | q.s. | Preservative |
| | 1.0 | Laureth-3 |
| | 2.0 | Sodium Chloride |

Al 5%

| | | |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume oil |
| | 0.1 | Phytantriol |
| | 40.6 | Aqua dem. |
| | 5.0 | Aqueous solution with about 5% keratin-binding |
| | | domain-reactive dye 14_264 |
| | 0.3 | Polyquaternium-10 |
| | 1.0 | Panthenol |
| | q.s. | Preservative |
| | 1.0 | Laureth-3 |
| | 2.0 | Sodium Chloride |

Preparation: Mix the components of phase A and dissolve. Adjust the pH to 6-7 with citric acid.

### Example 103

### Hair-Coloring Shampoo

| | |
|---|---|
| % | Ingredient (INCI) |

Al 1%

| | | |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 1.00 | Aqueous solution with about 5% keratin-binding |
| | | domain-reactive dye 14_264 |
| | 0.15 | Guar Hydroxypropyltrimonium Chloride |
| | 2.00 | Laureth-3 |
| | 58.00 | Aqua dem. |
| | q.s. | Citric Acid |
| B | 3.00 | PEG-150 Distearate |

Al 5%

| | | |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 5.00 | Aqueous solution with about 5% keratin-binding |
| | | domain-reactive dye 14_264 |
| | 0.15 | Guar Hydroxypropyltrimonium Chloride |
| | 2.00 | Laureth-3 |
| | 54.00 | Aqua dem. |
| | q.s. | Citric Acid |
| B | 3.00 | PEG-150 Distearate |

Preparation: Weigh in the components of phase A and dissolve. Adjust the pH to 6-7. Add phase B and heat to about 40° C. Cool to room temperature with stirring.

### Example 104

### Moisturizing Bodycare Cream

| | |
|---|---|
| % | Ingredient (INCI) |

Al 1%

| | | |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 1.0 | Panthenol |
| | 0.5 | Magnesium Aluminum Silicate |
| | q.s | Preservative |
| | 65.5 | Aqua dem. |
| C | q.s. | Perfume oil |
| | 1.0 | Aqueous solution with about 5% keratin-binding |
| | | domain-reactive dye 14_264 |
| D | q.s. | Citric Acid |

Al 5%

| | | |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 1.0 | Panthenol |
| | 0.5 | Magnesium Aluminum Silicate |
| | q.s | Preservative |
| | 61.5 | Aqua dem. |
| C | q.s. | Perfume oil |
| | 5.0 | Aqueous solution with about 5% keratin-binding |
| | | domain-reactive dye 14_264 |
| D | q.s. | Citric Acid |

Preparation: Heat phases A and B separately to about 80° C. Briefly prehomogenize phase B, then stir phase B into phase A and homogenize again. Cool to about 40° C., add phase C and homogenize thoroughly again. Adjust the pH to 6-7 with citric acid.

### Example 105

### Moisturizing Bodycare Cream

| | |
|---|---|
| % | Ingredient (INCI) |

Al 1%

| | | |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 62.9 | Aqua dem. |
| C | q.s. | Perfume oil |
| | 1.0 | Aqueous solution with about 5% keratin-binding |
| | | domain-reactive dye 14_264 |

Al 5%

| | | |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 58.9 | Aqua dem. |
| C | q.s. | Perfume oil |
| | 5.0 | Aqueous solution with about 5% keratin-binding |
| | | domain-reactive dye 14_264 |

Preparation: Heat phases A and B separately to about 80° C. Stir phase B into phase A and homogenize. Cool to about 40° C. with stirring, add phase C and homogenize again. Allow to cool to room temperature with stirring.

### Example 106

### Liquid Make-Up—O/W Type

| | |
|---|---|
| % | Ingredient (INCI) |

Al 1%

| | | |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 61.9 | Aqua dem. |
| C | 0.1 | Bisabolol |
| | 1.0 | Aqueous solution with about 5% keratin-binding |
| | | domain-reactive dye 14_264 |
| | q.s. | Perfume oil |
| D | 5.7 | C.I. 77 891, Titanium Dioxide |
| | 1.1 | Iron Oxides |

Al 5%

| | | |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 57.9 | Aqua dem. |
| C | 0.1 | Bisabolol |
| | 5.0 | Aqueous solution with about 5% keratin-binding |
| | | domain-reactive dye 14_264 |
| | q.s. | Perfume oil |
| D | 5.7 | C.I. 77 891, Titanium Dioxide |
| | 1.1 | Iron Oxides |

Preparation: Heat phases A and B separately to about 80° C.. Stir phase B into phase A and homogenize. Cool to about 40° C. with stirring, add phases C and D and thoroughly homogenize again. Allow to cool to room temperature with stirring.

### Example 107

Dermocosmetic preparations according to the invention are described below comprising the keratin-binding effector molecule prepared according to Example 19 (keratin-binding domain according to SEQ ID No.: ID 168 coupled with the dye 14_{—}264). Said keratin-binding effector molecule is referred to in the examples below as keratin-binding domain-reactive dye 14 264. It will be appreciated by the person skilled in the art that all of the other dyes described in Tables 6 and 7 can also be coupled with the KBD according to Example 19, 19 a or 19 b and can be used in the preparations given below.

The specified keratin-binding domain-reactive dye 14_{—}264 KBD-D (SEQ ID No.: 168) is used as an approximately 5% strength by weight aqueous solution. The following data are parts by weight.

Clear Hair-Coloring Shampoo

| Ingredients (INCI) | 1 | 2 | 3 | 4 | 5 |
|---|---|---|---|---|---|
| Sodium Laureth Sulfate | — | 15.00 | 10.50 | 12.50 | — |
| Codamidopropyl Betaine | 7.50 | 7.00 | 5.00 | 5.50 | 10.00 |
| PEG-7 Glyceryl Cocoate | 2.00 | 2.50 | 3.50 | 5.00 | 2.30 |
| Perfume oil | 0.10 | 0.10 | 0.10 | 0.10 | 0.10 |
| Keratin-binding domain- | 1.0 | 5.0 | 0.1 | 0.5 | 10.0 |
| reactive dye 14_264 | | | | | |
| D-Panthenol USP | 1.00 | 1.50 | 1.80 | 1.70 | 1.40 |
| Preservative | 0.10 | 0.10 | 0.10 | 0.10 | 0.10 |
| Citric Acid | 0.10 | 0.10 | 0.10 | 0.10 | 0.10 |
| Luviquat ® Ultra Care | 1.50 | 1.00 | 1.50 | 1.20 | 1.10 |
| Sodium Chloride | 1.50 | 1.40 | 1.40 | 1.30 | 1.50 |
| Aqua dem. | ad 100 | ad 100 | ad 100 | ad 100 | ad 100 |

Hair-Coloring Shampoo

| Ingredients (INCI) | 1 | 2 | 3 | 4 | 5 |
|---|---|---|---|---|---|
| Sodium Laureth Sulfate | 35.00 | — | 5.00 | 1.00 | — |
| Decyl Glucoside | 5.00 | 5.50 | 4.90 | 3.50 | 7.00 |
| Cocamidopropyl Betaine | 10.00 | 5.00 | 12.50 | 7.50 | 15.00 |
| Perfume oil | 0.10 | 0.10 | 0.10 | 0.10 | 0.10 |
| Keratin-binding domain- | 1.0 | 5.0 | 0.1 | 20.0 | 5.0 |
| reactive dye 14_264 | | | | | |
| D-Panthenol USP | 0.50 | 1.00 | 0.80 | 1.50 | 0.50 |
| Preservative | 0.10 | 0.10 | 0.10 | 0.10 | 0.10 |
| Citric Acid | 0.10 | 0.10 | 0.10 | 0.10 | 0.10 |
| Laureth-3 | 0.50 | 2.00 | 0.50 | 0.50 | 2.00 |
| Sodium Chloride | 1.50 | 1.50 | 1.50 | 1.50 | 1.50 |
| Aqua dem. | ad 100 | ad 100 | ad 100 | ad 100 | ad 100 |

Clear Conditioner Shampoo for Hair Coloring

| Ingredients (INCI) | 1 | 2 | 3 | 4 | 5 |
|---|---|---|---|---|---|
| ® Disodium | 10.00 | 15.00 | 20.00 | 12.00 | 17.00 |
| Cocoamphodiacetate | | | | | |
| ® Decyl Glucoside | 5.00 | 6.00 | 7.00 | 8.00 | 4.00 |
| ® Cocamidopropyl | 15.00 | 12.00 | 10.00 | 18.00 | 20.00 |
| Betaine | | | | | |
| Luviquat ® FC 550 | 0.30 | 0.20 | 0.20 | 0.20 | 0.30 |
| Perfume oil | 0.10 | 0.10 | 0.10 | 0.10 | 0.10 |
| Keratin-binding domain- | 20.0 | 5.0 | 1.0 | 0.5 | 10.0 |
| reactive dye 14_264 | | | | | |
| Cremophor ® PS 20 | 5.00 | 1.00 | 1.00 | 7.00 | 5.00 |
| Preservative | 0.10 | 0.10 | 0.10 | 0.10 | 0.10 |
| Laureth-3 | — | 1.00 | 0.50 | 2.00 | — |
| Citric Acid | 0.20 | 0.20 | 0.20 | 0.20 | 0.20 |
| PEG-12 Distearate | 3.00 | 2.00 | 2.00 | 3.00 | 2.50 |
| Aqua dem. | ad 100 | ad 100 | ad 100 | ad 100 | ad 100 |

Hair-Tinting Foam O/W Emulsions

| | Emulsion 1 | % by | Emulsion 2 | % by |
|---|---|---|---|---|
| | % by wt. | vol. | % by wt. | vol. |
| Stearic Acid | 5.00 | | 1.00 | |
| Cetyl Alcohol | 5.50 | | | |
| Cetearyl Alcohol | | | 2.00 | |
| PEG-40 Stearate | 8.50 | | | |
| PEG-20 Stearate | | | 1.00 | |
| Caprylic/Capric Triglyceride | 4.00 | | 2.00 | |
| C12-15 Alkyl Benzoate | 10.00 | | 15.00 | |
| Cyclomethicone | 4.00 | | | |
| Dimethicone | | | 0.50 | |
| Keratin-binding domain- | 5.0 | | 10.0 | |
| reactive dye 14_264 | | | | |
| Ethylhexyl Isostearate | | | 5.00 | |
| Myristyl Myristate | | | 2.00 | |
| Ceresin | 1.50 | | | |
| Glycerin | | | 3.00 | |
| Hydroxypropyl Starch | 1.00 | | 3.50 | |
| Phosphate | | | | |
| BHT | | | 0.02 | |
| Disodium EDTA | 0.50 | | 0.10 | |
| Perfume oil, Preservative | q.s. | | q.s. | |
| Potassium Hydroxide | q.s. | | q.s. | |
| Aqua dem. | ad 100 | | ad 100 | |
| | adjust pH | | adjust pH | |
| | to 6.5-7.5 | | to 5.0-6.0 | |
| Emulsion 1 | | 70 | | |
| Emulsion 2 | | | | 35 |
| Nitrogen | | 30 | | |
| Propane/Butane | | | | 65 |

Hair-Coloring Conditioner Shampoo with Pearlescence

| | 1 | 2 | 3 |
|---|---|---|---|
| Polyquaternium-10 | 0.50 | 0.50 | 0.40 |
| Sodium Laureth Sulfate | 9.00 | 8.50 | 8.90 |
| Cocamidopropyl Betaine | 2.50 | 2.60 | 3.00 |
| Uvinul ® MS 40 | 1.50 | 0.50 | 1.00 |
| Keratin-binding domain-reactive | 1.0 | 5.0 | 0.5 |
| dye 14_264 | | | |
| Pearlescent solution | 2.00 | 2.50 | |
| Disodium EDTA | 0.10 | 0.15 | 0.05 |
| Preservative, Perfume oil, Thickener | q.s. | q.s. | q.s. |
| Aqua dem. | ad 100 | ad 100 | ad 100 |

adjust pH to 6.0

Clear Hair-Coloring Conditioner Shampoo

| | 1 | 2 | 3 |
|---|---|---|---|
| Polyquaternium-10 | 0.50 | 0.50 | 0.50 |
| Sodium Laureth Sulfate | 0.2 | — | 0.1 |
| Keratin-binding domain-reactive | 5.0 | 10.0 | 20.0 |
| dye 14_264 | | | |
| Uvinul ® M 40 | 1.00 | 1.50 | 0.50 |
| Preservative, Perfume oil, Thickener | q.s. | q.s. | q.s. |
| Aqua dem. | ad 100 | ad 100 | ad 100 |

adjust pH to 6.0

Clear Conditioner Shampoo with Volume Effect for Hair Coloring

| | 1 | 2 | 3 |
|---|---|---|---|
| Sodium Laureth Sulfate | — | 0.5 | 1.00 |
| Uvinul ® MC 80 | 2.00 | 1.50 | 2.30 |
| Keratin-binding domain-reactive | 10.0 | 15.0 | 5.0 |
| dye 14_264 | | | |
| Cocamidopropyl Betaine | 2.50 | 2.60 | 2.20 |
| Disodium EDTA | 0.01 | 0.10 | 0.01 |
| Preservative, Perfume oil, Thickener | q.s. | q.s. | q.s. |
| Aqua dem. | ad 100 | ad 100 | ad 100 |

adjust pH to 6.0

Hair-Tinting Gel Cream

| | 1 | 2 | 3 | 4 |
|---|---|---|---|---|
| Acrylates/C10-30 Alkylacrylate Crosspolymer | 0.40 | 0.35 | 0.40 | 0.35 |
| Carbomer | 0.20 | 0.22 | 0.20 | 0.22 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Uvinul ® A Plus ™ | 2.00 | 1.50 | 0.75 | 1.00 |
| UvaSorb ® k2A | | 3.00 | | |
| Ethylhexyl Bis-Isopentylbenzoxazolylphenyl | | | | |
| Melamine | | | | |
| Uinul ® MC 80 | 3.00 | | 1.00 | |
| Bis-Ethylhexyloxyphenol Methoxyphenyl Triazine | | 1.50 | | 2.00 |
| Butyl Methoxydibenzoylmethane | | | 2.00 | |
| Disodium Phenyl Dibenzimidazole Tetrasulfonate | 2.50 | | 0.50 | 2.00 |
| Uvinul ® T 150 | 4.00 | | 3.00 | 4.00 |
| Uvinul ® N 539 T | | 4.00 | | |
| Diethylhexyl Butamido Triazone | 1.00 | | | 2.00 |
| Phenylbenzimidazole Sulfonic Acid | 0.50 | | 3.00 | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor Sulfonic Acid | | 1.50 | | 1.00 |
| Diethylhexyl 2,6-Naphthalate | 3.50 | 4.00 | 7.00 | 9.00 |
| Titanium Dioxide-microfine | 1.00 | | 3.00 | |
| Zinc Oxide-microfine | | | | 0.25 |
| Keratin-binding domain-reactive dye 14_264 | 5.0 | 20.0 | 1.0 | 10.0 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume oil | 0.20 | | 0.20 | |
| Aqua dem. | ad 100 | ad 100 | ad 100 | ad 100 |

adjust pH to 6.0

OW Sunscreen Formulation

| | 1 | 2 | 3 | 4 | 5 | 6 | 7 |
|---|---|---|---|---|---|---|---|
| Glyceryl Stearate SF | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Sodium Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Keratin-binding domain-reactive | 10.0 | 0.5 | 3.0 | 5.0 | 0.1 | 15.0 | 7.5 |
| dye 14_264 | | | | | | | |
| UvaSorb ® k2A | | | | | | | |
| Ethylhexyl Bis- | | | | | | | |
| Isopentylbenzoxazolylphenyl | | | | | | | |
| Melamine | | | | | | | |
| Ethylhexyl Methoxycinnamate | | | | | 5.00 | 6.00 | 8.00 |
| Uvinul ® MC 80 | | | | | | | |
| Bis-Ethylhexyloxyphenol | | 1.50 | | 2.00 | 2.50 | | 2.50 |
| Methoxyphenyl Triazine | | | | | | | |
| Butyl Methoxydibenzoylmethane | | | 2.00 | | 2.00 | 1.50 | |
| Disodium Phenyl Dibenzimidazole | 2.50 | | 0.50 | 2.00 | | 0.30 | |
| Tetrasulfonate | | | | | | | |
| Ethylhexyl Triazone Uvinul ® T 150 | 4.00 | | 3.00 | 4.00 | | 2.00 | |
| Octocrylene | | 4.00 | | | | | 7.50 |
| Uvinul ® N 539 T | | | | | | | |
| Diethylhexyl Butamido Triazone | 1.00 | | | 2.00 | 1.00 | | 1.00 |
| Phenylbenzimidazole Sulfonic Acid | 0.50 | | 3.00 | | | | |
| Methylene Bis-Benzotriazolyl | 2.00 | | 0.50 | 1.50 | 2.50 | | |
| Tetramethylbutylphenol | | | | | | | |
| Ethylhexyl Salicylate | | | 3.00 | | | | 5.00 |
| Drometrizole Trisiloxane | | | 0.50 | | | 1.00 | |
| Terephthalidene Dicamphor | | 1.50 | | 1.00 | 1.00 | | 0.50 |
| Sulfonic Acid | | | | | | | |
| Diethylhexyl 2,6-Naphthalate | 3.50 | | 7.00 | | 6.00 | 9.00 | |
| Titanium Dioxide-microfine | 1.00 | | 3.00 | | 3.50 | | 1.50 |
| Zinc Oxide-microfine | | | | 0.25 | | 2.00 | |
| C12-15 Alkyl Beozoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglyceride | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccharide Gum-1 | | 3.00 | 10.00 | | | | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium iminodisuccinate | | | | 0.25 | 1.00 | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad100 | ad 100 |

Hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Sodium Carbomer | | 0.20 | | 0.30 | |
| Acrylates/C10-30 Alkyl Acrylate | 0.50 | | 0.40 | 0.10 | 0.50 |
| Crosspolymer | | | | | |
| Xanthan Gum | | 0.30 | 0.15 | | |
| Keratin-binding domain-reactive dye | 5.0 | 0.5 | 3.0 | 0.1 | 10.0 |
| 14_264 | | | | | |
| UvaSorb ® k2A Ethylhexyl Bis- | | 3.50 | | | |
| Isopentylbenzoxazolylphenyl Melamine | | | | | |
| Ethylhexyl Methoxycinnamate Uvinul ® MC 80 | | | | | 5.00 |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethyhexyl Triazone Uvinul ® T 150 | 4.00 | | 3.00 | 4.00 | |
| Octocrylene | | 4.00 | | | |
| Uvinul ® N 539 T | | | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic Acid | | 1.50 | | 1.00 | 1.00 |
| Diethylhexyl 2,6-Naphthalate | | | 7.00 | | 9.00 |
| Titanium Dioxide-microfine | 1.00 | | 3.00 | | 3.50 |
| Zinc Oxide-microfine | | | | 0.25 | |
| C12-15 Alkyl Benzoate | 2.00 | 2.50 | | | |
| Dicaprylyl Ether | | 4.00 | | | |
| Butylene Glycol Dicaprylate/Dicaprate | 4.00 | | 2.00 | 6.00 | |
| Dicaprylyl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| Biosaccharide Gum-1 | | 2.50 | 0.50 | | 2.00 |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume oil | 0.20 | | 0.05 | 0.40 | |
| Aqua dem. | ad 100 | ad 100 | ad 100 | ad 100 | ad 100 |

WO Sunscreen Emulsion

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Keratin-binding domain-reactive dye 14_264 | 5.0 | 1.0 | 10.0 | 0.5 | 15.0 |
| UvaSorb ® k2A | | 2.00 | | | |
| Ethylhexyl Bis-Isopentylbenzoxazolylphenyl | | | | | |
| Melamine | | | | | |
| Ethylhexyl Methoxycinnamate Uvinul ® MC 80 | | | | | 5.00 |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethylhexyl Triazone Uvinul ® T 150 | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic Acid | | 1.50 | | 1.00 | 1.00 |
| Diethylhexyl 2,6-Naphthalate | | | 7.00 | | 4.00 |
| Titanium Dioxide-microfine | 1.00 | | 3.00 | | 3.50 |
| Zinc Oxide-microfine | | | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soja (Soybean) Oil | | 1.00 | 1.50 | | 1.00 |
| Magnesium Sulfate MgSO₄ | 1.00 | 0.50 | | 0.50 | |
| Magnesium Chloride MgCl₂ | | | 1.00 | | 0.70 |
| Vitamin E Acetate | 0.50 | | 0.25 | | 1.00 |
| Ascorbyl Palmitate | 0.50 | | | 2.00 | |
| Biosaccharide Gum-1 | | | | 3.50 | 1.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume oil | 0.20 | | 0.40 | 0.35 | |
| Aqua dem. | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol | | | | 12.00 |
| Dicaprylate/Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) | 1.50 | 2.00 | 2.00 | 1.50 |
| Wax | | | | |
| Cera Alba | 0.50 | 0.50 | 0.50 | 0.40 |
| C16-40 Alkyl Stearate | | 1.50 | 1.50 | 1.50 |
| Keratin-binding domain-reactive | 0.5 | 3.0 | 1.0 | 5.0 |
| dye 14_264 | | | | |
| UvaSorb ® k2A | | 2.00 | | 4.00 |
| Ethylhexyl Bis- | | | | |
| Isopentylbenzoxazolylphenyl | | | | |
| Melamine | | | | |
| Ethylhexyl Methoxycinnamate | | 3.00 | | |
| Uvinul ® MC 80 | | | | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Ethylhexyl Triazone Uvinul ® T | 4.00 | | 3.00 | 4.00 |
| 150 | | | | |
| Octocrylene | | 4.00 | | |
| Diethylhexyl Butamido Triazone | 1.00 | | | 2.00 |
| Phenylbenzimidazole Sulfonic | 0.50 | | 3.00 | |
| Acid | | | | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | | | 7.00 | |
| Titanium Dioxide-microfine | 1.00 | | 3.00 | |
| Zinc Oxide-microfine | | | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

PIT Emulsion

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone | | | | 0.50 | | 1.00 | | |
| Copolyol | | | | | | | | |
| Polyglyceryl-2 Dioleate | | | | 0.75 | 0.30 | | | |
| Keratin-binding domain- | 0.1 | 5.0 | 0.01 | 0.5 | 20.0 | 0.25 | 10.0 | 3.0 |
| reactive dye 14_264 | | | | | | | | |
| UvaSorb ® k2A | | | 4.00 | | | | 1.50 | |
| Ethylhexyl Bis- | | | | | | | | |
| Isopentylbenzoxazolyl- | | | | | | | | |
| phenyl Melamine | | | | | | | | |
| Ethylhexyl | | | | | 5.00 | 6.00 | 8.00 | 5.00 |
| Methoxycinnamate | | | | | | | | |
| Uvinul ® MC 80 | | | | | | | | |
| Bis-Ethylhexyloxyphenol | | 1.50 | | 2.00 | 2.50 | | 2.50 | 2.50 |
| Methoxyphenyl Triazine | | | | | | | | |
| Butyl Methoxydibenzoyl- | | | 2.00 | | 2.00 | 1.50 | | 2.00 |
| methane | | | | | | | | |
| Disodium Phenyl | 2.50 | | 0.50 | 2.00 | | 0.30 | | |
| Dibenzimidazole | | | | | | | | |
| Tetrasulfonate | | | | | | | | |
| Ethylhexyl Triazone | 4.00 | | 3.00 | 4.00 | | 2.00 | | |
| Uvinul ® T 150 | | | | | | | | |
| Octocrylene | | 4.00 | | | | | 7.50 | |
| Diethylhexyl Butamido | 1.00 | | | 2.00 | 1.00 | | 1.00 | 1.00 |
| Triazone | | | | | | | | |
| Phenylbenzimidazole | 0.50 | | 3.00 | | | | | |
| Sulfonic Acid | | | | | | | | |
| Methylene Bis- | 2.00 | | 0.50 | 1.50 | 2.50 | | | 2.50 |
| Benzotriazolyl | | | | | | | | |
| Tetramethylbutylphenol | | | | | | | | |
| Ethylhexyl Salicylate | | | 3.00 | | | | 5.00 | |
| Drometrizole Trisiloxane | | | 0.50 | | | 1.00 | | |
| Terephthalylidene | | 1.50 | | 1.00 | 1.00 | | 0.50 | 1.00 |
| Dicamphor Sulfonic Acid | | | | | | | | |
| Diethylhexyl 2,6- | | | 7.00 | | 10.00 | 7.50 | | 8.00 |
| Naphthalate | | | | | | | | |
| Titanium | 1.00 | | 3.00 | | 3.50 | | 1.50 | 3.50 |
| Dioxide-microfine | | | | | | | | |
| Zinc Oxide-microfine | | | | 0.25 | | 2.00 | | |
| C12-15 Alkyl Benzoate | 3.50 | | | 6.35 | | | | 0.10 |
| Cocoglyceride | | 3.00 | | 3.00 | | | | 1.00 |
| Dicaprylyl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C1-5 Alkyl | | 0.50 | | | 2.00 | | | |
| Galactomannan | | | | | | | | |
| Hydrogenated Coco- | | | | | 3.00 | 4.00 | | |
| glycerides | | | | | | | | |
| Behenoxy Dimethicone | | | | | | 1.50 | 2.00 | |
| VP/Hexadecene | | | | 1.00 | 1.20 | | | |
| Copolymer | | | | | | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii | | 2.00 | | 3.60 | | 2.00 | | |
| (Shea Butter) | | | | | | | | |
| Iodopropyl | 0.12 | | | | 0.20 | | | |
| Butylcarbamate | | | | | | | | |
| Biosaccharide Gum-1 | | | | 0.10 | | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume oil | 0.20 | | 0.20 | | 0.24 | 0.16 | 0.10 | 0.10 |
| Aqua dem. | ad 100 | ad 100 | ad 100 | ad 100 | ad 100 | ad 100 | ad 100 | ad 100 |

Skin- or Hair-Tinting Gel Cream

| | 1 | 2 | 3 | 4 |
|---|---|---|---|---|
| Acrylates/C10-30 Alkylacrylate | 0.40 | 0.35 | 0.40 | 0.35 |
| Crosspolymer | | | | |
| Carbomer | 0.20 | 0.22 | 0.20 | 0.22 |
| Luvigel ® EM | 1.50 | 2.50 | 2.80 | 3.50 |
| Xanthan Gum | 0.10 | 0.13 | 0.10 | 0.13 |
| Cetearyl Alcohol | 3.00 | 2.50 | 3.00 | 2.50 |
| C12-15 Alkyl benzoate | 4.00 | 4.50 | 4.00 | 4.50 |
| Caprylic/Capric Triglyceride | 3.00 | 3.50 | 3.00 | 3.50 |
| Titanium Dioxide-microfine | 1.00 | | 1.50 | |
| Zinc Oxide-microfine | | 2.00 | | 0.25 |
| Keratin-binding domain-reactive | 0.5 | 10.0 | 3.0 | 5.0 |
| dye 14_264 | | | | |
| Dihydroxyacetone | | | 3.00 | 5.00 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume oil | 0.20 | | 0.20 | |
| Aqua dem. | ad 100 | ad 100 | ad 100 | ad 100 |

adjust pH to 6.0

OW Self-Tanning Formulation

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Keratin-binding domain- | 0.1 | 0.5 | 0.025 | 5.0 | 3.0 | 10.0 | 1.0 |
| reactive dye 14_264 | | | | | | | |
| Dihydroxyacetone | | | 3.00 | 5.00 | | 4 | |
| Titanium Dioxide-microfine | 1.00 | | | | 1.50 | | 1.50 |
| Zinc Oxide-microfine | | | | 0.25 | | 2.00 | |
| C12-15 Alkyl Benzoate | | 0.25 | | | 4.00 | 7.00 | |
| Dicaprylyl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccharide Gum-1 | | 3.00 | 10.00 | | | | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad 100 | ad 100 |

OW Make Up

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Keratin binding domain- | 3.0 | 5.0 | 2.0 | 0.5 | 1.0 | 5.0 | 10.0 |
| reactive dye 14_264 | | | | | | | |
| Titanium Dioxide | 10.00 | 12.00 | 9.00 | 8.50 | 11.00 | 9.50 | 10.00 |
| Iron Oxide | 2.00 | — | 3.00 | 5.00 | 0.2 | 6.00 | 4.40 |
| Zinc Oxide | | — | | 2.00 | | 3.00 | |
| C12-15 Alkyl Benzoate | | 0.25 | | | 4.00 | 7.00 | |
| Dicaprylyl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad 100 | ad 100 |

Self-Tanning Hydrodispersion with Tinting Effect

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl Acrylate | 0.50 | | 0.40 | 0.10 | 0.50 |
| Crosspolymer | | | | | |
| Xanthan Gum | | 0.30 | 0.15 | | |
| Keratin-binding domain-reactive dye | 3.0 | 1.0 | 0.5 | 0.1 | 5.0 |
| 14_264 | | | | | |
| Dihydroxyacetone | | | 3.00 | 5.00 | |
| Titanium Dioxide-microfine | 1.00 | | 1.00 | | 1.00 |
| Zinc Oxide-microfine | | 1.90 | | 0.25 | |
| C12-15 Alkyl Benzoate | 2.00 | 2.50 | | | |
| Dicaprylyl Ether | | 4.00 | | | |
| Butylene Glycol Dicaprylate/Dicaprate | 4.00 | | 2.00 | 6.00 | |
| Dicaprylyl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume oil | 0.20 | | 0.05 | 0.40 | |
| Aqua dem. | ad 100 | ad 100 | ad 100 | ad 100 | ad 100 |

After-Sun Hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl Acrylate | 0.50 | 0.30 | 0.40 | 0.10 | 0.50 |
| Crosspolymer | | | | | |
| Xanthan Gum | | 0.30 | 0.15 | | |
| Keratin-binding domain-reactive dye | 0.1 | 5.0 | 0.5 | 3.0 | 10.0 |
| 14_264 | | | | | |
| C12-15 Alkyl Benzoate | 2.00 | 2.50 | | | |
| Dicaprylyl Ether | | 4.00 | | | |
| Butylene Glycol Dicaprylate/Dicaprate | 4.00 | | 2.00 | 6.00 | |
| Dicaprylyl Carbonate | | 2.00 | 6.00 | | |
| Dimethicone | | 0.50 | 1.00 | | |
| Phenyl Trimethicone | 2.00 | | 0.50 | | |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| Trisodium EDTA | | 0.01 | 0.05 | | 0.10 |
| Ethanol | 15.00 | 10.00 | 8.00 | 12.00 | 9.00 |
| Perfume oil | 0.20 | | 0.05 | 0.40 | |
| Aqua dem. | ad 100 | ad 100 | ad 100 | ad 100 | ad 100 |

WO Emulsion

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Keratin-binding domain-reactive dye 14_264 | 7.0 | 12.0 | 0.1 | 0.5 | 1.0 |
| Titanium Dioxide-microfine | 1.00 | | 3.00 | | 3.50 |
| Zinc Oxide-microfine | | 0.90 | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soja (Soybean) Oil | | 1.00 | 1.50 | | 1.00 |
| Magnesium Sulfate | 1.00 | 0.50 | | 0.50 | |
| Magnesium Chloride | | | 1.00 | | 0.70 |
| Vitamin E Acetate | 0.50 | | 0.25 | | 1.00 |
| Ascorbyl Palmitate | 0.50 | | | 2.00 | |
| Biosaccharide Gum-1 | | | | 3.50 | 7.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume oil | 0.20 | | 0.40 | 0.35 | |
| Aqua dem. | ad 100 | ad 100 | ad 100 | ad 100 | ad 100 |

Solids-Stabilized Emulsion (Pickering Emulsions)

| | 1 | 2 | 3 | 4 | 5 |
|---|---|---|---|---|---|
| Mineral Oil | | | 16.00 | 16.00 | |
| Octyldodecanol | 9.00 | 9.00 | 5.00 | | |
| Caprylic/Capric Triglyceride | 9.00 | 9.00 | 6.00 | | |
| C12-15 Alkyl Benzoate | | | | 5.00 | 8.00 |
| Butylene Glycol Dicaprylate/Dicaprate | | | | | 8.00 |
| Dicaprylyl Ether | 9.00 | | | 4.00 | |
| Dicaprylyl Carbonate | | 9.00 | | | |
| Hydroxyoctacosanyl Hydroxystearate | 2.00 | 2.00 | 2.20 | 2.50 | 1.50 |
| Disteardimonium Hectorite | 1.00 | 0.75 | | 0.50 | 0.25 |
| Cera Microcristalline + Paraffinum Liquidum | | 0.35 | | | 5.00 |
| Hydroxypropyl Methylcellulose | | | 0.10 | | 0.05 |
| Dimethicone | | | | | 3.00 |
| Keratin-binding domain-reactive dye 14_264 | 1.0 | 15.0 | 0.1 | 3.0 | 5.0 |
| Titanium Dioxide + Alumina + Simethicone + | | 3.00 | | | |
| Aqua | | | | | |
| Titanium Dioxide + Trimethoxycaprylylsilane | | 2.00 | 4.00 | 2.00 | 4.00 |
| Silica Dimethyl Silylate | 2.50 | | | 6.00 | 2.50 |
| Boron Nitride | | | 1.00 | | |
| Starch/Sodium Metaphosphate Polymer | 2.00 | | | | |
| Tapioca Starch | | 0.50 | | | |
| Sodium Chloride | 5.00 | 7.00 | 8.50 | 3.00 | 4.50 |
| Glycerin | | | | 1.00 | |
| Trisodium EDTA | 1.00 | 1.00 | 1.00 | 1.00 | 1.00 |
| Vitamin E Acetate | 5.00 | 10.00 | 3.00 | 6.00 | 10.00 |
| Ascorbyl Palmitate | 1.00 | 1.00 | | 1.00 | |
| Methylparaben | | 0.60 | | | 0.20 |
| Propylparaben | | | | | 0.20 |
| Phenoxyethanol | | | 0.20 | | |
| Hexamidine Diisethionate | | | 0.40 | 0.50 | 0.40 |
| Diazolidinyl Urea | | | | | 0.08 |
| Ethanol | | | 0.23 | 0.20 | |
| Perfume oil | 5.00 | | 3.00 | 4.00 | |
| Aqua dem. | 0.20 | | 0.30 | 0.10 | |
| | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol | | | | 12.00 |
| Dicaprylate/Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) | 1.50 | 2.00 | 2.00 | 1.50 |
| Wax | | | | |
| Cera Alba | 0.50 | 0.50 | 0.50 | 0.40 |
| C16-40 Alkyl Stearate | | 1.50 | 1.50 | 1.50 |
| Keratin-binding domain-reactive | 10.0 | 1.0 | 3.0 | 0.1 |
| dye 14_264 | | | | |
| Uvinul ® A Plus ™ | 2.00 | 1.50 | 0.75 | 9.00 |
| Titanium Dioxide-microfine | 1.00 | | 3.00 | |
| Zinc Oxide-microfine | | 1.00 | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

Self-Tanning PIT Emulsions

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone Copolyol | | | | 0.50 | | 1.00 | | |
| Polyglyceryl-2 | | | | 0.75 | 0.30 | | | |
| Keratin-binding domain-reactive | 0.1 | 0.5 | 0.01 | 5.0 | 0.5 | 3.0 | 0.025 | 10.0 |
| dye 14_264 | | | | | | | | |
| Dihydroxyacetone | | | 3.00 | 5.00 | | | 4.00 | |
| Titanium Dioxide-microfine | 1.00 | | 1.50 | | 3.50 | | 1.50 | 1.00 |
| Zinc Oxide-microfine | | 1.00 | | 0.25 | | 2.00 | | 1.50 |
| C12-15 Alkyl Benzoate | 3.50 | | | 6.35 | | | | 0.10 |
| Cocoglycerides | | 3.00 | | 3.00 | | | | 1.00 |
| Dicaprylyl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C1-5 Alkyl Galactomannan | | 0.50 | | | 2.00 | | | |
| Hydrogenated Cocoglycerides | | | | | 3.00 | 4.00 | | |
| Behenoxy Dimethicone | | | | | | 1.50 | 2.00 | |
| VP/Hexadecene Copolymer | | | | 1.00 | 1.20 | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii (Shea | | 2.00 | | 3.60 | | 2.00 | | |
| Butter) | | | | | | | | |
| Iodopropyl Butylcarbamate | 0.12 | | | | 0.20 | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume oil | 0.20 | | 0.20 | | 0.24 | 0.16 | 0.10 | 0.10 |
| Aqua dem. | ad 100 | ad 100 | ad 100 | ad 100 | ad 100 | ad 100 | ad 100 | ad 100 |

Oil Gel

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol | | | | 12.00 |
| Dicaprylate/Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Myristyl Myristate | 3.50 | 3.00 | 4.00 | 3.00 |
| Quaternium-18 Bentonite | 5.00 | 5.00 | 6.00 | 6.00 |
| Propylene Carbonate | 15.00 | 20.00 | 18.00 | 19.50 |
| Keratin-binding domain-reactive | 1.0 | 0.5 | 3.0 | 5.0 |
| dye 14_264 | | | | |
| Vitamin E Acetate | 0.50 | 1.00 | | |
| Ascorbyl Palmitate | 0.05 | | 0.05 | |
| Buxus Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

### Example 108

In the following formulations, cosmetic sunscreen preparations comprising a combination of at least one inorganic pigment, preferably zinc oxide and/or titanium oxide, Uvinul A Plus and further organic UV-A and UV-B tilters are described.

The inorganic pigments here may be present in coated form, i.e. that they are treated superficially. This surface treatment can, for example, consist in providing the pigments with a thin hydrophobic layer by a method known per se, as described in DE-A-33 14 742.

The formulations given below are prepared in a customary way known to the person skilled in the art.

Dermocosmetic preparations according to the invention are described below comprising the keratin-binding effector molecule prepared according to Example 19 (keratin-binding domain according to SEQ ID No.: ID 168 coupled with the dye 14_{—}264). Said keratin-binding effector molecule is referred to in the examples below as keratin-binding domain-reactive dye 14_{—}264. It goes without saying for the person skilled in the art that all of the other dyes described in Tables 6 and 7 can also be coupled with the KBD according to Example 19, 19 a or 19 b and can be used in the preparations given below.

The content of keratin-binding domain-reactive dye 14_{—}264 KBD-D (SEQ ID No.: 168) refers to 100% of active ingredient. The active ingredient according to the invention can be used either in pure form or as an aqueous solution. In the case of aqueous solution, the content of water dem. in the particular formulation must be adjusted.

| | | | |
|---|---|---|---|
| A | 7.50 | Uvinul ® MC 80 | Ethylhexyl Methoxycinnamate |
| | 2.00 | Uvinul ® M 40 | Benzophenone-3 |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | T-Lite ® SF | Titanium Dioxide, Alumina Hydrate, |
| | | | Dimethicone/Methicone Copolymer |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 1.00 | Sepigel 305 | Polyacrylamide, C13-14 Isoparaffin, Laureth-7 |
| | 1.00 | Keratin-binding domain- | |
| | | reactive dye 14_264 | |
| | q.s. | Preservative | |
| A | 7.50 | Uvinul ® MC 80 | Ethylhexyl Methoxycinnamate |
| | 2.00 | Uvinul ® M 40 | Benzophenone-3 |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 7.50 | Uvinul ® MC 80 | Ethylhexyl Methoxycinnamate |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 4.00 | Uvinul ® MC 80 | Ethylhexyl Methoxycinnamate |
| | 2.00 | Uvinul ® T 150 | Ethylhexyl Triazone |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 4.00 | Uvinul ® MC 80 | Ethylhexyl Methoxycinnamate |
| | 2.00 | Uvinul ® T 150 | Ethylhexyl Triazone |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | T-Lite ® SF | Titanium Dioxide, Alumina Hydrate, |
| | | | Dimethicone/Methicone Copolymer |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 4.00 | Uvinul ® MC 80 | Ethylhexyl Methoxycinnamate |
| | 2.00 | Uvinul T ® 150 | Ethylhexyl Triazone |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 4.00 | Uvinul ® MC 80 | Ethylhexyl Methoxycinnamate |
| | 1.00 | Uvinul ® T 150 | Ethylhexyl Triazone |
| | 2.00 | Parsol ® 1789 | Butyl Methoxydibenzoylmethane |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | T-Lite ® SF-S | Titanium Dioxide, Silica Hydrate, Alumina |
| | | | Hydrate, Methicone/Dimethicone Copolymer |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.70 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 0.50 | Keratin-binding domain- | |
| | | reactive dye 14_264 | |
| | q.s. | Preservative | |
| A | 4.00 | Uvinul ® MC 80 | Ethylhexyl Methoxycinnamate |
| | 1.00 | Uvinul ® T 150 | Ethylhexyl Triazone |
| | 2.00 | Parsol ® 1789 | Butyl Methoxydibenzoylmethane |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 67.10 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 0.10 | Keratin-binding domain- | |
| | | reactive dye 14_264 | |
| | q.s. | Preservative | |
| A | 5.00 | Uvinul ® N 539 T | Octocrylene |
| | 2.00 | Parsol ® 1789 | Butyl Methoxydibenzoylmethane |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | T-Lite ® SF-S | Titanium Dioxide, Silica Hydrate, Alumina |
| | | | Hydrate, Methicone/Dimethicone Copolymer |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 5.00 | Uvinul N ® 539 T | Octocrylene |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | T-Lite ® SF | Titanium Dioxide, Alumina Hydrate, |
| | | | Dimethicone/Methicone Copolymer |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 65.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 2.00 | Keratin-binding domain- | |
| | | reactive dye 14_264 | |
| | q.s. | Preservative | |
| A | 5.00 | Uvinul ® N 539 T | Octocrylene |
| | 2.00 | Uvinul ® A Plus | Diethylamino Hydroxybenzoyl Hexyl |
| | | | Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.70 | Water demin. | Aqua dem. |
| D | 2.00 | Simulgel ® NS | Hydroxyethyl Acrytate/Sodium |
| | | | Acryloyldimethyl Taurate Copolymer, |
| | | | Squalane, Polysorbate 60 |
| | 0.50 | Keratin-binding domain- | |
| | | reactive dye 14_264 | |
| | q.s. | Preservative | |
| A | 5.00 | Uvinul ® N 539 T | Octocrylene |
| | 2.00 | Uvinul ® A Plus | Diethylamino Hydroxybenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 2.0 | Mexoryl ® SX | Terephthalidene Dicamphor Sulfonic Acid |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 5.00 | Uvinul ® N 539 T | Octocrylene |
| | 2.00 | Uvinul ® A Plus | Diethylamino Hydrobenzoyl Hexyl Benzoate |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 2.0 | Mexoryl ® SX | Terephthalidene Dicamphor Sulfonic Acid |
| | 65.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 2.00 | Keratin-binding domain- | |
| | | reactive dye 14_264 | |
| | q.s. | Preservative | |
| A | 5.00 | Uvinul ® N 539 T | Octocrylene |
| | 2.00 | Uvinul ® A Plus | Diethylamino Hydroxybenzoyl Hexyl |
| | | | Benzoate |
| | 2.00 | Mexoryl ® XL | Drometrizole Trisiloxane |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 67.10 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 0.10 | Keratin-binding domain- | |
| | | reactive dye 14_264 | |
| | q.s. | Preservative | |
| A | 5.00 | Uvinul ® N 539 T | Octocrylene |
| | 2.00 | Tinosorb ® S | Bis-Ethylhexyloxyphenol Methoxyphenyl |
| | | | Triazine |
| | 0.80 | Rylo PG 11 | Polyglyceryl Dimer Soyate |
| | 1.00 | Span ™ 60 | Sorbitan Stearate |
| | 0.50 | Vitamin E Acetate | Tocopheryl Acetate |
| | 3.00 | Dracorin ® 100 SE | Glyceryl Stearate, PEG-100 Stearate |
| | 1.00 | Cremophor ® CO 410 | PEG-40 Hydrogenated Castor Oil |
| B | 3.00 | Z-COTE ® MAX | Zinc Oxide, Diphenyl Capryl Methicone |
| | 1.00 | Cetiol ® SB 45 | Butyrospermum Parkii (Shea Butter) |
| | 6.50 | Finsolv ® TN | C12-15 Alkyl Benzoate |
| C | 5.00 | Butylene Glycol | Butylene Glycol |
| | 0.30 | Keltrol ® | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel ® NS | Hydroxyethyl Acrylate/Sodium |
| | | | Acryloyldimethyl Taurate Copolymer, |
| | | | Squalane, Polysorbate 60 |
| | 1.00 | Keratin-binding domain- | |
| | | reactive dye 14_264 | |
| | q.s. | Preservative | |
| A | 7.50 | Uvinul ® MC 80 | Ethylhexyl Methoxycinnamate |
| | 5.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | Z-COTE ® MAX | Zinc Oxide, Diphenyl Capryl |
| | | | Methicone |
| C | 4.00 | 1,2-Propylene Glycol Care | Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 65.30 | Water demin. | Aqua dem. |
| | q.s. | Preservative | |
| | 0.50 | Vitamin E Acetate | Tocopheryl Acetate |
| | 2.00 | Keratin-binding domain-reactive | |
| | | dye 14_264 | |
| | 1.00 | RetiSTAR ™ | Caprylic/Capric Triglyceride, Sodium |
| | | | Ascorbate, Tocopherol, Retinol |
| A | 7.50 | Uvinul ® MC 80 | Ethylhexyl Methoxycinnamate |
| | 2.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | T-Lite ® SF-S | Titanium Dioxide, Silica Hydrate, |
| | | | Alumina Hydrate, |
| | | | Methicone/Dimethicone Copolymer |
| C | 4.00 | 1,2-Propylene Glycol Care | Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 68.3 | Water demin. | Aqua dem. |
| | q.s. | Preservative | |
| | 0.50 | Vitamin E Acetate | Tocopheryl Acetate |
| | 1.00 | Keratin-binding domain-reactive | |
| | | dye 14_264 | |
| | 2.00 | RetiSTAR ™ | Caprylic/Capric Triglyceride, Sodium |
| | | | Ascorbate, Tocopherol, Retinol |
| A | 7.50 | Uvinul ® MC 80 | Ethylhexyl Methoxycinnamate |
| | 5.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | T-Lite ® SF | Titanium Dioxide, Alumina Hydrate, |
| | | | Dimethicone/Methicone Copolymer |
| C | 4.00 | 1,2-Propylene Glycol Care | Propylene Glycol |
| | 1.00 | D-Panthenol 50 P | Panthenol, Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 65.30 | Water demin. | Aqua dem. |
| | q.s. | Preservative | |
| | 0.50 | Vitamin E Acetate | Tocopheryl Acetate |
| | 1.00 | Keratin-binding domain-reactive | |
| | | dye 14_264 | |
| | 1.00 | RetiSTAR ™ | Caprylic/Capric Triglyceride, Sodium |
| | | | Ascorbate, Tocopherol, Retinol |
| A | 5.50 | Uvinul ® MC 80 | Ethylhexyl Methoxycinnamate |
| | 2.00 | Uvinul ® A Plus | Diethylamino Hydroxybenzoyl Hexyl |
| | | | Benzoate |
| | 3.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | T-Lite ® SF | Titanium Dioxide, Alumina Hydrate, |
| | | | Dimethicone/Methicone Copolymer |
| C | 4.00 | 1,2-Propylene Glycol Care | Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 67.30 | Water demin. | Aqua dem. |
| | q.s. | Preservative | |
| | 0.50 | Vitamin E Acetate | Tocopheryl Acetate |
| | 2.00 | Keratin-binding domain-reactive | |
| | | dye 14_264 | |
| | 1.00 | RetiSTAR ™ | Caprylic/Capric Triglyceride, Sodium |
| | | | Ascorbate, Tocopherol, Retinol |
| A | 1.00 | Abil ® Care 85 | Bis-PEG/PPG-16/16 PEG/PPG-16/16 |
| | | | Dimethicone, Caprylic/Capric |
| | | | Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 2.00 | Parsol 1789 | Butyl Methoxydibenzoylmethane |
| | 2.00 | Mexoryl ® XL | Drometrizole Trisilioxane |
| | 10.00 | Witconol APM | PPG-3 Myristyl Ether |
| | 1.00 | Uvinul ® T 150 | Ethylhexyl Triazone |
| | 1.00 | Dow Corning 345 Fluid | Cyclopentasiloxane, Cyclohexasiloxane |
| | 5.00 | Uvinul ® N 539 T | Octocrylene |
| B | 3.00 | Z-COTE ® MAX | Zinc Oxide, Diphenyl Capryl Methicone |
| C | 5.00 | 1,2-Propylene Glycol | Propylene Glycol |
| | 1.00 | Mexoryl ® SX | Terephthalidene Dicamphor Sulfonic |
| | | | Acid |
| | 0.20 | Keltrol ® | Xanthan Gum |
| | 0.06 | Edeta BD | Disodium EDTA |
| | 0.04 | TEA | Triethanolamine |
| | 0.30 | Carbopol ® Ultrez 10P | Carbomer |
| | 63.50 | Water demin. | Aqua dem. |
| | 1.00 | Keratin-binding domain-reactive | |
| | | dye 14_264 | |
| D | q.s. | Perfume oil | |
| | 0.50 | Glydant | DMDM Hydantoin |
| A | 5.50 | Uvinul ® MC 80 | Ethylhexyl Methoxycinnamate |
| | 2.00 | Uvinul ® A Plus | Diethylamino Hydroxybenzoyl Hexyl |
| | | | Benzoate |
| | 3.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | T-Lite ® SF-S | Titanium Dioxide, Silica Hydrate, |
| | | | Alumina Hydrate, |
| | | | Methicone/Dimethicone Copolymer |
| C | 4.00 | 1,2-Propylene Glycol Care | Propylene Glycol |
| | 0.20 | Keltrol ® | Xanthan Gum |
| | 2.00 | Simulgel ® NS | Hydroxyethyl Acrylate/Sodium |
| | | | Acrylodimethyl Taurate Copolymer, |
| | | | Squalane, Polysorbate 60 |
| | 66.30 | Water demin. | Aqua dem. |
| | q.s. | Preservative | |
| | 0.50 | Vitamin E Acetate | Tocopheryl Acetate |
| | 0.50 | Keratin-binding domain-reactive | |
| | | dye 14_264 | |
| | 1.00 | RetiSTAR ™ | Caprylic/Capric Triglyceride, Sodium |
| | | | Ascorbate, Tocopherol, Retinol |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 5.00 | Uvinul ® MC 80 | Ethylhexyl Methoxycinnamate |
| | 1.00 | Ganex ® V-220 | VP/Eicosene Copolymer |
| | 7.00 | Isopropyl Palmitate | Isopropyl Palmitate |
| B | 3.00 | Z-COTE ® MAX | Zinc Oxide, Diphenyl Capryl |
| | | | Methicone |
| C | 0.20 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 65.10 | Water demin. | Aqua dem. |
| | 0.20 | Edeta BD | Disodium EDTA |
| | 5.00 | 1,2-Propylene Glycol Care | Propylene Glycol |
| | 0.50 | Keratin-binding domain-reactive dye | |
| | | 14_264 | |
| D | 1.00 | Vitamin E Acetate | Tocopheryl Acetate |
| | q.s. | Preservative | |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 5.00 | Uvinul ® MC 80 | Ethylhexyl Methoxycinnamate |
| | 1.00 | Ganex ® V-220 | VP/Eicosene Copolymer |
| | 7.00 | Isopropyl Palmitate | Isopropyl Palmitate |
| B | 3.00 | T-Lite ® SF | Titanium Dioxide, Alumina Hydrate, |
| | | | Dimethicone/Methicone Copolymer |
| C | 0.50 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 65.10 | Water demin. | Aqua dem. |
| | 0.20 | Edeta BD | Disodium EDTA |
| | 5.00 | 1,2-Propylene Glycol Care | Propylene Glycol |
| | 0.50 | Keratin-binding domain-reactive dye | |
| | | 14_264 | |
| D | 1.00 | Vitamin E Acetate | Tocopheryl Acetate |
| | q.s. | Preservative | |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 5.00 | Uvinul ® MC 80 | Ethylhexyl Methoxycinnamate |
| | 1.00 | Ganex ® V-220 | VP/Eicosene Copolymer |
| | 7.00 | Isopropyl Palmitate | Isopropyl Palmitate |
| B | 3.00 | T-Lite ® SF-S | Titanium Dioxide, Silica Hydrate, |
| | | | Alumina Hydrate, |
| | | | Methicone/Dimethicone Copolymer |
| C | 0.50 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 64.60 | Water demin. | Aqua dem. |
| | 0.20 | Edeta BD | Disodium EDTA |
| | 5.00 | 1,2-Propylene Glycol Care | Propylene Glycol |
| | 1.00 | Keratin-binding domain-reactive dye | |
| | | 14_264 | |
| D | 1.00 | Vitamin E Acetate | Tocopheryl Acetate |
| | q.s. | Preservative | |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 5.00 | Uvinul ® MC 80 | Ethylhexyl Methoxycinnamate |
| | 1.00 | Ganex ® V-220 | VP/Eicosene Copolymer |
| | 7.00 | Isopropyl Palmitate | Isopropyl Palmitate |
| B | 3.00 | T-Lite ® SF-S | Titanium Dioxide, Silica Hydrate, |
| | | | Alumina Hydrate, |
| | | | Methicone/Dimethicone Copolymer |
| C | 0.50 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 64.10 | Water demin. | Aqua dem. |
| | 2.00 | Neo Heliopan ® AP | Disodium Phenyl Dibenzimidazole |
| | | | Tetrasulfonate |
| | 0.20 | Edeta BD | Disodium EDTA |
| | 5.00 | 1,2-Propylene Glycol Care | Propylene Glycol |
| D | 1.00 | Vitamin E Acetate | Tocopheryl Acetate |
| | 1.00 | Keratin-binding domain-reactive dye | |
| | | 14_264 | |
| | q.s. | Preservative | |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 7.00 | Uvinul ® N 539 T | Octocrylene |
| | 1.00 | Ganex ® V-220 | VP/Eicosene Copolymer |
| | 7.00 | Isopropyl Palmitate | Isopropyl Palmitate |
| B | 3.00 | T-Lite ® SF-S | Titanium Dioxide, Silica Hydrate. |
| | | | Alumina Hydrate, |
| | | | Methicone/Dimethicone Copolymer |
| C | 0.50 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 64.60 | Water demin. | Aqua dem. |
| | 1.00 | Mexoryl ® SX | Terephthalidene Dicamphor Sulfonic |
| | | | Acid |
| | 0.20 | Edeta BD | Disodium EDTA |
| | 5.00 | 1,2-Propylene Glycol Care | Propylene Glycol |
| D | 1.00 | Vitamin E Acetate | Tocopheryl Acetate |
| | 1.00 | Keratin-binding domain-reactive dye | |
| | | 14_264 | |
| | q.s. | Preservative | |
| A | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 2.00 | Mexoryl ® XL | Drometrizole Trisiloxane |
| | 5.00 | Uvinul ® N 539 T | Octocrylene |
| | 1.00 | Ganex ® V-220 | VP/Eicosene Copolymer |
| | 7.00 | Isopropyl Palmitate | Isopropyl Palmitate |
| B | 3.00 | T-Lite ® SF | Titanium Dioxide, Alumina Hydrate, |
| | | | Dimethicone/Methicone Copolymer |
| C | 0.50 | Keltrol ® | Xanthan Gum |
| | 0.50 | Simulgel ® NS | Hydroxyethyl Acrylate/Sodium |
| | | | Acryloyldimethyl Taurate Copolymer, |
| | | | Squalane, Polysorbate 60 |
| | 65.10 | Water demin. | Aqua dem. |
| | 0.20 | Edeta BD | Disodium EDTA |
| | 5.00 | 1,2-Propylene Glycol Care | Propylene Glycol |
| D | 1.00 | Vitamin E Acetate | Tocopheryl Acetate |
| | 0.50 | Keratin-binding domain-reactive dye | |
| | | 14_264 | |
| | q.s. | Preservative | |
| | 1.00 | Urea | Urea |
| | 2.00 | Neo Heliopan ® AP | Disodium Phenyl Dibenzimidazole |
| | | | Tetrasulfonate |
| | 2.00 | Glycerin 87% | Glycerin |
| | 1.20 | Aristoflex ™ AVC | Ammonium |
| | | | Acryloyldimethyltaurate/VP |
| | | | Copolymer |
| | 71.30 | Water demin. | Aqua dem. |
| B | 3.00 | Cremophor ® CO 40 | PEG-40 Hydrogenated Castor Oil |
| | q.s. | Perfume oil | |
| | 5.00 | Miglyol 812 | Caprylic/Capric Triglyceride |
| | 2.00 | Uvinul ® N 539 T | Octocrylene |
| | 8.00 | Z-COTE ® MAX | Zinc Oxide, Diphenyl Capryl |
| | | | Methicone |
| | 5.00 | Keratin-binding domain-reactive dye | |
| | | 14_264 | |
| | q.s. | Preservative | |
| | 5.00 | Z-COTE ® MAX | Zinc Oxide, Diphenyl Capryl |
| | | | Methicone |
| | 2.00 | Parsol ® 1789 | Butyl Methoxydibenzoylmethane |
| | 1.00 | Uvinul ® T 150 | Ethylhexyl Triazone |
| | 5.00 | Uvinul ® MC 80 | Ethylhexyl Methoxycinnamate |
| | 2.00 | Uvinul ® T 539 N | Octocrylene |
| | 0.50 | Abil ® 350 | Dimethicone |
| | 2.75 | Carnico wax LT 20 | Copernica Cerifera (Carnauba) Wax, |
| | | | Paraffin |
| | 3.70 | Candelilla wax LT 281 LJ | Euphorbia Cerifera (Candelilla) Wax |
| | 1.80 | Beeswax 3050 PH | Beeswax |
| | 3.20 | TeCero wax 30445 | Microcrystalline Wax |
| | 3.20 | TeCero wax 1030 K | Microcrystalline Wax |
| | 1.34 | Cutina CP | Cetyl Palmitate |
| | 6.40 | Vaseline | Petrolatum |
| | 7.30 | Softisan 100 | Hydrogenated Coco-Glycerides |
| | 10.00 | Luvitol ® EHO | Cetearyl Ethylhexanoate |
| | 0.17 | Bisabolol nat. | Bisabolol |
| | 1.84 | Vitamin E Acetate | Tocopheryl Acetate |
| | 0.42 | D,L-Alpha-Tocopherol | Tocopherol |
| | 1.00 | Keratin-binding domain-reactive dye | |
| | | 14_264 | |
| | 40.38 | Castor Oil | Ricinus Communis (Castor) Oil |
| | 5.00 | T-Lite ® SF | Titanium Dioxide, Alumina Hydrate, |
| | | | Dimethicone/Methicone Copolymer |
| | 2.00 | Parsol ® 1789 | Butyl Methoxydibenzoylmethane |
| | 1.00 | Uvinul ® T 150 | Ethylhexyl Triazone |
| | 3.00 | Uvinul ® MC 80 | Ethylhexyl Methoxycinnamate |
| | 2.00 | Uvinul ® N 539 T | Octocrylene |
| | 0.50 | Abil ® 350 | Dimethicone |
| | 2.75 | Carnico wax LT 20 | Copernica Cerifera (Carnauba) Wax, |
| | | | Paraffin |
| | 3.70 | Candelilla wax LT 281 LJ | Euphorbia Cerifera (Candelilla) Wax |
| | 1.80 | Beeswax 3050 PH | Beeswax |
| | 3.20 | TeCero wax 30445 | Microcrystalline Wax |
| | 3.20 | TeCero wax 1030 K | Microcrystalline Wax |
| | 1.34 | Cutina CP | Cetyl Palmitate |
| | 6.40 | Vaseline | Petrolatum |
| | 7.30 | Softisan 100 | Hydrogenated Coco-Glycerides |
| | 10.00 | Luvitol ® EHO | Cetearyl Ethylhexanoate |
| | 0.17 | Bisabolol nat. | Bisabolol |
| | 1.84 | Vitamin E Acetate | Tocopheryl Acetate |
| | 0.42 | D,L-Alpha-Tocopherol | Tocopherol |
| | 1.00 | Keratin-binding domain-reactive dye | |
| | | 14_264 | |
| | 40.38 | Castor Oil | Ricinus Communis (Castor) Oil |
| | 5.00 | T-Lite ® SF-S | Titanium Dioxide, Silica Hydrate, |
| | | | Alumina Hydrate, |
| | | | Methicone/Dimethicone Copolymer |
| | 2.00 | Uvinul ® A Plus | Diethylamino Hydroxybenzoyl Hexyl |
| | | | Benzoate |
| | 2.00 | Mexoryl ® XL | Drometrizole Trisiloxane |
| | 3.00 | Uvinul ® MC 80 | Ethylhexyl Methoxycinnamate |
| | 0.50 | Abil ® 350 | Dimethicone |
| | 2.75 | Carnico wax LT 20 | Copernica Cerifera (Carnauba) |
| | | | Wax, Paraffin |
| | 3.70 | Candelilla wax LT 281 LJ | Euphorbia Cerifera (Candelilla) |
| | | | Wax |
| | 1.80 | Beeswax 3050 PH | Beeswax |
| | 3.20 | TeCero wax 30445 | Microcrystalline Wax |
| | 3.20 | TeCero wax 1030 K | Microcrystalline Wax |
| | 1.34 | Cutina CP | Cetyl Palmitate |
| | 6.40 | Vaseline | Petrolatum |
| | 7.30 | Softisan 100 | Hydrogenated Coco-Glycerides |
| | 10.00 | Luvitol ® EHO | Cetearyl Ethylhexanoate |
| | 0.17 | Bisabolol nat. | Bisabolol |
| | 1.84 | Vitamin E Acetate | Tocopheryl Acetate |
| | 0.42 | D,L-Alpha-Tocopherol | Tocopherol |
| | 2.00 | Keratin-binding domain-reactive dye | |
| | | 14_264 | |
| | 39.38 | Castor Oil | Ricinus Communis (Castor) Oil |
| A | 6.00 | Cremophor ® WO 7 | PEG-7 Hydrogenated Castor Oil |
| | 2.00 | Elfacos ® ST 9 | PEG-45/Dodecyl Glycol Copolymer |
| | 3.00 | Isopropyl myristate | Isopropyl Myristate |
| | 8.00 | Jojoba oil | Simmondsia Chinensis (Jojoba) Seed |
| | | | Oil |
| | 4.00 | Uvinul ® MC 80 | Ethylhexyl Methoxycinnamate |
| | 2.00 | Uvinul ® A Plus | Diethylamino Hydroxybenzoyl Hexyl |
| | | | Benzoate |
| | 1.00 | Abil ® 350 | Dimethicone |
| B | 5.00 | Z-COTE ® MAX | Zinc Oxide, Diphenyl Capryl Methicone |
| | 3.00 | T-Lite ® SF | Titanium Dioxide, Alumina Hydrate, |
| | | | Dimethicone/Methicone Copolymer |
| C | 0.20 | Edeta BD | Disodium EDTA |
| | 5.00 | Glycerin 87% | Glycerin |
| | 0.30 | Chemag 2000 | Imidazolidinyl Urea |
| | 59.00 | Water demin. | Aqua dem. |
| D | q.s. | Perfume oil | |
| | 1.00 | Keratin-binding domain-reactive | |
| | | dye 14_264 | |
| | q.s. | Preservative | |
| | 2.00 | Cremophor ® A 6 | Ceteareth-6, Stearyl Alcohol |
| | 2.00 | Cremophor ® A 25 | Ceteareth-25 |
| | 3.00 | Syncrowax HRC | Tribehenin |
| | 2.00 | Lanette O | Cetearyl Alcohol |
| | 2.00 | Luvitol ® EHO | Cetearyl Ethylhexanoate |
| | 5.00 | Uvinul ® MC 80 | Ethylhexyl Methoxycinnamate |
| | 1.00 | Uvinul ® T 150 | Ethylhexyl Triazone |
| | 1.00 | Ganex ® V-220 | VP/Eicosene Copolymer |
| | 7.00 | Isopropyl myristate | Isopropyl Myristate |
| B | 5.00 | Z-COTE ® MAX | Zinc Oxide, Diphenyl Capryl |
| | | | Methicone |
| C | 0.20 | Keltrol ® | Xanthan Gum |
| | 0.50 | Simulgel ® NS | Hydroxyethyl Acrylate/Sodium |
| | | | Acryloyldimethyl Taurate |
| | | | Copolymer, Squalane, |
| | | | Polysorbate 60 |
| | 61.10 | Water demin. | Aqua dem. |
| | 0.20 | Edeta BD | Disodium EDTA |
| | 5.00 | 1,2-Propylene Glycol Care | Propylene Glycol |
| D | 1.00 | Vitamin E Acetate | Tocopheryl Acetate |
| | 1.50 | Keratin-binding domain-reactive dye | |
| | | 14_264 | |
| | q.s. | Preservative | |
| A | 2.00 | Luvitol ® EHO | Cetearyl Ethylhexanoate |
| | 5.00 | Paraffin oil | Mineral Oil |
| | 5.00 | Z-COTE ® MAX | Zinc Oxide, Diphenyl Capryl |
| | | | Methicone |
| B | 1.00 | Cremophor ® A 6 | Ceteareth-6, Stearyl Alcohol |
| | 2.00 | Cremophor ® A 25 | Ceteareth-25 |
| | 3.00 | Glycerinmonostearate | Glyceryl Stearate |
| | 4.00 | Cetylstearyl alcohol | Cetearyl Alcohol |
| C | 5.00 | 1,2-Propylene Glycol Care | Propylene Glycol |
| | 7.50 | Luviquat Care | Polyquaternium-44 |
| | q.s. | Preservative | |
| | q.s. | Perfume oil | |
| | 1.00 | Keratin-binding domain-reactive dye | |
| | | 14_264 | |
| | 64.50 | Water demin. | Aqua dem. |
| | 5.00 | T-Lite ® SF | Titanium Dioxide, Alumina |
| | | | Hydrate, Dimethicone/Methicone |
| | | | Copolymer |
| | 10.00 | Z-COTE ® MAX | Zinc Oxide, Diphenyl Capryl |
| | | | Methicone |
| | 12.00 | Beeswax 3044 PH | Beeswax |
| | 8.00 | Candelilla wax LT 281 LJ | Euphorbia Cerifera (Candelilla) |
| | | | Wax |
| | 5.00 | Tegin | Glyceryl Stearate SE |
| | 5.00 | Dow Corning ® 345 Fluid | Cyclopentasiloxane, |
| | | | Cyclohexasiloxane |
| | 5.00 | Witconol ® APM | PPG-3 Myristyl Ether |
| | 5.00 | Softisan 154 | Hydrogenated Palm Oil |
| | 8.00 | Paraffin oil, high viscosity | Mineral Oil |
| | 3.00 | Vaseline | Petrolatum |
| | 2.00 | Keratin-binding domain-reactive dye | |
| | | 14_264 | |
| | 32.00 | Castor Oil | Ricinus Communis (Castor) Oil |
| | 3.00 | Uvinul ® MC 80 | Ethylhexyl Methoxycinnamate |
| | 2.00 | Uvinul ® T 150 | Ethylhexyl Triazone |
| | 2.00 | Uvinul ® A Plus | Diethylamino Hydroxybenzoyl |
| | | | Hexyl Benzoate |
| | 10.00 | Z-COTE ® MAX | Zinc Oxide, Diphenyl Capryl |
| | | | Methicone |
| | 12.00 | Beeswax 3044 PH | Beeswax |
| | 3.00 | Vaseline | Petrolatum |
| | 8.00 | Candelilla wax LT 281 LJ | Euphorbia Cerifera (Candelilla) |
| | | | Wax |
| | 8.00 | Paraffin oil, high viscosity | Mineral Oil |
| | 5.00 | Tegin | Glyceryl Stearate SE |
| | 5.00 | Softisan 154 | Hydrogenated Palm Oil |
| | 5.00 | Witconol APM | PPG-3 Myristyl Ether |
| | 5.00 | Dow Corning 345 Fluid | Cyclopentasiloxane, |
| | | | Cyclohexasiloxane |
| | 0.10 | Keratin-binding domain-reactive dye | |
| | | 14_264 | |
| | 28.90 | Castor Oil | Ricinus Communis (Castor) Oil |
| | 5.00 | T-Lite ® SF | Titanium Dioxide, Alumina |
| | | | Hydrate, Dimethicone/Methicone |
| | | | Copolymer |
| | 6.00 | Finsolv ® TN | C12-15 Alkyl Benzoate |
| | 10.00 | Uvinul ® MC 80 | Ethylhexyl Methoxycinnamate |
| | 6.00 | Miglyol ® 812 | Caprylic/Capric Triglyceride |
| | 5.00 | Arlacel ® P 135 | PEG-30 Dipolyhydroxystearate |
| | 2.00 | Ganex ® V 216 | PVP/Hexadecene Copolymer |
| | 2.00 | Elfacos ® ST 9 | PEG-45/Dodecyl Glycol |
| | | | Copolymer |
| B | 3.00 | 1,2-Propylene Glycol Care | Propylene Glycol |
| | 0.10 | Edeta BD | Disodium EDTA |
| | 1.00 | Magnesium sulfate 7-hydrate | Magnesium Sulfate |
| | 59.40 | Water demin. | Aqua dem. |
| | 0.50 | Keratin-binding domain-reactive dye | |
| | | 14_264 | |
| | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 4.00 | Uvinul ® MC 80 | Ethylhexyl Methoxycinnamate |
| | 2.00 | Uvinul ® N 539 T | Octocrylene |
| B | 5.00 | Z-COTE ® MAX | Zinc Oxide, Diphenyl Capryl |
| | | | Methicone |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 66.20 | Water demin. | Aqua dem. |
| | 1.50 | Keratin-binding domain-reactive dye | |
| | | 14_264 | |
| D | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 4.00 | Uvinul ® MC 80 | Ethylhexyl Methoxycinnamate |
| | 2.00 | Uvinul ® N 539 T | Octocrylene |
| B | 5.00 | T-Lite ® SF-S | Titanium Dioxide, Silica Hydrate, |
| | | | Alumina Hydrate, |
| | | | Methicone/Dimethicone Copolymer |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 1.00 | Keratin-binding domain-reactive dye | |
| | | 14_264 | |
| | 66.70 | Water demin. | Aqua dem. |
| D | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 8.00 | Uvinul ® A Plus B | Ethylhexyl Methoxycinnamate and |
| | | | Diethylamino Hydroxybenzoyl |
| | | | hexyl Benzoate |
| | 2.00 | Uvinul ® N 539 T | Octocrylene |
| B | 5.00 | Z-COTE ® MAX | Zinc Oxide, Diphenyl Capryl |
| | | | Methicone |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 0.10 | Keratin-binding domain-reactive dye | |
| | | 14_264 | |
| | 63.60 | Water demin. | Aqua dem. |
| | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 8.00 | Uvinul ® A Plus B | Ethylhexyl Methoxycinnamate and |
| | | | Diethylamino Hydroxybenzoyl |
| | | | Hexyl Benzoate |
| | 2.00 | Uvinul ® N 539 T | Octocrylene |
| B | 5.00 | T-Lite ® SF | Titanium Dioxide, Alumina Hydrate, |
| | | | Dimethicone/Methicene Copolymor |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 63.50 | Water demin. | Aqua dem. |
| | 0.20 | Keratin-binding domain-reactive dye | |
| | | 14_264 | |
| | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 8.00 | Uvinul ® A Plus B | Ethylhexyl Methoxycinnamate and |
| | | | Diethylamino Hydroxybenzoyl |
| | | | hexyl Benzoate |
| | 2.00 | Uvinul ® N 539 T | Octocrylene |
| B | 5.00 | T-Lite ® SF-S | Titanium Dioxide, Silica Hydrate, |
| | | | Alumina Hydrate, |
| | | | Methicone/Dimethicone Copolymer |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 0.50 | Keratin-binding domain-reactive dye | |
| | | 14_264 | |
| | 63.20 | Water demin. | Aqua dem. |
| | q.s. | Preservative | |
| A | 4.00 | Dehymuls ® SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 8.00 | Finsolv ® TN | C12-15 Alkyl Benzoate |
| | 4.00 | Miglyol ® 812 | Caprylic/Capric Triglyceride |
| | 7.00 | Uvinul ® MC 80 | Ethylhexyl Methoxycinnamate |
| | 2.00 | Mexoryl ® XL | Drometrizole Trisiloxane |
| B | 5.00 | Z-COTE ® MAX | Zinc Oxide, Diphenyl Capryl |
| | | | Methicone |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 64.69 | Water demin. | Aqua dem. |
| | 0.01 | Keratin-binding domain-reactive dye | |
| | | 14_264 | |
| | q.s. | Preservative | |
| A | 7.50 | Uvinul ® MC 80 | Ethylhexyl Methoxycinnamate |
| | 3.00 | Uvinul ® N 539 T | Octocrylene |
| | 2.00 | Uvinul ® A Plus | Diethylamino Hydroxybenzoyl |
| | | | Hexyl Benzoate |
| | 1.00 | Cremophor ® CO 40 | PEG-40 Hydrogenated Castor Oil |
| | 10.00 | Miglyol 812 | Caprylic/Capric Triglyceride |
| | 1.50 | Dow Corning ® 345 Fluid | Cyclopentasiloxane, |
| | | | Cyclohexasiloxane |
| B | 3.50 | Luvigel ® EM | Caprylic/Capric Triglyceride, |
| | | | Sodium Acrylates Copolymer |
| C | 46.00 | Water demin. | Aqua dem. |
| D | 5.00 | 1,2-Propylene Glycol Care | Propylene Glycol |
| | 0.50 | Cremophor ® A 25 | Ceteareth-25 |
| | 0.05 | Keratin-binding domain-reactive dye | |
| | | 14_264 | |
| | 20.00 | Ethanol 96% | Alcohol |
| A | 1.00 | Uvinul ® A Plus | Diethylamino Hydroxybenzoyl |
| | | | Hexyl Benzoate |
| | 1.00 | Tinosorb ® S | Bis-Ethylhexyloxyphenol |
| | | | Methoxyphenyl Triazine |
| | 3.00 | Uvinul ® MC 80 | Ethylhexyl Methoxycinnamate |
| | 8.00 | Miglyol ® 812 | Caprylic/Capric Triglyceride |
| | 1.50 | Dow Corning ® 350 Fluid | Dimethicone |
| | 3.00 | Z-COTE ® MAX | Zinc Oxide, Diphenyl Capryl |
| | | | Methicone |
| | 3.00 | Finsolv ® TN | C12-15 Alkyl Benzoate |
| | 1.00 | Cremophor ® CO 40 | PEG-40 Hydrogenated Castor Oil |
| B | 2.00 | Luvigel ® EM | Caprylic/Capric Triglyceride, |
| | | | Sodium Acrylates Copolymer |
| C | 54.80 | Water demin. | Aqua dem. |
| D | 15.00 | Ethanol 96% | Alcohol |
| | 5.00 | 1,2-Propylene Glycol Care | Propylene Glycol |
| | 0.50 | Cremophor ® A 25 | Ceteareth-25 |
| | 1.00 | Keratin-binding domain-reactive dye | |
| | | 14_264 | |
| | 1.00 | Vitamin E Acetate | Tocopheryl Acetate |
| | 0.20 | Bisabolol rac. | Bisabolol |
| A | 4.00 | Dehymuls ® SBL | Polyglyceryl-2 |
| | | | Dipolyhydroxystearate, |
| | | | Dicaprylyl Ether, |
| | | | Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls ® PGPH | Polyglyceryl-2 |
| | | | Dipolyhydroxystearate |
| | 6.00 | Finsolv ® TN | C12-15 Alkyl Benzoate |
| | 6.00 | Miglyol ® 812 | Caprylic/Capric Triglyceride |
| | 5.00 | Uvinul ® MC 80 | Ethylhexyl Methoxycinnamate |
| | 3.00 | Neo Heliopan ® HMS | Homosalate |
| B | 5.00 | Z-COTE ® MAX | Zinc Oxide, Diphenyl Capryl |
| | | | Methicone |
| C | 3.00 | 1,2-Propylene Glycol Care | Propylene Glycol |
| | 0.30 | Chemag 2000 | Imidazolidinyl Urea |
| | 1.00 | Magnesium sulfate 7-hydrate | Magnesium Sulfate |
| | 65.10 | Water demin. | Aqua dem. |
| | 0.10 | Keratin-binding domain-reactive dye | |
| | | 14_264 | |
| | q.s. | Preservative | |
| A | 4.00 | Tego ® Care 450 | Polyglyceryl-3 Methyl Glucose |
| | | | Distearate |
| | 4.50 | Uvinul ® MC 80 | Ethylhexyl Methoxycinnamate |
| | 3.00 | Uvinul ® N 539 T | Octocrylene |
| | 2.00 | Uvinul ® T 150 | Ethylhexyl Triazone |
| | 0.50 | Vitamin E Acetate | Tocopheryl Acetate |
| B | 5.00 | Isohexadecane | Isohexadecane |
| | 5.00 | Cosmacol ® EMI | Di-C12-13 Alkyl Malate |
| | 3.50 | Cetiol ® SN | Cetearyl Isononanoate |
| | 1.00 | Ganex ® V-220 | VP/Eicosene Copolymer |
| | 5.00 | T-Lite ® SF | Titanium Dioxide, Alumina |
| | | | Hydrate, Dimethicone/Methicone |
| | | | Copolymer |
| C | 60.00 | Water demin. | Water |
| | 5.00 | Glycerin 87% | Glycerin |
| | 1.00 | Lanette E | Sodium Cetearyl Sulfate |
| | 0.30 | Keltrol ® | Xanthan Gum |
| | 0.20 | Keratin-binding domain-reactive dye | |
| | | 14_264 | |
| D | q.s. | Preservative | |
| A | 4.00 | Tego ® Care 450 | Polyglyceryl-3 Methyl Glucose |
| | | | Distearate |
| | 4.50 | Uvinul ® MC 80 | Ethylhexyl Methoxycinnamate |
| | 3.00 | Uvinul ® N 539 T | Octocrylene |
| | 2.00 | Neo Heliopan ® HMS | Homosalate |
| | 0.50 | Vitamin E Acetate | Tocopheryl Acetate |
| B | 5.00 | Isohexadecane | Isohexadecane |
| | 5.00 | Cosmacol ® EMI | Di-C12-13 Alkyl Malate |
| | 3.50 | Cetiol ® SN | Cetearyl Isononanoate |
| | 1.00 | Ganex ® V-220 | VP/Eicosene Copolymer |
| | 5.00 | T-Lite ® SF | Titanium Dioxide, Alumina |
| | | | Hydrate, |
| | | | Dimethicone/Methicone |
| | | | Copolymer |
| C | 59.20 | Water demin. | Aqua dem. |
| | 5.00 | Glycerin 87% | Glycerin |
| | 1.00 | Lanette E | Sodium Cetearyl Sulfate |
| | 0.30 | Keltrol ® | Xanthan Gum |
| | 1.00 | Keratin-binding domain-reactive dye | |
| | | 14_264 | |
| D | q.s. | Preservative | |
| A | 4.00 | Tego ® Care 450 | Polyglyceryl-3 Methyl Glucose |
| | | | Distearate |
| | 4.50 | Uvinul ® MC 80 | Ethylhexyl Methoxycinnamate |
| | 3.00 | Neo Heliopan ® OS | Octisalate |
| | 2.00 | Neo Heliopan ® HMS | Homosalate |
| | 0.50 | Vitamin E Acetate | Tocopheryl Acetate |
| B | 5.00 | Isohexadecane | Isohexadecane |
| | 5.00 | Cosmacol ® EMI | Di-C12-13 Alkyl Malate |
| | 3.50 | Cetiol ® SN | Cetearyl Isononanoate |
| | 1.00 | Ganex ® V-220 | VP/Eicosene Copolymer |
| | 5.00 | Z-COTE ® MAX | Zinc Oxide, Diphenyl Capryl |
| | | | Methicone |
| C | 60.15 | Water demin. | Aqua dem |
| | 5.00 | Glycerin 87% | Glycerin |
| | 1.00 | Lanette E | Sodium Cetearyl Sulfate |
| | 0.30 | Keltrol ® | Xanthan Gum |
| | 0.05 | Keratin-binding domain-reactive dye | |
| | | 14_264 | |
| D | q.s. | Preservative | |
| A | 4.00 | Tego ® Care 450 | Polyglyceryl-3 Methyl Glucose |
| | | | Distearate |
| | 4.50 | Uvinul ® N 539 T | Octocrylene |
| | 3.00 | Neo Heliopan ® OS | Octisalate |
| | 2.00 | Neo Heliopan ® HMS | Homosalate |
| | 0.50 | Vitamin E Acetate | Tocopheryl Acetate |
| B | 5.00 | Isohexadecane | Isohexadecane |
| | 5.00 | Cosmacol ® EMI | Di-C12-13 Alkyl Malate |
| | 3.50 | Cetiol ® SN | Cetearyl Isononanoate |
| | 1.00 | Ganex ® V-220 | VP/Eicosene Copolymer |
| | 5.00 | Z-COTE ® MAX | Zinc Oxide, Diphenyl Capryl |
| | | | Methicone |
| C | 60.19 | Water demin. | Aqua dem. |
| | 5.00 | Glycerin 87% | Glycerin |
| | 1.00 | Lanette E | Sodium Cetearyl Sulfate |
| | 0.30 | Keltrol ® | Xanthan Gum |
| | 0.01 | Keratin-binding domain-reactive dye | |
| | | 14_264 | |
| D | q.s. | Preservative | |
| A | 4.00 | Tego ® Care 450 | Polyglyceryl-3 Methyl Glucose |
| | | | Distearate |
| | 4.50 | Uvinul ® N 539 T | Octocrylene |
| | 3.00 | Neo Heliopan ® OS | Octisalate |
| | 2.00 | Neo Heliopan ® HMS | Homosalate |
| | 0.50 | Vitamin E Acetate | Tocopheryl Acetate |
| B | 5.00 | Isohexadecane | Isohexadecane |
| | 5.00 | Cosmacol ® EMI | Di-C12-13 Alkyl Malate |
| | 3.50 | Cetiol ® SN | Cetearyl Isononanoate |
| | 1.00 | Ganex ® V-220 | VP/Eicosene Copolymer |
| | 5.00 | T-Lite ® SF-S | Titanium Dioxide, Silica |
| | | | Hydrate, Alumina Hydrate, |
| | | | Methicone/Dimethicone |
| | | | Copolymer |
| C | 59.20 | Water demin. | Aqua dem. |
| | 5.00 | Glycerin 87% | Glycerin |
| | 1.00 | Lanette E | Sodium Cetearyl Sulfate |
| | 0.30 | Keltrol ® | Xanthan Gum |
| | 1.00 | Keratin-binding domain-reactive dye | |
| | | 14_264 | |
| D | q.s. | Preservative | |
| A | 8.00 | Cetiol ® B | Dibutyl Adipate |
| | 8.00 | Finsolv ® TN | C12-15 Alkyl Benzoate |
| | 12.00 | Myritol ® 331 | Cocoglycerides |
| | 1.00 | Lanette E | Sodium Cetearyl Sulfate |
| | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 3.00 | Tinosorb ® S | Bis-Ethylhexyloxyphenol |
| | | | Methoxyphenyl Triazine |
| | 1.00 | Vitamin E Acetate | Tocopheryl Acetate |
| B | 4.00 | Z-COTE ® MAX | Zinc Oxide, Diphenyl Capryl |
| | | | Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 1.50 | Veegum Ultra | Magnesium Aluminum Silicate |
| | 49.45 | Water demin. | Aqua dem. |
| | 1.00 | Keratin-binding domain-reactive dye | |
| | | 14_264 | |
| D | q.s. | Preservative | |
| A | 8.00 | Cetiol ® B | Dibutyl Adipate |
| | 8.00 | Finsolv ® TN | C12-15 Alkyl Benzoate |
| | 12.00 | Myritol ® 331 | Cocoglycerides |
| | 1.00 | Lanette E | Sodium Cetearyl Sulfate |
| | 4.00 | Eumulgin ® VL 75 | Lauryl Glucoside, Polyglyceryl-2 |
| | | | Dipolyhydroxystearete, Glycerin |
| | 2.00 | Lanette O | Cetearyl Alcohol |
| | 3.00 | Tinosorb ® S | Bis-Ethylhexyloxyphenol |
| | | | Methoxyphenyl Triazine |
| | 1.00 | Vitamin E Acetate | Tocopheryl Acetate |
| B | 4.00 | T-Lite ® SF | Titanium Dioxide, Alumina |
| | | | Hydrate, Dimethicone/Methicone |
| | | | Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 1.50 | Veegum Ultra | Magnesium Aluminum Silicate |
| | 50.25 | Water demin. | Aqua dem. |
| D | 0.50 | Citric acid | Citric Acid |
| | q.s. | Perfume oil | |
| | 0.20 | Keratin-binding domain-reactive dye | |
| | | 14_264 | |
| | q.s. | Preservative | |
| A | 5.00 | Cosmacol ® EMI | Di-C12-13 Alkyl Malate |
| | 4.50 | Uvinul ® MC 80 | Ethylhexyl Methoxycinnamate |
| | 3.00 | Uvinul ® N 539 T | Octocrylene |
| | 2.00 | Uvinul ® T 150 | Ethylhexyl Triazone |
| B | 4.00 | Tego ® Care 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 2.50 | T-Lite ® SF | Titanium Dioxide, Alumina |
| | | | Hydrate, |
| | | | Dimethicone/Methicone |
| | | | Copolymer |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 42.00 | Water demin. | Aqua dem |
| D | 5.00 | Eusolex ® 232 | Phenylbenzimidazole Sulfonic |
| | | | Acid |
| | 10.00 | Water demin. | Aqua dem |
| E | 0.70 | Sodium hydroxide | Sodium Hydroxide |
| | 1.00 | Keratin-binding domain-reactive dye | |
| | | 14_264 | |
| | q.s. | Preservative | |
| A | 5.00 | Cosmacol ® EMI | Di-C12-13 Alkyl Malate |
| | 4.50 | Uvinul ® MC 80 | Ethylhexyl Methoxycinnamate |
| | 3.00 | Uvinul ® N 539 T | Octocrylene |
| | 2.00 | Uvinul ® A Plus | Diethylamino Hydrobenzoyl |
| | | | Hexyl Benzoate |
| B | 4.00 | Tego ® Care 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 2.50 | T-Lite ® SF | Titanium Dioxide, Alumina |
| | | | Hydrate, |
| | | | Dimethicone/Methicone |
| | | | Copolymer |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 41.00 | Water demin. | Aqua dem |
| D | 5.00 | Eusolex ® 232 | Phenylbenzimidazole Sulfonic |
| | | | Acid |
| | 2.00 | Keratin-binding domain-reactive dye | |
| | | 14_264 | |
| | 10.00 | Water demin. | Aqua dem |
| E | 0.70 | Sodium hydroxide | Sodium Hydroxide |
| | q.s. | Preservative | |
| A | 5.00 | Cosmacol ® EMI | Di-C12-13 Alkyl Malate |
| | 4.50 | Uvinul ® MC 80 | Ethylhexyl Methoxycinnamate |
| | 3.00 | Uvinul ® N 539 T | Octocrylene |
| | 2.00 | Uvinul ® A Plus | Diethylamino Hydrobenzoyl |
| | | | Hexyl Benzoate |
| B | 4.00 | Tego Care 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 2.50 | Z-COTE ® MAX | Zinc Oxide, Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 42.50 | Water demin. | Aqua dem |
| D | 5.00 | Eusolex ® 232 | Phenylbenzimidazole Sulfonic |
| | | | Acid |
| | 0.50 | Keratin-binding domain-reactive dye | |
| | | 14_264 | |
| | 10.00 | Water demin. | Aqua dem |
| E | 0.70 | Sodium hydroxide | Sodium Hydroxide |
| | q.s. | Preservative | |
| A | 5.00 | Cosmacol ® EMI | Di-C12-13 Alkyl Malate |
| | 7.50 | Uvinul ® N 539 T | Octocrylene |
| | 2.00 | Mexoryl ® XL | Drometrizole Trisiloxane |
| B | 4.00 | Tego ® Care 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 5.00 | Z-COTE ® MAX | Zinc Oxide, Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 40.50 | Water demin. | Aqua dem |
| D | 5.00 | Eusolex ® 232 | Phenylbenzimidazole Sulfonic |
| | | | Acid |
| | 1.00 | Keratin-binding domain-reactive dye | |
| | | 14_264 | |
| | 10.00 | Water demin. | Aqua dem |
| E | 0.70 | Sodium hydroxide | Sodium Hydroxide |
| | q.s. | Preservative | |
| A | 5.00 | Cosmacol ® EMI | Di-C12-13 Alkyl Malate |
| | 5.50 | Uvinul ® N 539 T | Octocrylene |
| | 2.00 | Uvinul ® A Plus | Diethylamino Hydrobenzoyl |
| | | | Hexyl Benzoate |
| | 2.00 | Mexoryl ® XL | Drometrizole Trisiloxane |
| B | 4.00 | Tego ® Care 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 5.00 | Z-COTE ® MAX | Zinc Oxide, Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 41.50 | Water demin. | Aqua dem |
| D | 5.00 | Eusolex ® 232 | Phenylbenzimidazole Sulfonic |
| | | | Acid |
| | 0.50 | Keratin-binding domain-reactive dye | |
| | | 14_264 | |
| | 9.50 | Water demin. | Aqua dem |
| E | 0.70 | Sodium hydroxide | Sodium Hydroxide |
| | q.s. | Preservative | |
| A | 5.00 | Cosmacol ® EMI | Di-C12-13 Alkyl Malate |
| | 5.50 | Uvinul ® N 539 T | Octocrylene |
| | 2.00 | Uvinul ® A Plus | Diethylamino Hydrobenzoyl |
| | | | Hexyl Benzoate |
| | 2.00 | Mexoryl ® XL | Drometrizole Trisiloxane |
| B | 4.00 | Tego Care ® 450 | Polyglyceryl-3 Methyl Glucose |
| | | | Distearate |
| | 5.00 | Isohexadecane | Isohexadecane |
| | 3.50 | Cetiol ® SN | Cetearyl Isononanoate |
| | 0.50 | Vitamin E Acetate | Tocopheryl Acetate |
| | 1.00 | Ganex ® V-220 | VP/Eicosene Copolymer |
| | 5.00 | T-Lite ® SF-S | Titanium Dioxide, Silica |
| | | | Hydrate, Alumina Hydrate, |
| | | | Methicone/Dimethicone |
| | | | Copolymer |
| C | 5.00 | Glycerin 87% | Glycerin |
| | 2.00 | Lanette E | Sodium Cetearyl Sulfate |
| | 0.30 | Keltrol ® | Xanthan Gum |
| | 1.00 | Pationic ® 138 C | Sodium Lauroyl Lactylate |
| | 1.00 | Pationic ® SSL | Sodium Stearoyl Lactylate |
| | 40.50 | Water demin. | Aqua dem |
| D | 5.00 | Eusolex ® 232 | Phenylbenzimidazole Sulfonic |
| | | | Acid |
| | 1.00 | Keratin-binding domain-reactive dye | |
| | | 14_264 | |
| | 10.00 | Water demin. | Aqua dem |
| E | 0.70 | Sodium hydroxide | Sodium Hydroxide |
| | q.s. | Preservative | |
| A | 8.00 | Cetiol ® B | Dibutyl Adipate |
| | 8.00 | Finsolv ® TN | C12-15 Alkyl Benzoate |
| | 10.00 | Myritol ® 331 | Cocoglycerides |
| | 1.00 | Lanette E | Sodium Cetearyl Sulfate |
| | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb ® S | Bis-Ethylhexyloxyphenol |
| | | | Methoxyphenyl Triazine |
| | 1.00 | Uvinul ® T 150 | Ethylhexyl Triazone |
| | 1.00 | Vitamin E Acetate | Tocopheryl Acetate |
| B | 5.00 | T-Lite ® SF | Titanium Dioxide, Alumina |
| | | | Hydrate, |
| | | | Dimethicone/Methicone |
| | | | Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 2.00 | Keratin-binding domain-reactive dye | |
| | | 14_264 | |
| | 1.50 | Veegum Ultra | Magnesium Aluminum Silicate |
| | 51.95 | Water demin. | Aqua dem |
| D | q.s. | Perfume oil | |
| | q.s. | Preservative | |
| A | 8.00 | Cetiol ® B | Dibutyl Adipate |
| | 8.00 | Finsolv ® TN | C12-15 Alkyl Benzoate |
| | 10.00 | Myritol ® 331 | Cocoglycerides |
| | 1.00 | Lanette E | Sodium Cetearyl Sulfate |
| | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb ® S | Bis-Ethylhexyloxyphenol |
| | | | Methoxyphenyl triazine |
| | 1.00 | Uvinul ® T 150 | Ethylhexyl Triazone |
| | 1.00 | Vitamin E Acetate | Tocopheryl Acetate |
| B | 5.00 | Z-COTE ® MAX | Zinc Oxide, Diphenyl Capryl |
| | | | Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 1.00 | Keratin-binding domain-reactive dye | |
| | | 14_264 | |
| | 1.50 | Veegum Ultra | Magnesium Aluminum Silicate |
| | 52.95 | Water demin. | Aqua dem. |
| D | q.s. | Perfume oil | |
| | q.s. | Preservative | |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare ® 2000 | Decyl Glucoside |
| | 0.30 | Keltrol ® T | Xanthan Gum |
| | 57.00 | Water demin. | Aqua dem. |
| B | 5.00 | T-Lite ® SF-S | Titanium Dioxide, Silica |
| | | | Hydrate, Alumina Hydrate, |
| | | | Methicone/Dimethicone |
| | | | Copolymer |
| C | 7.50 | Uvinul ® MC 80 | Ethylhexyl Methoxycinnamate |
| | 2.00 | Uvinul ® A Plus | Diethylamino Hydroxybenzoyl |
| | | | Hexyl Benzoate |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol ® 812 | Caprylic/Capric Triglyceride |
| D | 1.00 | Vitamin E Acetate | Tocopheryl Acetate |
| | 2.00 | Keratin-binding domain-reactive dye | |
| | | 14_264 | |
| | 0.20 | Bisabolol rac. | Bisabolol |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol ® T | Xanthan Gum |
| | 58.00 | Water demin. | Aqua dem. |
| B | 5.00 | Z-COTE ® MAX | Zinc Oxide, Diphenyl Capryl |
| | | | Methicone |
| C | 7.50 | Uvinul ® MC 80 | Ethylhexyl Methoxycinnamate |
| | 2.00 | Uvinul ® A Plus | Diethylamino Hydroxybenzoyl |
| | | | Hexyl Benzoate |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol ® 812 | Caprylic/Capric Triglyceride |
| D | 1.00 | Vitamin E Acetate | Tocopheryl Acetate |
| | 1.00 | Keratin-binding domain-reactive dye | |
| | | 14_264 | |
| | 0.20 | Bisabolol rac. | Bisabolol |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare ® 2000 | Decyl Glucoside |
| | 0.30 | Keltrol ® T | Xanthan Gum |
| | 58.50 | Water demin. | Aqua dem. |
| B | 5.00 | T-Lite ® SF-S | Titanium Dioxide, Silica |
| | | | Hydrate, Alumina Hydrate, |
| | | | Methicone/Dimethicone |
| | | | Copolymer |
| C | 7.50 | Uvinul ® MC 80 | Ethylhexyl Methoxycinnamate |
| | 5.00 | Uvinul ® N 539 T | Octocrylene |
| | 2.00 | Parsol ® 1789 | Butyl |
| | | | Methoxydibenzoylmethane |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol ® 812 | Caprylic/Capric Triglyceride |
| D | 1.00 | Vitamin E Acetate | Tocopheryl Acetate |
| | 0.50 | Keratin-binding domain-reactive dye | |
| | | 14_264 | |
| | 0.20 | Bisabolol rac. | Bisabolol |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol ® T | Xanthan Gum |
| | 2.00 | Mexoryl ® SL | Terephthalidene Dicamphor |
| | | | Sulfonic Acid |
| | 58.40 | Water demin. | Aqua dem. |
| B | 6.00 | T-Lite ® SF-S | Titanium Dioxide, Silica |
| | | | Hydrate, Alumina Hydrate, |
| | | | Methicone/Dimethicone |
| | | | Copolymer |
| | 6.00 | Uvinul ® N 539 T | Octocrylene |
| | 2.00 | Parsol ® 1789 | Butyl |
| | | | Methoxydibenzoylmethane |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 0.10 | Keratin-binding domain-reactive dye | |
| | | 14_264 | |
| | 0.20 | Bisabolol rac. | Bisabolol |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare ® 2000 | Decyl Glucoside |
| | 0.30 | Keltrol ® T | Xanthan Gum |
| | 2.00 | Mexoryl ® SL | Terephthalidene Dicamphor |
| | | | Sulfonic Acid |
| | 58.45 | Water demin. | Aqua dem. |
| B | 6.00 | Z-COTE ® MAX | Zinc Oxide, Diphenyl Capryl |
| | | | Methicone |
| | 6.00 | Uvinul ® N 539 T | Octocrylene |
| | 2.00 | Parsol ® 1789 | Butyl |
| | | | Methoxydibenzoylmethane |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 0.05 | Keratin-binding domain-reactive dye | |
| | | 14_264 | |
| | 0.20 | Bisabolol rac. | Bisabolol |
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare ® 2000 | Decyl Glucoside |
| | 0.30 | Keltrol ® T | Xanthan Gum |
| | 2.00 | Neo Heliopan ® AP | Disodium Phenyl |
| | | | Dibenzimidazole Tetrasulfonate |
| | 57.50 | Water demin. | Aqua dem. |
| B | 6.00 | Z-COTE ® MAX | Zinc Oxide, Diphenyl Capryl |
| | | | Methicone |
| | 6.00 | Uvinul ® N 539 T | Octocrylene |
| | 2.00 | Parsol ® 1789 | Butyl |
| | | | Methoxydibenzoylmethane |
| | 2.00 | Dow Corning ® 345 Fluid | Cyclopentasiloxane, |
| | | | Cyclohexasiloxane |
| | 3.50 | Cremophor ® A 6 | Ceteareth-6, Stearyl Alcohol |
| | 1.50 | Cremophor ® A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |
| | 1.00 | Keratin-binding domain-reactive dye | |
| | | 14_264 | |
| | 0.20 | Bisabolol rac. | Bisabolol |
| A | 25.00 | Dow Corning ® 345 Fluid | Cyclopentasiloxane, |
| | | | Cyclohexasiloxane |
| | 20.00 | Dow Corning ® 245 Fluid | Cyclopentasiloxane |
| | 8.00 | Uvinul ® MC 80 | Ethylhexyl Methoxycinnamate |
| | 4.00 | Abil ® EM 90 | Cetyl PEG/PPG-10/1 |
| | | | Dimethicone |
| | 7.00 | T-Lite ® SF | Titanium Dioxide, Alumina |
| | | | Hydrate, |
| | | | Dimethicone/Methicone |
| | | | Copolymer |
| B | 17.00 | Ethanol 95% | Alcohol |
| | 10.00 | Z-COTE ® MAX | Zinc Oxide, Diphenyl Capryl |
| | | | Methicone |
| | 4.50 | Water demin. | Aqua dem. |
| | 3.00 | Glycerin 87% | Glycerin |
| | 0.50 | Keratin-binding domain-reactive dye | |
| | | 14_264 | |
| | 1.00 | Talc | Talc |
| A | 25.00 | Dow Corning ® 345 Fluid | Cyclopentasiloxane, |
| | | | Cyclohexasiloxane |
| | 20.00 | Dow Corning ® 245 Fluid | Cyclopentasiloxane |
| | 8.00 | Uvinul ® MC 80 | Ethylhexyl Methoxycinnamate |
| | 4.00 | Abil ® EM 90 | Cetyl PEG/PPG-10/1 |
| | | | Dimethicone |
| | 7.00 | T-Lite ® SF-S | Titanium Dioxide, Silica |
| | | | Hydrate, Alumina Hydrate, |
| | | | Methicone/Dimethicone |
| | | | Copolymer |
| B | 17.00 | Ethanol 95% | Alcohol |
| | 9.99 | Z-COTE ® MAX | Zinc Oxide, Diphenyl Capryl |
| | | | Methicone |
| | 5.00 | Water demin. | Aqua dem. |
| | 3.00 | Glycerin 87% | Glycerin |
| | 0.01 | Keratin-binding domain-reactive dye | |
| | | 14_264 | |
| | 1.00 | Talc | Talc |
| A | 20.00 | Dow Corning ® 345 Fluid | Cyclopentasiloxane, |
| | | | Cyclohexasiloxane |
| | 10.00 | Dow Corning ® 245 Fluid | Cyclopentasiloxane |
| | 7.50 | Uvinul ® MC 80 | Ethylhexyl Methoxycinnamate |
| | 2.00 | Uvinul ® T 150 | Ethylhexyl Triazone |
| | 12.00 | Cosmacol ® EMI | Di-C12-13 Alkyl Malate |
| | 4.00 | Abil ® EM 90 | Cetyl PEG/PPG-10/1 |
| | | | Dimethicone |
| B | 7.00 | T-Lite ® SF | Titanium Dioxide, Alumina |
| | | | Hydrate, |
| | | | Dimethicone/Methicone |
| | | | Copolymer |
| C | 15.00 | Ethanol 95% | Alcohol |
| | 10.00 | Z-COTE ® MAX | Zinc Oxide, Diphenyl Capryl |
| | | | Methicone |
| | 3.00 | Glycerin 87% | Glycerin |
| | 1.50 | Keratin-binding domain-reactive dye | |
| | | 14_264 | |
| | 8.00 | Water demin. | Aqua dem. |
| A | 20.00 | Dow Corning ® 345 Fluid | Cyclopentasiloxane, |
| | | | Cyclohexasiloxane |
| | 10.00 | Dow Corning ® 245 Fluid | Cyclopentasiloxane |
| | 7.50 | Uvinul ® MC 80 | Ethylhexyl Methoxycinnamate |
| | 2.00 | Tinosorb ® S | Bis-Ethylhexyloxyphenol |
| | | | Methoxyphenyl Triazine |
| | 12.00 | Cosmacol ® EMI | Di-C12-13 Alkyl Malate |
| | 4.00 | Abil ® EM 90 | Cetyl PEG/PPG-10/1 |
| | | | Dimethicone |
| B | 7.00 | T-Lite ® SF | Titanium Dioxide, Alumina |
| | | | Hydrate, |
| | | | Dimethicone/Methicone |
| | | | Copolymer |
| C | 15.00 | Ethanol 95% | Alcohol |
| | 10.00 | Z-COTE ® MAX | Zinc Oxide, Diphenyl Capryl |
| | | | Methicone |
| | 3.00 | Glycerin 87% | Glycerin |
| | 0.50 | Keratin-binding domain-reactive dye | |
| | | 14_264 | |
| | 9.00 | Water demin. | Aqua dem. |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 3.00 | T-Lite ® SF | Titanium Dioxide, Alumina |
| | | | Hydrate, |
| | | | Dimethicone/Methicone |
| | | | Copolymer |
| | 4.00 | Isopropyl Palmitate | Isopropyl Palmitate |
| | 1.00 | Abil ® 350 | Dimethicone |
| B | 7.00 | Uvinul ® MC 80 | Ethylhexyl Methoxycinnamate |
| | 1.00 | Uvinul ® T 150 | Ethylhexyl Triazone |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 61.30 | Water demin. | Aqua dem |
| D | 1.00 | Vitamin E Acetate | Tocopheryl Acetate |
| | 1.00 | Keratin-binding domain-reactive dye | |
| | | 14_264 | |
| | q.s. | Preservative | |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 3.00 | Z-COTE ® MAX | Zinc Oxide, Diphenyl Capryl |
| | | | Methicone |
| | 4.00 | Isopropyl Palmitate | Isopropyl Palmitate |
| | 1.00 | Abil ® 350 | Dimethicone |
| B | 7.00 | Uvinul ® MC 80 | Ethylhexyl Methoxycinnamate |
| | 1.00 | Uvinul ® T 150 | Ethylhexyl Triazone |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 62.20 | Water demin. | Aqua dem |
| D | 1.00 | Vitamin E Acetate | Tocopheryl Acetate |
| | 0.10 | Keratin-binding domain-reactive dye | |
| | | 14_264 | |
| | q.s. | Preservative | |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 3.00 | Z-COTE ® MAX | Zinc Oxide, Diphenyl Capryl |
| | | | Methicone |
| | 4.00 | Isopropyl Palmitate | Isopropyl Palmitate |
| | 1.00 | Abil ® 350 | Dimethicone |
| B | 7.00 | Uvinul ® MC 80 | Ethylhexyl Methoxycinnamate |
| | 1.00 | Tinosorb ® S | Bis-Ethylhexyloxyphenol |
| | | | Methoxyphenyl Triazine |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 60.30 | Water demin. | Aqua dem |
| D | 1.00 | Vitamin E Acetate | Tocopheryl Acetate |
| | 2.00 | Keratin-binding domain-reactive dye | |
| | | 14_264 | |
| | q.s. | Preservative | |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 6.00 | Z-COTE ® MAX | Zinc Oxide, Diphenyl Capryl |
| | | | Methicone |
| | 4.00 | Isopropyl Palmitate | Isopropyl Palmitate |
| | 1.00 | Abil ® 350 | Dimethicone |
| B | 4.00 | Uvinul ® N 539 T | Octocrylene |
| | 1.00 | Tinosorb ® S | Bis-Ethylhexyloxyphenol |
| | | | Methoxyphenyl Triazine |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 62.10 | Water demin. | Aqua dem |
| D | 1.00 | Vitamin E Acetate | Tocopheryl Acetate |
| | 0.20 | Keratin-binding domain-reactive dye | |
| | | 14_264 | |
| | q.s. | Preservative | |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 6.00 | Z-COTE ® MAX | Zinc Oxide, Diphenyl Capryl |
| | | | Methicone |
| | 4.00 | Isopropyl Palmitate | Isopropyl Palmitate |
| | 1.00 | Abil ® 350 | Dimethicone |
| B | 3.00 | Uvinul ® N 539 T | Octocrylene |
| | 1.00 | Mexoryl ® XL | Drometrizole Trisiloxane |
| | 1.00 | Tinosorb ® S | Bis-Ethylhexyloxyphenol |
| | | | Methoxyphenyl Triazine |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 1.00 | Keratin-binding domain-reactive dye | |
| | | 14_264 | |
| | 61.30 | Water demin. | Aqua dem |
| D | 1.00 | Vitamin E Acetate | Tocopheryl Acetate |
| | q.s. | Preservative | |
| A | 2.00 | Cremophor ® WO 7 | PEG-7 Hydrogenated Castor Oil |
| | 6.00 | Abil ® B 8839 | Cyclopentasiloxane, |
| | | | Cyclohexasiloxane |
| | 6.00 | T-Lite ® SF-S | Titanium Dioxide, Silica |
| | | | Hydrate, Alumina Hydrate, |
| | | | Methicone/Dimethicone |
| | | | Copolymer |
| | 4.00 | Isopropyl Palmitate | Isopropyl Palmitate |
| | 1.00 | Abil ® 350 | Dimethicone |
| B | 3.00 | Uvinul ® N 539 T | Octocrylene |
| | 1.00 | Mexoryl ® XL | Drometrizole trisiloxane |
| | 1.00 | Tinosorb ® S | Bis-Ethylhexyloxyphenol |
| | | | Methoxyphenyl Triazine |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, |
| | | | Hexyl Laurate |
| C | 0.50 | Sodium chloride | Sodium Chloride |
| | 0.20 | Edeta BD | Disodium EDTA |
| | 61.80 | Water demin. | Aqua dem |
| D | 1.00 | Vitamin E Acetate | Tocopheryl Acetate |
| | 0.5% | Keratin-binding domain-reactive dye 14_264 | |
| | q.s. | Preservative | |
| A | 4.50 | Uvinul ® MC 80 | Ethylhexyl Methoxycinnamate |
| | 3.00 | Uvinul ® N 539 T | Octocrylene |
| | 1.00 | Uvinul ® T 150 | Ethylhexyl Triazone |
| | 2.50 | Cosmacol ® EMI | Di-C12-13 Alkyl Malate |
| B | 0.50 | Vitamin E Acetate | Tocopheryl Acetate |
| | 4.00 | Tego Care ® 450 | Polyglyceryl-3 Methyl Glucose |
| | | | Distearate |
| C | 3.50 | Cetiol ® SN | Cetearyl Isononanoate |
| | 1.00 | Ganex ® V-220 | VP/Eicosene Copolymer |
| | 5.00 | Isohexadecane | Isohexadecane |
| | 2.50 | Cosmacol ® EMI | Di-C12-13 Alkyl Malate |
| | 5.00 | Z-COTE ® MAX | Zinc Oxide, Diphenyl Capryl |
| | | | Methicone |
| D | 44.00 | Water demin. | Aqua dem. |
| | 5.00 | Glycerin 87% | Glycerin |
| | 1.00 | Lanette E | Sodium Cetearyl Sulfate |
| | 15.00 | Luviquat ® Care | Polyquaternium-44 |
| | 1.00 | Keratin-binding domain-reactive dye | |
| | | 14_264 | |
| | 0.50 | Keltrol ® | Xanthan Gum |
| E | 1.00 | Phenonip | Phenoxyethanol, |
| | | | Methylparaben, Ethylparaben, |
| | | | Butylparaben, Propylparaben, |
| | | | Isobutylparaben |
| A | 4.50 | Uvinul ® MC 80 | Ethylhexyl Methoxycinnamate |
| | 3.00 | Uvinul ® N 539 T | Octocrylene |
| | 1.00 | Tinosorb ® S | Bis-Ethoxylhexyloxyphenol |
| | | | Methoxyphenyl Triazine |
| | 2.50 | Cosmacol ® EMI | Di-C12-13 Alkyl Malate |
| B | 0.50 | Vitamin E Acetate | Tocopheryl Acetate |
| | 4.00 | Tego Care ® 450 | Polyglyceryl-3 Methyl Glucose |
| | | | Distearate |
| C | 3.50 | Cetiol ® SN | Cetearyl Isononanoate |
| | 1.00 | Ganex ® V-220 | VP/Eicosene Copolymer |
| | 5.00 | Isohexadecane | Isohexadecane |
| | 2.50 | Cosmacol ® EMI | Di-C12-13 Alkyl Malate |
| | 5.00 | Z-COTE ® MAX | Zinc Oxide, Diphenyl Capryl |
| | | | Methicone |
| D | 44.90 | Water demin. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 0.10 | Keratin-binding domain-reactive dye | |
| | | 14_264 | |
| | 0.50 | Keltrol ® | Xanthan Gum |
| E | 1.00 | Phenonip | Phenoxyethanol, |
| | | | Methylparaben, Ethylparaben, |
| | | | Butylparaben, Propylparaben, |
| | | | Isobutylparaben |
| A | 1.50 | Mexoryl ® XL | Drometrizole Trisoloxane |
| | 5.00 | Uvinul ® N 539 T | Octocrylene |
| | 1.00 | Tinosorb ® S | Bis-Ethylhexyloxyphenol |
| | | | Methoxyphenyl Triazine |
| | 2.50 | Cosmacol ® EMI | Di-C12-13 Alkyl Malate |
| B | 0.50 | Vitamin E Acetate | Tocopheryl Acetate |
| | 4.00 | Tego Care ® 450 | Polyglyceryl-3 Methyl Glucose |
| | | | Distearate |
| C | 3.50 | Cetiol ® SN | Cetearyl Isononanoate |
| | 1.00 | Ganex ® V-220 | VP/Eicosene Copolymer |
| | 5.00 | Isohexadecane | Isohexadecane |
| | 2.50 | Cosmacol ® EMI | Di-C12-13 Alkyl Malate |
| | 5.00 | Z-COTE ® MAX | Zinc Oxide, Diphenyl Capryl |
| | | | Methicone |
| D | 44.50 | Water demin. | Aqua dem. |
| | 5.00 | Glycerin 87% | Glycerin |
| | 1.00 | Lanette E | Sodium Cetearyl Sulfate |
| | 15.00 | Luviquat ® Care | Polyquaternium-44 |
| | 1.50 | Keratin-binding domain-reactive dye | |
| | | 14_264 | |
| | 0.50 | Keltrol ® | Xanthan Gum |
| E | 1.00 | Phenonip | Phenoxyethanol, |
| | | | Methylparaben, Ethylparaben, |
| | | | Butylparaben, Propylparaben, |
| | | | Isobutylparaben |
| A | 1.50 | Mexoryl ® XL | Drometrizole Trisoloxane |
| | 5.00 | Uvinul ® N 539 T | Octocrylene |
| | 1.00 | Tinosorb ® S | Bis-Ethylhexyloxyphenol |
| | | | Methoxyphenyl Triazine |
| | 2.50 | Cosmacol ® EMI | Di-C12-13 Alkyl Malate |
| B | 0.50 | Vitamin E Acetate | Tocopheryl Acetate |
| | 4.00 | Tego Care ® 450 | Polyglyceryl-3 Methyl Glucose |
| | | | Distearate |
| C | 3.50 | Cetiol ® SN | Cetearyl Isononanoate |
| | 1.00 | Ganex ® V-220 | VP/Eicosene Copolymer |
| | 5.00 | Isohexadecane | Isohexadecane |
| | 2.50 | Cosmacol ® EMI | Di-C12-13 Alkyl Malate |
| | 5.00 | T-Lite ® SF | Titanium Dioxide, Alumina |
| | | | Hydrate, |
| | | | Dimethicone/Methicone |
| | | | Copolymer |
| D | 45.00 | Water demin. | Aqua dem. |
| | 5.00 | Glycerin 87% | Glycerin |
| | 1.00 | Lanette E | Sodium Cetearyl Sulfate |
| | 15.00 | Luviquat ® Care | Polyquaternium-44 |
| | 1.00 | Keratin-binding domain-reactive dye | |
| | | 14_264 | |
| | 0.50 | Keltrol ® | Xanthan Gum |
| E | 1.00 | Phenonip | Phenoxyethanol, |
| | | | Methylparaben, Ethylparaben, |
| | | | Butylparaben, Propylparaben, |
| | | | Isobutylparaben |
| A | 1.00 | Abil ® Care 85 | Bis-PEG/PPG-16/16 PEG/PPG-16/16 |
| | | | Dimethicone, Caprylic/Capric |
| | | | Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 5.00 | Uvinul ® MC 80 | Ethylhexyl Methoxycinnamate |
| | 10.00 | Witconol APM | PPG-3 Myristyl Ether |
| | 2.00 | Uvinul ® T 150 | Ethylhexyl Triazone |
| | 1.00 | Dow Corning ® 345 Fluid | Cyclopentasiloxane, Cyclohexasiloxane |
| | 5.00 | Uvinul ® N 539 T | Octocrylene |
| B | 5.00 | Z-COTE ® MAX | Zinc Oxide, Diphenyl Capryl Methicone |
| C | 5.00 | 1.2-Propylenglykol | Propylene Glycol |
| | 0.20 | Keltrol ® | Xanthan Gum |
| | 0.10 | Edeta BD | Disodium EDTA |
| | 2.0% | Keratin-binding domain-reactive dye | |
| | | 14_264 | |
| | 1.50 | Simulgel ® 600 | Acrylamide/Sodium |
| | | | Acryloyldimethyltaurate Copolymer, |
| | | | Isohexadecane, Polysorbate 80 |
| | 58.40 | Water demin. | Aqua dem. |
| D | q.s. | Perfume oil | |
| | 0.50 | Glydant | DMDM Hydantoin |
| A | 2.00 | Abil ® Care 85 | Bis-PEG/PPG-16/16 PEG/PPG-16/16 |
| | | | Dimethicone, Caprylic/Capric |
| | | | Triglyceride |
| | 4.00 | Finsolv ® TN | C12-15 Alkyl Benzoate |
| | 1.50 | Miglyol ® 812 | Caprylic/Capric Triglyceride |
| | 0.50 | Vitamin E Acetate | Tocopheryl Acetate |
| | 7.50 | Uvinul ® MC 80 | Ethylhexyl Methoxycinnamate |
| | 4.00 | Cetiol ® B | Dibutyl Adipate |
| | 3.00 | Luvitol ® EHO | Cetearyl Ethylhexanoate |
| | 1.00 | Cremophor ® CO 40 | PEG-40 Hydrogenated Castor Oil |
| | 1.00 | Parafin oil, low viscosity | Mineral Oil |
| | 3.00 | Plantacare ® 2000 | Decyl Glucoside |
| | 0.50 | Phenonip | Phenoxyethanol, Methylparaben, |
| | | | Ethylparaben, Butylparaben, |
| | | | Propylparaben, Isobutylparaben |
| | q.s. | Perfume oil | |
| B | 4.00 | Z-COTE ® MAX | Zinc Oxide, Diphenyl Capryl |
| | | | Methicone |
| C | 2.00 | Simulgel ® NS | Hydroxyethyl Acrylate/Sodium |
| | | | Acryloyldimethyl Taurate Copolymer, |
| | | | Squalane, Polysorbate 60 |
| | 0.20 | Keltrol ® | Xanthan Gum |
| | 0.10 | Edeta BD | Disodium EDTA |
| | 1.00 | Keratin-binding domain-reactive | |
| | | dye 14_264 | |
| | 64.20 | Water demin. | Aqua dem |
| A | 6.00 | Gilugel ® SIL 5 | Cyclomethicone, Aluminium/Magnesium |
| | | | Hydroxide Stearate |
| | 5.00 | Uvinul ® MC 80 | Ethylhexyl Methoxycinnamate |
| | 2.00 | Uvinul ® A Plus | Diethylamino Hydroxybenzoyl Hexyl |
| | | | Benzoate |
| | 1.00 | Uvinul ® T 150 | Ethylhexyl Triazone |
| | 7.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, Hexyl |
| | | | Laurate |
| | 2.00 | Cosmacol ® EMI | Di-C12-13 Alkyl Malate |
| | 3.00 | Isopropyl palmitate | Isopropyl Palmitate |
| | 5.00 | Abil ® B 8839 | Cyclopentasiloxane, Cyclohexasiloxane |
| | 0.50 | Abil ® 350 | Dimethicone |
| B | 0.50 | Sodium Chloride | Sodium Chloride |
| | 0.20 | Edeta BD | Disodium EDTA |
| | 0.20 | Keratin-binding domain-reactive | |
| | | dye 14_264 | |
| | 62.10 | Water demin. | Aqua dem. |
| C | 1.00 | Vitamin E Acetate | Tocopheryl Acetate |
| | 0.50 | Phenonip | Phenoxyethanol, Methylparaben, |
| | | | Ethylparaben, Butylparaben, |
| | | | Propylparaben, Isobutylparaben |
| A | 4.50 | Uvinul ® MC 80 | Ethylhexyl Methoxycinnamate |
| | 2.00 | Tinosorb ® S | Bis-Ethylhexyloxyphenol Methoxyphenyl |
| | | | Triazine |
| | 3.00 | Uvinul ® N 539 T | Octocrylene |
| | 2.50 | Cosmacol ® EMI | Di-C12-13 Alkyl Malate |
| | 0.50 | Vitamin E Acetate | Tocopheryl Acetate |
| | 4.00 | Tego Care ® 450 | Polyglyceryl-3 Methyl Glucose Distearate |
| B | 3.50 | Cetiol ® SN | Cetearyl Isononanoate |
| | 1.00 | Ganex ® V-220 | VP/Eicosene Copolymer |
| | 5.00 | Isohexadecane | Isohexadecane |
| | 2.50 | Cosmacol ® EMI | Di-C12-13 Alkyl Malate |
| | 3.00 | T-Lite ® SF | Titanium Dioxide, Alumina Hydrate, |
| | | | Dimethicone/Methicone Copolymer |
| C | 5.00 | Glycerin 87% | Glycerin |
| | 1.00 | Lanette E | Sodium Cetearyl Sulfate |
| | 0.50 | Keltrol ® | Xanthan Gum |
| | 1.00 | Keratin-binding domain- | |
| | | reactive dye 14_264 | |
| | 59.70 | Water demin. | Aqua dem. |
| D | 1.00 | Phenonip | Phenoxyethanol, Methylparaben, |
| | | | Ethylparaben, Butylparaben, |
| | | | Propylparaben, Isobutylparaben |
| | 0.30 | Bisabolol rac. | Bisabolol |
| A | 2.00 | Abil ® Care 85 | Bis-PEG/PPG-16/16 PEG/PPG-16/16 |
| | | | Dimethicone, Caprylic/Capric |
| | | | Triglyceride |
| | 4.00 | Finsolv ® TN | C12-15 Alkyl Benzoate |
| | 1.50 | Miglyol ® 812 | Caprylic/Capric Triglyceride |
| | 0.50 | Vitamin E Acetate | Tocopheryl Acetate |
| | 7.50 | Uvinul ® MC 80 | Ethylhexyl Methoxycinnamate |
| | 4.00 | Cetiol ® B | Dibutyl Adipate |
| | 1.00 | Luvitol ® EHO | Cetearyl Ethylhexanoate |
| | 1.00 | Cremophor ® CO 40 | PEG-40 Hydrogenated Castor Oil |
| | 1.00 | Paraffin oil, low viscosity | Mineral Oil |
| | 3.00 | Plantacare ® 2000 | Decyl Glucoside |
| | 1.00 | Uvinul ® A Plus | Diethylamino Hydroxybenzoyl Hexyl |
| | | | Benzoate |
| | 0.50 | Phenonip | Phenoxyethanol, Methylparaben, |
| | | | Ethylparaben, Butylparaben, |
| | | | Propylparaben, Isobutylparaben |
| | 2.50 | Uvinul ® T 150 | Ethylhexyl Triazone |
| | q.s. | Perfume oil | |
| B | 4.00 | Z-COTE ® MAX | Zinc Oxide, Diphenyl Capryl |
| | | | Methicone |
| C | 2.00 | Simulgel ® NS | Hydroxyethyl Acrylate/Sodium |
| | | | Acryloyldimethyl Taurate Copolymer, |
| | | | Squalane, Polysorbate 60 |
| | 0.10 | Keltrol ® | Xanthan Gum |
| | 0.10 | Edeta BD | Disodium EDTA |
| | 0.50 | Keratin-binding domain-reactive | |
| | | dye 14_264 | |
| | 59.90 | Water demin. | Aqua dem. |
| A | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol ® 331 | Cocoglycerides |
| | 8.00 | Finsolv ® TN | C12-15 Alkyl Benzoate |
| | 8.00 | Cetiol ® B | Dibutyl Adipate |
| B | 6.00 | T-Lite ® SF | Titanium Dioxide, Alumina Hydrate, |
| | | | Dimethicone/Methicone Copolymer |
| | 5.00 | Z-COTE ® MAX | Zinc Oxide, Diphenyl Capryl Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 2.00 | Keratin-binding domain- | |
| | | reactive dye 14_264 | |
| | 0.30 | Keltrol ® | Xanthan Gum |
| | 45.10 | Water demin. | Aqua dem. |
| D | 1.00 | Phenonip | Phenoxyethanol, Methylparaben, |
| | | | Ethylparaben, Butylparaben, |
| | | | Propylparaben, Isobutylparaben |
| A | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol ® 331 | Cocoglycerides |
| | 8.00 | Finsolv ® TN | C12-15 Alkyl Benzoate |
| | 8.00 | Cetiol ® B | Dibutyl Adipate |
| B | 6.00 | Uvinul ® N | Octocrylene |
| | 5.00 | Z-COTE ® MAX | Zinc Oxide, Diphenyl Capryl Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 0.30 | Carbopol ® Ultrez 10 P | Carbomer |
| | 1.00 | Keratin-binding domain- | |
| | | reactive dye 14_264 | |
| | 46.10 | Water demin. | Aqua dem. |
| D | 1.00 | Phenonip | Phenoxyethanol, Methylparaben, |
| | | | Ethylparaben, Butylparaben, |
| | | | Propylparaben, Isobutylparaben |
| A | 4.00 | Eumulgin ® VL 75 | Lauryl Glucoside, Polyglyceryl-2 |
| | | | Dipolyhydroxystearate, Glycerin |
| | 2.00 | Lanette O | Cetearyl Alcohol |
| | 10.00 | Myritol ® 331 | Cocoglycerides |
| | 8.00 | Finsolv ® TN | C12-15 Alkyl Benzoate |
| | 8.00 | Cetiol ® B | Dibutyl Adipate |
| B | 2.00 | Uvinul ® A Plus | Diethylamino Hydroxybenzoyl Hexyl |
| | | | Benzoate |
| | 5.00 | Z-COTE ® MAX | Zinc Oxide, Diphenyl Capryl Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 5.00 | Keratin-binding domain- | |
| | | reactive dye 14_264 | |
| | 0.30 | Carbopol ® Ultrez 10 P | Carbomer |
| | 48.60 | Water demin. | Aqua dem. |
| D | 1.00 | Phenonip | Phenoxyethanol, Methylparaben, |
| | | | Ethylparaben, Butylparaben, |
| | | | Propylparaben, Isobutylparaben |
| A | 3.50 | Cremophor ® A 6 | Ceteareth-6, Stearyl Alcohol |
| | 1.50 | Cremophor ® A 25 | Ceteareth-25 |
| | 7.50 | Uvinul ® MC 80 | Ethylhexyl Methoxycinnamate |
| | 2.00 | Uvinul ® A Plus | Diethylamino Hydroxybenzoyl Hexyl |
| | | | Benzoate |
| | 2.00 | Dow Corning ® 345 | Cyclopentasiloxane, Cyclohexasiloxane |
| | | | Fluid |
| | 0.50 | Beeswax 3044 RH | Beeswax |
| | 3.00 | Lanette O | Cetearyl Alcohol |
| | 10.00 | Miglyol 812 | Caprylic/Capric Triglyceride |
| B | 5.00 | T-Lite ® SF-S | Titanium Dioxide, Silica Hydrate, Alumina |
| | | | Hydrate, Methicone/Dimethicone |
| | | | Copolymer |
| C | 3.00 | Glycerin 87% | Glycerin |
| | 0.20 | Edeta BD | Disodium EDTA |
| | 0.30 | Keltrol ® T | Xanthan Gum |
| | 1.00 | Plantacare ® 2000 | Decyl Glucoside |
| | 2.00 | Keratin-binding domain- | |
| | | reactive dye 14_264 | |
| | 57.30 | Water demin. | Aqua dem. |
| D | 1.00 | Vitamin E Acetate | Tocopheryl Acetate |
| | 0.20 | Bisabolol rac. | Bisabolol |
| A | 10.00 | Uvinul ® A Plus B | Ethylhexyl Methoxycinnamate, |
| | | | Diethylamino Hydroxybenzoyl Hexyl |
| | | | Benzoate |
| | 10.00 | Uvinul ® N 539 T | Octocrylene |
| | 4.00 | Eumulgin ® VL 75 | Lauryl Glucoside, Polyglyceryl-2 |
| | | | Dipolyhydroxystearate, Glycerin |
| | 8.00 | Cetiol ® B | Dibutyl Adipate |
| | 8.00 | Finsolv ® TN | C12-15 Alkyl Benzoate |
| | 12.00 | Myritol ® 331 | Cocoglycerides |
| | 1.00 | Lanette E | Sodium Cetearyl Sulfate |
| | 2.00 | Lanette O | Cetearyl Alcohol |
| B | 3.00 | Z-COTE ® MAX | Zinc Oxide, Diphenyl Capryl Methicone |
| C | 35.08 | Water demin. | Aqua dem. |
| | 0.38 | Citric acid | Citric Acid |
| | 2.90 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 0.10 | Keratin-binding domain- | |
| | | reactive dye 14_264 | |
| | 1.50 | Veegum Ultra | Magnesium Aluminum Silicate |
| D | 0.50 | Phenonip | Phenoxyethanol, Methylparaben, |
| | | | Ethylparaben, Butylparaben, |
| | | | Propylparaben, Isobutylparaben |
| | 1.00 | Vitamin E Acetate | Tocopheryl Acetate |
| A | 0.70 | Cremophor ® A 25 | Ceteareth-25 |
| | 1.70 | Cremophor ® A 6 | Ceteareth-6, Stearyl Alcohol |
| | 2.00 | Uvinul ® A Plus | Diethylamino Hydroxybenzoyl Hexyl |
| | | | Benzoate |
| | 3.00 | Uvinul ® N 539 T | Octocrylene |
| | 3.00 | Z-COTE ® MAX | Zinc Oxide, Diphenyl Capryl Methicone |
| | 2.00 | Abil ® B 8843 | PEG-14 Dimethicone |
| | 3.60 | Lanette O | Cetearyl Alcohol |
| | 4.00 | Uvinul ® MC 80 | Ethylhexyl Methoxycinnamate |
| | 2.00 | Cetiol ® B | Dibutyl Adipate |
| B | 4.00 | Glycerin 87% | Glycerin |
| | 0.20 | Edeta BD | Disodium EDTA |
| | 71.00 | Water demin. | Panthenol |
| C | 4.00 | Luvigel EM | Caprylic/Capric Triglyceride, Sodium |
| | | | Acrylates Copolymer |
| D | 1.00 | Vitamin E Acetate | Tocopheryl Acetate |
| | 1.00 | Keratin-binding domain- | |
| | | reactive dye 14_264 | |
| | 0.20 | Bisabolol rac. | Bisabolol |
| | q.s. | Preservative | |
| A | 7.50 | Uvinul ® MC 80 | Ethylhexyl Methoxycinnamate |
| | 5.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.50 | Cremophor ® WO 7 | PEG-7 Hydrogenated Castor Oil |
| | 0.50 | Cremophor ® PS 20 | Polysorbate 20 |
| | 2.00 | Dry Flo Pure | Aluminum Starch Octenylsuccinate |
| B | 5.00 | Z-COTE ® MAX | Zinc Oxide, Diphenyl Capryl |
| | | | Methicone |
| | 0.03 | Sicomet ® blue P 77 007 | C.I. 77 007. Ultramarines |
| C | 4.00 | 1,2-Propylene Glycol Care | Propylene Glycol |
| | 2.00 | D-Panthenol 50 P | Panthenol, Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 0.50 | Keratin-binding domain-reactive | |
| | | dye 14_264 | |
| | 65.27 | Water demin. | Aqua dem. |
| | q.s. | Preservative | |
| | 0.50 | Vitamin E Acetate | Tocopheryl Acetate |
| A | 6.00 | Gilugel ® SIL 5 | Cyclomethicone (and) |
| | | | Aluminium/Magnesium Hydroxide |
| | | | Stearate |
| | 5.00 | Uvinul ® N 539 T | Octocrylene |
| | 2.00 | Mexoryl ® XL | Drometrizole Trisiloxane |
| | 1.00 | Uvinul ® T 150 | Ethylhexyl Triazone |
| | 3.00 | T-Lite ® SF-S | Titanium Dioxide, Silica Hydrate, Alumina |
| | | | Hydrate, Methicone/Dimethicone |
| | | | Copolymer |
| | 5.00 | Finsolv ® TN | C12-15 Alkyl Benzoate |
| | 4.00 | Abil ® WE 09 | Polyglyceryl-4 Isostearate, Cetyl |
| | | | PEG/PPG-10/1 Dimethicone, Hexyl |
| | | | Laurate |
| | 2.00 | Cosmacol ® EMI | Di-C12-13 Alkyl Malate |
| | 3.00 | Isopropyl palmitate | Isopropyl Palmitate |
| | 5.00 | Abil ® B 8839 | Cyclopentasiloxane, Cyclohexasiloxane |
| | 0.50 | Abil ® 350 | Dimethicone |
| B | 0.50 | Sodium chloride | Sodium Chloride |
| | 0.20 | Edeta BD | Disodium EDTA |
| | 1.00 | Keratin-binding domain-reactive | |
| | | dye 14_264 | |
| | 61.30 | Water demin. | Aqua dem. |
| C | 1.00 | Vitamin E Acetate | Tocopheryl Acetate |
| | 0.50 | Phenonip | Phenoxyethanol, Methylparaben, |
| | | | Ethylparaben, Butylparaben, |
| | | | Propylparaben, Isobutylparaben |
| A | 1.00 | Abil ® Care 85 | Bis-PEG/PPG-16/16 PEG/PPG-16/16 |
| | | | Dimethicone, Caprylic/Capric Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 5.00 | Uvinul ® N 539 T | Octocrylene |
| | 10.00 | Witconol APM | PPG-3 Myristyl Ether |
| | 2.00 | Uvinul ® T 150 | Ethylhexyl Triazone |
| | 1.00 | Dow Corning ® 345 Fluid | Cyclopentasiloxane, Cyclohexasiloxane |
| | 2.00 | Uvinul ® A Plus | Diethylamino Hydroxybenzoyl Hexyl |
| | | | Benzoate |
| B | 5.00 | Z-COTE ® MAX | Zinc Oxide, Diphenyl Capryl Methicone |
| C | 5.00 | 1,2-Propylene glycol | Propylene Glycol |
| | 0.20 | Keltrol ® | Xanthan Gum |
| | 0.10 | Edeta BD | Disodium EDTA |
| | 1.00 | Keratin-binding domain-reactive | |
| | | dye 14_264 | |
| | 1.50 | Simulgel ® 600 | Acrylamide/Sodium |
| | | | Acryloyldimethyltaurate Copolymer, |
| | | | Isohexadecane, Polysorbate 80 |
| | 59.40 | Water demin. | Aqua dem. |
| D | q.s. | Perfume oil | |
| | 0.50 | Glydant | DMDM Hydantoin |
| A | 7.00 | Uvinul ® A Plus B | Ethylhexyl Methoxycinnamate, |
| | | | Diethylamino Hydroxybenzoyl Hexyl |
| | | | Benzoate |
| | 1.00 | Tinosorb ® S | Bis-Ethylhexyloxyphenol Methoxyphenyl |
| | | | Triazine |
| | 1.00 | Uvinul ® T 150 | Ethylhexyl Triazone |
| | 7.00 | Uvinul ® N 539 T | Octocrylene |
| | 4.00 | Eumulgin ® VL 75 | Lauryl Glucoside, Polyglyceryl-2 |
| | | | Dipolyhydroxystearate, Glycerin |
| | 8.00 | Cetiol ® B | Dibutyl Adipate |
| | 8.00 | Finsolv ® TN | C12-15 Alkyl Benzoate |
| | 12.00 | Myritol ® 331 | Cocoglycerides |
| | 1.00 | Lanette E | Sodium Cetearyl Sulfate |
| | 2.00 | Lanette O | Cetearyl Alcohol |
| B | 5.00 | Z-COTE ® MAX | Zinc Oxide, Diphenyl Capryl Methicone |
| C | 35.35 | Water demin. | Aqua dem. |
| | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 0.1% | Keratin-binding domain-reactive | |
| | | dye 14_264 | |
| | 1.50 | Veegum Ultra | Magnesium Aluminum Silicate |
| D | 0.50 | Phenonip | Phenoxyethanol, Methylparaben, |
| | | | Ethylparaben, Butylparaben, |
| | | | Propylparaben, Isobutylparaben |
| | 1.00 | Vitamin E Acetate | Tocopheryl Acetate |
| A | 7.00 | Uvinul ® A Plus B | Ethylhexyl Methoxycinnamate, |
| | | | Diethylamino Hydroxybenzoyl Hexyl |
| | | | Benzoate |
| | 1.00 | Tinosorb ® S | Bis-Ethylhexyloxyphenol Methoxyphenyl |
| | | | Triazine |
| | 1.00 | Uvinul ® T 150 | Ethylhexyl Triazone |
| | 7.00 | Uvinul ® N 539 T | Octocrylene |
| | 4.00 | Eumulgin ® VL 75 | Lauryl Glucoside, Polyglyceryl-2 |
| | | | Dipolyhydroxystearate, Glycerin |
| | 8.00 | Cetiol ® B | Dibutyl Adipate |
| | 8.00 | Finsolv ® TN | C12-15 Alkyl Benzoate |
| | 12.00 | Myritol ® 331 | Cocoglycerides |
| | 1.00 | Lanette E | Sodium Cetearyl Sulfate |
| | 2.00 | Lanette O | Cetearyl Alcohol |
| B | 5.00 | T-Lite ® SF | Titanium Dioxide, Alumina Hydrate, |
| | | | Dimethicone/Methicone Copolymer |
| C | 30.45 | Water demin. | Aqua dem. |
| | 3.00 | Glycerin 87% | Glycerin |
| | 0.05 | Edeta BD | Disodium EDTA |
| | 0.20 | Allantoin | Allantoin |
| | 0.30 | Keltrol ® | Xanthan Gum |
| | 5.00 | Keratin-binding domain- | |
| | | reactive dye 14_264 | |
| | 1.50 | Veegum Ultra | Magnesium Aluminum Silicate |
| D | 0.50 | Phenonip | Phenoxyethanol, Methylparaben, |
| | | | Ethylparaben, Butylparaben, |
| | | | Propylparaben, Isobutylparaben |
| | 1.00 | Vitamin E Acetate | Tocopheryl Acetate |
| | 5.00 | Z-COTE ® MAX | Zinc Oxide, Diphenyl Capryl |
| | | | Methicone |
| | 2.00 | Uvinul ® A Plus | Diethylamino Hydroxybenzoyl |
| | | | Hexyl Benzoate |
| | 2.00 | Mexoryl ® XL | Drometrizole Trisiloxane |
| | 3.00 | Uvinul ® MC 80 | Ethylhexyl Methoxycinnamate |
| | 0.50 | Abil ® 350 | Dimethicone |
| | 2.75 | Carnico wax LT 20 | Copernica Cerifera (Carnauba) |
| | | | Wax, Paraffin |
| | 3.70 | Candelilla wax LT 281 LJ | Euphorbia Cerifera (Candelilla) |
| | | | Wax |
| | 1.80 | Beeswax 3050 PH | Beeswax |
| | 3.20 | TeCero wax 30445 | Microcrystalline Wax |
| | 3.20 | TeCero wax 1030 K | Microcrystalline Wax |
| | 1.34 | Cutina CP | Cetyl Palmitate |
| | 6.40 | Vaseline | Petrolatum |
| | 7.30 | Softisan 100 | Hydrogenated Coco-Glycerides |
| | 10.00 | Luvitol ® EHO | Cetearyl Ethylhexanoate |
| | 0.17 | Bisabolol nat. | Bisabolol |
| | 1.84 | Vitamin E Acetate | Tocopheryl Acetate |
| | 1.00 | Keratin-binding domain-reactive dye | |
| | | 14_264 | |
| | 0.42 | D,L-Alpha-Tocopherol | Tocopherol |
| | 40.38 | Castor Oil | Ricinus Communis (Castor) Oil |
| A | 1.00 | Abil ® Care 85 | Bis-PEG/PPG-16/16 PEG/PPG-16/16 |
| | | | Dimethicone, Caprylic/Capric Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 2.00 | Mexoryl ® XL | Drometrizole Trisilioxane |
| | 10.00 | Witconol ® APM | PPG-3 Myristyl Ether |
| | 1.00 | Uvinul ® T 150 | Ethylhexyl Triazone |
| | 1.00 | Dow Corning ® 345 Fluid | Cyclopentasiloxane, Cyclohexasiloxane |
| | 5.00 | Uvinul ® N 539 T | Octocrylene |
| B | 3.00 | T-Lite ® SF-S | Titanium Dioxide, Silica Hydrate, Alumina |
| | | | Hydrate, Methicone/Dimethicone |
| | | | Copolymer |
| C | 5.00 | 1,2-Propylene glycol | Propylene Glycol |
| | 1.00 | Mexoryl ® SX | Terephthalidene Dicamphor Sulfonic Acid |
| | 0.20 | Keltrol ® | Xanthan Gum |
| | 0.10 | Edeta BD | Disodium EDTA |
| | 1.00 | Keratin-binding domain-reactive | |
| | | dye 14_264 | |
| | 1.50 | Simulgel ® 600 | Acrylamide/Sodium |
| | | | Acryloyldimethyltaurate Copolymer, |
| | | | Isohexadecane, Polysorbate 80 |
| | 69.50 | Water demin. | Aqua dem. |
| D | q.s. | Perfume oil | |
| | 0.50 | Glydant | DMDM Hydantoin |
| A | 1.00 | Abil ® Care 85 | Bis-PEG/PPG-16/16 PEG/PPG-16/16 |
| | | | Dimethicone, Caprylic/Capric Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 2.00 | Parsol ® 1789 | Butyl Methoxydibenzoylmethane |
| | 2.00 | Mexoryl ® XL | Drometrizole Trisilioxane |
| | 10.00 | Witconol ® APM | PPG-3 Myristyl Ether |
| | 1.00 | Uvinul ® T 150 | Ethylhexyl Triazone |
| | 1.00 | Dow Corning ® 345 Fluid | Cyclopentasiloxane, Cyclohexasiloxane |
| | 5.00 | Uvinul ® N 539 T | Octocrylene |
| B | 3.00 | T-Lite ® SF-S | Titanium Dioxide, Silica Hydrate, Alumina |
| | | | Hydrate, Methicone/Dimethicone |
| | | | Copolymer |
| C | 5.00 | 1,2-Propylene glycol | Propylene Glycol |
| | 1.00 | Mexoryl ® SX | Terephthalidene Dicamphor Sulfonic Acid |
| | 0.20 | Keltrol ® | Xanthan Gum |
| | 0.10 | Edeta BD | Disodium EDTA |
| | 0.20 | Keratin-binding domain-reactive | |
| | | dye 14_264 | |
| | 1.50 | Simulgel ® 600 | Acrylamide/Sodium |
| | | | Acryloyldimethyltaurate Copolymer, |
| | | | Isohexadecane, Polysorbate 80 |
| | 68.30 | Water demin. | Aqua dem. |
| D | q.s. | Perfume oil | |
| | 0.50 | Glydant | DMDM Hydantoin |
| A | 0.70 | Cremophor ® A 25 | Ceteareth-25 |
| | 1.70 | Cremophor ® A 6 | Ceteareth-6, Stearyl Alcohol |
| | 2.00 | Parsol ® 1789 | Butyl Methoxydibenzoylmethane |
| | 5.00 | Uvinul ® N 539 T | Octocrylene |
| | 4.00 | Z-COTE ® MAX | Zinc Oxide, Diphenyl Capryl Methicone |
| | 2.00 | Abil ® B 8843 | PEG-14 Dimethicone |
| | 3.60 | Lanette O | Cetearyl Alcohol |
| | 1.00 | Uvinul ® T150 | Ethylhexyl Triazone |
| | 2.00 | Cetiol ® B | Dibutyl Adipate |
| B | 4.00 | Glycerin 87% | Glycerin |
| | 0.20 | Edeta BD | Disodium EDTA |
| | 71.00 | Water demin. | Panthenol |
| C | 4.00 | Luvigel ® EM | Caprylic/Capric Triglyceride, Sodium |
| | | | Acrylates Copolymer |
| | 1.00 | Keratin-binding domain- | |
| | | reactive dye 14_264 | |
| D | 1.00 | Vitamin E Acetate | Tocopheryl Acetate |
| | 0.20 | Bisabolol rac. | Bisabolol |
| | q.s. | Preservative | |
| A | 1.00 | Abil ® Care 85 | Bis-PEG/PPG-16/16 PEG/PPG-16/16 |
| | | | Dimethicone, Caprylic/Capric Triglyceride |
| | 3.00 | Cremophor ® CO 40 | PEG-40 Hydrogenated Castor Oil |
| | 0.30 | Cremophor ® WO 7 | PEG-7 Hydrogenated Castor Oil |
| | 2.00 | Parsol ® 1789 | Butyl Methoxydibenzoylmethane |
| | 2.00 | Mexoryl ® XL | Drometrizole Trisilioxane |
| | 10.00 | Witconol APM | PPG-3 Myristyl Ether |
| | 1.00 | Uvinul ® T 150 | Ethylhexyl Triazone |
| | 1.00 | Dow Corning ® 345 Fluid | Cyclopentasiloxane, Cyclohexasiloxane |
| | 5.00 | Uvinul ® N 539 T | Octocrylene |
| B | 3.00 | Z-COTE ® MAX | Zinc Oxide, Diphenyl Capryl Methicone |
| C | 5.00 | 1,2-Propylene glycol | Propylene Glycol |
| | 1.00 | Mexoryl ® SX | Terephthalidene Dicamphor Sulfonic Acid |
| | 0.20 | Keltrol ® | Xanthan Gum |
| | 0.10 | Edeta BD | Disodium EDTA |
| | 1.50 | Simulgel ® 600 | Acrylamide/Sodium |
| | | | Acryloyldimethyltaurate Copolymer, |
| | | | Isohexadecane, Polysorbate 80 |
| | 0.50 | Keratin-binding domain-reactive | |
| | | dye 14_264 | |
| | 68.00 | Water demin. | Aqua dem. |
| D | q.s. | Perfume oil | |
| | 0.50 | Glydant | DMDM Hydantoin |
| A | 2.00 | Uvinul ® A Plus | Diethylamino Hydroxybenzoyl Hexyl |
| | | | Benzoate |
| | 3.00 | Uvinul ® N 539 T | Octocrylene |
| | 3.00 | Emulgade ® PL 68/50 | Cetearyl Glucoside, Cetearyl Alcohol |
| | 2.00 | Dracorin ® 100 SE | Glyceryl Stearate, PEG-100 Stearate |
| | 1.00 | Fitoderm | Squalane |
| | 0.5 | Cremophor ® WO 7 | PEG-7 Hydrogenated Castor Oil |
| | 0.5 | Cremophor ® PS 20 | Polysorbate 20 |
| | 2.00 | Dry Flo Pure | Aluminum Starch Octenylsuccinate |
| B | 5.00 | Z-COTE ® MAX | Zinc Oxide, Diphenyl Capryl Methicone |
| C | 4.00 | 1,2-Propylene Glycol Care | Propylene Glycol |
| | 0.20 | Keltrol ® | Xanthan Gum |
| | 0.50 | Simulgel ® 600 | Acrylamide/Sodium |
| | | | Acryloyldimethyltaurate Copolymer, |
| | | | Isohexadecane, Polysorbate 80 |
| | 66.30 | Water demin. | Aqua dem. |
| D | q.s. | Preservative | |
| | 0.50 | Vitamin E-Acetat | Tocopheryl Acetate |
| | 1.00 | Keratin-binding domain- | |
| | | reactive dye 14_264 | |
| | 1.00 | RetiSTAR ™ | Caprylic/Capric Triglyceride, Sodium |
| | | | Ascorbate, Tocopherol, Retinol |

1. A keratin-binding effector molecule comprising
(a) at least one reactive dye (i) and
(b) at least one keratin-binding polypeptide (ii).

2. The keratin-binding effector molecule according to claim 1, where the keratin-binding polypeptide (ii) has binding affinity to human hair keratin, nail keratin or skin keratin.

3. The keratin-binding effector molecule according to claim 1, where the keratin-binding polypeptide (ii) used
(a) comprises at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170, or
(b) corresponds to a polypeptide which is at least 40% identical to at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170 and is able to bind keratin.

4. The keratin-binding effector molecule according to claim 1, where the keratin-binding polypeptide (ii) used is encoded by nucleic acid molecule comprising at least one nucleic acid molecule chosen from the group consisting of:
a) a nucleic acid molecule which encodes a polypeptide comprising the sequence shown in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170;
b) a nucleic acid molecule which comprises at least one polynucleotide of the sequence shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 167 or 169;
c) a nucleic acid molecule which encodes a polypeptide according to the sequence SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170;
d) a nucleic acid molecule with a nucleic acid sequence corresponding to at least one of the sequences according to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 167 or 169 or a nucleic acid molecule derived therefrom by substitution, deletion or insertion which encodes a polypeptide which is at least 40% identical to at least one of the sequences according to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170 and is able to bind to keratin;
e) a nucleic acid molecule which encodes a polypeptide which is recognized by a monoclonal antibody directed toward a polypeptide which is encoded by the nucleic acid molecule as in (a) to (c);
f) a nucleic acid molecule coding for a keratin-binding protein which hybridizes under stringent conditions with a nucleic acid molecule as in (a) to (c);
g) a nucleic acid molecule coding for a keratin-binding protein which can be isolated from a DNA bank using a nucleic acid molecule as in (a) to (c) or part fragments thereof comprising at least 15 nucleotides as probe under stringent hybridization conditions; and
h) a nucleic acid molecule which can be produced by back-translation of one of the amino acid sequences shown in the sequences SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170.

5. The keratin-binding effector molecule according to claim 1, where the reactive dye (i) has at least one reactive anchor chosen from the group consisting of
(a) a group of the formula 1 activatable under alkaline conditions,
formula 1 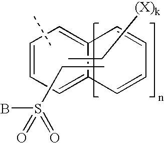
in which [symbol]
means the bond to the dye molecule (D),
X is an electron-withdrawing radical or is an alkyl, —O-alkyl, cyano, hydroxy, halogen radical or H,
k is 1, 2 or 3,
n is 0 or 1, and
B is a group CH═CH₂ or a group CH₂—CH₂-Q or —NH—CH₂—CH₂-Q or —NH—CH═CH₂, in which Q is a group which can be cleaved off under alkaline conditions,
(b) an acrylamido anchor of formula 2, 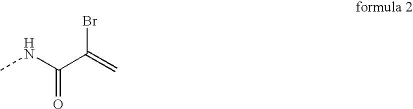
in which [symbol]
means the bond to the dye molecule D, and
(c) one of the compounds according to formulae 3, 4 or 5, 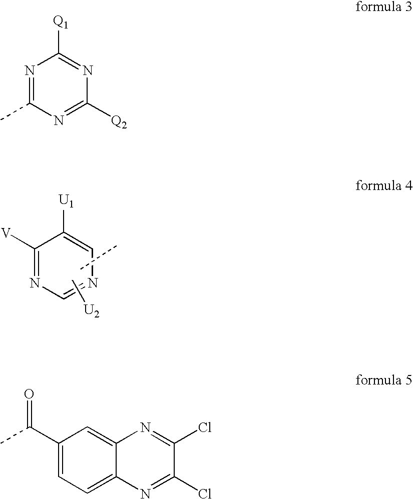
in which [symbol]
means the bond to the dye molecule (D),
V is fluorine or chlorine;
U₁, U₂, independently of one another, are fluoro, chloro or hydrogen; and
Q₁, Q₂, independently of one another, are chloro, fluoro, cyanamide, hydroxy, (C₁-C₆)-alkoxy, phenoxy, sulfophenoxy, mercapto, (C₁-C₆)-alkylmercapto, pyridino, carboxypyridino, carbamoylpyridino, or a group of the general formula (6) or (7), 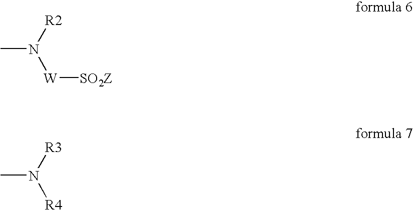
in which R² is hydrogen or (C₁-C₆)-alkyl, sulfo-(C₁-C₆)-alkyl, or phenyl, which is unsubstituted or substituted by (C₁-C₄)-alkyl, (C₁-C₄)-alkoxy, sulfo, halogen, carboxy, acetamido, ureido;
R³ and R⁴, independently of one another, have one of the meanings of R², or are a group of the general formula (8), 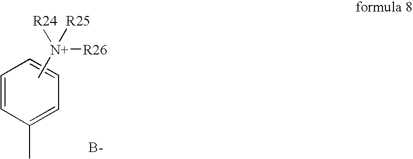
or form a cyclic ring system of the formula —(CH₂)ⱼ—, where j is 4 or 5, or alternatively —(CH₂)₂-E-(CH₂)₂—, where E is oxygen, sulfur, sulfo, —NR⁵— where R^{5′}═(C₁-C₆)-alkyl;
W is phenylene which is unsubstituted or substituted by 1 or 2 substituents, such as (C₁-C₄)-alkyl, (C₁-C₄)-alkoxy, carboxy, sulfo, chloro, bromo, or is (C₁-C₄)-alkylene-arylene or (C₂-C₆)-alkylene, which may be interrupted by oxygen, sulfur, sulfo, amino, carbonyl, carbonamido, or is phenylene-CONH-phenylene which is unsubstituted or substituted by (C₁-C₄)-alkyl, (C₁-C₄)-alkoxy, hydroxy, sulfo, carboxy, amido, ureido or halogen, or is naphthylene which is unsubstituted or substituted by one or two sulfo groups;
Z is a group CH═CH₂ or a group CH₂—CH₂-Q or —NH—CH₂—CH₂-Q or —NH—CH═CH₂, in which Q is a group which can be cleaved off under alkaline conditions;
R²⁴, R²⁵ and R²⁶ are (C₁-C₄)-alkyl or (C₁-C₄)-hydroxyalkyl, and
B— is the equivalent of an anion, such as hydrogensulfate, sulfate, fluoride, chloride, bromide, dihydrogenphosphate, hydrogenphosphate, phosphate, hydroxide or acetate.

6. The keratin-binding effector molecule according to claim 5, where at least one of the radicals X in formula 1 is a group SO₃H.

7. The keratin-binding effector molecule according to claim 5, in which B in formula 1 is —CH═CH₂, a group —CH₂—CH₂—O—SO₃H or is —CH₂—CH₂Cl.

8. The keratin-binding effector molecule according to claim 5, in which the activatable group of formula 1 is bonded to the dye molecule (D) via a group —NH—, —N═N—, —NH—C(O)—, —NH—SO₂— or —N(R)—, where R is alkyl.

9. The keratin-binding effector molecule according to claim 8, where the dye (D) is chosen from dyes of the phthalocyanine series, anthraquinone dyes, azo dyes, formazane dyes, dioxazine dyes, actidine dyes, xanthene dyes, polymethine dyes, stilbene dyes, sulfur dyes, triarylmethane dyes, benzopyran dyes, dibenzanthrone dyes and the metal complexes of these dyes.

10. The keratin-binding effector molecule according to claim 5, in which “n” in formula 1 is zero.

11. The keratin-binding effector molecule according to claim 5, comprising at least one reactive dye, in which the reactive anchor according to formulae 1 to 5 is chosen from the following radicals (1-1) to (1-43):

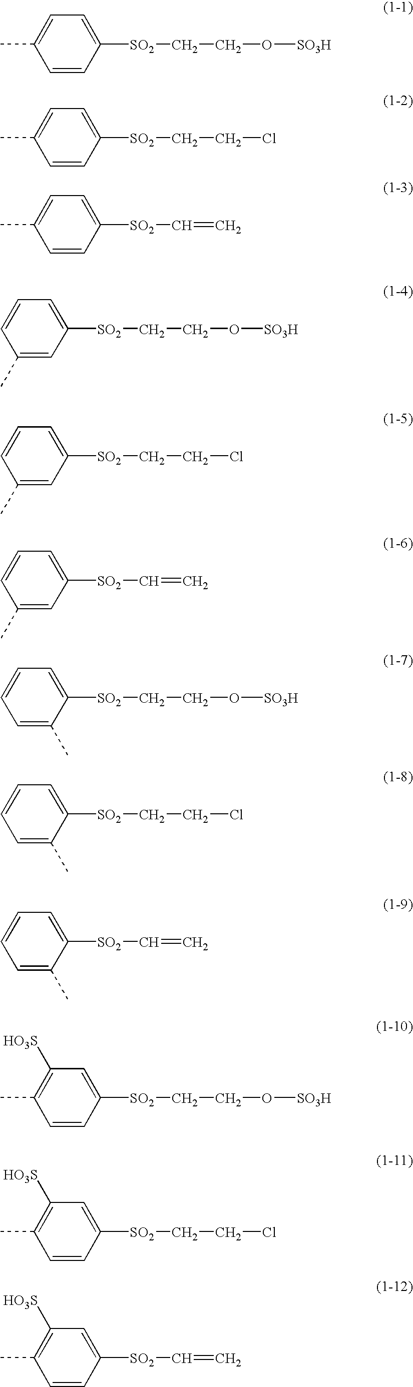

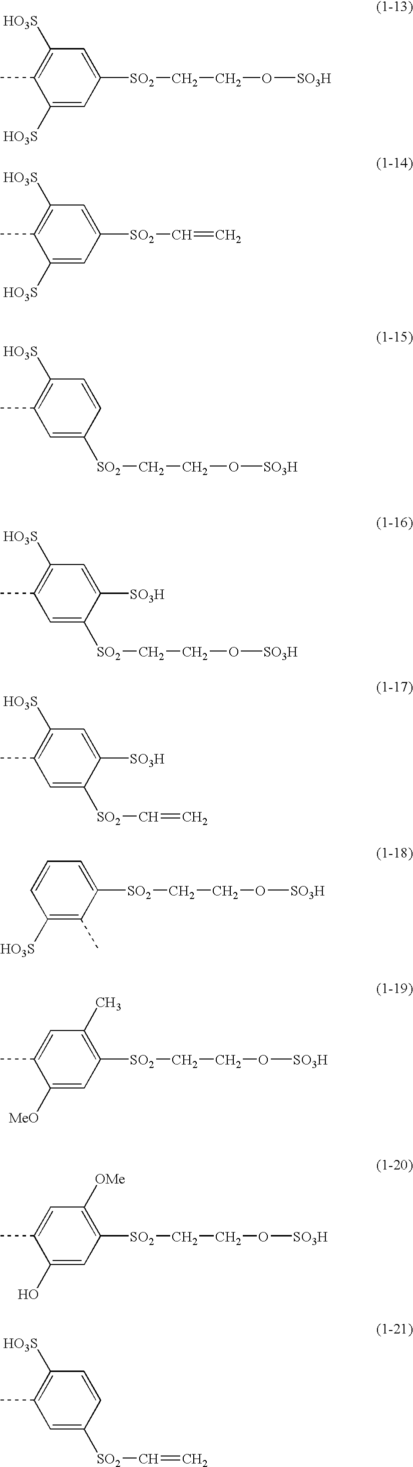

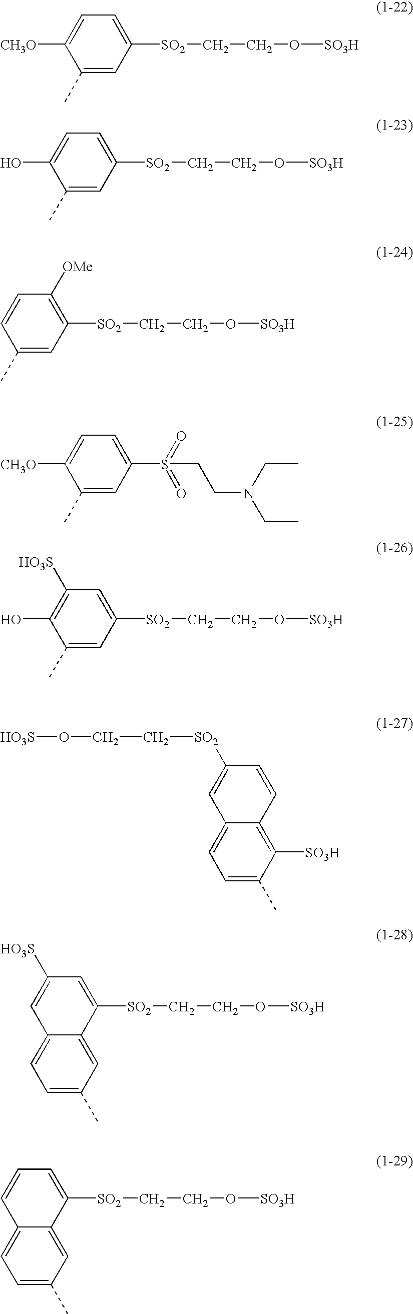

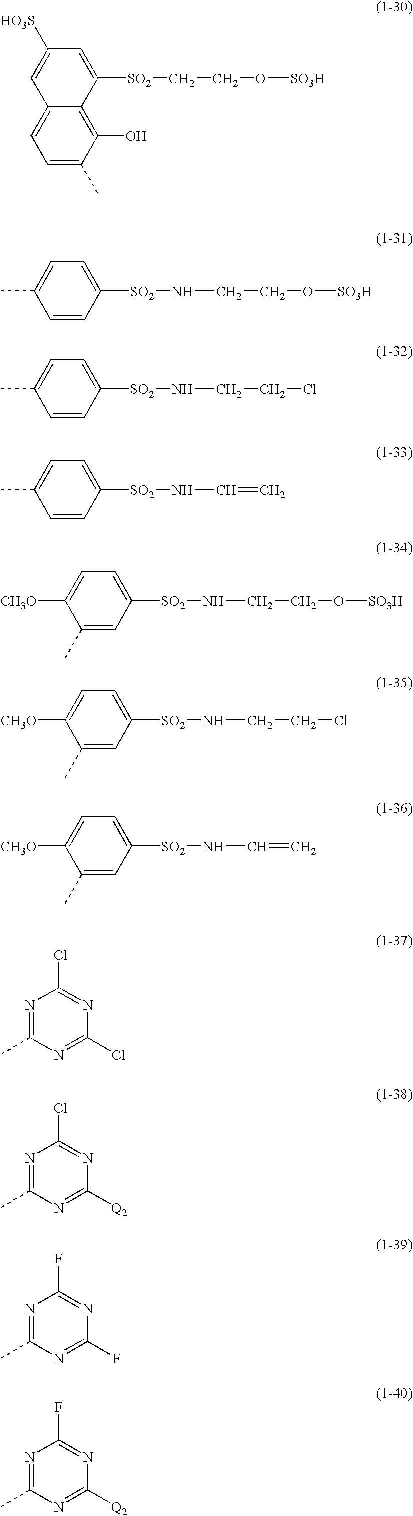

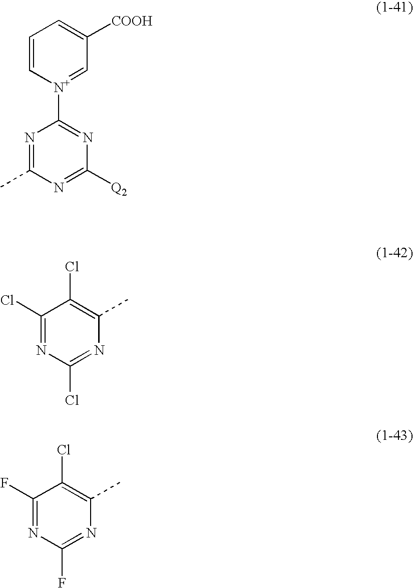

12. The keratin-binding effector molecule according to claim 1, where the reactive dye (i) is coupled to the keratin-binding polypeptide (ii) indirectly via a linker molecule.

13. The keratin-binding effector molecule according to claim 12, where a linker molecule (iii) according to formula 9 or 10 is used, 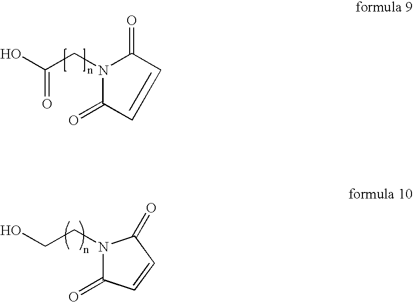
where “n” is an integer between 0 and 40.

14. A hair colorant comprising the keratin-binding effector molecule of claim 1.

15. A hair colorant comprising the keratin-binding effector molecule claim 4.

16. A method of coloring hair or skin comprising using at least one keratin-binding effector molecule comprising
(a) at least one keratin-binding polypeptide (ii) and
(b) a reactive dye (i) or dye.

17. The method according to claim 16, where the at least one keratin-binding polypeptide (ii) is encoded by a nucleic acid molecule comprising at least one nucleic acid molecule chosen from the group consisting of:
a) a nucleic acid molecule which encodes a polypeptide comprising the sequence shown in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170,
b) a nucleic acid molecule which comprises at least one polynucleotide of the sequence shown in SEQ ID NO: 1, 3 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 167 or 169;
c) a nucleic acid molecule which encodes a polypeptide according to the sequence SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170;
d) a nucleic acid molecule with a nucleic acid sequence corresponding to at least one of the sequences according to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 167 or 169 or a nucleic acid molecule derived therefrom by substitution, deletion or insertion which encodes a polypeptide which is at least 40% identical to at least one of the sequences according to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170 and is able to bind to keratin;
e) a nucleic acid molecule which encodes a polypeptide which is recognized by a monoclonal antibody directed toward a polypeptide which is encoded by the nucleic acid molecule as in (a) to (c);
f) a nucleic acid molecule coding for a keratin-binding protein which hybridizes under stringent conditions with a nucleic acid molecule as in (a) to (c);
g) a nucleic acid molecule coding for a keratin-binding protein which can be isolated from a DNA bank using a nucleic acid molecule as in (a) to (c) or part fragments thereof comprising at least 15 nucleotides as probe under stringent hybridization conditions; and
h) a nucleic acid molecule which can be produced by back-translation of one of the amino acid sequences shown in the sequences SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 168 or 170.

18. The method according to claim 16, where the dyeing is reversible, in which the keratin-binding effector molecule is removed from skin or hair in a displacement reaction by treatment with a solution comprising
i. keratin-binding polypeptides, or
ii. skin or hair keratin, or
iii. detergent.

19. A method of coloring hair or skin comprising utilizing at least one keratin-binding effector molecule of claim 5.
